SEQUENCE LISTING (1190 sequences)

SEQ ID NO: 1 - protein, 342 aa, Homo sapiens
FGVKCHNTDSEPLKNEDLLKNLLTMGVDIDMARKRQPGVFHRMITNEQDLKMFLLSKGASKEVIASIISRYPRAITRTPENLSKRWDLWRKIVTSDLEIVNILERSPESFFRSNNNLNLENNIKFLYSVGLTRKCLCRLLTNAPRTFSNSLDLNKQMVEFLQAAGLSLGHNDPADFVRKIIFKNPFILIQSTKRVKANIEFLRSTFNLNSEELLVLICGPGAEILDLSNDYARRSYANIKEKLFSLGCTEEEVQKFVLSYPDVIFLAEKKFNDKIDCLMEENISISQIIENPRVLDSSISTLKSRIKELVNAGCNLSTLNITLLSWSKKRYEAKLKKLSRFA

SEQ ID NO: 2 - DNA, 1026 bp, Homo sapiens
  source 1..1026: mol_type = genomic DNA
tttggtgtgaagtgtcataatacagacagtgagcctttgaaaaatgaggacctactgaaaaacttacttactatgggagtagatattgacatggcaaggaaacgacagcctggagtttttcataggatgattaccaatgagcaggacctgaagatgttccttctttccaaaggagctagcaaagaagtgatcgctagcatcatatcaagatatccacgagcaataacacgtactcccgagaatctttcaaaacggtgggatctgtggagaaagattgtgacatcagaccttgaaattgtaaatattttggaacgttctcctgaatccttttttcggtccaataacaacctaaacttagagaataatataaagttcctctactcagttggattgacccgtaaatgcctttgtcgattgttgaccaatgcccctcgtaccttctccaatagtcttgatctgaataaacagatggttgaatttttgcaggcagccggtttgtcattgggtcacaatgatcccgccgacttcgtgcgtaaaattatattcaagaatcccttcatacttatccagtcaaccaagcgggtgaaagctaacattgaattcttacggtcaactttcaatttgaacagtgaggaactgctggttctgatatgtggtccaggagctgaaatcctagacctttccaatgactatgccagaagaagctacgcaaacatcaaagagaagctgttttctcttggatgtactgaagaagaggtacagaagtttgtcttaagctatccagatgtgatcttcttggcagagaaaaagtttaatgataaaatagactgcctcatggaagaaaacattagcatttcacaaataatcgaaaatcctcgggttctggattcaagcataagtactttaaaaagtcgaatcaaagaattggtaaatgctggctgtaacttgagtactttaaacatcactcttctatcttggagtaaaaaaagatatgaagctaaattgaaaaagttaagcagatttgcc

SEQ ID NO: 3 - protein, 122 aa, Homo sapiens
QAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 4 - DNA, 366 bp, Homo sapiens
  source 1..366: mol_type = genomic DNA
caggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcc

SEQ ID NO: 5 - DNA, 1029 bp, synthetic construct
  source 1..1029: mol_type = other DNA
atgttcggcgtgaagtgccacaacaccgacagcgagcccctgaagaacgaggacctgctgaagaacctgctgaccatgggcgtggacatcgacatggcccgcaagcgccagcccggcgtgttccaccgcatgatcaccaacgagcaggacctgaagatgttcctgctgagcaagggcgccagcaaggaggtgatcgccagcatcatcagccgctacccccgcgccatcacccgcacccccgagaacctgagcaagcgctgggacctgtggcgcaagatcgtgaccagcgacctggagatcgtgaacatcctggagcgcagccccgagagcttcttccgcagcaacaacaacctgaacctggagaacaacatcaagttcctgtatagcgtgggcctgacccgcaagtgcctgtgccgcctgctgaccaacgccccccgcaccttcagcaacagcctggacctgaacaagcagatggtggagttcctgcaggccgccggcctgagcctgggccacaacgaccccgccgacttcgtgcgcaagatcatcttcaagaaccccttcatcctgatccagagcaccaagcgcgtgaaggccaacatcgagttcctgcgcagcaccttcaacctgaacagcgaggagctgctggtgctgatctgcggccccggcgccgagatcctggacctgagcaacgactacgcccgccgcagctacgccaacatcaaggagaagctgttcagcctgggctgcaccgaggaggaggtgcagaagttcgtgctgagctaccccgacgtgatcttcctggccgagaagaagttcaacgacaagatcgactgcctgatggaggagaacatcagcatcagccagatcatcgagaacccccgcgtgctggacagcagcatcagcaccctgaagagccgcatcaaggagctggtgaacgccggctgcaacctgagcaccctgaacatcaccctgctgagctggagcaagaagcgctacgaggccaagctgaagaagctgagccgcttcgcc

SEQ ID NO: 6 - DNA, 366 bp, synthetic construct
  source 1..366: mol_type = other DNA
caggccggcgagggcaccctgagcgaggccctgctgcagctgcagttcgacgacgaggacctgggcgccctgctgggcaacagcaccgaccccgccgtgttcaccgacctggccagcgtggacaacagcgagttccagcagctgctgaaccagggcatccccgtggccccccacaccaccgagcccatgctgatggagtaccccgaggccatcacccgcctggtgaccggagcacaaagaccccctgatcctgcgccagcacctcttggagcacctggcctgcccaacggcctgctgagcggcgacgaggacttcagcagcatcgccgacatggacttcagcgccctgctgagccagatcagcagc

SEQ ID NO: 7 - protein, 327 aa, Homo sapiens
EDLLKNLLTMGVDIDMARKRQPGVFHRMITNEQDLKMFLLSKGASKEVIASIISRYPRAITRTPENLSKRWDLWRKIVTSDLEIVNILERSPESFFRSNNNLNLENNIKFLYSVGLTRKCLCRLLTNAPRTFSNSLDLNKQMVEFLQAAGLSLGHNDPADFVRKIIFKNPFILIQSTKRVKANIEFLRSTFNLNSEELLVLICGPGAEILDLSNDYARRSYANIKEKLFSLGCTEEEVQKFVLSYPDVIFLAEKKFNDKIDCLMEENISISQIIENPRVLDSSISTLKSRIKELVNAGCNLSTLNITLLSWSKKRYEAKLKKLSRFA

SEQ ID NO: 8 - DNA, 981 bp, Homo sapiens
  source 1..981: mol_type = genomic DNA
gaggacctgctgaagaacctgctgaccatgggcgtggacatcgacatggcccgcaagcgccagcccggcgtgttccaccgcatgatcaccaacgagcaggacctgaagatgttcctgctgagcaagggcgccagcaaggaggtgatcgccagcatcatcagccgctacccccgcgccatcacccgcacccccgagaacctgagcaagcgctgggacctgtggcgcaagatcgtgaccagcgacctggagatcgtgaacatcctggagcgcagccccgagagcttcttccgcagcaacaacaacctgaacctggagaacaacatcaagttcctgtatagcgtgggcctgacccgcaagtgcctgtgccgcctgctgaccaacgccccccgcaccttcagcaacagcctggacctgaacaagcagatggtggagttcctgcaggccgccggcctgagcctgggccacaacgaccccgccgacttcgtgcgcaagatcatcttcaagaaccccttcatcctgatccagagcaccaagcgcgtgaaggccaacatcgagttcctgcgcagcaccttcaacctgaacagcgaggagctgctggtgctgatctgcggccccggcgccgagatcctggacctgagcaacgactacgcccgccgcagctacgccaacatcaaggagaagctgttcagcctgggctgcaccgaggaggaggtgcagaagttcgtgctgagctaccccgacgtgatcttcctggccgagaagaagttcaacgacaagatcgactgcctgatggaggagaacatcagcatcagccagatcatcgagaacccccgcgtgctggacagcagcatcagcaccctgaagagccgcatcaaggagctggtgaacgccggctgcaacctgagcaccctgaacatcaccctgctgagctggagcaagaagcgctacgaggccaagctgaagaagctgagccgcttcgcc

SEQ ID NO: 9 - protein, 296 aa, Homo sapiens
EQDLKMFLLSKGASKEVIASIISRYPRAITRTPENLSKRWDLWRKIVTSDLEIVNILERSPESFFRSNNNLNLENNIKFLYSVGLTRKCLCRLLTNAPRTFSNSLDLNKQMVEFLQAAGLSLGHNDPADFVRKIIFKNPFILIQSTKRVKANIEFLRSTFNLNSEELLVLICGPGAEILDLSNDYARRSYANIKEKLFSLGCTEEEVQKFVLSYPDVIFLAEKKFNDKIDCLMEENISISQIIENPRVLDSSISTLKSRIKELVNAGCNLSTLNITLLSWSKKRYEAKLKKLSRFA

SEQ ID NO: 10 - DNA, 888 bp, Homo sapiens
  source 1..888: mol_type = genomic DNA
gagcaggacctgaagatgttcctgctgagcaagggcgccagcaaggaggtgatcgccagcatcatcagccgctacccccgcgccatcacccgcacccccgagaacctgagcaagcgctgggacctgtggcgcaagatcgtgaccagcgacctggagatcgtgaacatcctggagcgcagccccgagagcttcttccgcagcaacaacaacctgaacctggagaacaacatcaagttcctgtatagcgtgggcctgacccgcaagtgcctgtgccgcctgctgaccaacgccccccgcaccttcagcaacagcctggacctgaacaagcagatggtggagttcctgcaggccgccggcctgagcctgggccacaacgaccccgccgacttcgtgcgcaagatcatcttcaagaaccccttcatcctgatccagagcaccaagcgcgtgaaggccaacatcgagttcctgcgcagcaccttcaacctgaacagcgaggagctgctggtgctgatctgcggccccggcgccgagatcctggacctgagcaacgactacgcccgccgcagctacgccaacatcaaggagaagctgttcagcctgggctgcaccgaggaggaggtgcagaagttcgtgctgagctaccccgacgtgatcttcctggccgagaagaagttcaacgacaagatcgactgcctgatggaggagaacatcagcatcagccagatcatcgagaacccccgcgtgctggacagcagcatcagcaccctgaagagccgcatcaaggagctggtgaacgccggctgcaacctgagcaccctgaacatcaccctgctgagctggagcaagaagcgctacgaggccaagctgaagaagctgagccgcttcgcc

SEQ ID NO: 11 - protein, 265 aa, Homo sapiens
TPENLSKRWDLWRKIVTSDLEIVNILERSPESFFRSNNNLNLENNIKFLYSVGLTRKCLCRLLTNAPRTFSNSLDLNKQMVEFLQAAGLSLGHNDPADFVRKIIFKNPFILIQSTKRVKANIEFLRSTFNLNSEELLVLICGPGAEILDLSNDYARRSYANIKEKLFSLGCTEEEVQKFVLSYPDVIFLAEKKFNDKIDCLMEENISISQIIENPRVLDSSISTLKSRIKELVNAGCNLSTLNITLLSWSKKRYEAKLKKLSRFA

SEQ ID NO: 12 - DNA, 795 bp, Homo sapiens
  source 1..795: mol_type = genomic DNA
acccccgagaacctgagcaagcgctgggacctgtggcgcaagatcgtgaccagcgacctggagatcgtgaacatcctggagcgcagccccgagagcttcttccgcagcaacaacaacctgaacctggagaacaacatcaagttcctgtatagcgtgggcctgacccgcaagtgcctgtgccgcctgctgaccaacgccccccgcaccttcagcaacagcctggacctgaacaagcagatggtggagttcctgcaggccgccggcctgagcctgggccacaacgaccccgccgacttcgtgcgcaagatcatcttcaagaaccccttcatcctgatccagagcaccaagcgcgtgaaggccaacatcgagttcctgcgcagcaccttcaacctgaacagcgaggagctgctggtgctgatctgcggccccggcgccgagatcctggacctgagcaacgactacgcccgccgcagctacgccaacatcaaggagaagctgttcagcctgggctgcaccgaggaggaggtgcagaagttcgtgctgagctaccccgacgtgatcttcctggccgagaagaagttcaacgacaagatcgactgcctgatggaggagaacatcagcatcagccagatcatcgagaacccccgcgtgctggacagcagcatcagcaccctgaagagccgcatcaaggagctggtgaacgccggctgcaacctgagcaccctgaacatcaccctgctgagctggagcaagaagcgctacgaggccaagctgaagaagctgagccgcttcgcc

SEQ ID NO: 13 - protein, 50 aa, synthetic construct
DALDDFDLDMLGSDALDDFDLDMLGSDALDDFDLDMLGSDALDDFDLDML

SEQ ID NO: 14 - DNA, 150 bp, synthetic construct
  source 1..150: mol_type = other DNA
gacgcattggacgattttgatttggatatgctgggaagtgacgccctcgatgattttgaccttgacatgcttggttcggatgcccttgatgactttgacctcgacatgctcggcagtgacgcccttgatgatttcgacctggacatgctg

SEQ ID NO: 15 - protein, 11 aa, unidentified
  source 1..11: note = Herpes simplex Virus Type 1
DALDDFDLDML

SEQ ID NO: 16 - DNA, 33 bp, unidentified
  source 1..33: mol_type = genomic DNA; note = Herpes simplex Virus Type 1
gacgcattggacgattttgatttggatatgctg

SEQ ID NO: 17 - protein, 41 aa, Homo sapiens
HEKFPSDLDLDMFNGSLECDMESIIRSELMDADGLDFNFDS

SEQ ID NO: 18 - DNA, 123 bp, Homo sapiens
  source 1..123: mol_type = genomic DNA
cacgagaagttccccagcgacctggacctggacatgttcaacggcagcctggagtgcgacatggagagcatcatcagaagcgagctgatggacgccgacggcctggacttcaacttcgacagc

SEQ ID NO: 19 - protein, 80 aa, Homo sapiens
EAQNVSSHVPYPVALHVNIVNVPQPAAAAIQRHYNDEDPEKEKRIKELELLLMSTENELKGQQVLPTQNHTCSYPGWHST

SEQ ID NO: 20 - DNA, 240 bp, Homo sapiens
  source 1..240: mol_type = genomic DNA
gaggcccagaacgtgagcagccacgtgccctaccccgtggccctgcacgtgaacatcgtgaacgtgccccagcccgccgccgccgccatccagagacactacaacgacgaggaccccgagaaggagaagagaatcaaggagctggagctgctgctgatgagcaccgagaacgagctgaagggccagcaggtgctgcccacccagaaccacacctgcagctaccccggctggcacagcacc

SEQ ID NO: 21 - protein, 80 aa, Homo sapiens
PRPELPLPEGWEEARDFDGKVYYIDHTNRTTSWIDPRDRYTKPLTFADCISDELPLGWEEAYDPQVGDYFIDHNTKTTQI

SEQ ID NO: 22 - DNA, 240 bp, Homo sapiens
  source 1..240: mol_type = genomic DNA
cccagacccgagctgcccctgcccgagggctgggaggaggccagagacttcgacggcaaggtgtactacatcgaccacaccaacagaaccaccagctggatcgaccccagagacagatacaccaagcccctgaccttcgccgactgcatcagcgacgagctgcccctgggctgggaggaggcctacgacccccaggtgggagactacttcatcgaccacaacaccaagaccacccagatc

SEQ ID NO: 23 - protein, 44 aa, Homo sapiens
FVTLKDVGMDFTLGDWEQLGLEQGDTFWDTALDNCQDLFLLDPP

SEQ ID NO: 24 - DNA, 132 bp, Homo sapiens
  source 1..132: mol_type = genomic DNA
ttcgtgaccctgaaggacgtgggcatggacttcaccctgggcgactgggagcagctgggcctggagcagggcgacaccttctgggacaccgccctggacaactgccaggacctgttcctgctggaccccccc

SEQ ID NO: 25 - protein, 48 aa, Homo sapiens
EGQSDERALLDQLHTLLSNTDATGLEEIDRALGIPELVNQGQALEPKQ

SEQ ID NO: 26 - DNA, 144 bp, Homo sapiens
  source 1..144: mol_type = genomic DNA
gagggccagagcgacgagagagccctgctggaccagctgcacaccctgctgagcaacaccgacgccaccggcctggaggagatcgacagagccctgggcatccccgagctggtgaaccagggccaggccctggagcccaagcag

SEQ ID NO: 27 - protein, 191 aa, Homo sapiens
DEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 28 - DNA, 573 bp, Homo sapiens
  source 1..573: mol_type = genomic DNA
gatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcc

SEQ ID NO: 29 - protein, 210 aa, Homo sapiens
PKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 30 - DNA, 630 bp, Homo sapiens
  source 1..630: mol_type = genomic DNA
cccaagccagcaccccagccctatccctttacgtcatccctgagcaccatcaactatgatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcc

SEQ ID NO: 31 - protein, 31 aa, Homo sapiens
PGLPNGLLSGDEDFSSIADMDFSALLSQISS

SEQ ID NO: 32 - DNA, 93 bp, Homo sapiens
  source 1..93: mol_type = genomic DNA
ccggggctccccaatggcctcctttcaggagatgaagacttctcctccattgcggacatggacttctcagccctgctgagtcagatcagctcc

SEQ ID NO: 33 - protein, 82 aa, synthetic construct
HEKFPSDLDLDMFNGSLECDMESIIRSELMDADGLDFNFDSHEKFPSDLDLDMFNGSLECDMESIIRSELMDADGLDFNFDS

SEQ ID NO: 34 - DNA, 246 bp, synthetic construct
  source 1..246: mol_type = other DNA
cacgagaagttccccagcgacctggacctggacatgttcaacggcagcctggagtgcgacatggagagcatcatcagaagcgagctgatggacgccgacggcctggacttcaacttcgacagccacgagaagttcccctccgacctggatctcgatatgtttaatggaagtctggaatgcgacatggaatctattatcaggtccgaactgatggatgccgacggcctggatttcaactttgatagc

SEQ ID NO: 35 - protein, 62 aa, synthetic construct
PGLPNGLLSGDEDFSSIADMDFSALLSQISSPGLPNGLLSGDEDFSSIADMDFSALLSQISS

SEQ ID NO: 36 - DNA, 186 bp, synthetic construct
  source 1..186: mol_type = other DNA
ccggggctccccaatggcctcctttcaggagatgaagacttctcctccattgcggacatggacttctcagccctgctgagtcagatcagctcccctggactgcctaacggcctgctgagcggcgatgaagatttctcctccatcgccgacatggacttcagcgctctgctgagccagatttctagc

SEQ ID NO: 37 - protein, 57 aa, Homo sapiens
MQSLSLGQTSISKGLNYLTIMAPGNLWHMRNNFLFGSRCWMTRFSAENIFKSVSFRL

SEQ ID NO: 38 - DNA, 171 bp, Homo sapiens
  source 1..171: mol_type = genomic DNA
atgcagagcctttccttaggacaaacaagcatttcaaaaggtttgaactacctaaccattatggcaccaggaaacctctggcatatgagaaataactttctctttggttcaagatgttggatgactcgattttcagcagaaaacatcttcaaatcagtttcatttaggctt

SEQ ID NO: 39 - protein, 15 aa, Homo sapiens
FGVKCHNTDSEPLKN

SEQ ID NO: 40 - DNA, 45 bp, Homo sapiens
  source 1..45: mol_type = genomic DNA
tttggtgtgaagtgtcataatacagacagtgagcctttgaaaaat

SEQ ID NO: 41 - DNA, 78 bp, synthetic construct
  source 1..78: mol_type = other DNA
taagatggcagagcccggtaattgcgattaagatggcagagcccggtaattgcgattaagatggcagagcccggtaat

SEQ ID NO: 42 - DNA, 22 bp, Homo sapiens
  source 1..22: mol_type = genomic DNA
taagatggcagagcccggtaat

SEQ ID NO: 43 - skipped

SEQ ID NO: 44 - DNA, 134 bp, synthetic construct
  source 1..134: mol_type = other DNA
taagatggcagagcccggtaattgcgattaagatggcagagcccggtaattgcgattaagatggcagagcccggtaattgcgattaagatggcagagcccggtaattgcgattaagatggcagagcccggtaat

SEQ ID NO: 45 - DNA, 190 bp, synthetic construct
  source 1..190: mol_type = other DNA
taagatggcagagcccggtaattgcgattaagatggcagagcccggtaattgcgattaagatggcagagcccggtaattgcgattaagatggcagagcccggtaattgcgattaagatggcagagcccggtaattgcgattaagatggcagagcccggtaattgcgattaagatggcagagcccggtaat

SEQ ID NO: 46 - DNA, 246 bp, synthetic construct
  source 1..246: mol_type = other DNA
taagatggcagagcccggtaattgcgattaagatggcagagcccggtaattgcgattaagatggcagagcccggtaattgcgattaagatggcagagcccggtaattgcgattaagatggcagagcccggtaattgcgattaagatggcagagcccggtaattgcgattaagatggcagagcccggtaattgcgattaagatggcagagcccggtaattgcgattaagatggcagagcccggtaat

SEQ ID NO: 47 - DNA, 302 bp, synthetic construct
  source 1..302: mol_type = other DNA
taagatggcagagcccggtaattgcgattaagatggcagagcccggtaattgcgattaagatggcagagcccggtaattgcgattaagatggcagagcccggtaattgcgattaagatggcagagcccggtaattgcgattaagatggcagagcccggtaattgcgattaagatggcagagcccggtaattgcgattaagatggcagagcccggtaattgcgattaagatggcagagcccggtaattgcgattaagatggcagagcccggtaattgcgattaagatggcagagcccggtaat

SEQ ID NO: 48 - DNA, 110 bp, synthetic construct
  source 1..110: mol_type = other DNA
taagatggcagagcccggtaattaagatggcagagcccggtaattaagatggcagagcccggtaattaagatggcagagcccggtaattaagatggcagagcccggtaat

SEQ ID NO: 49 - DNA, 150 bp, synthetic construct
  source 1..150: mol_type = other DNA
taagatggcagagcccggtaataaggtgcgattaagatggcagagcccggtaataaggtgcgattaagatggcagagcccggtaataaggtgcgattaagatggcagagcccggtaataaggtgcgattaagatggcagagcccggtaat

SEQ ID NO: 50 - DNA, 10 bp, synthetic construct
  source 1..10: mol_type = other DNA
aaggtgcgat

SEQ ID NO: 51 - DNA, 150 bp, synthetic construct
  source 1..150: mol_type = other DNA
attctaccgtctcgggccattaaaggcatatgattctaccgtctcgggccattaaaggcatatgattctaccgtctcgggccattaaaggcatatgattctaccgtctcgggccattaaaggcatatgattctaccgtctcgggccatta

SEQ ID NO: 52 - protein, 152 aa, synthetic construct
HEKFPSDLDLDMFNGSLECDMESIIRSELMDADGLDFNFDSEAQNVSSHVPYPVALHVNIVNVPQPAAAAIQRHYNDEDPEKEKRIKELELLLMSTENELKGQQVLPTQNHTCSYPGWHSTPGLPNGLLSGDEDFSSIADMDFSALLSQISS

SEQ ID NO: 53 - DNA, 456 bp, synthetic construct
  source 1..456: mol_type = other DNA
cacgagaagttccccagcgacctggacctggacatgttcaacggcagcctggagtgcgacatggagagcatcatcagaagcgagctgatggacgccgacggcctggacttcaacttcgacagcgaggcccagaacgtgagcagccacgtgccctaccccgtggccctgcacgtgaacatcgtgaacgtgccccagcccgccgccgccgccatccagagacactacaacgacgaggaccccgagaaggagaagagaatcaaggagctggagctgctgctgatgagcaccgagaacgagctgaagggccagcaggtgctgcccacccagaaccacacctgcagctaccccggctggcacagcaccccggggctccccaatggcctcctttcaggagatgaagacttctcctccattgcggacatggacttctcagccctgctgagtcagatcagctcc

SEQ ID NO: 54 - protein, 121 aa, synthetic construct
HEKFPSDLDLDMFNGSLECDMESIIRSELMDADGLDFNFDSEAQNVSSHVPYPVALHVNIVNVPQPAAAAIQRHYNDEDPEKEKRIKELELLLMSTENELKGQQVLPTQNHTCSYPGWHST

SEQ ID NO: 55 - DNA, 363 bp, synthetic construct
  source 1..363: mol_type = other DNA
cacgagaagttccccagcgacctggacctggacatgttcaacggcagcctggagtgcgacatggagagcatcatcagaagcgagctgatggacgccgacggcctggacttcaacttcgacagcgaggcccagaacgtgagcagccacgtgccctaccccgtggccctgcacgtgaacatcgtgaacgtgccccagcccgccgccgccgccatccagagacactacaacgacgaggaccccgagaaggagaagagaatcaaggagctggagctgctgctgatgagcaccgagaacgagctgaagggccagcaggtgctgcccacccagaaccacacctgcagctaccccggctggcacagcacc

SEQ ID NO: 56 - protein, 111 aa, synthetic construct
EAQNVSSHVPYPVALHVNIVNVPQPAAAAIQRHYNDEDPEKEKRIKELELLLMSTENELKGQQVLPTQNHTCSYPGWHSTPGLPNGLLSGDEDFSSIADMDFSALLSQISS

SEQ ID NO: 57 - DNA, 333 bp, synthetic construct
  source 1..333: mol_type = other DNA
gaggcccagaacgtgagcagccacgtgccctaccccgtggccctgcacgtgaacatcgtgaacgtgccccagcccgccgccgccgccatccagagacactacaacgacgaggaccccgagaaggagaagagaatcaaggagctggagctgctgctgatgagcaccgagaacgagctgaagggccagcaggtgctgcccacccagaaccacacctgcagctaccccggctggcacagcaccccggggctccccaatggcctcctttcaggagatgaagacttctcctccattgcggacatggacttctcagccctgctgagtcagatcagctcc

SEQ ID NO: 58 - protein, 72 aa, synthetic construct
HEKFPSDLDLDMFNGSLECDMESIIRSELMDADGLDFNFDSPGLPNGLLSGDEDFSSIADMDFSALLSQISS

SEQ ID NO: 59 - DNA, 216 bp, synthetic construct
  source 1..216: mol_type = other DNA
cacgagaagttccccagcgacctggacctggacatgttcaacggcagcctggagtgcgacatggagagcatcatcagaagcgagctgatggacgccgacggcctggacttcaacttcgacagcccggggctccccaatggcctcctttcaggagatgaagacttctcctccattgcggacatggacttctcagccctgctgagtcagatcagctcc

SEQ ID NO: 60 - protein, 78 aa, synthetic construct
APPTDVSLGDELHLDGEDVAMAHADALDDFDLDMLGDGDSPGPGFTPHDSAPYGALDMADFEFEQMFTDALGIDEYGG

SEQ ID NO: 61 - DNA, 234 bp, synthetic construct
  source 1..234: mol_type = other DNA
gcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtggg

SEQ ID NO: 62 - protein, 37 aa, synthetic construct
DALDDFDLDMLPADALDDFDLDMLPADALDDFDLDML

SEQ ID NO: 63 - DNA, 111 bp, synthetic construct
  source 1..111: mol_type = other DNA
gacgcccttgacgattttgacttagacatgctcccagccgatgcccttgacgactttgaccttgatatgctgcctgctgacgctcttgacgattttgaccttgacatgctc

SEQ ID NO: 64 - protein, 76 aa, synthetic construct
DALDDFDLDMLGSDALDDFDLDMLGSDALDDFDLDMLGSDALDDFDLDMLGSDALDDFDLDMLGSDALDDFDLDML

SEQ ID NO: 65 - DNA, 228 bp, synthetic construct
  source 1..228: mol_type = other DNA
gacgcgctggacgatttcgatctcgacatgctgggttctgatgccctcgatgactttgacctggatatgttgggaagcgacgcattggatgactttgatctggacatgctcggctccgatgctctggacgatttcgatctcgatatgttagggtcagacgcactggatgatttcgaccttgatatgttgggaagcgatgcccttgatgatttcgatttggatatgttg

SEQ ID NO: 66 - protein, 128 aa, synthetic construct
DALDDFDLDMLGSDALDDFDLDMLGSDALDDFDLDMLGSDALDDFDLDMLGSDALDDFDLDMLGSDALDDFDLDMLGSDALDDFDLDMLGSDALDDFDLDMLGSDALDDFDLDMLGSDALDDFDLDML

SEQ ID NO: 67 - DNA, 384 bp, synthetic construct
  source 1..384: mol_type = other DNA
gacgcgctggacgatttcgatctcgacatgctgggttctgatgccctcgatgactttgacctggatatgttgggaagcgacgcattggatgactttgatctggacatgctcggctccgatgctctggacgatttcgatctcgatatgttagggtcagacgcactggatgatttcgaccttgatatgttgggaagcgatgcccttgatgatttcgacctggacatgctcggcagcgacgccctggacgatttcgatctggacatgctggggtccgatgccttggatgattttgacttggatatgctggggagtgatgccctggacgactttgacctggacatgctgggctccgatgcgctcgatgacttcgatttggatatgttg

SEQ ID NO: 68 - protein, 564 aa, synthetic construct
SRADPKKKRKVSPGIRRLDALISTSLYKKAGYKEASGSGRADALDDFDLDMLGSDALDDFDLDMLGSDALDDFDLDMLGSDALDDFDLDMLINSRSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 69 - DNA, 1692 bp, synthetic construct
  source 1..1692: mol_type = other DNA
agcagggctgaccccaagaagaagaggaaggtgtcgccagggatccgtcgacttgacgcgttaatatcaacaagtttgtacaaaaaagcaggctacaaagaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctaggagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttt

SEQ ID NO: 70 - protein, 122 aa, Homo sapiens
MDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVSSRSIFKDKQSCDIKMEGMARNDLW

SEQ ID NO: 71 - DNA, 366 bp, Homo sapiens
  source 1..366: mol_type = genomic DNA
atggatgctaagtcactaactgcctggtcccggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagtttccagcaggagcatttttaaagataagcaatcctgtgacattaaaatggaaggaatggcaaggaatgatctctgg

SEQ ID NO: 72 - protein, 311 aa, Homo sapiens
PSRLQMFFANNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 73 - DNA, 936 bp, Homo sapiens
  source 1..936: mol_type = genomic DNA
ccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgagaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtcttcatgaatgagaaagaggacatcttatggtgcactgaaatggaaagggtatttggtttcccagtccactatactgacgtctccaacatgagccgcttggcgaggcagagactgctgggccggtcatggagcgtgccagtcatccgccacctcttcgctccgctgaaggagtattttgcgtgtgtgtaa

SEQ ID NO: 74 - protein, 1083 aa, Homo sapiens
SSQSHPDGLSGRDQPVELLNPARVNHMPSTVDVATALPLQVAPSAVPMDLRLDHQFSLPVAEPALREQQLQQELLALKQKQQIQRQILIAEFQRQHEQLSRQHEAQLHEHIKQQQEMLAMKHQQELLEHQRKLERHRQEQELEKQHREQKLQQLKNKEKGKESAVASTEVKMKLQEFVLNKKKALAHRNLNHCISSDPRYWYGKTQHSSLDQSSPPQSGVSTSYNHPVLGMYDAKDDFPLRKTASEPNLKLRSRLKQKVAERRSSPLLRRKDGPVVTALKKRPLDVTDSACSSAPGSGPSSPNNSSGSVSAENGIAPAVPSIPAETSLAHRLVAREGSAAPLPLYTSPSLPNITLGLPATGPSAGTAGQQDAERLTLPALQQRLSLFPGTHLTPYLSTSPLERDGGAAHSPLLQHMVLLEQPPAQAPLVTGLGALPLHAQSLVGADRVSPSIHKLRQHRPLGRTQSAPLPQNAQALQHLVIQQQHQQFLEKHKQQFQQQQLQMNKIIPKPSEPARQPESHPEETEEELREHQALLDEPYLDRLPGQKEAHAQAGVQVKQEPIESDEEEAEPPREVEPGQRQPSEQELLFRQQALLLEQQRIHQLRNYQASMEAAGIPVSFGGHRPLSRAQSSPASATFPVSVQEPPTKPRFTTGLVYDTLMLKHQCTCGSSSSHPEHAGRIQSIWSRLQETGLRGKCECIRGRKATLEELQTVHSEAHTLLYGTNPLNRQKLDSKKLLGSLASVFVRLPCGGVGVDSDTIWNEVHSAGAARLAVGCVVELVFKVATGELKNGFAVVRPPGHHAEESTPMGFCYFNSVAVAAKLLQQRLSVSKILIVDWDVHHGNGTQQAFYSDPSVLYMSLHRYDDGNFFPGSGAPDEVGTGPGVGFNVNMAFTGGLDPPMGDAEYLAAFRTVVMPIASEFAPDVVLVSSGFDAVEGHPTPLGGYNLSARCFGYLTKQLMGLAGGRIVLALEGGHDLTAICDASEACVSALLGNELDPLPEKVLQQRPNANAVRSMEKVMEIHSKYWRCLQRTTSTAGRSLIEAQTCENEEAETVTAMASLSVGVKPAEKRPDEEPMEEEPPL

SEQ ID NO: 75 - DNA, 3249 bp, Homo sapiens
  source 1..3249: mol_type = genomic DNA
agctcccaaagccatccagatggactttctggccgagaccagccagtggagctgctgaatcctgcccgcgtgaaccacatgcccagcacggtggatgtggccacggcgctgcctctgcaagtggccccctcggcagtgcccatggacctgcgcctggaccaccagttctcactgcctgtggcagagccggccctgcgggagcagcagctgcagcaggagctcctggcgctcaagcagaagcagcagatccagaggcagatcctcatcgctgagttccagaggcagcacgagcagctctcccggcagcacgaggcgcagctccacgagcacatcaagcaacaacaggagatgctggccatgaagcaccagcaggagctgctggaacaccagcggaagctggagaggcaccgccaggagcaggagctggagaagcagcaccgggagcagaagctgcagcagctcaagaacaaggagaagggcaaagagagtgccgtggccagcacagaagtgaagatgaagttacaagaatttgtcctcaataaaaagaaggcgctggcccaccggaatctgaaccactgcatttccagcgaccctcgctactggtacgggaaaacgcagcacagttcccttgaccagagttctccaccccagagcggagtgtcgacctcctataaccacccggtcctgggaatgtacgacgccaaagatgacttccctcttaggaaaacagcttctgaaccgaatctgaaattacggtccaggctaaagcagaaagtggccgaaagacggagcagccccctgttacgcaggaaagacgggccagtggtcactgctctaaaaaagcgtccgttggatgtcacagactccgcgtgcagcagcgccccaggctccggacccagctcacccaacaacagctccgggagcgtcagcgcggagaacggtatcgcgcccgccgtccccagcatcccggcggagacgagtttggcgcacagacttgtggcacgagaaggctcggccgctccacttcccctctacacatcgccatccttgcccaacatcacgctgggcctgcctgccaccggcccctctgcgggcacggcgggccagcaggacgccgagagactcacccttcccgccctccagcagaggctctcccttttccccggcacccacctcactccctacctgagcacctcgcccttggagcgggacggaggggcagcgcacagccctcttctgcagcacatggtcttactggagcagccgccggcacaagcacccctcgtcacaggcctgggagcactgcccctccacgcacagtccttggttggtgcagaccgggtgtccccctccatccacaagctgcggcagcaccgcccactggggcggacccagtcggccccgctgccccagaacgcccaggctctgcagcacctggtcatccagcagcagcatcagcagtttctggagaaacacaagcagcagttccagcagcagcaactgcagatgaacaagatcatccccaagccaagcgagccagcccggcagccggagagccacccggaggagacggaggaggagctccgtgagcaccaggctctgctggacgagccctacctggaccggctgccggggcagaaggaggcgcacgcacaggccggcgtgcaggtgaagcaggagcccattgagagcgatgaggaagaggcagagcccccacgggaggtggagccgggccagcgccagcccagtgagcaggagctgctcttcagacagcaagccctcctgctggagcagcagcggatccaccagctgaggaactaccaggcgtccatggaggccgccggcatccccgtgtccttcggcggccacaggcctctgtcccgggcgcagtcctcacccgcgtctgccaccttccccgtgtctgtgcaggagccccccaccaagccgaggttcacgacaggcctcgtgtatgacacgctgatgctgaagcaccagtgcacctgcgggagtagcagcagccaccccgagcacgccgggaggatccagagcatctggtcccgcctgcaggagacgggcctccggggcaaatgcgagtgcatccgcggacgcaaggccaccctggaggagctacagacggtgcactcggaagcccacaccctcctgtatggcacgaaccccctcaaccggcagaaactggacagtaagaaacttctaggctcgctcgcctccgtgttcgtccggctcccttgcggtggtgttggggtggacagtgacaccatatggaacgaggtgcactcggcgggggcagcccgcctggctgtgggctgcgtggtagagctggtcttcaaggtggccacaggggagctgaagaatggctttgctgtggtccgcccccctggacaccatgcggaggagagcacgcccatgggcttttgctacttcaactccgtggccgtggcagccaagcttctgcagcagaggttgagcgtgagcaagatcctcatcgtggactgggacgtgcaccatggaaacgggacccagcaggctttctacagcgaccctagcgtcctgtacatgtccctccaccgctacgacgatgggaacttcttcccaggcagcggggctcctgatgaggtgggcacagggcccggcgtgggtttcaacgtcaacatggctttcaccggcggcctggacccccccatgggagacgctgagtacttggcggccttcagaacggtggtcatgccgatcgccagcgagtttgccccggatgtggtgctggtgtcatcaggcttcgatgccgtggagggccaccccacccctcttgggggctacaacctctccgccagatgcttcgggtacctgacgaagcagctgatgggcctggctggcggccggattgtcctggccctcgagggaggccacgacctgaccgccatttgcgacgcctcggaagcatgtgtttctgccttgctgggaaacgagcttgatcctctcccagaaaaggttttacagcaaagacccaatgcaaacgctgtccgttccatggagaaagtcatggagatccacagcaagtactggcgctgcctgcagcgcacaacctccacagcggggcgttctctgatcgaggctcagacttgcgagaacgaagaagccgagacggtcaccgccatggcctcgctgtccgtgggcgtgaagcccgccgaaaagagaccagatgaggagcccatggaagaggagccgcccctg

SEQ ID NO: 76 - protein, 440 aa, Homo sapiens
SEREVSTAPAGTDMPAAKKQKLSSDENSNPDLSGDENDDAVSIESGTNTERPDTPTNTPNAPGRKSWGKGKWKSKKCKYSFKCVNSLKEDHNQPLFGVQFNWHSKEGDPLVFATVGSNRVTLYECHSQGEIRLLQSYVDADADENFYTCAWTYDSNTSHPLLAVAGSRGIIRIINPITMQCIKHYVGHGNAINELKFHPRDPNLLLSVSKDHALRLWNIQTDTLVAIFGGVEGHRDEVLSADYDLLGEKIMSCGMDHSLKLWRINSKRMMNAIKESYDYNPNKTNRPFISQKIHFPDFSTRDIHRNYVDCVRWLGDLILSKSCENAIVCWKPGKMEDDIDKIKPSESNVTILGRFDYSQCDIWYMRFSMDFWQKMLALGNQVGKLYVWDLEVEDPHKAKCTTLTHHKCGAAIRQTSFSRDSSILIAVCDDASIWRWDRLR

SEQ ID NO: 77 - DNA, 1320 bp, Homo sapiens
  source 1..1320: mol_type = genomic DNA
tccgagagggaagtgtcgactgcgccggcgggaacagacatgcccgcggccaagaagcagaagttgagcagcgacgagaacagcaacccggacctctcgggagacgaaaatgacgatgctgtcagtattgagagtggcacaaacacagaacgcccggacacgcccacaaatacgccaaatgcaccaggaaggaaaagctggggaaagggaaaatggaagtcaaagaaatgcaaatattctttcaaatgtgtgaacagcctcaaggaagatcataaccagccattgtttggagttcagtttaactggcacagtaaagaaggagaccctctggtgtttgcaactgtgggaagcaacagagtaaccttatacgaatgccattcacagggggagatacggttattgcagtcctatgtagatgctgatgcagatgaaaacttttacacttgtgcatggacctatgatagcaacaccagccaccctctattagcagttgctggatctagaggcattataagaataattaatcctataacaatgcagtgtataaagcactatgttggccatggaaatgctatcaatgagctgaaattccacccacgagacccaaaccttctcctgtcagtaagtaaagatcatgctttacggttatggaatatccaaacagacactcttgtggcaatattcggaggtgtggaagggcacagagatgaagttctgagtgctgattatgatcttttgggtgaaaaaataatgtcctgtggtatggatcactctcttaaactgtggagaatcaactcaaagaggatgatgaatgcaattaaggagtcttatgattataacccaaacaaaactaacaggccatttatttcccagaaaatccactttcctgacttttctaccagagacatacataggaattatgttgattgtgtgcgatggttaggcgatttgatactttccaagtcttgtgaaaatgccattgtatgctggaaacctggcaaaatggaggatgatatagataaaattaaacctagtgagtctaatgtgactattcttgggcgatttgattacagccagtgtgacatttggtacatgaggttttctatggatttctggcaaaagatgcttgcattgggcaatcaggttggcaaactgtatgtttgggatttagaagtagaagatcctcataaagccaaatgcacaacactgacccatcataaatgtggcgcggctattcgacaaaccagtttcagtagggatagcagcatcctcatagctgtctgcgatgatgccagcatttggcgctgggatcgacttcga

SEQ ID NO: 78 - protein, 80 aa, Homo sapiens
KVAQYVVREEDKIEEIEREIIKQEENVDPDYWEKLLRHHYEQQQEDLARNLGKGKRVRKQVNYNDAAQEDQDNQSEYSVG

SEQ ID NO: 79 - DNA, 240 bp, Homo sapiens
  source 1..240: mol_type = genomic DNA
aaggtggcccagtacgtggtgagagaggaggacaagatcgaggagatcgagagagagatcatcaagcaggaggagaacgtggaccccgactactgggagaagctgctgagacaccactacgagcagcagcaggaggacctggccagaaacctgggcaagggcaagagagtgagaaagcaggtgaactacaacgacgccgcccaggaggaccaggacaaccagagcgagtacagcgtgggc

SEQ ID NO: 80 - protein, 80 aa, Homo sapiens
EITRLAVWAAVQAVERKLEAQAMRLLTLEGRTGTNEKKIADCEKTAVEFANHLESKWVVLGTLLQEYGLLQRRLENMENL

SEQ ID NO: 81 - DNA, 240 bp, Homo sapiens
  source 1..240: mol_type = genomic DNA
gagatcaccagactggccgtgtgggccgccgtgcaggccgtggagagaaagctggaggcccaggccatgagactgctgaccctggagggcagaaccggcaccaacgagaagaagatcgccgactgcgagaagaccgccgtggagttcgccaaccacctggagagcaagtgggtggtgctgggcaccctgctgcaggagtacggcctgctgcagagaagactggagaacatggagaacctg

SEQ ID NO: 82 - protein, 80 aa, Homo sapiens
PSEANSIQSANATTKTSETNHTSRPRLKNVDRSTAQQLAVTVGNVTVIITDFKEKTRSSSTSSSTVTSSAGSEQQNQSSS

SEQ ID NO: 83 - DNA, 240 bp, Homo sapiens
  source 1..240: mol_type = genomic DNA
cccagcgaggccaacagcatccagagcgccaacgccaccaccaagaccagcgagaccaaccacaccagcagacccagactgaagaacgtggacagaagcaccgcccagcagctggccgtgaccgtgggcaacgtgaccgtgatcatcaccgacttcaaggagaagaccagaagcagcagcaccagcagcagcaccgtgaccagcagcgccggcagcgagcagcagaaccagagcagcagc

SEQ ID NO: 84 - protein, 80 aa, Homo sapiens
ENDHINLKVAGQDGSVVQFKIKRHTPLSKLMKAYCERQGLSMRQIRFRFDGQPINETDTPAQLEMEDEDTIDVFQQQTGG

SEQ ID NO: 85 - DNA, 240 bp, Homo sapiens
  source 1..240: mol_type = genomic DNA
gaaaacgatcatattaacctgaaagtggcaggccaagatgggtctgtagtacaatttaagatcaaaaggcataccccactgtctaaacttatgaaggcttactgtgagagacaaggactttctatgaggcagattcgattccgctttgatgggcagccaatcaacgaaaccgatactccggcccaactggagatggaagacgaggataccatagacgtttttcaacagcagaccggggga

SEQ ID NO: 86 - protein, 80 aa, Homo sapiens
AEAFGDSEEDGEDVFEVEKILDMKTEGGKVLYKVRWKGYTSDDDTWEPEIHLEDCKEVLLEFRKKIAENKAKAVRKDIQR

SEQ ID NO: 87 - DNA, 240 bp, Homo sapiens
  source 1..240: mol_type = genomic DNA
gcagaggcttttggcgactcagaggaggacggtgaggatgtgtttgaggtagagaagatactggatatgaagacagaaggaggcaaggtcctctataaagtcagatggaagggctacacgtcagatgatgatacatgggagccagagattcatcttgaagattgcaaagaagtgctcctggagtttagaaaaaaaatagctgaaaacaaagcgaaagcggtcagaaaggacatacaaaga

SEQ ID NO: 88 - protein, 60 aa, Homo sapiens
FEVEKILDMKTEGGKVLYKVRWKGYTSDDDTWEPEIHLEDCKEVLLEFRKKIAENKAKAV

SEQ ID NO: 89 - DNA, 180 bp, Homo sapiens
  source 1..180: mol_type = genomic DNA
tttgaagtagagaaaattttggatatgaaaacggaaggcggtaaagttctgtataaggtgagatggaagggatatacaagcgacgatgatacttgggaaccggaaatccatctggaagattgtaaggaggtcctccttgagtttcggaagaaaattgcagaaaataaggctaaggcagtg

SEQ ID NO: 90 - protein, 80 aa, Homo sapiens
EESEKPRGFARGLEPERIIGATDSSGELMFLMKWKNSDEADLVPAKEANVKCPQVVISFYEERLTWHSYPSEDDDKKDDK

SEQ ID NO: 91 - DNA, 240 bp, Homo sapiens
  source 1..240: mol_type = genomic DNA
gaggagagcgagaagcccagaggcttcgccagaggcctggagcccgagagaatcatcggcgccaccgacagcagcggcgagctgatgttcctgatgaagtggaagaacagcgacgaggccgacctggtgcccgccaaggaggccaacgtgaagtgcccccaggtggtgatcagcttctacgaggagagactgacctggcacagctaccccagcgaggacgacgacaagaaggacgacaag

SEQ ID NO: 92 - protein, 80 aa, Homo sapiens
DASRLSGRDPSSWTVEDVMQFVREADPQLGPHADLFRKHEIDGKALLLLRSDMMMKYMGLKLGPALKLSYHIDRLKQGKF

SEQ ID NO: 93 - DNA, 240 bp, Homo sapiens
  source 1..240: mol_type = genomic DNA
gacgccagcagactgagcggcagagaccccagcagctggaccgtggaggacgtgatgcagttcgtgagagaggccgacccccagctgggcccccacgccgacctgttcagaaagcacgagatcgacggcaaggccctgctgctgctgagaagcgacatgatgatgaagtacatgggcctgaagctgggccccgccctgaagctgagctaccacatcgacagactgaagcagggcaagttc

SEQ ID NO: 94 - protein, 80 aa, Homo sapiens
TLIRKADLENHNKDGGFWTVIDGKVYDIKDFQTQSLTGNSILAQFAGEDPVVALEAALQFEDTRESMHAFCVGQYLEPDQ

SEQ ID NO: 95 - DNA, 240 bp, Homo sapiens
  source 1..240: mol_type = genomic DNA
accctgatcagaaaggccgacctggagaaccacaacaaggacggcggcttctggaccgtgatcgacggcaaggtgtacgacatcaaggacttccagacccagagcctgaccggcaacagcatcctggcccagttcgccggcgaggaccccgtggtggccctggaagctgctctgcagttcgaggacaccagagagagcatgcacgccttctgcgtgggccagtacctggagcccgaccag

SEQ ID NO: 96 - protein, 80 aa, Homo sapiens
RLDLPPGFMFKVQAQHDYTATDTDELQLKAGDVVLVIPFQNPEEQDEGWLMGVKESDWNQHKELEKCRGVFPENFTERVP

SEQ ID NO: 97 - DNA, 240 bp, Homo sapiens
  source 1..240: mol_type = genomic DNA
agactggacctgccccccggcttcatgttcaaggtgcaggcccagcacgactacaccgccaccgacaccgacgagctgcagctgaaggccggcgacgtggtgctggtgatccccttccagaaccccgaggagcaggacgagggctggctgatgggcgtgaaggagagcgactggaaccagcacaaggagctggagaagtgcagaggcgtgttccccgagaacttcaccgagagagtgccc

SEQ ID NO: 98 - protein, 80 aa, Homo sapiens
KDPNEPQKPVSAYALFFRDTQAAIKGQNPNATFGEVSKIVASMWDGLGEEQKQVYKKKTEAAKKEYLKQLAAYRASLVSK

SEQ ID NO: 99 - DNA, 240 bp, Homo sapiens
  source 1..240: mol_type = genomic DNA
aaggaccccaacgagccccagaagcccgtgagcgcctacgccctgttcttcagagacacccaggccgccatcaagggccagaaccccaacgccaccttcggcgaggtgagcaagatcgtggccagcatgtgggacggcctgggcgaggagcagaagcaggtgtacaagaagaagaccgaggccgccaagaaggagtacctgaagcagctggccgcctacagagccagcctggtgagcaag

SEQ ID NO: 100 - protein, 465 aa, synthetic construct
MFGVKCHNTDSEPLKNEDLLKNLLTMGVDIDMARKRQPGVFHRMITNEQDLKMFLLSKGASKEVIASIISRYPRAITRTPENLSKRWDLWRKIVTSDLEIVNILERSPESFFRSNNNLNLENNIKFLYSVGLTRKCLCRLLTNAPRTFSNSLDLNKQMVEFLQAAGLSLGHNDPADFVRKIIFKNPFILIQSTKRVKANIEFLRSTFNLNSEELLVLICGPGAEILDLSNDYARRSYANIKEKLFSLGCTEEEVQKFVLSYPDVIFLAEKKFNDKIDCLMEENISISQIIENPRVLDSSISTLKSRIKELVNAGCNLSTLNITLLSWSKKRYEAKLKKLSRFAQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 101 - DNA, 1395 bp, synthetic construct
  source 1..1395: mol_type = other DNA
atgtttggtgtgaagtgtcataatacagacagtgagcctttgaaaaatgaggacctactgaaaaacttacttactatgggagtagatattgacatggcaaggaaacgacagcctggagtttttcataggatgattaccaatgagcaggacctgaagatgttccttctttccaaaggagctagcaaagaagtgatcgctagcatcatatcaagatatccacgagcaataacacgtactcccgagaatctttcaaaacggtgggatctgtggagaaagattgtgacatcagaccttgaaattgtaaatattttggaacgttctcctgaatccttttttcggtccaataacaacctaaacttagagaataatataaagttcctctactcagttggattgacccgtaaatgcctttgtcgattgttgaccaatgcccctcgtaccttctccaatagtcttgatctgaataaacagatggttgaatttttgcaggcagccggtttgtcattgggtcacaatgatcccgccgacttcgtgcgtaaaattatattcaagaatcccttcatacttatccagtcaaccaagcgggtgaaagctaacattgaattcttacggtcaactttcaatttgaacagtgaggaactgctggttctgatatgtggtccaggagctgaaatcctagacctttccaatgactatgccagaagaagctacgcaaacatcaaagagaagctgttttctcttggatgtactgaagaagaggtacagaagtttgtcttaagctatccagatgtgatcttcttggcagagaaaaagtttaatgataaaatagactgcctcatggaagaaaacattagcatttcacaaataatcgaaaatcctcgggttctggattcaagcataagtactttaaaaagtcgaatcaaagaattggtaaatgctggctgtaacttgagtactttaaacatcactcttctatcttggagtaaaaaaagatatgaagctaaattgaaaaagttaagcagatttgcccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggcgcacaacgcccacctgatccggcaccagcaccccttggagctcccggtctccccaatggcctcctttcaggagatgaagacttctcctccattgcggacatggacttctcagccctgctgagtcagatcagctcc

SEQ ID NO: 102 - DNA, 1395 bp, synthetic construct
  source 1..1395: mol_type = other DNA
atgttcggcgtgaagtgccacaacaccgacagcgagcccctgaagaacgaggacctgctgaagaacctgctgaccatgggcgtggacatcgacatggcccgcaagcgccagcccggcgtgttccaccgcatgatcaccaacgagcaggacctgaagatgttcctgctgagcaagggcgccagcaaggaggtgatcgccagcatcatcagccgctacccccgcgccatcacccgcacccccgagaacctgagcaagcgctgggacctgtggcgcaagatcgtgaccagcgacctggagatcgtgaacatcctggagcgcagccccgagagcttcttccgcagcaacaacaacctgaacctggagaacaacatcaagttcctgtatagcgtgggcctgacccgcaagtgcctgtgccgcctgctgaccaacgccccccgcaccttcagcaacagcctggacctgaacaagcagatggtggagttcctgcaggccgccggcctgagcctgggccacaacgaccccgccgacttcgtgcgcaagatcatcttcaagaaccccttcatcctgatccagagcaccaagcgcgtgaaggccaacatcgagttcctgcgcagcaccttcaacctgaacagcgaggagctgctggtgctgatctgcggccccggcgccgagatcctggacctgagcaacgactacgcccgccgcagctacgccaacatcaaggagaagctgttcagcctgggctgcaccgaggaggaggtgcagaagttcgtgctgagctaccccgacgtgatcttcctggccgagaagaagttcaacgacaagatcgactgcctgatggaggagaacatcagcatcagccagatcatcgagaacccccgcgtgctggacagcagcatcagcaccctgaagagccgcatcaaggagctggtgaacgccggctgcaacctgagcaccctgaacatcaccctgctgagctggagcaagaagcgctacgaggccaagctgaagaagctgagccgcttcgcccaggccggcgagggcaccctgagcgaggccctgctgcagctgcagttcgacgacgaggacctgggcgccctgctgggcaacagcaccgaccccgccgtgttcaccgacctggccagcgtggacaacagcgagttccagcagctgctgaaccagggcatccccgtggccccccacaccaccgagcccatgctgatggagtaccccgaggccatcacccgcctggtgaccggagcacaaagaccccctgatcctgcgccagcacctcttggagcacctggcctgcccaacggcctgctgagcggcgacgaggacttcagcagcatcgccgacatggacttcagcgccctgctgagccagatcagcagc

SEQ ID NO: 103 - DNA, 25 bp, synthetic construct
  source 1..25: mol_type = other DNA
ctctagagggtatataatgggggcc

SEQ ID NO: 104 - protein, 26 aa, Homo sapiens
EDLLKNLLTMGVDIDMARKRQPGVFH

SEQ ID NO: 105 - DNA, 78 bp, Homo sapiens
  source 1..78: mol_type = genomic DNA
gaggacctactgaaaaacttacttactatgggagtagatattgacatggcaaggaaacgacagcctggagtttttcat

SEQ ID NO: 106 - protein, 31 aa, Homo sapiens
EQDLKMFLLSKGASKEVIASIISRYPRAITR

SEQ ID NO: 107 - DNA, 93 bp, Homo sapiens
  source 1..93: mol_type = genomic DNA
gagcaggacctgaagatgttccttctttccaaaggagctagcaaagaagtgatcgctagcatcatatcaagatatccacgagcaataacacgt

SEQ ID NO: 108 - protein, 399 aa, Homo sapiens
MQSLSLGQTSISKGLNYLTIMAPGNLWHMRNNFLFGSRCWMTRFSAENIFKSVSFRLFGVKCHNTDSEPLKNEDLLKNLLTMGVDIDMARKRQPGVFHRMITNEQDLKMFLLSKGASKEVIASIISRYPRAITRTPENLSKRWDLWRKIVTSDLEIVNILERSPESFFRSNNNLNLENNIKFLYSVGLTRKCLCRLLTNAPRTFSNSLDLNKQMVEFLQAAGLSLGHNDPADFVRKIIFKNPFILIQSTKRVKANIEFLRSTFNLNSEELLVLICGPGAEILDLSNDYARRSYANIKEKLFSLGCTEEEVQKFVLSYPDVIFLAEKKFNDKIDCLMEENISISQIIENPRVLDSSISTLKSRIKELVNAGCNLSTLNITLLSWSKKRYEAKLKKLSRFA

SEQ ID NO: 109 - DNA, 1197 bp, Homo sapiens
  source 1..1197: mol_type = genomic DNA
atgcagagcctttccttaggacaaacaagcatttcaaaaggtttgaactacctaaccattatggcaccaggaaacctctggcatatgagaaataactttctctttggttcaagatgttggatgactcgattttcagcagaaaacatcttcaaatcagtttcatttaggctttttggtgtgaagtgtcataatacagacagtgagcctttgaaaaatgaggacctactgaaaaacttacttactatgggagtagatattgacatggcaaggaaacgacagcctggagtttttcataggatgattaccaatgagcaggacctgaagatgttccttctttccaaaggagctagcaaagaagtgatcgctagcatcatatcaagatatccacgagcaataacacgtactcccgagaatctttcaaaacggtgggatctgtggagaaagattgtgacatcagaccttgaaattgtaaatattttggaacgttctcctgaatccttttttcggtccaataacaacctaaacttagagaataatataaagttcctctactcagttggattgacccgtaaatgcctttgtcgattgttgaccaatgcccctcgtaccttctccaatagtcttgatctgaataaacagatggttgaatttttgcaggcagccggtttgtcattgggtcacaatgatcccgcagattttgtcagaaagataatttttaaaaacccttttatcttaattcagagcaccaagcgggtgaaagctaacattgaattcttacggtcaactttcaatttgaacagtgaggaactgctggttctgatatgtggtccaggagctgaaatcctagacctttccaatgactatgccagaagaagctacgcaaacatcaaagagaagctgttttctcttggatgtactgaagaagaggtacagaagtttgtcttaagctatccagatgtgatcttcttggcagagaaaaagtttaatgataaaatagactgcctcatggaagaaaacattagcatttcacaaataatcgaaaatcctcgggttctggattcaagcataagtactttaaaaagtcgaatcaaagaattggtaaatgctggctgtaacttgagtactttaaacatcactcttctatcttggagtaaaaaaagatatgaagctaaattgaaaaagttaagcagatttgcc

SEQ ID NO: 110 - DNA, 141 bp, synthetic construct
  source 1..141: mol_type = other DNA
taagatggcagagcccggtaattaagatggcagagcccggtaattaagatggcagagcccggtaattaagatggcagagcccggtaattaagatggcagagcccggtaatcatatgctctagagggtatataatgggggcc

SEQ ID NO: 111 - protein, 379 aa, Mus musculus
MASRNIWCVRRNFLFDLRDWMLQYSAEVFLKSISFRPFSAECDSKDKESLEEEREDLLSNLVTMGVDIDMARRRQPGVFNKAVTNEQELKLFLLSKGASDKVIGSIISRYPRAITRTPESLSKRWDLWRKIMASDLEIVNILERSPESFFRSNNNLNLENNIKFLCSVGLTHKCLCRLLTNAPRTFSNSLNLNKQMVEFLQETGMSLGHNDPRDFVRKIISKNPSILIQSTKRVKTNIEFLQSTFNLNKRDLLLLICGPGARILDLSNDCTKKNYTNIRERLLSLGCSEEEVQRFVLSYLNMVFLSEKKFNDKIDCLIEEKISASQIIENPRILDSSINTLKTRIRELSHAGYDLSTSSIALLSWSQRRYEAKLKRLCG

SEQ ID NO: 112 - DNA, 1140 bp, Mus musculus
  source 1..1140: mol_type = genomic DNA
atggcatcaagaaacatctggtgtgtgagaagaaactttctctttgatttaagagactggatgcttcaatattcagcagaagttttcctcaagtcgatttcattcaggccgtttagtgcggaatgcgacagtaaggacaaggagtctttggaggaggagagggaggacctgctgagcaacttagtcaccatgggcgttgatatcgacatggcaaggaggcgacagcctggagtttttaacaaggctgttacgaatgagcaggagctgaagttattccttctgtccaagggagctagtgacaaagtgattggtagcatcatatcaagatatccacgagctataacacgcactcctgaaagtctttcaaaacggtgggatctgtggagaaagattatggcatcagaccttgaaattgtaaatattttggagcgttctcctgaatccttttttcgatctaataacaatctaaacttagagaataatataaagttcctttgctctgttggattgactcataaatgcctctgcagactgttgaccaatgctcccagaaccttctccaacagtctgaatttgaataagcaaatggttgaatttttgcaggagactggtatgtccttaggtcacaatgatcccagagattttgtcaggaagataatttctaaaaacccttccatcttaattcagagcaccaaacgtgtgaaaactaacattgaatttttacaatcaactttcaacttgaacaaacgggacctgctgcttctgatatgtggcccaggagctagaatcttagacctttccaatgactgtaccaaaaaaaattacacaaacataagagagaggctgctttctcttggatgcagcgaggaggaggtacagaggtttgtcctaagctatctgaatatggtcttcttgtcagagaaaaagtttaatgataaaatagactgccttatagaagaaaaaatcagtgcttcacaaataattgaaaatccgaggattttagactccagcataaatactttaaaaactcgcatccgagagctgtcccatgctgggtatgacttgagcacatccagcattgctctgctgtcctggagtcagagaagatatgaagctaaactgaagagattatgtggatag

SEQ ID NO: 113 - protein, 379 aa, Mus musculus
MASRNIWCVRRNFLFDLRGWMLQYSAEVFLKSISFRTFSVECDSKDKESLEEEREDLLSNLVTMGVDIDMARRRQPGVFNKAVTNEQELKIFLLSKGASDKVIGSIISRYPRAITRTPESLSKRWDLWRKIMASDLEIVNILERSPESFFRSNNNLNLENNIKFLCSVGLTHKCLCRLLTNAPRTFSNSLNLNKQMVEFLQETGMSLGHNDPRDFVRKIISKNPSILIQSTKRVKTNIEFLQSTFNLNKQDLLLLICGPGARILDLSNDCTKKNYTNIRERLLSLGCSEEEVQRFVLSYLNMVFLSEKKFNDKIDCLIEEKISASQIIENPRILDSSINTLKTRIRELSHAGYDLSTSSIALLSWSQRRYEAKLKRLCG

SEQ ID NO: 114 - DNA, 1143 bp, Mus musculus
  source 1..1143: mol_type = genomic DNA
atgattatggcatcaagaaacatctggtgtgtgagaagaaactttctctttgatttaagaggctggatgcttcagtattcagcagaagttttcctcaagtcgatttcattcaggacgtttagtgtggaatgcgacagtaaggacaaggagtctttggaggaggagagggaggacctgctgagcaacttagtcaccatgggcgttgatatcgacatggcaaggaggcgacagcctggagtttttaacaaggctgttacgaatgagcaggagctgaagatattccttctgtccaagggagctagtgacaaagtgattggtagcatcatatcaagatatccacgagctataacacgcactcctgaaagtctttcaaaacggtgggatctgtggagaaagattatggcatcagaccttgaaattgtaaatattttggagcgttctcctgaatccttttttcgatctaataacaatctaaacttagagaataatataaagttcctttgctctgttggattgactcataaatgcctctgcagactgttgaccaatgctcccagaaccttctccaacagtctgaatttgaataagcaaatggttgaatttttgcaggagactggtatgtccttaggtcacaatgatcccagagattttgtcaggaagataatttctaaaaacccttccatcttaattcagagcaccaagcgtgtaaaaactaacattgaatttttacaatcaactttcaacttgaacaaacaggacctgctgcttctgatatgtggcccaggagctagaatcttagacctttccaatgactgtaccaaaaaaaattacacaaacataagagagaggctgctttctcttgggtgcagtgaggaggaggtacagaggtttgtcctaagctatctgaatatggtcttcttgtcagaaaaaaagtttaatgataaaatagactgccttatagaagaaaaaatcagtgcttcacaaataattgaaaatccgaggattttagactccagcataaatactttaaaaactcgcatccgagagctgtcccatgctgggtatgacttgagcacatccagcattgctctgctgtcctggagtcagagaagatatgaagctaaactgaagagattatgtgga

SEQ ID NO: 115 - protein, 394 aa, Canis lupus
  source 1..394: note = Canis lupus familiaris
MQSGLSIPKSLGYLIIMAPRSFFCMRSNFLFGSRCWMTQFSTEVFLKSISFRLFSVKCGNADSESSENEELLNNLLTMGVDVDKAKKRQPGVFNRMGTKEQDLKMFLLSKGASKEVVASIISRYPRAITRTPESLSERWDLWRRIMTSDLEIINILERSPEAFFRSSNNRNLENNIKFLYSVGLTHKCLCRLLTNAPRTFSNSLDLNKQMIKFLQEVCLSLNHNKPRDFIGKIIFKNPFILIQSTKRVKTNIEFLQSTFNLNNEKLLVLLCGPGAKILDLSNDYVNRNYTNIKEKLFSLGCTEEEVHKFILSYPDVIFLGEKKFNDKIDYLIEEKFSISQIIENPRILDSSISTLKSRIKELVNAGYNFSTSNITLLSWSQKRYKAKLKKLNIE

SEQ ID NO: 116 - DNA, 2632 bp, Canis lupus
  source 1..2632: mol_type = genomic DNA; sub_species = Canis lupus familiaris
ctggtccatttccgcctcccggtcccgccccagagaggagaggcggaagtaaaaaggctgtctgtcggctagtattcatgcagtccggtttgagcattccaaaaagtttgggatacctgatcattatggcaccaagaagcttcttttgtatgagaagtaactttctctttggttcaagatgttggatgacccaattttcaacagaagtcttccttaaatcaatttcatttaggctttttagtgtgaaatgtggtaatgcagacagtgagtcttcggagaatgaagaattactgaataacttacttacaatgggagtagatgttgataaggcgaaaaaacgacagcctggagtttttaataggatgggaactaaagagcaggacctgaagatgttccttctatccaaaggagctagtaaagaagtggttgctagcattatatcaagatatccacgagccataacacgcacacctgaaagtctttcagaacggtgggatctgtggagaagaattatgacatcagaccttgagattataaatattttggaacgttctcccgaagctttttttcggtccagtaacaaccgaaacttagagaataatataaaattcctctactcagttggattgactcataaatgcctttgtcggttgttgaccaatgccccacgtacattctccaacagtctagatttgaataaacagatgattaaatttttgcaagaagtctgtttgtctttgaatcacaataagcctagagattttattgggaagataatttttaaaaaccctttcatcttaattcagagtaccaaacgggtaaaaactaatattgagtttttacagtcaacttttaatttgaacaatgagaaactgcttgttttgctctgtggtccgggagctaaaattctagacctttccaatgactatgtcaacagaaactacacaaatatcaaagagaagctgttttctcttgggtgtactgaagaagaggtacacaagtttatcttaagttatccagatgtgatcttcttaggagaaaaaaagttcaatgacaaaatagactatctcatagaagaaaaatttagcatatcacaaataattgaaaatcctcggattttggattcaagcataagtactttaaaaagtcgaatcaaggaattggtgaatgctggctataacttcagtacatcaaacatcactcttttatcttggagtcaaaaaagatacaaagctaaattgaaaaagttaaacattgaatgaaacgttgttctggggaattacaaaatgtcagtcttataatgtagtgatacttcatgttgaattgcctttcagaaagagctttggtttggtaaccatatatatatacataatgatcctttggatattttactgtaaaggtttataaaactcatggccagctacctgtctctagtttgaattaatacaacatccagttatatgatacaggctgtgattgtcctatgaatggtagttcctgagtgccagggcggaacaatatatagaaggaaagtacaaaataagcttttttggtttctgttcagatttaaaaaaaatcaacagattcagttggatggttcctttgagggttttgaatggcaaatgatttctcctatcatgcccacatatttaattgtggtttatcttcaaaatagaatgccagtaaaaatctgacaaaaaaggaggggttttcatctcaaattctgcattctccatttaaaccattagtatgttccccttaccctccaacataggctcaattcactgtgggtatatcctgattttttattttttatttttattttttttatcctgatttttaaaaagacaatggcaactatgatgggtagaaaaatggccacctaaagatgtcagtgtcttgattcccagaatctgaatatgttctgttacactggaaaaggactttgcagatgcaattaagttaaggaccctaagacagggagattatcccagattatcttgtgggtccagtgtcatcatatgggtccttaaaatggaagagggatgcaatatagggacttgacactgtttactgggtttgaaggaggaagggatcatgaaccaaggaatgcaaatggactttaagaaaatggaaaaggcaaggaaacactctctcctgtggcccccagaaaggaacacagccctgctggtaccttggatttagcccactgagacctgttgccaaacttctggcctacagaacccgaagataataaatgtatgtagttttaagccactgaatatgtgaatttgttaagatagcaaataaatgaatataacaactaaataatgattagtcgcattatgggtggaggagtcacaatacatgatttttttagtgtatctgtgtttcttcctcttgtaattacctgttttgtccacatacctttctggaatgactagtaagttgttgtttgtaaagacctaaatgtataaaaaactaaatgtaataaaatctaaataaaggaatatttttacttaataatgtagtttaaagaaatggttatagtctgtatatttatattttatggtcattttaatataaataaagaacaataaatttttagatgtactgaccttaaaatgacaaa

SEQ ID NO: 117 - DNA, 73 bp, Homo sapiens
  source 1..73: mol_type = genomic DNA
gttaagatggcagagcccggtaatcgcataaacttaaactttacagtcagaggttcaattcctcttcttaaca

SEQ ID NO: 118 - protein, 10 aa, synthetic construct
GGGGSGGGGS

SEQ ID NO: 119 - DNA, 30 bp, synthetic construct
  source 1..30: mol_type = other DNA
ggcggtggcggcagtggcggaggcggttcc

SEQ ID NO: 120 - protein, 12 aa, synthetic construct
APAPAPAPAPAP

SEQ ID NO: 121 - DNA, 36 bp, synthetic construct
  source 1..36: mol_type = other DNA
gcccctgcaccagctcctgcccctgcaccggctcca

SEQ ID NO: 122 - protein, 9 aa, synthetic construct
PAAKRVKLD

SEQ ID NO: 123 - DNA, 27 bp, synthetic construct
  source 1..27: mol_type = other DNA
cctgccgctaagagagtgaagctggac

SEQ ID NO: 124 - protein, 5 aa, synthetic construct
EAAAK

SEQ ID NO: 125 - DNA, 15 bp, synthetic construct
  source 1..15: mol_type = other DNA
gaagcagcagcaaaa

SEQ ID NO: 126 - protein, 7 aa, synthetic construct
PKKKRKV

SEQ ID NO: 127 - DNA, 21 bp, synthetic construct
  source 1..21: mol_type = other DNA
ccaaaaaagaagagaaaggta

SEQ ID NO: 128 - protein, 487 aa, synthetic construct
MFGVKCHNTDSEPLKNEDLLKNLLTMGVDIDMARKRQPGVFHRMITNEQDLKMFLLSKGASKEVIASIISRYPRAITRTPENLSKRWDLWRKIVTSDLEIVNILERSPESFFRSNNNLNLENNIKFLYSVGLTRKCLCRLLTNAPRTFSNSLDLNKQMVEFLQAAGLSLGHNDPADFVRKIIFKNPFILIQSTKRVKANIEFLRSTFNLNSEELLVLICGPGAEILDLSNDYARRSYANIKEKLFSLGCTEEEVQKFVLSYPDVIFLAEKKFNDKIDCLMEENISISQIIENPRVLDSSISTLKSRIKELVNAGCNLSTLNITLLSWSKKRYEAKLKKLSRFAQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSDYKDHDGDYKDHDIDYKDDDDK

SEQ ID NO: 129 - DNA, 1461 bp, synthetic construct
  source 1..1461: mol_type = other DNA
atgttcggcgtgaagtgccacaacaccgacagcgagcccctgaagaacgaggacctgctgaagaacctgctgaccatgggcgtggacatcgacatggcccgcaagcgccagcccggcgtgttccaccgcatgatcaccaacgagcaggacctgaagatgttcctgctgagcaagggcgccagcaaggaggtgatcgccagcatcatcagccgctacccccgcgccatcacccgcacccccgagaacctgagcaagcgctgggacctgtggcgcaagatcgtgaccagcgacctggagatcgtgaacatcctggagcgcagccccgagagcttcttccgcagcaacaacaacctgaacctggagaacaacatcaagttcctgtatagcgtgggcctgacccgcaagtgcctgtgccgcctgctgaccaacgccccccgcaccttcagcaacagcctggacctgaacaagcagatggtggagttcctgcaggccgccggcctgagcctgggccacaacgaccccgccgacttcgtgcgcaagatcatcttcaagaaccccttcatcctgatccagagcaccaagcgcgtgaaggccaacatcgagttcctgcgcagcaccttcaacctgaacagcgaggagctgctggtgctgatctgcggccccggcgccgagatcctggacctgagcaacgactacgcccgccgcagctacgccaacatcaaggagaagctgttcagcctgggctgcaccgaggaggaggtgcagaagttcgtgctgagctaccccgacgtgatcttcctggccgagaagaagttcaacgacaagatcgactgcctgatggaggagaacatcagcatcagccagatcatcgagaacccccgcgtgctggacagcagcatcagcaccctgaagagccgcatcaaggagctggtgaacgccggctgcaacctgagcaccctgaacatcaccctgctgagctggagcaagaagcgctacgaggccaagctgaagaagctgagccgcttcgcccaggccggcgagggcaccctgagcgaggccctgctgcagctgcagttcgacgacgaggacctgggcgccctgctgggcaacagcaccgaccccgccgtgttcaccgacctggccagcgtggacaacagcgagttccagcagctgctgaaccagggcatccccgtggccccccacaccaccgagcccatgctgatggagtaccccgaggccatcacccgcctggtgaccggagcacaaagaccccctgatcctgcgccagcacctcttggagcacctggcctgcccaacggcctgctgagcggcgacgaggacttcagcagcatcgccgacatggacttcagcgccctgctgagccagatcagcagcgactataaggaccacgacggagactacaaggatcacgacatcgactataaggatgacgatgacaaa

SEQ ID NO: 130 - protein, 421 aa, synthetic construct
MQSLSLGQTSISKGLNYLTIMAPGNLWHMRNNFLFGSRCWMTRFSAENIFKSVSFRLFGVKCHNTDSEPLKNEDLLKNLLTMGVDIDMARKRQPGVFHRMITNEQDLKMFLLSKGASKEVIASIISRYPRAITRTPENLSKRWDLWRKIVTSDLEIVNILERSPESFFRSNNNLNLENNIKFLYSVGLTRKCLCRLLTNAPRTFSNSLDLNKQMVEFLQAAGLSLGHNDPADFVRKIIFKNPFILIQSTKRVKANIEFLRSTFNLNSEELLVLICGPGAEILDLSNDYARRSYANIKEKLFSLGCTEEEVQKFVLSYPDVIFLAEKKFNDKIDCLMEENISISQIIENPRVLDSSISTLKSRIKELVNAGCNLSTLNITLLSWSKKRYEAKLKKLSRFADYKDHDGDYKDHDIDYKDDDDK

SEQ ID NO: 131 - DNA, 1263 bp, synthetic construct
  source 1..1263: mol_type = other DNA
atgcagagcctttccttaggacaaacaagcatttcaaaaggtttgaactacctaaccattatggcaccaggaaacctctggcatatgagaaataactttctctttggttcaagatgttggatgactcgattttcagcagaaaacatcttcaaatcagtttcatttaggctttttggtgtgaagtgtcataatacagacagtgagcctttgaaaaatgaggacctactgaaaaacttacttactatgggagtagatattgacatggcaaggaaacgacagcctggagtttttcataggatgattaccaatgagcaggacctgaagatgttccttctttccaaaggagctagcaaagaagtgatcgctagcatcatatcaagatatccacgagcaataacacgtactcccgagaatctttcaaaacggtgggatctgtggagaaagattgtgacatcagaccttgaaattgtaaatattttggaacgttctcctgaatccttttttcggtccaataacaacctaaacttagagaataatataaagttcctctactcagttggattgacccgtaaatgcctttgtcgattgttgaccaatgcccctcgtaccttctccaatagtcttgatctgaataaacagatggttgaatttttgcaggcagccggtttgtcattgggtcacaatgatcccgcagattttgtcagaaagataatttttaaaaacccttttatcttaattcagagcaccaagcgggtgaaagctaacattgaattcttacggtcaactttcaatttgaacagtgaggaactgctggttctgatatgtggtccaggagctgaaatcctagacctttccaatgactatgccagaagaagctacgcaaacatcaaagagaagctgttttctcttggatgtactgaagaagaggtacagaagtttgtcttaagctatccagatgtgatcttcttggcagagaaaaagtttaatgataaaatagactgcctcatggaagaaaacattagcatttcacaaataatcgaaaatcctcgggttctggattcaagcataagtactttaaaaagtcgaatcaaagaattggtaaatgctggctgtaacttgagtactttaaacatcactcttctatcttggagtaaaaaaagatatgaagctaaattgaaaaagttaagcagatttgccgactataaggaccacgacggagactacaaggatcatgatattgattacaaagacgatgacgataag

SEQ ID NO: 132 - protein, 98 aa, Homo sapiens
PTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPN

SEQ ID NO: 133 - DNA, 294 bp, Homo sapiens
  source 1..294: mol_type = genomic DNA
cccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaat

SEQ ID NO: 134 - protein, 48 aa, Homo sapiens
PKPAPQPYPFTSSLSTINYDEFPTMVFPSGQISQASALAPAPPQVLPQ

SEQ ID NO: 135 - DNA, 144 bp, Homo sapiens
  source 1..144: mol_type = genomic DNA
cccaagccagcaccccagccctatccctttacgtcatccctgagcaccatcaactatgatgagtttcccaccatggtgtttccttctgggcagatcagccaggcctcggccttggccccggcccctccccaagtcctgccccag

SEQ ID NO: 136 - DNA, 38 bp, synthetic construct
  source 1..38: mol_type = other DNA
gtaggcgtgtacggtgggaggcctatataagcagagct

SEQ ID NO: 137 - DNA, 1182 bp, Homo sapiens
  source 1..1182: mol_type = genomic DNA
gctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 138 - DNA, 584 bp, unidentified
  source 1..584: mol_type = genomic DNA; note = Cytomegalovirus
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 139 - DNA, 1214 bp, Homo sapiens
  source 1..1214: mol_type = genomic DNA
ggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacga

SEQ ID NO: 140 - protein, 95 aa, Homo sapiens
MVAILWHEMWHEGLEEASRLYFGERNVKGMFEVLEPLHAMMERGPQTLKETSFNQAYGRDLMEAQEWCRKYMKSGNVKDLLQAWDLYYHVFRRIS

SEQ ID NO: 141 - DNA, 285 bp, Homo sapiens
  source 1..285: mol_type = genomic DNA
atggtagccatcctctggcatgagatgtggcatgaaggtctagaagaggcctctcgcttgtactttggggagaggaacgtcaaaggcatgtttgaggtgctggagcccctgcatgctatgatggaacgcggtccccagaccctgaaggaaacgtcctttaatcaggcatatggtcgagatttaatggaggcacaagaatggtgccgaaagtacatgaaatcagggaacgtcaaggacctcctgcaagcctgggacctctactatcacgtgttcagacggatatca

SEQ ID NO: 142 - protein, 108 aa, Homo sapiens
MGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 143 - DNA, 324 bp, Homo sapiens
  source 1..324: mol_type = genomic DNA
atgggcgtgcaggtggagactatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaa

SEQ ID NO: 144 - protein, 16 aa, synthetic construct
APAPAPAPAPAPAPAP

SEQ ID NO: 145 - DNA, 48 bp, synthetic construct
  source 1..48: mol_type = other DNA
gcaccagcaccagctcctgccccggcaccagcgccagcaccagcacca

SEQ ID NO: 146 - protein, 15 aa, synthetic construct
EAAAKEAAAKEAAAK

SEQ ID NO: 147 - DNA, 45 bp, synthetic construct
  source 1..45: mol_type = other DNA
gaagctgcggcaaaagaagccgctgcgaaggaagcggcagcaaaa

SEQ ID NO: 148 - protein, 10 aa, synthetic construct
EAAAKEAAAK

SEQ ID NO: 149 - DNA, 30 bp, synthetic construct
  source 1..30: mol_type = other DNA
gaagcagcggcaaaagaggcagcggcaaaa

SEQ ID NO: 150 - protein, 20 aa, synthetic construct
GGGGSGGGGSGGGGSGGGGS

SEQ ID NO: 151 - DNA, 60 bp, synthetic construct
  source 1..60: mol_type = other DNA
ggaggaggcggtagcggaggaggtggctccggtggaggcgggagcggcggagggggcagc

SEQ ID NO: 152 - protein, 313 aa, Homo sapiens
AGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEAADAHRLHAPTSRGGASVEETDQSHLATAGSTSSHSLQKYYITGEAEGFPATA

SEQ ID NO: 153 - DNA, 939 bp, Homo sapiens
  source 1..939: mol_type = genomic DNA
gctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagct

SEQ ID NO: 154 - protein, 47 aa, synthetic construct
NLVAQLENEVASLENENETLKKKNLHKKDLIAYLEKEIANLRKKIEE

SEQ ID NO: 155 - DNA, 141 bp, synthetic construct
  source 1..141: mol_type = other DNA
aacctggttgcgcagctcgaaaacgaagttgcgtctctggaaaatgagaacgaaaccctgaagaaaaagaacctgcacaaaaaagacctgatcgcgtacctggagaaagaaatcgcgaatctgcgtaagaaaatcgaagaa

SEQ ID NO: 156 - protein, 50 aa, synthetic construct
ARNAYLRKKIARLKKDNLQLERDEQNLEKIIANLRDEIARLENEVASHEQ

SEQ ID NO: 157 - DNA, 150 bp, synthetic construct
  source 1..150: mol_type = other DNA
gcgcgtaacgcgtatctgcgtaagaaaatcgcacgtctgaaaaaagacaacctgcagctggaacgtgatgaacagaacctggaaaaaatcatcgcgaacctgcgtgacgaaatcgcgcgtctcgaaaacgaagttgcgtctcacgaacag

SEQ ID NO: 158 - protein, 192 aa, unidentified
  source 1..192: note = Hepatitis C virus
APITAYAQQTRGLLGCIITSLTGRDKNQVEGEVQIVSTATQTFLATCINGVCWAVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTDNSSPPA

SEQ ID NO: 159 - DNA, 576 bp, unidentified
  source 1..576: mol_type = genomic DNA; note = Hepatitis C virus
gcgcccatcacggcgtacgcccagcagacgagaggcctcctagggtgtataatcaccagcctgactggccgggacaaaaaccaagtggagggtgaggtccagatcgtgtcaactgctacccaaaccttcctggcaacgtgcatcaatggggtatgctgggcagtctaccacggggccggaacgaggaccatcgcatcacccaagggtcctgtcatccagatgtataccaatgtggaccaagaccttgtgggctggcccgctcctcaaggttcccgctcattgacaccctgtacctgcggctcctcggacctttacctggtcacgaggcacgccgatgtcattcccgtgcgccggcgaggtgatagcaggggtagcctgctttcgccccggcccatttcctacttgaaaggctcctcggggggtccgctgttgtgccccgcgggacacgccgtgggcctattcagggccgcggtgtgcacccgtggagtggctaaagcggtggactttatccctgtggagaacctagagacaaccatgagatccccggtgttcacggacaactcctctccaccagca

SEQ ID NO: 160 - protein, 333 aa, synthetic construct
ILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRRQLCIQKL

SEQ ID NO: 161 - DNA, 999 bp, synthetic construct
  source 1..999: mol_type = other DNA
atcctggactacagcttcacaggtggcgctgggcgcgacattcccccaccgcagattgaggaggcctgtgagctgcctgagtgccaggtggatgcaggcaataaggtctgcaacctgcagtgtaataatcacgcatgtggctgggatggtggcgactgctccctcaacttcaatgacccctggaagaactgcacgcagtctctacagtgctggaagtattttagcgacggccactgtgacagccagtgcaactcggccggctgcctctttgatggcttcgactgccagctcaccgagggacagtgcaaccccctgtatgaccagtactgcaaggaccacttcagtgatggccactgcgaccagggctgtaacagtgccgaatgtgagtgggatggcctagactgtgctgagcatgtacccgagcggctggcagccggcaccctggtgctggtggtgctgcttccacccgaccagctacggaacaactccttccactttctgcgggagctcagccacgtgctgcacaccaacgtggtcttcaagcgtgatgcgcaaggccagcagatgatcttcccgtactatggccacgaggaagagctgcgcaagcacccaatcaagcgctctacagtgggttgggccacctcttcactgcttcctggtaccagtggtgggcgccagcgcagggagctggaccccatggacatccgtggctccattgtctacctggagatcgacaaccggcaatgtgtgcagtcatcctcgcagtgcttccagagtgccaccgatgtggctgccttcctaggtgctcttgcgtcacttggcagcctcaatattccttacaagattgaggccgtgaagagtgagccggtggagcctccgctgccctcgcagctgcacctcatgtacgtggcagcggccgccttcgtgctcctgttctttgtgggctgtggggtgctgctgtcccgcaagcgccggcggcaactttgtatacaaaagttg

SEQ ID NO: 162 - protein, 247 aa, synthetic construct
MVAILWHEMWHEGLEEASRLYFGERNVKGMFEVLEPLHAMMERGPQTLKETSFNQAYGRDLMEAQEWCRKYMKSGNVKDLLQAWDLYYHVFRRISHEKFPSDLDLDMFNGSLECDMESIIRSELMDADGLDFNFDSEAQNVSSHVPYPVALHVNIVNVPQPAAAAIQRHYNDEDPEKEKRIKELELLLMSTENELKGQQVLPTQNHTCSYPGWHSTPGLPNGLLSGDEDFSSIADMDFSALLSQISS

SEQ ID NO: 163 - DNA, 741 bp, synthetic construct
  source 1..741: mol_type = other DNA
atggtagccatcctctggcatgagatgtggcatgaaggtcttgaagaggcctctcgcttgtactttggggagaggaacgtcaaaggcatgtttgaggtgctggagcccctgcatgctatgatggaacgcggtccccagaccctgaaggaaacgtcctttaatcaggcatatggtcgagatttaatggaggcacaagaatggtgccgaaagtacatgaaatcagggaacgtcaaggacctcctgcaagcctgggacctctactatcacgtgttcagacggatatcacacgagaagttccccagcgacctggacctggacatgttcaacggcagcctggagtgcgacatggagagcatcatcagaagcgagctgatggacgccgacggcctggacttcaacttcgacagcgaggcccagaacgtgagcagccacgtgccctaccccgttgcgcttcacgttaacatcgtcaatgtcccacaacccgcggccgccgccatccagagacactacaacgacgaggaccccgagaaggagaagagaatcaaggagctggagctgctgctgatgagcaccgagaacgagctgaagggccagcaggtgctgcccacccagaaccacacctgcagctaccccggctggcacagcaccccggggctccccaatggcctcctttcaggagatgaagacttctcctccattgcggacatggacttctcagccctgctgagtcagatcagctcc

SEQ ID NO: 164 - protein, 305 aa, synthetic construct
MVAILWHEMWHEGLEEASRLYFGERNVKGMFEVLEPLHAMMERGPQTLKETSFNQAYGRDLMEAQEWCRKYMKSGNVKDLLQAWDLYYHVFRRISPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 165 - DNA, 915 bp, synthetic construct
  source 1..915: mol_type = other DNA
atggtagccatcctctggcatgagatgtggcatgaaggtcttgaagaggcctctcgcttgtactttggggagaggaacgtcaaaggcatgtttgaggtgctggagcccctgcatgctatgatggaacgcggtccccagaccctgaaggaaacgtcctttaatcaggcatatggtcgagatttaatggaggcacaagaatggtgccgaaagtacatgaaatcagggaacgtcaaggacctcctgcaagcctgggacctctactatcacgtgttcagacggatatcacccaagccagcaccccagccctatccctttacgtcatccctgagcaccatcaactatgatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggctcaacggccgccagacccagcaccagctccactgggcgcccctgggctccccaatggcctcctttcaggagatgaagacttctcctccattgcggacatggacttctcagccctgctgagtcagatcagctcc

SEQ ID NO: 166 - protein, 167 aa, synthetic construct
MVAILWHEMWHEGLEEASRLYFGERNVKGMFEVLEPLHAMMERGPQTLKETSFNQAYGRDLMEAQEWCRKYMKSGNVKDLLQAWDLYYHVFRRISHEKFPSDLDLDMFNGSLECDMESIIRSELMDADGLDFNFDSPGLPNGLLSGDEDFSSIADMDFSALLSQISS

SEQ ID NO: 167 - DNA, 501 bp, synthetic construct
  source 1..501: mol_type = other DNA
atggtagccatcctctggcatgagatgtggcatgaaggtcttgaagaggcctctcgcttgtactttggggagaggaacgtcaaaggcatgtttgaggtgctggagcccctgcatgctatgatggaacgcggtccccagaccctgaaggaaacgtcctttaatcaggcatatggtcgagatttaatggaggcacaagaatggtgccgaaagtacatgaaatcagggaacgtcaaggacctcctgcaagcctgggacctctactatcacgtgttcagacggatatcacacgagaagttccccagcgacctggacctggacatgttcaacggcagcctggagtgcgacatggagagcatcatcagaagcgagctgatggacgccgacggcctggacttcaacttcgacagcccggggctccccaatggcctcctttcaggagatgaagacttctcctccattgcggacatggacttctcagccctgctgagtcagatcagctcc

SEQ ID NO: 168 - protein, 177 aa, synthetic construct
MVAILWHEMWHEGLEEASRLYFGERNVKGMFEVLEPLHAMMERGPQTLKETSFNQAYGRDLMEAQEWCRKYMKSGNVKDLLQAWDLYYHVFRRISHEKFPSDLDLDMFNGSLECDMESIIRSELMDADGLDFNFDSHEKFPSDLDLDMFNGSLECDMESIIRSELMDADGLDFNFDS

SEQ ID NO: 169 - DNA, 531 bp, synthetic construct
  source 1..531: mol_type = other DNA
atggtagccatcctctggcatgagatgtggcatgaaggtcttgaagaggcctctcgcttgtactttggggagaggaacgtcaaaggcatgtttgaggtgctggagcccctgcatgctatgatggaacgcggtccccagaccctgaaggaaacgtcctttaatcaggcatatggtcgagatttaatggaggcacaagaatggtgccgaaagtacatgaaatcagggaacgtcaaggacctcctgcaagcctgggacctctactatcacgtgttcagacggatatcacacgagaagttccccagcgacctggacctggacatgttcaacggcagcctggagtgcgacatggagagcatcatcagaagcgagctgatggacgccgacggcctggacttcaacttcgacagccacgagaagttcccctccgacctggatctcgatatgtttaatggaagtctggaatgcgacatggaatctattatcaggtccgaactgatggatgccgacggcctggatttcaactttgatagc

SEQ ID NO: 170 - protein, 217 aa, synthetic construct
MVAILWHEMWHEGLEEASRLYFGERNVKGMFEVLEPLHAMMERGPQTLKETSFNQAYGRDLMEAQEWCRKYMKSGNVKDLLQAWDLYYHVFRRISQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 171 - DNA, 651 bp, synthetic construct
  source 1..651: mol_type = other DNA
atggtagccatcctctggcatgagatgtggcatgaaggtctagaagaggcctctcgcttgtactttggggagaggaacgtcaaaggcatgtttgaggtgctggagcccctgcatgctatgatggaacgcggtccccagaccctgaaggaaacgtcctttaatcaggcatatggtcgagatttaatggaggcacaagaatggtgccgaaagtacatgaaatcagggaacgtcaaggacctcctgcaagcctgggacctctactatcacgtgttcagacggatatcacaggccggcgagggcaccctgagcgaggccctgctgcagctgcagttcgacgacgaggacctgggcgccctgctgggcaacagcaccgaccccgccgtgttcaccgacctggccagcgtggacaacagcgagttccagcagctgctgaaccagggcatccccgtggccccccacaccaccgagcccatgctgatggagtaccccgaggccatcacccgcctggtgaccggagcacaaagaccccctgatcctgcgccagcacctcttggagcacctggcctgcccaacggcctgctgagcggcgacgaggacttcagcagcatcgccgacatggacttcagcgccctgctgagccagatcagcagc

SEQ ID NO: 172 - protein, 216 aa, synthetic construct
MVAILWHEMWHEGLEEASRLYFGERNVKGMFEVLEPLHAMMERGPQTLKETSFNQAYGRDLMEAQEWCRKYMKSGNVKDLLQAWDLYYHVFRRISHEKFPSDLDLDMFNGSLECDMESIIRSELMDADGLDFNFDSEAQNVSSHVPYPVALHVNIVNVPQPAAAAIQRHYNDEDPEKEKRIKELELLLMSTENELKGQQVLPTQNHTCSYPGWHST

SEQ ID NO: 173 - DNA, 648 bp, synthetic construct
  source 1..648: mol_type = other DNA
atggtagccatcctctggcatgagatgtggcatgaaggtcttgaagaggcctctcgcttgtactttggggagaggaacgtcaaaggcatgtttgaggtgctggagcccctgcatgctatgatggaacgcggtccccagaccctgaaggaaacgtcctttaatcaggcatatggtcgagatttaatggaggcacaagaatggtgccgaaagtacatgaaatcagggaacgtcaaggacctcctgcaagcctgggacctctactatcacgtgttcagacggatatcacacgagaagttccccagcgacctggacctggacatgttcaacggcagcctggagtgcgacatggagagcatcatcagaagcgagctgatggacgccgacggcctggacttcaacttcgacagcgaggcccagaacgtgagcagccacgtgccctaccccgtagctttgcatgtgaatattgtcaacgttccgcagcccgcggccgccgccatccagagacactacaacgacgaggaccccgagaaggagaagagaatcaaggagctggagctgctgctgatgagcaccgagaacgagctgaagggccagcaggtgctgcccacccagaaccacacctgcagctaccccggctggcacagcacc

SEQ ID NO: 174 - protein, 460 aa, synthetic construct
MFGVKCHNTDSEPLKNEDLLKNLLTMGVDIDMARKRQPGVFHRMITNEQDLKMFLLSKGASKEVIASIISRYPRAITRTPENLSKRWDLWRKIVTSDLEIVNILERSPESFFRSNNNLNLENNIKFLYSVGLTRKCLCRLLTNAPRTFSNSLDLNKQMVEFLQAAGLSLGHNDPADFVRKIIFKNPFILIQSTKRVKANIEFLRSTFNLNSEELLVLICGPGAEILDLSNDYARRSYANIKEKLFSLGCTEEEVQKFVLSYPDVIFLAEKKFNDKIDCLMEENISISQIIENPRVLDSSISTLKSRIKELVNAGCNLSTLNITLLSWSKKRYEAKLKKLSRFAPAAKRVKLDMGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 175 - DNA, 1380 bp, synthetic construct
  source 1..1380: mol_type = other DNA
atgttcggcgtgaagtgccacaacaccgacagcgagcccctgaagaacgaggacctgctgaagaacctgctgaccatgggcgtggacatcgacatggcccgcaagcgccagcccggcgtgttccaccgcatgatcaccaacgagcaggacctgaagatgttcctgctgagcaagggcgccagcaaggaggtgatcgccagcatcatcagccgctacccccgcgccatcacccgcacccccgagaacctgagcaagcgctgggacctgtggcgcaagatcgtgaccagcgacctggagatcgtgaacatcctggagcgcagccccgagagcttcttccgcagcaacaacaacctgaacctggagaacaacatcaagttcctgtatagcgtgggcctgacccgcaagtgcctgtgccgcctgctgaccaacgccccccgcaccttcagcaacagcctggacctgaacaagcagatggtggagttcctgcaggccgccggcctgagcctgggccacaacgaccccgccgacttcgtgcgcaagatcatcttcaagaaccccttcatcctgatccagagcaccaagcgcgtgaaggccaacatcgagttcctgcgcagcaccttcaacctgaacagcgaggagctgctggtgctgatctgcggccccggcgccgagatcctggacctgagcaacgactacgcccgccgcagctacgccaacatcaaggagaagctgttcagcctgggctgcaccgaggaggaggtgcagaagttcgtgctgagctaccccgacgtgatcttcctggccgagaagaagttcaacgacaagatcgactgcctgatggaggagaacatcagcatcagccagatcatcgagaacccccgcgtgctggacagcagcatcagcaccctgaagagccgcatcaaggagctggtgaacgccggctgcaacctgagcaccctgaacatcaccctgctgagctggagcaagaagcgctacgaggccaagctgaagaagctgagccgcttcgcccctgccgctaagagagtgaagctggacatgggcgtgcaggtggagactatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaa

SEQ ID NO: 176 - protein, 463 aa, synthetic construct
MFGVKCHNTDSEPLKNEDLLKNLLTMGVDIDMARKRQPGVFHRMITNEQDLKMFLLSKGASKEVIASIISRYPRAITRTPENLSKRWDLWRKIVTSDLEIVNILERSPESFFRSNNNLNLENNIKFLYSVGLTRKCLCRLLTNAPRTFSNSLDLNKQMVEFLQAAGLSLGHNDPADFVRKIIFKNPFILIQSTKRVKANIEFLRSTFNLNSEELLVLICGPGAEILDLSNDYARRSYANIKEKLFSLGCTEEEVQKFVLSYPDVIFLAEKKFNDKIDCLMEENISISQIIENPRVLDSSISTLKSRIKELVNAGCNLSTLNITLLSWSKKRYEAKLKKLSRFAAPAPAPAPAPAPMGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 177 - DNA, 1389 bp, synthetic construct
  source 1..1389: mol_type = other DNA
atgttcggcgtgaagtgccacaacaccgacagcgagcccctgaagaacgaggacctgctgaagaacctgctgaccatgggcgtggacatcgacatggcccgcaagcgccagcccggcgtgttccaccgcatgatcaccaacgagcaggacctgaagatgttcctgctgagcaagggcgccagcaaggaggtgatcgccagcatcatcagccgctacccccgcgccatcacccgcacccccgagaacctgagcaagcgctgggacctgtggcgcaagatcgtgaccagcgacctggagatcgtgaacatcctggagcgcagccccgagagcttcttccgcagcaacaacaacctgaacctggagaacaacatcaagttcctgtatagcgtgggcctgacccgcaagtgcctgtgccgcctgctgaccaacgccccccgcaccttcagcaacagcctggacctgaacaagcagatggtggagttcctgcaggccgccggcctgagcctgggccacaacgaccccgccgacttcgtgcgcaagatcatcttcaagaaccccttcatcctgatccagagcaccaagcgcgtgaaggccaacatcgagttcctgcgcagcaccttcaacctgaacagcgaggagctgctggtgctgatctgcggccccggcgccgagatcctggacctgagcaacgactacgcccgccgcagctacgccaacatcaaggagaagctgttcagcctgggctgcaccgaggaggaggtgcagaagttcgtgctgagctaccccgacgtgatcttcctggccgagaagaagttcaacgacaagatcgactgcctgatggaggagaacatcagcatcagccagatcatcgagaacccccgcgtgctggacagcagcatcagcaccctgaagagccgcatcaaggagctggtgaacgccggctgcaacctgagcaccctgaacatcaccctgctgagctggagcaagaagcgctacgaggccaagctgaagaagctgagccgcttcgccgcccctgcaccagctcctgcccctgcaccggctccaatgggcgtgcaggtggagactatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaa

SEQ ID NO: 178 - protein, 467 aa, synthetic construct
MFGVKCHNTDSEPLKNEDLLKNLLTMGVDIDMARKRQPGVFHRMITNEQDLKMFLLSKGASKEVIASIISRYPRAITRTPENLSKRWDLWRKIVTSDLEIVNILERSPESFFRSNNNLNLENNIKFLYSVGLTRKCLCRLLTNAPRTFSNSLDLNKQMVEFLQAAGLSLGHNDPADFVRKIIFKNPFILIQSTKRVKANIEFLRSTFNLNSEELLVLICGPGAEILDLSNDYARRSYANIKEKLFSLGCTEEEVQKFVLSYPDVIFLAEKKFNDKIDCLMEENISISQIIENPRVLDSSISTLKSRIKELVNAGCNLSTLNITLLSWSKKRYEAKLKKLSRFAAPAPAPAPAPAPAPAPMGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 179 - DNA, 1401 bp, synthetic construct
  source 1..1401: mol_type = other DNA
atgttcggcgtgaagtgccacaacaccgacagcgagcccctgaagaacgaggacctgctgaagaacctgctgaccatgggcgtggacatcgacatggcccgcaagcgccagcccggcgtgttccaccgcatgatcaccaacgagcaggacctgaagatgttcctgctgagcaagggcgccagcaaggaggtgatcgccagcatcatcagccgctacccccgcgccatcacccgcacccccgagaacctgagcaagcgctgggacctgtggcgcaagatcgtgaccagcgacctggagatcgtgaacatcctggagcgcagccccgagagcttcttccgcagcaacaacaacctgaacctggagaacaacatcaagttcctgtatagcgtgggcctgacccgcaagtgcctgtgccgcctgctgaccaacgccccccgcaccttcagcaacagcctggacctgaacaagcagatggtggagttcctgcaggccgccggcctgagcctgggccacaacgaccccgccgacttcgtgcgcaagatcatcttcaagaaccccttcatcctgatccagagcaccaagcgcgtgaaggccaacatcgagttcctgcgcagcaccttcaacctgaacagcgaggagctgctggtgctgatctgcggccccggcgccgagatcctggacctgagcaacgactacgcccgccgcagctacgccaacatcaaggagaagctgttcagcctgggctgcaccgaggaggaggtgcagaagttcgtgctgagctaccccgacgtgatcttcctggccgagaagaagttcaacgacaagatcgactgcctgatggaggagaacatcagcatcagccagatcatcgagaacccccgcgtgctggacagcagcatcagcaccctgaagagccgcatcaaggagctggtgaacgccggctgcaacctgagcaccctgaacatcaccctgctgagctggagcaagaagcgctacgaggccaagctgaagaagctgagccgcttcgccgcaccagcaccagctcctgccccggcaccagcgccagcaccagcaccaatgggcgtgcaggtggagactatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaa

SEQ ID NO: 180 - protein, 461 aa, synthetic construct
MFGVKCHNTDSEPLKNEDLLKNLLTMGVDIDMARKRQPGVFHRMITNEQDLKMFLLSKGASKEVIASIISRYPRAITRTPENLSKRWDLWRKIVTSDLEIVNILERSPESFFRSNNNLNLENNIKFLYSVGLTRKCLCRLLTNAPRTFSNSLDLNKQMVEFLQAAGLSLGHNDPADFVRKIIFKNPFILIQSTKRVKANIEFLRSTFNLNSEELLVLICGPGAEILDLSNDYARRSYANIKEKLFSLGCTEEEVQKFVLSYPDVIFLAEKKFNDKIDCLMEENISISQIIENPRVLDSSISTLKSRIKELVNAGCNLSTLNITLLSWSKKRYEAKLKKLSRFAGGGGSGGGGSMGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 181 - DNA, 1383 bp, synthetic construct
  source 1..1383: mol_type = other DNA
atgttcggcgtgaagtgccacaacaccgacagcgagcccctgaagaacgaggacctgctgaagaacctgctgaccatgggcgtggacatcgacatggcccgcaagcgccagcccggcgtgttccaccgcatgatcaccaacgagcaggacctgaagatgttcctgctgagcaagggcgccagcaaggaggtgatcgccagcatcatcagccgctacccccgcgccatcacccgcacccccgagaacctgagcaagcgctgggacctgtggcgcaagatcgtgaccagcgacctggagatcgtgaacatcctggagcgcagccccgagagcttcttccgcagcaacaacaacctgaacctggagaacaacatcaagttcctgtatagcgtgggcctgacccgcaagtgcctgtgccgcctgctgaccaacgccccccgcaccttcagcaacagcctggacctgaacaagcagatggtggagttcctgcaggccgccggcctgagcctgggccacaacgaccccgccgacttcgtgcgcaagatcatcttcaagaaccccttcatcctgatccagagcaccaagcgcgtgaaggccaacatcgagttcctgcgcagcaccttcaacctgaacagcgaggagctgctggtgctgatctgcggccccggcgccgagatcctggacctgagcaacgactacgcccgccgcagctacgccaacatcaaggagaagctgttcagcctgggctgcaccgaggaggaggtgcagaagttcgtgctgagctaccccgacgtgatcttcctggccgagaagaagttcaacgacaagatcgactgcctgatggaggagaacatcagcatcagccagatcatcgagaacccccgcgtgctggacagcagcatcagcaccctgaagagccgcatcaaggagctggtgaacgccggctgcaacctgagcaccctgaacatcaccctgctgagctggagcaagaagcgctacgaggccaagctgaagaagctgagccgcttcgccggcggtggcggcagtggcggaggcggttccatgggcgtgcaggtggagactatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaa

SEQ ID NO: 182 - protein, 466 aa, synthetic construct
MFGVKCHNTDSEPLKNEDLLKNLLTMGVDIDMARKRQPGVFHRMITNEQDLKMFLLSKGASKEVIASIISRYPRAITRTPENLSKRWDLWRKIVTSDLEIVNILERSPESFFRSNNNLNLENNIKFLYSVGLTRKCLCRLLTNAPRTFSNSLDLNKQMVEFLQAAGLSLGHNDPADFVRKIIFKNPFILIQSTKRVKANIEFLRSTFNLNSEELLVLICGPGAEILDLSNDYARRSYANIKEKLFSLGCTEEEVQKFVLSYPDVIFLAEKKFNDKIDCLMEENISISQIIENPRVLDSSISTLKSRIKELVNAGCNLSTLNITLLSWSKKRYEAKLKKLSRFAEAAAKEAAAKEAAAKMGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 183 - DNA, 1398 bp, synthetic construct
  source 1..1398: mol_type = other DNA
atgttcggcgtgaagtgccacaacaccgacagcgagcccctgaagaacgaggacctgctgaagaacctgctgaccatgggcgtggacatcgacatggcccgcaagcgccagcccggcgtgttccaccgcatgatcaccaacgagcaggacctgaagatgttcctgctgagcaagggcgccagcaaggaggtgatcgccagcatcatcagccgctacccccgcgccatcacccgcacccccgagaacctgagcaagcgctgggacctgtggcgcaagatcgtgaccagcgacctggagatcgtgaacatcctggagcgcagccccgagagcttcttccgcagcaacaacaacctgaacctggagaacaacatcaagttcctgtatagcgtgggcctgacccgcaagtgcctgtgccgcctgctgaccaacgccccccgcaccttcagcaacagcctggacctgaacaagcagatggtggagttcctgcaggccgccggcctgagcctgggccacaacgaccccgccgacttcgtgcgcaagatcatcttcaagaaccccttcatcctgatccagagcaccaagcgcgtgaaggccaacatcgagttcctgcgcagcaccttcaacctgaacagcgaggagctgctggtgctgatctgcggccccggcgccgagatcctggacctgagcaacgactacgcccgccgcagctacgccaacatcaaggagaagctgttcagcctgggctgcaccgaggaggaggtgcagaagttcgtgctgagctaccccgacgtgatcttcctggccgagaagaagttcaacgacaagatcgactgcctgatggaggagaacatcagcatcagccagatcatcgagaacccccgcgtgctggacagcagcatcagcaccctgaagagccgcatcaaggagctggtgaacgccggctgcaacctgagcaccctgaacatcaccctgctgagctggagcaagaagcgctacgaggccaagctgaagaagctgagccgcttcgccgaagctgcggcaaaagaagccgctgcgaaggaagcggcagcaaaaatgggcgtgcaggtggagactatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaa

SEQ ID NO: 184 - protein, 461 aa, synthetic construct
MFGVKCHNTDSEPLKNEDLLKNLLTMGVDIDMARKRQPGVFHRMITNEQDLKMFLLSKGASKEVIASIISRYPRAITRTPENLSKRWDLWRKIVTSDLEIVNILERSPESFFRSNNNLNLENNIKFLYSVGLTRKCLCRLLTNAPRTFSNSLDLNKQMVEFLQAAGLSLGHNDPADFVRKIIFKNPFILIQSTKRVKANIEFLRSTFNLNSEELLVLICGPGAEILDLSNDYARRSYANIKEKLFSLGCTEEEVQKFVLSYPDVIFLAEKKFNDKIDCLMEENISISQIIENPRVLDSSISTLKSRIKELVNAGCNLSTLNITLLSWSKKRYEAKLKKLSRFAEAAAKEAAAKMGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 185 - DNA, 1383 bp, synthetic construct
  source 1..1383: mol_type = other DNA
atgttcggcgtgaagtgccacaacaccgacagcgagcccctgaagaacgaggacctgctgaagaacctgctgaccatgggcgtggacatcgacatggcccgcaagcgccagcccggcgtgttccaccgcatgatcaccaacgagcaggacctgaagatgttcctgctgagcaagggcgccagcaaggaggtgatcgccagcatcatcagccgctacccccgcgccatcacccgcacccccgagaacctgagcaagcgctgggacctgtggcgcaagatcgtgaccagcgacctggagatcgtgaacatcctggagcgcagccccgagagcttcttccgcagcaacaacaacctgaacctggagaacaacatcaagttcctgtatagcgtgggcctgacccgcaagtgcctgtgccgcctgctgaccaacgccccccgcaccttcagcaacagcctggacctgaacaagcagatggtggagttcctgcaggccgccggcctgagcctgggccacaacgaccccgccgacttcgtgcgcaagatcatcttcaagaaccccttcatcctgatccagagcaccaagcgcgtgaaggccaacatcgagttcctgcgcagcaccttcaacctgaacagcgaggagctgctggtgctgatctgcggccccggcgccgagatcctggacctgagcaacgactacgcccgccgcagctacgccaacatcaaggagaagctgttcagcctgggctgcaccgaggaggaggtgcagaagttcgtgctgagctaccccgacgtgatcttcctggccgagaagaagttcaacgacaagatcgactgcctgatggaggagaacatcagcatcagccagatcatcgagaacccccgcgtgctggacagcagcatcagcaccctgaagagccgcatcaaggagctggtgaacgccggctgcaacctgagcaccctgaacatcaccctgctgagctggagcaagaagcgctacgaggccaagctgaagaagctgagccgcttcgccgaagcagcggcaaaagaggcagcggcaaaaatgggcgtgcaggtggagactatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaa

SEQ ID NO: 186 - protein, 456 aa, synthetic construct
MFGVKCHNTDSEPLKNEDLLKNLLTMGVDIDMARKRQPGVFHRMITNEQDLKMFLLSKGASKEVIASIISRYPRAITRTPENLSKRWDLWRKIVTSDLEIVNILERSPESFFRSNNNLNLENNIKFLYSVGLTRKCLCRLLTNAPRTFSNSLDLNKQMVEFLQAAGLSLGHNDPADFVRKIIFKNPFILIQSTKRVKANIEFLRSTFNLNSEELLVLICGPGAEILDLSNDYARRSYANIKEKLFSLGCTEEEVQKFVLSYPDVIFLAEKKFNDKIDCLMEENISISQIIENPRVLDSSISTLKSRIKELVNAGCNLSTLNITLLSWSKKRYEAKLKKLSRFAEAAAKMGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 187 - DNA, 1368 bp, synthetic construct
  source 1..1368: mol_type = other DNA
atgttcggcgtgaagtgccacaacaccgacagcgagcccctgaagaacgaggacctgctgaagaacctgctgaccatgggcgtggacatcgacatggcccgcaagcgccagcccggcgtgttccaccgcatgatcaccaacgagcaggacctgaagatgttcctgctgagcaagggcgccagcaaggaggtgatcgccagcatcatcagccgctacccccgcgccatcacccgcacccccgagaacctgagcaagcgctgggacctgtggcgcaagatcgtgaccagcgacctggagatcgtgaacatcctggagcgcagccccgagagcttcttccgcagcaacaacaacctgaacctggagaacaacatcaagttcctgtatagcgtgggcctgacccgcaagtgcctgtgccgcctgctgaccaacgccccccgcaccttcagcaacagcctggacctgaacaagcagatggtggagttcctgcaggccgccggcctgagcctgggccacaacgaccccgccgacttcgtgcgcaagatcatcttcaagaaccccttcatcctgatccagagcaccaagcgcgtgaaggccaacatcgagttcctgcgcagcaccttcaacctgaacagcgaggagctgctggtgctgatctgcggccccggcgccgagatcctggacctgagcaacgactacgcccgccgcagctacgccaacatcaaggagaagctgttcagcctgggctgcaccgaggaggaggtgcagaagttcgtgctgagctaccccgacgtgatcttcctggccgagaagaagttcaacgacaagatcgactgcctgatggaggagaacatcagcatcagccagatcatcgagaacccccgcgtgctggacagcagcatcagcaccctgaagagccgcatcaaggagctggtgaacgccggctgcaacctgagcaccctgaacatcaccctgctgagctggagcaagaagcgctacgaggccaagctgaagaagctgagccgcttcgccgaagcagcagcaaaaatgggcgtgcaggtggagactatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaa

SEQ ID NO: 188 - protein, 471 aa, synthetic construct
MFGVKCHNTDSEPLKNEDLLKNLLTMGVDIDMARKRQPGVFHRMITNEQDLKMFLLSKGASKEVIASIISRYPRAITRTPENLSKRWDLWRKIVTSDLEIVNILERSPESFFRSNNNLNLENNIKFLYSVGLTRKCLCRLLTNAPRTFSNSLDLNKQMVEFLQAAGLSLGHNDPADFVRKIIFKNPFILIQSTKRVKANIEFLRSTFNLNSEELLVLICGPGAEILDLSNDYARRSYANIKEKLFSLGCTEEEVQKFVLSYPDVIFLAEKKFNDKIDCLMEENISISQIIENPRVLDSSISTLKSRIKELVNAGCNLSTLNITLLSWSKKRYEAKLKKLSRFAGGGGSGGGGSGGGGSGGGGSMGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 189 - DNA, 1413 bp, synthetic construct
  source 1..1413: mol_type = other DNA
atgttcggcgtgaagtgccacaacaccgacagcgagcccctgaagaacgaggacctgctgaagaacctgctgaccatgggcgtggacatcgacatggcccgcaagcgccagcccggcgtgttccaccgcatgatcaccaacgagcaggacctgaagatgttcctgctgagcaagggcgccagcaaggaggtgatcgccagcatcatcagccgctacccccgcgccatcacccgcacccccgagaacctgagcaagcgctgggacctgtggcgcaagatcgtgaccagcgacctggagatcgtgaacatcctggagcgcagccccgagagcttcttccgcagcaacaacaacctgaacctggagaacaacatcaagttcctgtatagcgtgggcctgacccgcaagtgcctgtgccgcctgctgaccaacgccccccgcaccttcagcaacagcctggacctgaacaagcagatggtggagttcctgcaggccgccggcctgagcctgggccacaacgaccccgccgacttcgtgcgcaagatcatcttcaagaaccccttcatcctgatccagagcaccaagcgcgtgaaggccaacatcgagttcctgcgcagcaccttcaacctgaacagcgaggagctgctggtgctgatctgcggccccggcgccgagatcctggacctgagcaacgactacgcccgccgcagctacgccaacatcaaggagaagctgttcagcctgggctgcaccgaggaggaggtgcagaagttcgtgctgagctaccccgacgtgatcttcctggccgagaagaagttcaacgacaagatcgactgcctgatggaggagaacatcagcatcagccagatcatcgagaacccccgcgtgctggacagcagcatcagcaccctgaagagccgcatcaaggagctggtgaacgccggctgcaacctgagcaccctgaacatcaccctgctgagctggagcaagaagcgctacgaggccaagctgaagaagctgagccgcttcgccggaggaggcggtagcggaggaggtggctccggtggaggcgggagcggcggagggggcagcatgggcgtgcaggtggagactatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaa

SEQ ID NO: 190 - protein, 229 aa, synthetic construct
MVAILWHEMWHEGLEEASRLYFGERNVKGMFEVLEPLHAMMERGPQTLKETSFNQAYGRDLMEAQEWCRKYMKSGNVKDLLQAWDLYYHVFRRISAPAPAPAPAPAPQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 191 - DNA, 687 bp, synthetic construct
  source 1..687: mol_type = other DNA
atggtagccatcctctggcatgagatgtggcatgaaggtctagaagaggcctctcgcttgtactttggggagaggaacgtcaaaggcatgtttgaggtgctggagcccctgcatgctatgatggaacgcggtccccagaccctgaaggaaacgtcctttaatcaggcatatggtcgagatttaatggaggcacaagaatggtgccgaaagtacatgaaatcagggaacgtcaaggacctcctgcaagcctgggacctctactatcacgtgttcagacggatatcagcccctgcaccagctcctgcccctgcaccggctccacaggccggcgagggcaccctgagcgaggccctgctgcagctgcagttcgacgacgaggacctgggcgccctgctgggcaacagcaccgaccccgccgtgttcaccgacctggccagcgtggacaacagcgagttccagcagctgctgaaccagggcatccccgtggccccccacaccaccgagcccatgctgatggagtaccccgaggccatcacccgcctggtgaccggagcacaaagaccccctgatcctgcgccagcacctcttggagcacctggcctgcccaacggcctgctgagcggcgacgaggacttcagcagcatcgccgacatggacttcagcgccctgctgagccagatcagcagc

SEQ ID NO: 192 - protein, 451 aa, synthetic construct
MFGVKCHNTDSEPLKNEDLLKNLLTMGVDIDMARKRQPGVFHRMITNEQDLKMFLLSKGASKEVIASIISRYPRAITRTPENLSKRWDLWRKIVTSDLEIVNILERSPESFFRSNNNLNLENNIKFLYSVGLTRKCLCRLLTNAPRTFSNSLDLNKQMVEFLQAAGLSLGHNDPADFVRKIIFKNPFILIQSTKRVKANIEFLRSTFNLNSEELLVLICGPGAEILDLSNDYARRSYANIKEKLFSLGCTEEEVQKFVLSYPDVIFLAEKKFNDKIDCLMEENISISQIIENPRVLDSSISTLKSRIKELVNAGCNLSTLNITLLSWSKKRYEAKLKKLSRFAMGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 193 - DNA, 1353 bp, synthetic construct
  source 1..1353: mol_type = other DNA
atgttcggcgtgaagtgccacaacaccgacagcgagcccctgaagaacgaggacctgctgaagaacctgctgaccatgggcgtggacatcgacatggcccgcaagcgccagcccggcgtgttccaccgcatgatcaccaacgagcaggacctgaagatgttcctgctgagcaagggcgccagcaaggaggtgatcgccagcatcatcagccgctacccccgcgccatcacccgcacccccgagaacctgagcaagcgctgggacctgtggcgcaagatcgtgaccagcgacctggagatcgtgaacatcctggagcgcagccccgagagcttcttccgcagcaacaacaacctgaacctggagaacaacatcaagttcctgtatagcgtgggcctgacccgcaagtgcctgtgccgcctgctgaccaacgccccccgcaccttcagcaacagcctggacctgaacaagcagatggtggagttcctgcaggccgccggcctgagcctgggccacaacgaccccgccgacttcgtgcgcaagatcatcttcaagaaccccttcatcctgatccagagcaccaagcgcgtgaaggccaacatcgagttcctgcgcagcaccttcaacctgaacagcgaggagctgctggtgctgatctgcggccccggcgccgagatcctggacctgagcaacgactacgcccgccgcagctacgccaacatcaaggagaagctgttcagcctgggctgcaccgaggaggaggtgcagaagttcgtgctgagctaccccgacgtgatcttcctggccgagaagaagttcaacgacaagatcgactgcctgatggaggagaacatcagcatcagccagatcatcgagaacccccgcgtgctggacagcagcatcagcaccctgaagagccgcatcaaggagctggtgaacgccggctgcaacctgagcaccctgaacatcaccctgctgagctggagcaagaagcgctacgaggccaagctgaagaagctgagccgcttcgccatgggcgtgcaggtggagactatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaa

SEQ ID NO: 194 - protein, 93 aa, synthetic construct
PGLPNGLLSGDEDFSSIADMDFSALLSQISSPGLPNGLLSGDEDFSSIADMDFSALLSQISSPGLPNGLLSGDEDFSSIADMDFSALLSQISS

SEQ ID NO: 195 - DNA, 279 bp, synthetic construct
  source 1..279: mol_type = other DNA
ccggggctccccaatggcctcctttcaggagatgaagacttctcctccattgcggacatggacttctcagccctgctgagtcagatcagctcccctggactgcctaacggcctgctgagcggcgatgaagatttctcctccatcgccgacatggacttcagcgctctgctgagccagatttctagccccggcctgcccaacggcctgctctccggcgacgaggacttcagcagcatcgccgatatggatttcagcgccctgctgagccagatcagcagc

SEQ ID NO: 196 - protein, 160 aa, synthetic construct
EAQNVSSHVPYPVALHVNIVNVPQPAAAAIQRHYNDEDPEKEKRIKELELLLMSTENELKGQQVLPTQNHTCSYPGWHSTEAQNVSSHVPYPVALHVNIVNVPQPAAAAIQRHYNDEDPEKEKRIKELELLLMSTENELKGQQVLPTQNHTCSYPGWHST

SEQ ID NO: 197 - DNA, 480 bp, synthetic construct
  source 1..480: mol_type = other DNA
gaggcccagaacgtgagcagccacgtgccctaccccgtggccctgcacgtgaacatcgtgaacgtgccccagcccgccgccgccgccatccagagacactacaacgacgaggaccccgagaaggagaagagaatcaaggagctggagctgctgctgatgagcaccgagaacgagctgaagggccagcaggtgctgcccacccagaaccacacctgcagctaccccggctggcacagcaccgaggcacagaatgtctcctctcacgttccctaccccgtagctttgcatgttaatatcgtgaacgtgccgcagccggcggcagcggccatccaaagacactataatgatgaagaccccgagaaagagaaaagaatcaaagagctggagctgttgctgatgtccactgaaaatgagttgaaggggcagcaggtcctcccgactcagaatcatacgtgttcttatcctgggtggcattccaca

SEQ ID NO: 198 - DNA, 10 bp, synthetic construct
  source 1..10: mol_type = other DNA
agagtgcgag

SEQ ID NO: 199 - DNA, 10 bp, synthetic construct
  source 1..10: mol_type = other DNA
aaggtgcgat

SEQ ID NO: 200 - DNA, 22 bp, synthetic construct
  source 1..22: mol_type = other DNA
attaccgggctctgccatctta

SEQ ID NO: 201 - DNA, 47 bp, synthetic construct
  source 1..47: mol_type = other DNA
taagatggcagagcccggtaatctctagagggtatataatgggggcc

SEQ ID NO: 202 - DNA, 48 bp, synthetic construct
  source 1..48: mol_type = other DNA
taagatggcagagcccggtaatgctctagagggtatataatgggggcc

SEQ ID NO: 203 - DNA, 49 bp, synthetic construct
  source 1..49: mol_type = other DNA
taagatggcagagcccggtaatgactctagagggtatataatgggggcc

SEQ ID NO: 204 - DNA, 50 bp, synthetic construct
  source 1..50: mol_type = other DNA
taagatggcagagcccggtaatgacctctagagggtatataatgggggcc

SEQ ID NO: 205 - DNA, 51 bp, synthetic construct
  source 1..51: mol_type = other DNA
taagatggcagagcccggtaatgactctctagagggtatataatgggggcc

SEQ ID NO: 206 - DNA, 52 bp, synthetic construct
  source 1..52: mol_type = other DNA
taagatggcagagcccggtaatgactcctctagagggtatataatgggggcc

SEQ ID NO: 207 - DNA, 53 bp, synthetic construct
  source 1..53: mol_type = other DNA
taagatggcagagcccggtaatgactcactctagagggtatataatgggggcc

SEQ ID NO: 208 - DNA, 54 bp, synthetic construct
  source 1..54: mol_type = other DNA
taagatggcagagcccggtaatgactcatctctagagggtatataatgggggcc

SEQ ID NO: 209 - DNA, 55 bp, synthetic construct
  source 1..55: mol_type = other DNA
taagatggcagagcccggtaatgactcatactctagagggtatataatgggggcc

SEQ ID NO: 210 - DNA, 56 bp, synthetic construct
  source 1..56: mol_type = other DNA
taagatggcagagcccggtaatgactcatatctctagagggtatataatgggggcc

SEQ ID NO: 211 - DNA, 57 bp, synthetic construct
  source 1..57: mol_type = other DNA
taagatggcagagcccggtaatgactcatatgctctagagggtatataatgggggcc

SEQ ID NO: 212 - DNA, 69 bp, synthetic construct
  source 1..69: mol_type = other DNA
taagatggcagagcccggtaattaagatggcagagcccggtaatctctagagggtatataatgggggcc

SEQ ID NO: 213 - DNA, 70 bp, synthetic construct
  source 1..70: mol_type = other DNA
taagatggcagagcccggtaatataagatggcagagcccggtaatctctagagggtatataatgggggcc

SEQ ID NO: 214 - DNA, 71 bp, synthetic construct
  source 1..71: mol_type = other DNA
taagatggcagagcccggtaatagtaagatggcagagcccggtaatctctagagggtatataatgggggcc

SEQ ID NO: 215 - DNA, 72 bp, synthetic construct
  source 1..72: mol_type = other DNA
taagatggcagagcccggtaatagataagatggcagagcccggtaatctctagagggtatataatgggggcc

SEQ ID NO: 216 - DNA, 73 bp, synthetic construct
  source 1..73: mol_type = other DNA
taagatggcagagcccggtaatagagtaagatggcagagcccggtaatctctagagggtatataatgggggcc

SEQ ID NO: 217 - DNA, 74 bp, synthetic construct
  source 1..74: mol_type = other DNA
taagatggcagagcccggtaatagagttaagatggcagagcccggtaatctctagagggtatataatgggggcc

SEQ ID NO: 218 - DNA, 75 bp, synthetic construct
  source 1..75: mol_type = other DNA
taagatggcagagcccggtaatagagtgtaagatggcagagcccggtaatctctagagggtatataatgggggcc

SEQ ID NO: 219 - DNA, 76 bp, synthetic construct
  source 1..76: mol_type = other DNA
taagatggcagagcccggtaatagagtgctaagatggcagagcccggtaatctctagagggtatataatgggggcc

SEQ ID NO: 220 - DNA, 77 bp, synthetic construct
  source 1..77: mol_type = other DNA
taagatggcagagcccggtaatagagtgcgtaagatggcagagcccggtaatctctagagggtatataatgggggcc

SEQ ID NO: 221 - DNA, 78 bp, synthetic construct
  source 1..78: mol_type = other DNA
taagatggcagagcccggtaatagagtgcgataagatggcagagcccggtaatctctagagggtatataatgggggcc

SEQ ID NO: 222 - DNA, 79 bp, synthetic construct
  source 1..79: mol_type = other DNA
taagatggcagagcccggtaatagagtgcgagtaagatggcagagcccggtaatctctagagggtatataatgggggcc

SEQ ID NO: 223 - DNA, 70 bp, synthetic construct
  source 1..70: mol_type = other DNA
taagatggcagagcccggtaattaagatggcagagcccggtaatgctctagagggtatataatgggggcc

SEQ ID NO: 224 - DNA, 71 bp, synthetic construct
  source 1..71: mol_type = other DNA
taagatggcagagcccggtaatataagatggcagagcccggtaatgctctagagggtatataatgggggcc

SEQ ID NO: 225 - DNA, 72 bp, synthetic construct
  source 1..72: mol_type = other DNA
taagatggcagagcccggtaatagtaagatggcagagcccggtaatgctctagagggtatataatgggggcc

SEQ ID NO: 226 - DNA, 73 bp, synthetic construct
  source 1..73: mol_type = other DNA
taagatggcagagcccggtaatagataagatggcagagcccggtaatgctctagagggtatataatgggggcc

SEQ ID NO: 227 - DNA, 74 bp, synthetic construct
  source 1..74: mol_type = other DNA
taagatggcagagcccggtaatagagtaagatggcagagcccggtaatgctctagagggtatataatgggggcc

SEQ ID NO: 228 - DNA, 75 bp, synthetic construct
  source 1..75: mol_type = other DNA
taagatggcagagcccggtaatagagttaagatggcagagcccggtaatgctctagagggtatataatgggggcc

SEQ ID NO: 229 - DNA, 76 bp, synthetic construct
  source 1..76: mol_type = other DNA
taagatggcagagcccggtaatagagtgtaagatggcagagcccggtaatgctctagagggtatataatgggggcc

SEQ ID NO: 230 - DNA, 77 bp, synthetic construct
  source 1..77: mol_type = other DNA
taagatggcagagcccggtaatagagtgctaagatggcagagcccggtaatgctctagagggtatataatgggggcc

SEQ ID NO: 231 - DNA, 78 bp, synthetic construct
  source 1..78: mol_type = other DNA
taagatggcagagcccggtaatagagtgcgtaagatggcagagcccggtaatgctctagagggtatataatgggggcc

SEQ ID NO: 232 - DNA, 79 bp, synthetic construct
  source 1..79: mol_type = other DNA
taagatggcagagcccggtaatagagtgcgataagatggcagagcccggtaatgctctagagggtatataatgggggcc

SEQ ID NO: 233 - DNA, 80 bp, synthetic construct
  source 1..80: mol_type = other DNA
taagatggcagagcccggtaatagagtgcgagtaagatggcagagcccggtaatgctctagagggtatataatgggggcc

SEQ ID NO: 234 - DNA, 71 bp, synthetic construct
  source 1..71: mol_type = other DNA
taagatggcagagcccggtaattaagatggcagagcccggtaatgactctagagggtatataatgggggcc

SEQ ID NO: 235 - DNA, 72 bp, synthetic construct
  source 1..72: mol_type = other DNA
taagatggcagagcccggtaatataagatggcagagcccggtaatgactctagagggtatataatgggggcc

SEQ ID NO: 236 - DNA, 73 bp, synthetic construct
  source 1..73: mol_type = other DNA
taagatggcagagcccggtaatagtaagatggcagagcccggtaatgactctagagggtatataatgggggcc

SEQ ID NO: 237 - DNA, 74 bp, synthetic construct
  source 1..74: mol_type = other DNA
taagatggcagagcccggtaatagataagatggcagagcccggtaatgactctagagggtatataatgggggcc

SEQ ID NO: 238 - DNA, 75 bp, synthetic construct
  source 1..75: mol_type = other DNA
taagatggcagagcccggtaatagagtaagatggcagagcccggtaatgactctagagggtatataatgggggcc

SEQ ID NO: 239 - DNA, 76 bp, synthetic construct
  source 1..76: mol_type = other DNA
taagatggcagagcccggtaatagagttaagatggcagagcccggtaatgactctagagggtatataatgggggcc

SEQ ID NO: 240 - DNA, 77 bp, synthetic construct
  source 1..77: mol_type = other DNA
taagatggcagagcccggtaatagagtgtaagatggcagagcccggtaatgactctagagggtatataatgggggcc

SEQ ID NO: 241 - DNA, 78 bp, synthetic construct
  source 1..78: mol_type = other DNA
taagatggcagagcccggtaatagagtgctaagatggcagagcccggtaatgactctagagggtatataatgggggcc

SEQ ID NO: 242 - DNA, 79 bp, synthetic construct
  source 1..79: mol_type = other DNA
taagatggcagagcccggtaatagagtgcgtaagatggcagagcccggtaatgactctagagggtatataatgggggcc

SEQ ID NO: 243 - DNA, 80 bp, synthetic construct
  source 1..80: mol_type = other DNA
taagatggcagagcccggtaatagagtgcgataagatggcagagcccggtaatgactctagagggtatataatgggggcc

SEQ ID NO: 244 - DNA, 81 bp, synthetic construct
  source 1..81: mol_type = other DNA
taagatggcagagcccggtaatagagtgcgagtaagatggcagagcccggtaatgactctagagggtatataatgggggcc

SEQ ID NO: 245 - DNA, 72 bp, synthetic construct
  source 1..72: mol_type = other DNA
taagatggcagagcccggtaattaagatggcagagcccggtaatgacctctagagggtatataatgggggcc

SEQ ID NO: 246 - DNA, 73 bp, synthetic construct
  source 1..73: mol_type = other DNA
taagatggcagagcccggtaatataagatggcagagcccggtaatgacctctagagggtatataatgggggcc

SEQ ID NO: 247 - DNA, 74 bp, synthetic construct
  source 1..74: mol_type = other DNA
taagatggcagagcccggtaatagtaagatggcagagcccggtaatgacctctagagggtatataatgggggcc

SEQ ID NO: 248 - DNA, 75 bp, synthetic construct
  source 1..75: mol_type = other DNA
taagatggcagagcccggtaatagataagatggcagagcccggtaatgacctctagagggtatataatgggggcc

SEQ ID NO: 249 - DNA, 76 bp, synthetic construct
  source 1..76: mol_type = other DNA
taagatggcagagcccggtaatagagtaagatggcagagcccggtaatgacctctagagggtatataatgggggcc

SEQ ID NO: 250 - DNA, 77 bp, synthetic construct
  source 1..77: mol_type = other DNA
taagatggcagagcccggtaatagagttaagatggcagagcccggtaatgacctctagagggtatataatgggggcc

SEQ ID NO: 251 - DNA, 78 bp, synthetic construct
  source 1..78: mol_type = other DNA
taagatggcagagcccggtaatagagtgtaagatggcagagcccggtaatgacctctagagggtatataatgggggcc

SEQ ID NO: 252 - DNA, 79 bp, synthetic construct
  source 1..79: mol_type = other DNA
taagatggcagagcccggtaatagagtgctaagatggcagagcccggtaatgacctctagagggtatataatgggggcc

SEQ ID NO: 253 - DNA, 80 bp, synthetic construct
  source 1..80: mol_type = other DNA
taagatggcagagcccggtaatagagtgcgtaagatggcagagcccggtaatgacctctagagggtatataatgggggcc

SEQ ID NO: 254 - DNA, 81 bp, synthetic construct
  source 1..81: mol_type = other DNA
taagatggcagagcccggtaatagagtgcgataagatggcagagcccggtaatgacctctagagggtatataatgggggcc

SEQ ID NO: 255 - DNA, 82 bp, synthetic construct
  source 1..82: mol_type = other DNA
taagatggcagagcccggtaatagagtgcgagtaagatggcagagcccggtaatgacctctagagggtatataatgggggcc

SEQ ID NO: 256 - DNA, 73 bp, synthetic construct
  source 1..73: mol_type = other DNA
taagatggcagagcccggtaattaagatggcagagcccggtaatgactctctagagggtatataatgggggcc

SEQ ID NO: 257 - DNA, 74 bp, synthetic construct
  source 1..74: mol_type = other DNA
taagatggcagagcccggtaatataagatggcagagcccggtaatgactctctagagggtatataatgggggcc

SEQ ID NO: 258 - DNA, 75 bp, synthetic construct
  source 1..75: mol_type = other DNA
taagatggcagagcccggtaatagtaagatggcagagcccggtaatgactctctagagggtatataatgggggcc

SEQ ID NO: 259 - DNA, 76 bp, synthetic construct
  source 1..76: mol_type = other DNA
taagatggcagagcccggtaatagataagatggcagagcccggtaatgactctctagagggtatataatgggggcc

SEQ ID NO: 260 - DNA, 77 bp, synthetic construct
  source 1..77: mol_type = other DNA
taagatggcagagcccggtaatagagtaagatggcagagcccggtaatgactctctagagggtatataatgggggcc

SEQ ID NO: 261 - DNA, 78 bp, synthetic construct
  source 1..78: mol_type = other DNA
taagatggcagagcccggtaatagagttaagatggcagagcccggtaatgactctctagagggtatataatgggggcc

SEQ ID NO: 262 - DNA, 79 bp, synthetic construct
  source 1..79: mol_type = other DNA
taagatggcagagcccggtaatagagtgtaagatggcagagcccggtaatgactctctagagggtatataatgggggcc

SEQ ID NO: 263 - DNA, 80 bp, synthetic construct
  source 1..80: mol_type = other DNA
taagatggcagagcccggtaatagagtgctaagatggcagagcccggtaatgactctctagagggtatataatgggggcc

SEQ ID NO: 264 - DNA, 81 bp, synthetic construct
  source 1..81: mol_type = other DNA
taagatggcagagcccggtaatagagtgcgtaagatggcagagcccggtaatgactctctagagggtatataatgggggcc

SEQ ID NO: 265 - DNA, 82 bp, synthetic construct
  source 1..82: mol_type = other DNA
taagatggcagagcccggtaatagagtgcgataagatggcagagcccggtaatgactctctagagggtatataatgggggcc

SEQ ID NO: 266 - DNA, 83 bp, synthetic construct
  source 1..83: mol_type = other DNA
taagatggcagagcccggtaatagagtgcgagtaagatggcagagcccggtaatgactctctagagggtatataatgggggcc

SEQ ID NO: 267 - DNA, 74 bp, synthetic construct
  source 1..74: mol_type = other DNA
taagatggcagagcccggtaattaagatggcagagcccggtaatgactcctctagagggtatataatgggggcc

SEQ ID NO: 268 - DNA, 75 bp, synthetic construct
  source 1..75: mol_type = other DNA
taagatggcagagcccggtaatataagatggcagagcccggtaatgactcctctagagggtatataatgggggcc

SEQ ID NO: 269 - DNA, 76 bp, synthetic construct
  source 1..76: mol_type = other DNA
taagatggcagagcccggtaatagtaagatggcagagcccggtaatgactcctctagagggtatataatgggggcc

SEQ ID NO: 270 - DNA, 77 bp, synthetic construct
  source 1..77: mol_type = other DNA
taagatggcagagcccggtaatagataagatggcagagcccggtaatgactcctctagagggtatataatgggggcc

SEQ ID NO: 271 - DNA, 78 bp, synthetic construct
  source 1..78: mol_type = other DNA
taagatggcagagcccggtaatagagtaagatggcagagcccggtaatgactcctctagagggtatataatgggggcc

SEQ ID NO: 272 - DNA, 79 bp, synthetic construct
  source 1..79: mol_type = other DNA
taagatggcagagcccggtaatagagttaagatggcagagcccggtaatgactcctctagagggtatataatgggggcc

SEQ ID NO: 273 - DNA, 80 bp, synthetic construct
  source 1..80: mol_type = other DNA
taagatggcagagcccggtaatagagtgtaagatggcagagcccggtaatgactcctctagagggtatataatgggggcc

SEQ ID NO: 274 - DNA, 81 bp, synthetic construct
  source 1..81: mol_type = other DNA
taagatggcagagcccggtaatagagtgctaagatggcagagcccggtaatgactcctctagagggtatataatgggggcc

SEQ ID NO: 275 - DNA, 82 bp, synthetic construct
  source 1..82: mol_type = other DNA
taagatggcagagcccggtaatagagtgcgtaagatggcagagcccggtaatgactcctctagagggtatataatgggggcc

SEQ ID NO: 276 - DNA, 83 bp, synthetic construct
  source 1..83: mol_type = other DNA
taagatggcagagcccggtaatagagtgcgataagatggcagagcccggtaatgactcctctagagggtatataatgggggcc

SEQ ID NO: 277 - DNA, 84 bp, synthetic construct
  source 1..84: mol_type = other DNA
taagatggcagagcccggtaatagagtgcgagtaagatggcagagcccggtaatgactcctctagagggtatataatgggggcc

SEQ ID NO: 278 - DNA, 75 bp, synthetic construct
  source 1..75: mol_type = other DNA
taagatggcagagcccggtaattaagatggcagagcccggtaatgactcactctagagggtatataatgggggcc

SEQ ID NO: 279 - DNA, 76 bp, synthetic construct
  source 1..76: mol_type = other DNA
taagatggcagagcccggtaatataagatggcagagcccggtaatgactcactctagagggtatataatgggggcc

SEQ ID NO: 280 - DNA, 77 bp, synthetic construct
  source 1..77: mol_type = other DNA
taagatggcagagcccggtaatagtaagatggcagagcccggtaatgactcactctagagggtatataatgggggcc

SEQ ID NO: 281 - DNA, 78 bp, synthetic construct
  source 1..78: mol_type = other DNA
taagatggcagagcccggtaatagataagatggcagagcccggtaatgactcactctagagggtatataatgggggcc

SEQ ID NO: 282 - DNA, 79 bp, synthetic construct
  source 1..79: mol_type = other DNA
taagatggcagagcccggtaatagagtaagatggcagagcccggtaatgactcactctagagggtatataatgggggcc

SEQ ID NO: 283 - DNA, 80 bp, synthetic construct
  source 1..80: mol_type = other DNA
taagatggcagagcccggtaatagagttaagatggcagagcccggtaatgactcactctagagggtatataatgggggcc

SEQ ID NO: 284 - DNA, 81 bp, synthetic construct
  source 1..81: mol_type = other DNA
taagatggcagagcccggtaatagagtgtaagatggcagagcccggtaatgactcactctagagggtatataatgggggcc

SEQ ID NO: 285 - DNA, 82 bp, synthetic construct
  source 1..82: mol_type = other DNA
taagatggcagagcccggtaatagagtgctaagatggcagagcccggtaatgactcactctagagggtatataatgggggcc

SEQ ID NO: 286 - DNA, 83 bp, synthetic construct
  source 1..83: mol_type = other DNA
taagatggcagagcccggtaatagagtgcgtaagatggcagagcccggtaatgactcactctagagggtatataatgggggcc

SEQ ID NO: 287 - DNA, 84 bp, synthetic construct
  source 1..84: mol_type = other DNA
taagatggcagagcccggtaatagagtgcgataagatggcagagcccggtaatgactcactctagagggtatataatgggggcc

SEQ ID NO: 288 - DNA, 85 bp, synthetic construct
  source 1..85: mol_type = other DNA
taagatggcagagcccggtaatagagtgcgagtaagatggcagagcccggtaatgactcactctagagggtatataatgggggcc

SEQ ID NO: 289 - DNA, 76 bp, synthetic construct
  source 1..76: mol_type = other DNA
taagatggcagagcccggtaattaagatggcagagcccggtaatgactcatctctagagggtatataatgggggcc

SEQ ID NO: 290 - DNA, 77 bp, synthetic construct
  source 1..77: mol_type = other DNA
taagatggcagagcccggtaatataagatggcagagcccggtaatgactcatctctagagggtatataatgggggcc

SEQ ID NO: 291 - DNA, 78 bp, synthetic construct
  source 1..78: mol_type = other DNA
taagatggcagagcccggtaatagtaagatggcagagcccggtaatgactcatctctagagggtatataatgggggcc

SEQ ID NO: 292 - DNA, 79 bp, synthetic construct
  source 1..79: mol_type = other DNA
taagatggcagagcccggtaatagataagatggcagagcccggtaatgactcatctctagagggtatataatgggggcc

SEQ ID NO: 293 - DNA, 80 bp, synthetic construct
  source 1..80: mol_type = other DNA
taagatggcagagcccggtaatagagtaagatggcagagcccggtaatgactcatctctagagggtatataatgggggcc

SEQ ID NO: 294 - DNA, 81 bp, synthetic construct
  source 1..81: mol_type = other DNA
taagatggcagagcccggtaatagagttaagatggcagagcccggtaatgactcatctctagagggtatataatgggggcc

SEQ ID NO: 295 - DNA, 82 bp, synthetic construct
  source 1..82: mol_type = other DNA
taagatggcagagcccggtaatagagtgtaagatggcagagcccggtaatgactcatctctagagggtatataatgggggcc

SEQ ID NO: 296 - DNA, 83 bp, synthetic construct
  source 1..83: mol_type = other DNA
taagatggcagagcccggtaatagagtgctaagatggcagagcccggtaatgactcatctctagagggtatataatgggggcc

SEQ ID NO: 297 - DNA, 84 bp, synthetic construct
  source 1..84: mol_type = other DNA
taagatggcagagcccggtaatagagtgcgtaagatggcagagcccggtaatgactcatctctagagggtatataatgggggcc

SEQ ID NO: 298 - DNA, 85 bp, synthetic construct
  source 1..85: mol_type = other DNA
taagatggcagagcccggtaatagagtgcgataagatggcagagcccggtaatgactcatctctagagggtatataatgggggcc

SEQ ID NO: 299 - DNA, 86 bp, synthetic construct
  source 1..86: mol_type = other DNA
taagatggcagagcccggtaatagagtgcgagtaagatggcagagcccggtaatgactcatctctagagggtatataatgggggcc

SEQ ID NO: 300 - DNA, 77 bp, synthetic construct
  source 1..77: mol_type = other DNA
taagatggcagagcccggtaattaagatggcagagcccggtaatgactcatactctagagggtatataatgggggcc

SEQ ID NO: 301 - DNA, 78 bp, synthetic construct
  source 1..78: mol_type = other DNA
taagatggcagagcccggtaatataagatggcagagcccggtaatgactcatactctagagggtatataatgggggcc

SEQ ID NO: 302 - DNA, 79 bp, synthetic construct
  source 1..79: mol_type = other DNA
taagatggcagagcccggtaatagtaagatggcagagcccggtaatgactcatactctagagggtatataatgggggcc

SEQ ID NO: 303 - DNA, 80 bp, synthetic construct
  source 1..80: mol_type = other DNA
taagatggcagagcccggtaatagataagatggcagagcccggtaatgactcatactctagagggtatataatgggggcc

SEQ ID NO: 304 - DNA, 81 bp, synthetic construct
  source 1..81: mol_type = other DNA
taagatggcagagcccggtaatagagtaagatggcagagcccggtaatgactcatactctagagggtatataatgggggcc

SEQ ID NO: 305 - DNA, 82 bp, synthetic construct
  source 1..82: mol_type = other DNA
taagatggcagagcccggtaatagagttaagatggcagagcccggtaatgactcatactctagagggtatataatgggggcc

SEQ ID NO: 306 - DNA, 83 bp, synthetic construct
  source 1..83: mol_type = other DNA
taagatggcagagcccggtaatagagtgtaagatggcagagcccggtaatgactcatactctagagggtatataatgggggcc

SEQ ID NO: 307 - DNA, 84 bp, synthetic construct
  source 1..84: mol_type = other DNA
taagatggcagagcccggtaatagagtgctaagatggcagagcccggtaatgactcatactctagagggtatataatgggggcc

SEQ ID NO: 308 - DNA, 85 bp, synthetic construct
  source 1..85: mol_type = other DNA
taagatggcagagcccggtaatagagtgcgtaagatggcagagcccggtaatgactcatactctagagggtatataatgggggcc

SEQ ID NO: 309 - DNA, 86 bp, synthetic construct
  source 1..86: mol_type = other DNA
taagatggcagagcccggtaatagagtgcgataagatggcagagcccggtaatgactcatactctagagggtatataatgggggcc

SEQ ID NO: 310 - DNA, 87 bp, synthetic construct
  source 1..87: mol_type = other DNA
taagatggcagagcccggtaatagagtgcgagtaagatggcagagcccggtaatgactcatactctagagggtatataatgggggcc

SEQ ID NO: 311 - DNA, 78 bp, synthetic construct
  source 1..78: mol_type = other DNA
taagatggcagagcccggtaattaagatggcagagcccggtaatgactcatatctctagagggtatataatgggggcc

SEQ ID NO: 312 - DNA, 79 bp, synthetic construct
  source 1..79: mol_type = other DNA
taagatggcagagcccggtaatataagatggcagagcccggtaatgactcatatctctagagggtatataatgggggcc

SEQ ID NO: 313 - DNA, 80 bp, synthetic construct
  source 1..80: mol_type = other DNA
taagatggcagagcccggtaatagtaagatggcagagcccggtaatgactcatatctctagagggtatataatgggggcc

SEQ ID NO: 314 - DNA, 81 bp, synthetic construct
  source 1..81: mol_type = other DNA
taagatggcagagcccggtaatagataagatggcagagcccggtaatgactcatatctctagagggtatataatgggggcc

SEQ ID NO: 315 - DNA, 82 bp, synthetic construct
  source 1..82: mol_type = other DNA
taagatggcagagcccggtaatagagtaagatggcagagcccggtaatgactcatatctctagagggtatataatgggggcc

SEQ ID NO: 316 - DNA, 83 bp, synthetic construct
  source 1..83: mol_type = other DNA
taagatggcagagcccggtaatagagttaagatggcagagcccggtaatgactcatatctctagagggtatataatgggggcc

SEQ ID NO: 317 - DNA, 84 bp, synthetic construct
  source 1..84: mol_type = other DNA
taagatggcagagcccggtaatagagtgtaagatggcagagcccggtaatgactcatatctctagagggtatataatgggggcc

SEQ ID NO: 318 - DNA, 85 bp, synthetic construct
  source 1..85: mol_type = other DNA
taagatggcagagcccggtaatagagtgctaagatggcagagcccggtaatgactcatatctctagagggtatataatgggggcc

SEQ ID NO: 319 - DNA, 86 bp, synthetic construct
  source 1..86: mol_type = other DNA
taagatggcagagcccggtaatagagtgcgtaagatggcagagcccggtaatgactcatatctctagagggtatataatgggggcc

SEQ ID NO: 320 - DNA, 87 bp, synthetic construct
  source 1..87: mol_type = other DNA
taagatggcagagcccggtaatagagtgcgataagatggcagagcccggtaatgactcatatctctagagggtatataatgggggcc

SEQ ID NO: 321 - DNA, 88 bp, synthetic construct
  source 1..88: mol_type = other DNA
taagatggcagagcccggtaatagagtgcgagtaagatggcagagcccggtaatgactcatatctctagagggtatataatgggggcc

SEQ ID NO: 322 - DNA, 79 bp, synthetic construct
  source 1..79: mol_type = other DNA
taagatggcagagcccggtaattaagatggcagagcccggtaatgactcatatgctctagagggtatataatgggggcc

SEQ ID NO: 323 - DNA, 80 bp, synthetic construct
  source 1..80: mol_type = other DNA
taagatggcagagcccggtaatataagatggcagagcccggtaatgactcatatgctctagagggtatataatgggggcc

SEQ ID NO: 324 - DNA, 81 bp, synthetic construct
  source 1..81: mol_type = other DNA
taagatggcagagcccggtaatagtaagatggcagagcccggtaatgactcatatgctctagagggtatataatgggggcc

SEQ ID NO: 325 - DNA, 82 bp, synthetic construct
  source 1..82: mol_type = other DNA
taagatggcagagcccggtaatagataagatggcagagcccggtaatgactcatatgctctagagggtatataatgggggcc

SEQ ID NO: 326 - DNA, 83 bp, synthetic construct
  source 1..83: mol_type = other DNA
taagatggcagagcccggtaatagagtaagatggcagagcccggtaatgactcatatgctctagagggtatataatgggggcc

SEQ ID NO: 327 - DNA, 84 bp, synthetic construct
  source 1..84: mol_type = other DNA
taagatggcagagcccggtaatagagttaagatggcagagcccggtaatgactcatatgctctagagggtatataatgggggcc

SEQ ID NO: 328 - DNA, 85 bp, synthetic construct
  source 1..85: mol_type = other DNA
taagatggcagagcccggtaatagagtgtaagatggcagagcccggtaatgactcatatgctctagagggtatataatgggggcc

SEQ ID NO: 329 - DNA, 86 bp, synthetic construct
  source 1..86: mol_type = other DNA
taagatggcagagcccggtaatagagtgctaagatggcagagcccggtaatgactcatatgctctagagggtatataatgggggcc

SEQ ID NO: 330 - DNA, 87 bp, synthetic construct
  source 1..87: mol_type = other DNA
taagatggcagagcccggtaatagagtgcgtaagatggcagagcccggtaatgactcatatgctctagagggtatataatgggggcc

SEQ ID NO: 331 - DNA, 88 bp, synthetic construct
  source 1..88: mol_type = other DNA
taagatggcagagcccggtaatagagtgcgataagatggcagagcccggtaatgactcatatgctctagagggtatataatgggggcc

SEQ ID NO: 332 - DNA, 89 bp, synthetic construct
  source 1..89: mol_type = other DNA
taagatggcagagcccggtaatagagtgcgagtaagatggcagagcccggtaatgactcatatgctctagagggtatataatgggggcc

SEQ ID NO: 333 - DNA, 91 bp, synthetic construct
  source 1..91: mol_type = other DNA
taagatggcagagcccggtaattaagatggcagagcccggtaattaagatggcagagcccggtaatctctagagggtatataatgggggcc

SEQ ID NO: 334 - DNA, 93 bp, synthetic construct
  source 1..93: mol_type = other DNA
taagatggcagagcccggtaatataagatggcagagcccggtaatataagatggcagagcccggtaatctctagagggtatataatgggggcc

SEQ ID NO: 335 - DNA, 95 bp, synthetic construct
  source 1..95: mol_type = other DNA
taagatggcagagcccggtaatagtaagatggcagagcccggtaatagtaagatggcagagcccggtaatctctagagggtatataatgggggcc

SEQ ID NO: 336 - DNA, 97 bp, synthetic construct
  source 1..97: mol_type = other DNA
taagatggcagagcccggtaatagataagatggcagagcccggtaatagataagatggcagagcccggtaatctctagagggtatataatgggggcc

SEQ ID NO: 337 - DNA, 99 bp, synthetic construct
  source 1..99: mol_type = other DNA
taagatggcagagcccggtaatagagtaagatggcagagcccggtaatagagtaagatggcagagcccggtaatctctagagggtatataatgggggcc

SEQ ID NO: 338 - DNA, 101 bp, synthetic construct
  source 1..101: mol_type = other DNA
taagatggcagagcccggtaatagagttaagatggcagagcccggtaatagagttaagatggcagagcccggtaatctctagagggtatataatgggggcc

SEQ ID NO: 339 - DNA, 103 bp, synthetic construct
  source 1..103: mol_type = other DNA
taagatggcagagcccggtaatagagtgtaagatggcagagcccggtaatagagtgtaagatggcagagcccggtaatctctagagggtatataatgggggcc

SEQ ID NO: 340 - DNA, 105 bp, synthetic construct
  source 1..105: mol_type = other DNA
taagatggcagagcccggtaatagagtgctaagatggcagagcccggtaatagagtgctaagatggcagagcccggtaatctctagagggtatataatgggggcc

SEQ ID NO: 341 - DNA, 107 bp, synthetic construct
  source 1..107: mol_type = other DNA
taagatggcagagcccggtaatagagtgcgtaagatggcagagcccggtaatagagtgcgtaagatggcagagcccggtaatctctagagggtatataatgggggcc

SEQ ID NO: 342 - DNA, 109 bp, synthetic construct
  source 1..109: mol_type = other DNA
taagatggcagagcccggtaatagagtgcgataagatggcagagcccggtaatagagtgcgataagatggcagagcccggtaatctctagagggtatataatgggggcc

SEQ ID NO: 343 - DNA, 111 bp, synthetic construct
  source 1..111: mol_type = other DNA
taagatggcagagcccggtaatagagtgcgagtaagatggcagagcccggtaatagagtgcgagtaagatggcagagcccggtaatctctagagggtatataatgggggcc

SEQ ID NO: 344 - DNA, 92 bp, synthetic construct
  source 1..92: mol_type = other DNA
taagatggcagagcccggtaattaagatggcagagcccggtaattaagatggcagagcccggtaatgctctagagggtatataatgggggcc

SEQ ID NO: 345 - DNA, 94 bp, synthetic construct
  source 1..94: mol_type = other DNA
taagatggcagagcccggtaatataagatggcagagcccggtaatataagatggcagagcccggtaatgctctagagggtatataatgggggcc

SEQ ID NO: 346 - DNA, 96 bp, synthetic construct
  source 1..96: mol_type = other DNA
taagatggcagagcccggtaatagtaagatggcagagcccggtaatagtaagatggcagagcccggtaatgctctagagggtatataatgggggcc

SEQ ID NO: 347 - DNA, 98 bp, synthetic construct
  source 1..98: mol_type = other DNA
taagatggcagagcccggtaatagataagatggcagagcccggtaatagataagatggcagagcccggtaatgctctagagggtatataatgggggcc

SEQ ID NO: 348 - DNA, 100 bp, synthetic construct
  source 1..100: mol_type = other DNA
taagatggcagagcccggtaatagagtaagatggcagagcccggtaatagagtaagatggcagagcccggtaatgctctagagggtatataatgggggcc

SEQ ID NO: 349 - DNA, 102 bp, synthetic construct
  source 1..102: mol_type = other DNA
taagatggcagagcccggtaatagagttaagatggcagagcccggtaatagagttaagatggcagagcccggtaatgctctagagggtatataatgggggcc

SEQ ID NO: 350 - DNA, 104 bp, synthetic construct
  source 1..104: mol_type = other DNA
taagatggcagagcccggtaatagagtgtaagatggcagagcccggtaatagagtgtaagatggcagagcccggtaatgctctagagggtatataatgggggcc

SEQ ID NO: 351 - DNA, 106 bp, synthetic construct
  source 1..106: mol_type = other DNA
taagatggcagagcccggtaatagagtgctaagatggcagagcccggtaatagagtgctaagatggcagagcccggtaatgctctagagggtatataatgggggcc

SEQ ID NO: 352 - DNA, 108 bp, synthetic construct
  source 1..108: mol_type = other DNA
taagatggcagagcccggtaatagagtgcgtaagatggcagagcccggtaatagagtgcgtaagatggcagagcccggtaatgctctagagggtatataatgggggcc

SEQ ID NO: 353 - DNA, 110 bp, synthetic construct
  source 1..110: mol_type = other DNA
taagatggcagagcccggtaatagagtgcgataagatggcagagcccggtaatagagtgcgataagatggcagagcccggtaatgctctagagggtatataatgggggcc

SEQ ID NO: 354 - DNA, 112 bp, synthetic construct
  source 1..112: mol_type = other DNA
taagatggcagagcccggtaatagagtgcgagtaagatggcagagcccggtaatagagtgcgagtaagatggcagagcccggtaatgctctagagggtatataatgggggcc

SEQ ID NO: 355 - DNA, 93 bp, synthetic construct
  source 1..93: mol_type = other DNA
taagatggcagagcccggtaattaagatggcagagcccggtaattaagatggcagagcccggtaatgactctagagggtatataatgggggcc

SEQ ID NO: 356 - DNA, 95 bp, synthetic construct
  source 1..95: mol_type = other DNA
taagatggcagagcccggtaatataagatggcagagcccggtaatataagatggcagagcccggtaatgactctagagggtatataatgggggcc

SEQ ID NO: 357 - DNA, 97 bp, synthetic construct
  source 1..97: mol_type = other DNA
taagatggcagagcccggtaatagtaagatggcagagcccggtaatagtaagatggcagagcccggtaatgactctagagggtatataatgggggcc

SEQ ID NO: 358 - DNA, 99 bp, synthetic construct
  source 1..99: mol_type = other DNA
taagatggcagagcccggtaatagataagatggcagagcccggtaatagataagatggcagagcccggtaatgactctagagggtatataatgggggcc

SEQ ID NO: 359 - DNA, 101 bp, synthetic construct
  source 1..101: mol_type = other DNA
taagatggcagagcccggtaatagagtaagatggcagagcccggtaatagagtaagatggcagagcccggtaatgactctagagggtatataatgggggcc

SEQ ID NO: 360 - DNA, 103 bp, synthetic construct
  source 1..103: mol_type = other DNA
taagatggcagagcccggtaatagagttaagatggcagagcccggtaatagagttaagatggcagagcccggtaatgactctagagggtatataatgggggcc

SEQ ID NO: 361 - DNA, 105 bp, synthetic construct
  source 1..105: mol_type = other DNA
taagatggcagagcccggtaatagagtgtaagatggcagagcccggtaatagagtgtaagatggcagagcccggtaatgactctagagggtatataatgggggcc

SEQ ID NO: 362 - DNA, 107 bp, synthetic construct
  source 1..107: mol_type = other DNA
taagatggcagagcccggtaatagagtgctaagatggcagagcccggtaatagagtgctaagatggcagagcccggtaatgactctagagggtatataatgggggcc

SEQ ID NO: 363 - DNA, 109 bp, synthetic construct
  source 1..109: mol_type = other DNA
taagatggcagagcccggtaatagagtgcgtaagatggcagagcccggtaatagagtgcgtaagatggcagagcccggtaatgactctagagggtatataatgggggcc

SEQ ID NO: 364 - DNA, 111 bp, synthetic construct
  source 1..111: mol_type = other DNA
taagatggcagagcccggtaatagagtgcgataagatggcagagcccggtaatagagtgcgataagatggcagagcccggtaatgactctagagggtatataatgggggcc

SEQ ID NO: 365 - DNA, 113 bp, synthetic construct
  source 1..113: mol_type = other DNA
taagatggcagagcccggtaatagagtgcgagtaagatggcagagcccggtaatagagtgcgagtaagatggcagagcccggtaatgactctagagggtatataatgggggcc

SEQ ID NO: 366 - DNA, 94 bp, synthetic construct
  source 1..94: mol_type = other DNA
taagatggcagagcccggtaattaagatggcagagcccggtaattaagatggcagagcccggtaatgacctctagagggtatataatgggggcc

SEQ ID NO: 367 - DNA, 96 bp, synthetic construct
  source 1..96: mol_type = other DNA
taagatggcagagcccggtaatataagatggcagagcccggtaatataagatggcagagcccggtaatgacctctagagggtatataatgggggcc

SEQ ID NO: 368 - DNA, 98 bp, synthetic construct
  source 1..98: mol_type = other DNA
taagatggcagagcccggtaatagtaagatggcagagcccggtaatagtaagatggcagagcccggtaatgacctctagagggtatataatgggggcc

SEQ ID NO: 369 - DNA, 100 bp, synthetic construct
  source 1..100: mol_type = other DNA
taagatggcagagcccggtaatagataagatggcagagcccggtaatagataagatggcagagcccggtaatgacctctagagggtatataatgggggcc

SEQ ID NO: 370 - DNA, 102 bp, synthetic construct
  source 1..102: mol_type = other DNA
taagatggcagagcccggtaatagagtaagatggcagagcccggtaatagagtaagatggcagagcccggtaatgacctctagagggtatataatgggggcc

SEQ ID NO: 371 - DNA, 104 bp, synthetic construct
  source 1..104: mol_type = other DNA
taagatggcagagcccggtaatagagttaagatggcagagcccggtaatagagttaagatggcagagcccggtaatgacctctagagggtatataatgggggcc

SEQ ID NO: 372 - DNA, 106 bp, synthetic construct
  source 1..106: mol_type = other DNA
taagatggcagagcccggtaatagagtgtaagatggcagagcccggtaatagagtgtaagatggcagagcccggtaatgacctctagagggtatataatgggggcc

SEQ ID NO: 373 - DNA, 108 bp, synthetic construct
  source 1..108: mol_type = other DNA
taagatggcagagcccggtaatagagtgctaagatggcagagcccggtaatagagtgctaagatggcagagcccggtaatgacctctagagggtatataatgggggcc

SEQ ID NO: 374 - DNA, 110 bp, synthetic construct
  source 1..110: mol_type = other DNA
taagatggcagagcccggtaatagagtgcgtaagatggcagagcccggtaatagagtgcgtaagatggcagagcccggtaatgacctctagagggtatataatgggggcc

SEQ ID NO: 375 - DNA, 112 bp, synthetic construct
  source 1..112: mol_type = other DNA
taagatggcagagcccggtaatagagtgcgataagatggcagagcccggtaatagagtgcgataagatggcagagcccggtaatgacctctagagggtatataatgggggcc

SEQ ID NO: 376 - DNA, 114 bp, synthetic construct
  source 1..114: mol_type = other DNA
taagatggcagagcccggtaatagagtgcgagtaagatggcagagcccggtaatagagtgcgagtaagatggcagagcccggtaatgacctctagagggtatataatgggggcc

SEQ ID NO: 377 - DNA, 95 bp, synthetic construct
  source 1..95: mol_type = other DNA
taagatggcagagcccggtaattaagatggcagagcccggtaattaagatggcagagcccggtaatgactctctagagggtatataatgggggcc

SEQ ID NO: 378 - DNA, 97 bp, synthetic construct
  source 1..97: mol_type = other DNA
taagatggcagagcccggtaatataagatggcagagcccggtaatataagatggcagagcccggtaatgactctctagagggtatataatgggggcc

SEQ ID NO: 379 - DNA, 99 bp, synthetic construct
  source 1..99: mol_type = other DNA
taagatggcagagcccggtaatagtaagatggcagagcccggtaatagtaagatggcagagcccggtaatgactctctagagggtatataatgggggcc

SEQ ID NO: 380 - DNA, 101 bp, synthetic construct
  source 1..101: mol_type = other DNA
taagatggcagagcccggtaatagataagatggcagagcccggtaatagataagatggcagagcccggtaatgactctctagagggtatataatgggggcc

SEQ ID NO: 381 - DNA, 103 bp, synthetic construct
  source 1..103: mol_type = other DNA
taagatggcagagcccggtaatagagtaagatggcagagcccggtaatagagtaagatggcagagcccggtaatgactctctagagggtatataatgggggcc

SEQ ID NO: 382 - DNA, 105 bp, synthetic construct
  source 1..105: mol_type = other DNA
taagatggcagagcccggtaatagagttaagatggcagagcccggtaatagagttaagatggcagagcccggtaatgactctctagagggtatataatgggggcc

SEQ ID NO: 383 - DNA, 107 bp, synthetic construct
  source 1..107: mol_type = other DNA
taagatggcagagcccggtaatagagtgtaagatggcagagcccggtaatagagtgtaagatggcagagcccggtaatgactctctagagggtatataatgggggcc

SEQ ID NO: 384 - DNA, 109 bp, synthetic construct
  source 1..109: mol_type = other DNA
taagatggcagagcccggtaatagagtgctaagatggcagagcccggtaatagagtgctaagatggcagagcccggtaatgactctctagagggtatataatgggggcc

SEQ ID NO: 385 - DNA, 111 bp, synthetic construct
  source 1..111: mol_type = other DNA
taagatggcagagcccggtaatagagtgcgtaagatggcagagcccggtaatagagtgcgtaagatggcagagcccggtaatgactctctagagggtatataatgggggcc

SEQ ID NO: 386 - DNA, 113 bp, synthetic construct
  source 1..113: mol_type = other DNA
taagatggcagagcccggtaatagagtgcgataagatggcagagcccggtaatagagtgcgataagatggcagagcccggtaatgactctctagagggtatataatgggggcc

SEQ ID NO: 387 - DNA, 115 bp, synthetic construct
  source 1..115: mol_type = other DNA
taagatggcagagcccggtaatagagtgcgagtaagatggcagagcccggtaatagagtgcgagtaagatggcagagcccggtaatgactctctagagggtatataatgggggcc

SEQ ID NO: 388 - DNA, 96 bp, synthetic construct
  source 1..96: mol_type = other DNA
taagatggcagagcccggtaattaagatggcagagcccggtaattaagatggcagagcccggtaatgactcctctagagggtatataatgggggcc

SEQ ID NO: 389 - DNA, 98 bp, synthetic construct
  source 1..98: mol_type = other DNA
taagatggcagagcccggtaatataagatggcagagcccggtaatataagatggcagagcccggtaatgactcctctagagggtatataatgggggcc

SEQ ID NO: 390 - DNA, 100 bp, synthetic construct
  source 1..100: mol_type = other DNA
taagatggcagagcccggtaatagtaagatggcagagcccggtaatagtaagatggcagagcccggtaatgactcctctagagggtatataatgggggcc

SEQ ID NO: 391 - DNA, 102 bp, synthetic construct
  source 1..102: mol_type = other DNA
taagatggcagagcccggtaatagataagatggcagagcccggtaatagataagatggcagagcccggtaatgactcctctagagggtatataatgggggcc

SEQ ID NO: 392 - DNA, 104 bp, synthetic construct
  source 1..104: mol_type = other DNA
taagatggcagagcccggtaatagagtaagatggcagagcccggtaatagagtaagatggcagagcccggtaatgactcctctagagggtatataatgggggcc

SEQ ID NO: 393 - DNA, 106 bp, synthetic construct
  source 1..106: mol_type = other DNA
taagatggcagagcccggtaatagagttaagatggcagagcccggtaatagagttaagatggcagagcccggtaatgactcctctagagggtatataatgggggcc

SEQ ID NO: 394 - DNA, 108 bp, synthetic construct
  source 1..108: mol_type = other DNA
taagatggcagagcccggtaatagagtgtaagatggcagagcccggtaatagagtgtaagatggcagagcccggtaatgactcctctagagggtatataatgggggcc

SEQ ID NO: 395 - DNA, 110 bp, synthetic construct
  source 1..110: mol_type = other DNA
taagatggcagagcccggtaatagagtgctaagatggcagagcccggtaatagagtgctaagatggcagagcccggtaatgactcctctagagggtatataatgggggcc

SEQ ID NO: 396 - DNA, 112 bp, synthetic construct
  source 1..112: mol_type = other DNA
taagatggcagagcccggtaatagagtgcgtaagatggcagagcccggtaatagagtgcgtaagatggcagagcccggtaatgactcctctagagggtatataatgggggcc

SEQ ID NO: 397 - DNA, 114 bp, synthetic construct
  source 1..114: mol_type = other DNA
taagatggcagagcccggtaatagagtgcgataagatggcagagcccggtaatagagtgcgataagatggcagagcccggtaatgactcctctagagggtatataatgggggcc

SEQ ID NO: 398 - DNA, 116 bp, synthetic construct
  source 1..116: mol_type = other DNA
taagatggcagagcccggtaatagagtgcgagtaagatggcagagcccggtaatagagtgcgagtaagatggcagagcccggtaatgactcctctagagggtatataatgggggcc

SEQ ID NO: 399 - DNA, 97 bp, synthetic construct
  source 1..97: mol_type = other DNA
taagatggcagagcccggtaattaagatggcagagcccggtaattaagatggcagagcccggtaatgactcactctagagggtatataatgggggcc

SEQ ID NO: 400 - DNA, 99 bp, synthetic construct
  source 1..99: mol_type = other DNA
taagatggcagagcccggtaatataagatggcagagcccggtaatataagatggcagagcccggtaatgactcactctagagggtatataatgggggcc

SEQ ID NO: 401 - DNA, 101 bp, synthetic construct
  source 1..101: mol_type = other DNA
taagatggcagagcccggtaatagtaagatggcagagcccggtaatagtaagatggcagagcccggtaatgactcactctagagggtatataatgggggcc

SEQ ID NO: 402 - DNA, 103 bp, synthetic construct
  source 1..103: mol_type = other DNA
taagatggcagagcccggtaatagataagatggcagagcccggtaatagataagatggcagagcccggtaatgactcactctagagggtatataatgggggcc

SEQ ID NO: 403 - DNA, 105 bp, synthetic construct
  source 1..105: mol_type = other DNA
taagatggcagagcccggtaatagagtaagatggcagagcccggtaatagagtaagatggcagagcccggtaatgactcactctagagggtatataatgggggcc

SEQ ID NO: 404 - DNA, 107 bp, synthetic construct
  source 1..107: mol_type = other DNA
taagatggcagagcccggtaatagagttaagatggcagagcccggtaatagagttaagatggcagagcccggtaatgactcactctagagggtatataatgggggcc

SEQ ID NO: 405 - DNA, 109 bp, synthetic construct
  source 1..109: mol_type = other DNA
taagatggcagagcccggtaatagagtgtaagatggcagagcccggtaatagagtgtaagatggcagagcccggtaatgactcactctagagggtatataatgggggcc

SEQ ID NO: 406 - DNA, 111 bp, synthetic construct
  source 1..111: mol_type = other DNA
taagatggcagagcccggtaatagagtgctaagatggcagagcccggtaatagagtgctaagatggcagagcccggtaatgactcactctagagggtatataatgggggcc

SEQ ID NO: 407 - DNA, 113 bp, synthetic construct
  source 1..113: mol_type = other DNA
taagatggcagagcccggtaatagagtgcgtaagatggcagagcccggtaatagagtgcgtaagatggcagagcccggtaatgactcactctagagggtatataatgggggcc

SEQ ID NO: 408 - DNA, 115 bp, synthetic construct
  source 1..115: mol_type = other DNA
taagatggcagagcccggtaatagagtgcgataagatggcagagcccggtaatagagtgcgataagatggcagagcccggtaatgactcactctagagggtatataatgggggcc

SEQ ID NO: 409 - DNA, 117 bp, synthetic construct
  source 1..117: mol_type = other DNA
taagatggcagagcccggtaatagagtgcgagtaagatggcagagcccggtaatagagtgcgagtaagatggcagagcccggtaatgactcactctagagggtatataatgggggcc

SEQ ID NO: 410 - DNA, 98 bp, synthetic construct
  source 1..98: mol_type = other DNA
taagatggcagagcccggtaattaagatggcagagcccggtaattaagatggcagagcccggtaatgactcatctctagagggtatataatgggggcc

SEQ ID NO: 411 - DNA, 100 bp, synthetic construct
  source 1..100: mol_type = other DNA
taagatggcagagcccggtaatataagatggcagagcccggtaatataagatggcagagcccggtaatgactcatctctagagggtatataatgggggcc

SEQ ID NO: 412 - DNA, 102 bp, synthetic construct
  source 1..102: mol_type = other DNA
taagatggcagagcccggtaatagtaagatggcagagcccggtaatagtaagatggcagagcccggtaatgactcatctctagagggtatataatgggggcc

SEQ ID NO: 413 - DNA, 104 bp, synthetic construct
  source 1..104: mol_type = other DNA
taagatggcagagcccggtaatagataagatggcagagcccggtaatagataagatggcagagcccggtaatgactcatctctagagggtatataatgggggcc

SEQ ID NO: 414 - DNA, 106 bp, synthetic construct
  source 1..106: mol_type = other DNA
taagatggcagagcccggtaatagagtaagatggcagagcccggtaatagagtaagatggcagagcccggtaatgactcatctctagagggtatataatgggggcc

SEQ ID NO: 415 - DNA, 108 bp, synthetic construct
  source 1..108: mol_type = other DNA
taagatggcagagcccggtaatagagttaagatggcagagcccggtaatagagttaagatggcagagcccggtaatgactcatctctagagggtatataatgggggcc

SEQ ID NO: 416 - DNA, 110 bp, synthetic construct
  source 1..110: mol_type = other DNA
taagatggcagagcccggtaatagagtgtaagatggcagagcccggtaatagagtgtaagatggcagagcccggtaatgactcatctctagagggtatataatgggggcc

SEQ ID NO: 417 - DNA, 112 bp, synthetic construct
  source 1..112: mol_type = other DNA
taagatggcagagcccggtaatagagtgctaagatggcagagcccggtaatagagtgctaagatggcagagcccggtaatgactcatctctagagggtatataatgggggcc

SEQ ID NO: 418 - DNA, 114 bp, synthetic construct
  source 1..114: mol_type = other DNA
taagatggcagagcccggtaatagagtgcgtaagatggcagagcccggtaatagagtgcgtaagatggcagagcccggtaatgactcatctctagagggtatataatgggggcc

SEQ ID NO: 419 - DNA, 116 bp, synthetic construct
  source 1..116: mol_type = other DNA
taagatggcagagcccggtaatagagtgcgataagatggcagagcccggtaatagagtgcgataagatggcagagcccggtaatgactcatctctagagggtatataatgggggcc

SEQ ID NO: 420 - DNA, 118 bp, synthetic construct
  source 1..118: mol_type = other DNA
taagatggcagagcccggtaatagagtgcgagtaagatggcagagcccggtaatagagtgcgagtaagatggcagagcccggtaatgactcatctctagagggtatataatgggggcc

SEQ ID NO: 421 - DNA, 99 bp, synthetic construct
  source 1..99: mol_type = other DNA
taagatggcagagcccggtaattaagatggcagagcccggtaattaagatggcagagcccggtaatgactcatactctagagggtatataatgggggcc

SEQ ID NO: 422 - DNA, 101 bp, synthetic construct
  source 1..101: mol_type = other DNA
taagatggcagagcccggtaatataagatggcagagcccggtaatataagatggcagagcccggtaatgactcatactctagagggtatataatgggggcc

SEQ ID NO: 423 - DNA, 103 bp, synthetic construct
  source 1..103: mol_type = other DNA
taagatggcagagcccggtaatagtaagatggcagagcccggtaatagtaagatggcagagcccggtaatgactcatactctagagggtatataatgggggcc

SEQ ID NO: 424 - DNA, 105 bp, synthetic construct
  source 1..105: mol_type = other DNA
taagatggcagagcccggtaatagataagatggcagagcccggtaatagataagatggcagagcccggtaatgactcatactctagagggtatataatgggggcc

SEQ ID NO: 425 - DNA, 107 bp, synthetic construct
  source 1..107: mol_type = other DNA
taagatggcagagcccggtaatagagtaagatggcagagcccggtaatagagtaagatggcagagcccggtaatgactcatactctagagggtatataatgggggcc

SEQ ID NO: 426 - DNA, 109 bp, synthetic construct
  source 1..109: mol_type = other DNA
taagatggcagagcccggtaatagagttaagatggcagagcccggtaatagagttaagatggcagagcccggtaatgactcatactctagagggtatataatgggggcc

SEQ ID NO: 427 - DNA, 111 bp, synthetic construct
  source 1..111: mol_type = other DNA
taagatggcagagcccggtaatagagtgtaagatggcagagcccggtaatagagtgtaagatggcagagcccggtaatgactcatactctagagggtatataatgggggcc

SEQ ID NO: 428 - DNA, 113 bp, synthetic construct
  source 1..113: mol_type = other DNA
taagatggcagagcccggtaatagagtgctaagatggcagagcccggtaatagagtgctaagatggcagagcccggtaatgactcatactctagagggtatataatgggggcc

SEQ ID NO: 429 - DNA, 115 bp, synthetic construct
  source 1..115: mol_type = other DNA
taagatggcagagcccggtaatagagtgcgtaagatggcagagcccggtaatagagtgcgtaagatggcagagcccggtaatgactcatactctagagggtatataatgggggcc

SEQ ID NO: 430 - DNA, 117 bp, synthetic construct
  source 1..117: mol_type = other DNA
taagatggcagagcccggtaatagagtgcgataagatggcagagcccggtaatagagtgcgataagatggcagagcccggtaatgactcatactctagagggtatataatgggggcc

SEQ ID NO: 431 - DNA, 119 bp, synthetic construct
  source 1..119: mol_type = other DNA
taagatggcagagcccggtaatagagtgcgagtaagatggcagagcccggtaatagagtgcgagtaagatggcagagcccggtaatgactcatactctagagggtatataatgggggcc

SEQ ID NO: 432 - DNA, 100 bp, synthetic construct
  source 1..100: mol_type = other DNA
taagatggcagagcccggtaattaagatggcagagcccggtaattaagatggcagagcccggtaatgactcatatctctagagggtatataatgggggcc

SEQ ID NO: 433 - DNA, 102 bp, synthetic construct
  source 1..102: mol_type = other DNA
taagatggcagagcccggtaatataagatggcagagcccggtaatataagatggcagagcccggtaatgactcatatctctagagggtatataatgggggcc

SEQ ID NO: 434 - DNA, 104 bp, synthetic construct
  source 1..104: mol_type = other DNA
taagatggcagagcccggtaatagtaagatggcagagcccggtaatagtaagatggcagagcccggtaatgactcatatctctagagggtatataatgggggcc

SEQ ID NO: 435 - DNA, 106 bp, synthetic construct
  source 1..106: mol_type = other DNA
taagatggcagagcccggtaatagataagatggcagagcccggtaatagataagatggcagagcccggtaatgactcatatctctagagggtatataatgggggcc

SEQ ID NO: 436 - DNA, 108 bp, synthetic construct
  source 1..108: mol_type = other DNA
taagatggcagagcccggtaatagagtaagatggcagagcccggtaatagagtaagatggcagagcccggtaatgactcatatctctagagggtatataatgggggcc

SEQ ID NO: 437 - DNA, 110 bp, synthetic construct
  source 1..110: mol_type = other DNA
taagatggcagagcccggtaatagagttaagatggcagagcccggtaatagagttaagatggcagagcccggtaatgactcatatctctagagggtatataatgggggcc

SEQ ID NO: 438 - DNA, 112 bp, synthetic construct
  source 1..112: mol_type = other DNA
taagatggcagagcccggtaatagagtgtaagatggcagagcccggtaatagagtgtaagatggcagagcccggtaatgactcatatctctagagggtatataatgggggcc

SEQ ID NO: 439 - DNA, 114 bp, synthetic construct
  source 1..114: mol_type = other DNA
taagatggcagagcccggtaatagagtgctaagatggcagagcccggtaatagagtgctaagatggcagagcccggtaatgactcatatctctagagggtatataatgggggcc

SEQ ID NO: 440 - DNA, 116 bp, synthetic construct
  source 1..116: mol_type = other DNA
taagatggcagagcccggtaatagagtgcgtaagatggcagagcccggtaatagagtgcgtaagatggcagagcccggtaatgactcatatctctagagggtatataatgggggcc

SEQ ID NO: 441 - DNA, 118 bp, synthetic construct
  source 1..118: mol_type = other DNA
taagatggcagagcccggtaatagagtgcgataagatggcagagcccggtaatagagtgcgataagatggcagagcccggtaatgactcatatctctagagggtatataatgggggcc

SEQ ID NO: 442 - DNA, 120 bp, synthetic construct
  source 1..120: mol_type = other DNA
taagatggcagagcccggtaatagagtgcgagtaagatggcagagcccggtaatagagtgcgagtaagatggcagagcccggtaatgactcatatctctagagggtatataatgggggcc

SEQ ID NO: 443 - DNA, 101 bp, synthetic construct
  source 1..101: mol_type = other DNA
taagatggcagagcccggtaattaagatggcagagcccggtaattaagatggcagagcccggtaatgactcatatgctctagagggtatataatgggggcc

SEQ ID NO: 444 - DNA, 103 bp, synthetic construct
  source 1..103: mol_type = other DNA
taagatggcagagcccggtaatataagatggcagagcccggtaatataagatggcagagcccggtaatgactcatatgctctagagggtatataatgggggcc

SEQ ID NO: 445 - DNA, 105 bp, synthetic construct
  source 1..105: mol_type = other DNA
taagatggcagagcccggtaatagtaagatggcagagcccggtaatagtaagatggcagagcccggtaatgactcatatgctctagagggtatataatgggggcc

SEQ ID NO: 446 - DNA, 107 bp, synthetic construct
  source 1..107: mol_type = other DNA
taagatggcagagcccggtaatagataagatggcagagcccggtaatagataagatggcagagcccggtaatgactcatatgctctagagggtatataatgggggcc

SEQ ID NO: 447 - DNA, 109 bp, synthetic construct
  source 1..109: mol_type = other DNA
taagatggcagagcccggtaatagagtaagatggcagagcccggtaatagagtaagatggcagagcccggtaatgactcatatgctctagagggtatataatgggggcc

SEQ ID NO: 448 - DNA, 111 bp, synthetic construct
  source 1..111: mol_type = other DNA
taagatggcagagcccggtaatagagttaagatggcagagcccggtaatagagttaagatggcagagcccggtaatgactcatatgctctagagggtatataatgggggcc

SEQ ID NO: 449 - DNA, 113 bp, synthetic construct
  source 1..113: mol_type = other DNA
taagatggcagagcccggtaatagagtgtaagatggcagagcccggtaatagagtgtaagatggcagagcccggtaatgactcatatgctctagagggtatataatgggggcc

SEQ ID NO: 450 - DNA, 115 bp, synthetic construct
  source 1..115: mol_type = other DNA
taagatggcagagcccggtaatagagtgctaagatggcagagcccggtaatagagtgctaagatggcagagcccggtaatgactcatatgctctagagggtatataatgggggcc

SEQ ID NO: 451 - DNA, 117 bp, synthetic construct
  source 1..117: mol_type = other DNA
taagatggcagagcccggtaatagagtgcgtaagatggcagagcccggtaatagagtgcgtaagatggcagagcccggtaatgactcatatgctctagagggtatataatgggggcc

SEQ ID NO: 452 - DNA, 119 bp, synthetic construct
  source 1..119: mol_type = other DNA
taagatggcagagcccggtaatagagtgcgataagatggcagagcccggtaatagagtgcgataagatggcagagcccggtaatgactcatatgctctagagggtatataatgggggcc

SEQ ID NO: 453 - DNA, 121 bp, synthetic construct
  source 1..121: mol_type = other DNA
taagatggcagagcccggtaatagagtgcgagtaagatggcagagcccggtaatagagtgcgagtaagatggcagagcccggtaatgactcatatgctctagagggtatataatgggggcc

SEQ ID NO: 454 - DNA, 113 bp, synthetic construct
  source 1..113: mol_type = other DNA
taagatggcagagcccggtaattaagatggcagagcccggtaattaagatggcagagcccggtaattaagatggcagagcccggtaatctctagagggtatataatgggggcc

SEQ ID NO: 455 - DNA, 116 bp, synthetic construct
  source 1..116: mol_type = other DNA
taagatggcagagcccggtaatataagatggcagagcccggtaatataagatggcagagcccggtaatataagatggcagagcccggtaatctctagagggtatataatgggggcc

SEQ ID NO: 456 - DNA, 119 bp, synthetic construct
  source 1..119: mol_type = other DNA
taagatggcagagcccggtaatagtaagatggcagagcccggtaatagtaagatggcagagcccggtaatagtaagatggcagagcccggtaatctctagagggtatataatgggggcc

SEQ ID NO: 457 - DNA, 122 bp, synthetic construct
  source 1..122: mol_type = other DNA
taagatggcagagcccggtaatagataagatggcagagcccggtaatagataagatggcagagcccggtaatagataagatggcagagcccggtaatctctagagggtatataatgggggcc

SEQ ID NO: 458 - DNA, 125 bp, synthetic construct
  source 1..125: mol_type = other DNA
taagatggcagagcccggtaatagagtaagatggcagagcccggtaatagagtaagatggcagagcccggtaatagagtaagatggcagagcccggtaatctctagagggtatataatgggggcc

SEQ ID NO: 459 - DNA, 128 bp, synthetic construct
  source 1..128: mol_type = other DNA
taagatggcagagcccggtaatagagttaagatggcagagcccggtaatagagttaagatggcagagcccggtaatagagttaagatggcagagcccggtaatctctagagggtatataatgggggcc

SEQ ID NO: 460 - DNA, 131 bp, synthetic construct
  source 1..131: mol_type = other DNA
taagatggcagagcccggtaatagagtgtaagatggcagagcccggtaatagagtgtaagatggcagagcccggtaatagagtgtaagatggcagagcccggtaatctctagagggtatataatgggggcc

SEQ ID NO: 461 - DNA, 134 bp, synthetic construct
  source 1..134: mol_type = other DNA
taagatggcagagcccggtaatagagtgctaagatggcagagcccggtaatagagtgctaagatggcagagcccggtaatagagtgctaagatggcagagcccggtaatctctagagggtatataatgggggcc

SEQ ID NO: 462 - DNA, 137 bp, synthetic construct
  source 1..137: mol_type = other DNA
taagatggcagagcccggtaatagagtgcgtaagatggcagagcccggtaatagagtgcgtaagatggcagagcccggtaatagagtgcgtaagatggcagagcccggtaatctctagagggtatataatgggggcc

SEQ ID NO: 463 - DNA, 140 bp, synthetic construct
  source 1..140: mol_type = other DNA
taagatggcagagcccggtaatagagtgcgataagatggcagagcccggtaatagagtgcgataagatggcagagcccggtaatagagtgcgataagatggcagagcccggtaatctctagagggtatataatgggggcc

SEQ ID NO: 464 - DNA, 143 bp, synthetic construct
  source 1..143: mol_type = other DNA
taagatggcagagcccggtaatagagtgcgagtaagatggcagagcccggtaatagagtgcgagtaagatggcagagcccggtaatagagtgcgagtaagatggcagagcccggtaatctctagagggtatataatgggggcc

SEQ ID NO: 465 - DNA, 114 bp, synthetic construct
  source 1..114: mol_type = other DNA
taagatggcagagcccggtaattaagatggcagagcccggtaattaagatggcagagcccggtaattaagatggcagagcccggtaatgctctagagggtatataatgggggcc

SEQ ID NO: 466 - DNA, 117 bp, synthetic construct
  source 1..117: mol_type = other DNA
taagatggcagagcccggtaatataagatggcagagcccggtaatataagatggcagagcccggtaatataagatggcagagcccggtaatgctctagagggtatataatgggggcc

SEQ ID NO: 467 - DNA, 120 bp, synthetic construct
  source 1..120: mol_type = other DNA
taagatggcagagcccggtaatagtaagatggcagagcccggtaatagtaagatggcagagcccggtaatagtaagatggcagagcccggtaatgctctagagggtatataatgggggcc

SEQ ID NO: 468 - DNA, 123 bp, synthetic construct
  source 1..123: mol_type = other DNA
taagatggcagagcccggtaatagataagatggcagagcccggtaatagataagatggcagagcccggtaatagataagatggcagagcccggtaatgctctagagggtatataatgggggcc

SEQ ID NO: 469 - DNA, 126 bp, synthetic construct
  source 1..126: mol_type = other DNA
taagatggcagagcccggtaatagagtaagatggcagagcccggtaatagagtaagatggcagagcccggtaatagagtaagatggcagagcccggtaatgctctagagggtatataatgggggcc

SEQ ID NO: 470 - DNA, 129 bp, synthetic construct
  source 1..129: mol_type = other DNA
taagatggcagagcccggtaatagagttaagatggcagagcccggtaatagagttaagatggcagagcccggtaatagagttaagatggcagagcccggtaatgctctagagggtatataatgggggcc

SEQ ID NO: 471 - DNA, 132 bp, synthetic construct
  source 1..132: mol_type = other DNA
taagatggcagagcccggtaatagagtgtaagatggcagagcccggtaatagagtgtaagatggcagagcccggtaatagagtgtaagatggcagagcccggtaatgctctagagggtatataatgggggcc

SEQ ID NO: 472 - DNA, 135 bp, synthetic construct
  source 1..135: mol_type = other DNA
taagatggcagagcccggtaatagagtgctaagatggcagagcccggtaatagagtgctaagatggcagagcccggtaatagagtgctaagatggcagagcccggtaatgctctagagggtatataatgggggcc

SEQ ID NO: 473 - DNA, 138 bp, synthetic construct
  source 1..138: mol_type = other DNA
taagatggcagagcccggtaatagagtgcgtaagatggcagagcccggtaatagagtgcgtaagatggcagagcccggtaatagagtgcgtaagatggcagagcccggtaatgctctagagggtatataatgggggcc

SEQ ID NO: 474 - DNA, 141 bp, synthetic construct
  source 1..141: mol_type = other DNA
taagatggcagagcccggtaatagagtgcgataagatggcagagcccggtaatagagtgcgataagatggcagagcccggtaatagagtgcgataagatggcagagcccggtaatgctctagagggtatataatgggggcc

SEQ ID NO: 475 - DNA, 144 bp, synthetic construct
  source 1..144: mol_type = other DNA
taagatggcagagcccggtaatagagtgcgagtaagatggcagagcccggtaatagagtgcgagtaagatggcagagcccggtaatagagtgcgagtaagatggcagagcccggtaatgctctagagggtatataatgggggcc

SEQ ID NO: 476 - DNA, 115 bp, synthetic construct
  source 1..115: mol_type = other DNA
taagatggcagagcccggtaattaagatggcagagcccggtaattaagatggcagagcccggtaattaagatggcagagcccggtaatgactctagagggtatataatgggggcc

SEQ ID NO: 477 - DNA, 118 bp, synthetic construct
  source 1..118: mol_type = other DNA
taagatggcagagcccggtaatataagatggcagagcccggtaatataagatggcagagcccggtaatataagatggcagagcccggtaatgactctagagggtatataatgggggcc

SEQ ID NO: 478 - DNA, 121 bp, synthetic construct
  source 1..121: mol_type = other DNA
taagatggcagagcccggtaatagtaagatggcagagcccggtaatagtaagatggcagagcccggtaatagtaagatggcagagcccggtaatgactctagagggtatataatgggggcc

SEQ ID NO: 479 - DNA, 124 bp, synthetic construct
  source 1..124: mol_type = other DNA
taagatggcagagcccggtaatagataagatggcagagcccggtaatagataagatggcagagcccggtaatagataagatggcagagcccggtaatgactctagagggtatataatgggggcc

SEQ ID NO: 480 - DNA, 127 bp, synthetic construct
  source 1..127: mol_type = other DNA
taagatggcagagcccggtaatagagtaagatggcagagcccggtaatagagtaagatggcagagcccggtaatagagtaagatggcagagcccggtaatgactctagagggtatataatgggggcc

SEQ ID NO: 481 - DNA, 130 bp, synthetic construct
  source 1..130: mol_type = other DNA
taagatggcagagcccggtaatagagttaagatggcagagcccggtaatagagttaagatggcagagcccggtaatagagttaagatggcagagcccggtaatgactctagagggtatataatgggggcc

SEQ ID NO: 482 - DNA, 133 bp, synthetic construct
  source 1..133: mol_type = other DNA
taagatggcagagcccggtaatagagtgtaagatggcagagcccggtaatagagtgtaagatggcagagcccggtaatagagtgtaagatggcagagcccggtaatgactctagagggtatataatgggggcc

SEQ ID NO: 483 - DNA, 136 bp, synthetic construct
  source 1..136: mol_type = other DNA
taagatggcagagcccggtaatagagtgctaagatggcagagcccggtaatagagtgctaagatggcagagcccggtaatagagtgctaagatggcagagcccggtaatgactctagagggtatataatgggggcc

SEQ ID NO: 484 - DNA, 139 bp, synthetic construct
  source 1..139: mol_type = other DNA
taagatggcagagcccggtaatagagtgcgtaagatggcagagcccggtaatagagtgcgtaagatggcagagcccggtaatagagtgcgtaagatggcagagcccggtaatgactctagagggtatataatgggggcc

SEQ ID NO: 485 - DNA, 142 bp, synthetic construct
  source 1..142: mol_type = other DNA
taagatggcagagcccggtaatagagtgcgataagatggcagagcccggtaatagagtgcgataagatggcagagcccggtaatagagtgcgataagatggcagagcccggtaatgactctagagggtatataatgggggcc

SEQ ID NO: 486 - DNA, 145 bp, synthetic construct
  source 1..145: mol_type = other DNA
taagatggcagagcccggtaatagagtgcgagtaagatggcagagcccggtaatagagtgcgagtaagatggcagagcccggtaatagagtgcgagtaagatggcagagcccggtaatgactctagagggtatataatgggggcc

SEQ ID NO: 487 - DNA, 116 bp, synthetic construct
  source 1..116: mol_type = other DNA
taagatggcagagcccggtaattaagatggcagagcccggtaattaagatggcagagcccggtaattaagatggcagagcccggtaatgacctctagagggtatataatgggggcc

SEQ ID NO: 488 - DNA, 119 bp, synthetic construct
  source 1..119: mol_type = other DNA
taagatggcagagcccggtaatataagatggcagagcccggtaatataagatggcagagcccggtaatataagatggcagagcccggtaatgacctctagagggtatataatgggggcc

SEQ ID NO: 489 - DNA, 122 bp, synthetic construct
  source 1..122: mol_type = other DNA
taagatggcagagcccggtaatagtaagatggcagagcccggtaatagtaagatggcagagcccggtaatagtaagatggcagagcccggtaatgacctctagagggtatataatgggggcc

SEQ ID NO: 490 - DNA, 125 bp, synthetic construct
  source 1..125: mol_type = other DNA
taagatggcagagcccggtaatagataagatggcagagcccggtaatagataagatggcagagcccggtaatagataagatggcagagcccggtaatgacctctagagggtatataatgggggcc

SEQ ID NO: 491 - DNA, 128 bp, synthetic construct
  source 1..128: mol_type = other DNA
taagatggcagagcccggtaatagagtaagatggcagagcccggtaatagagtaagatggcagagcccggtaatagagtaagatggcagagcccggtaatgacctctagagggtatataatgggggcc

SEQ ID NO: 492 - DNA, 131 bp, synthetic construct
  source 1..131: mol_type = other DNA
taagatggcagagcccggtaatagagttaagatggcagagcccggtaatagagttaagatggcagagcccggtaatagagttaagatggcagagcccggtaatgacctctagagggtatataatgggggcc

SEQ ID NO: 493 - DNA, 134 bp, synthetic construct
  source 1..134: mol_type = other DNA
taagatggcagagcccggtaatagagtgtaagatggcagagcccggtaatagagtgtaagatggcagagcccggtaatagagtgtaagatggcagagcccggtaatgacctctagagggtatataatgggggcc

SEQ ID NO: 494 - DNA, 137 bp, synthetic construct
  source 1..137: mol_type = other DNA
taagatggcagagcccggtaatagagtgctaagatggcagagcccggtaatagagtgctaagatggcagagcccggtaatagagtgctaagatggcagagcccggtaatgacctctagagggtatataatgggggcc

SEQ ID NO: 495 - DNA, 140 bp, synthetic construct
  source 1..140: mol_type = other DNA
taagatggcagagcccggtaatagagtgcgtaagatggcagagcccggtaatagagtgcgtaagatggcagagcccggtaatagagtgcgtaagatggcagagcccggtaatgacctctagagggtatataatgggggcc

SEQ ID NO: 496 - DNA, 143 bp, synthetic construct
  source 1..143: mol_type = other DNA
taagatggcagagcccggtaatagagtgcgataagatggcagagcccggtaatagagtgcgataagatggcagagcccggtaatagagtgcgataagatggcagagcccggtaatgacctctagagggtatataatgggggcc

SEQ ID NO: 497 - DNA, 146 bp, synthetic construct
  source 1..146: mol_type = other DNA
taagatggcagagcccggtaatagagtgcgagtaagatggcagagcccggtaatagagtgcgagtaagatggcagagcccggtaatagagtgcgagtaagatggcagagcccggtaatgacctctagagggtatataatgggggcc

SEQ ID NO: 498 - DNA, 117 bp, synthetic construct
  source 1..117: mol_type = other DNA
taagatggcagagcccggtaattaagatggcagagcccggtaattaagatggcagagcccggtaattaagatggcagagcccggtaatgactctctagagggtatataatgggggcc

SEQ ID NO: 499 - DNA, 120 bp, synthetic construct
  source 1..120: mol_type = other DNA
taagatggcagagcccggtaatataagatggcagagcccggtaatataagatggcagagcccggtaatataagatggcagagcccggtaatgactctctagagggtatataatgggggcc

SEQ ID NO: 500 - DNA, 123 bp, synthetic construct
  source 1..123: mol_type = other DNA
taagatggcagagcccggtaatagtaagatggcagagcccggtaatagtaagatggcagagcccggtaatagtaagatggcagagcccggtaatgactctctagagggtatataatgggggcc

SEQ ID NO: 501 - DNA, 126 bp, synthetic construct
  source 1..126: mol_type = other DNA
taagatggcagagcccggtaatagataagatggcagagcccggtaatagataagatggcagagcccggtaatagataagatggcagagcccggtaatgactctctagagggtatataatgggggcc

SEQ ID NO: 502 - DNA, 129 bp, synthetic construct
  source 1..129: mol_type = other DNA
taagatggcagagcccggtaatagagtaagatggcagagcccggtaatagagtaagatggcagagcccggtaatagagtaagatggcagagcccggtaatgactctctagagggtatataatgggggcc

SEQ ID NO: 503 - DNA, 132 bp, synthetic construct
  source 1..132: mol_type = other DNA
taagatggcagagcccggtaatagagttaagatggcagagcccggtaatagagttaagatggcagagcccggtaatagagttaagatggcagagcccggtaatgactctctagagggtatataatgggggcc

SEQ ID NO: 504 - DNA, 135 bp, synthetic construct
  source 1..135: mol_type = other DNA
taagatggcagagcccggtaatagagtgtaagatggcagagcccggtaatagagtgtaagatggcagagcccggtaatagagtgtaagatggcagagcccggtaatgactctctagagggtatataatgggggcc

SEQ ID NO: 505 - DNA, 138 bp, synthetic construct
  source 1..138: mol_type = other DNA
taagatggcagagcccggtaatagagtgctaagatggcagagcccggtaatagagtgctaagatggcagagcccggtaatagagtgctaagatggcagagcccggtaatgactctctagagggtatataatgggggcc

SEQ ID NO: 506 - DNA, 141 bp, synthetic construct
  source 1..141: mol_type = other DNA
taagatggcagagcccggtaatagagtgcgtaagatggcagagcccggtaatagagtgcgtaagatggcagagcccggtaatagagtgcgtaagatggcagagcccggtaatgactctctagagggtatataatgggggcc

SEQ ID NO: 507 - DNA, 144 bp, synthetic construct
  source 1..144: mol_type = other DNA
taagatggcagagcccggtaatagagtgcgataagatggcagagcccggtaatagagtgcgataagatggcagagcccggtaatagagtgcgataagatggcagagcccggtaatgactctctagagggtatataatgggggcc

SEQ ID NO: 508 - DNA, 147 bp, synthetic construct
  source 1..147: mol_type = other DNA
taagatggcagagcccggtaatagagtgcgagtaagatggcagagcccggtaatagagtgcgagtaagatggcagagcccggtaatagagtgcgagtaagatggcagagcccggtaatgactctctagagggtatataatgggggcc

SEQ ID NO: 509 - DNA, 118 bp, synthetic construct
  source 1..118: mol_type = other DNA
taagatggcagagcccggtaattaagatggcagagcccggtaattaagatggcagagcccggtaattaagatggcagagcccggtaatgactcctctagagggtatataatgggggcc

SEQ ID NO: 510 - DNA, 121 bp, synthetic construct
  source 1..121: mol_type = other DNA
taagatggcagagcccggtaatataagatggcagagcccggtaatataagatggcagagcccggtaatataagatggcagagcccggtaatgactcctctagagggtatataatgggggcc

SEQ ID NO: 511 - DNA, 124 bp, synthetic construct
  source 1..124: mol_type = other DNA
taagatggcagagcccggtaatagtaagatggcagagcccggtaatagtaagatggcagagcccggtaatagtaagatggcagagcccggtaatgactcctctagagggtatataatgggggcc

SEQ ID NO: 512 - DNA, 127 bp, synthetic construct
  source 1..127: mol_type = other DNA
taagatggcagagcccggtaatagataagatggcagagcccggtaatagataagatggcagagcccggtaatagataagatggcagagcccggtaatgactcctctagagggtatataatgggggcc

SEQ ID NO: 513 - DNA, 130 bp, synthetic construct
  source 1..130: mol_type = other DNA
taagatggcagagcccggtaatagagtaagatggcagagcccggtaatagagtaagatggcagagcccggtaatagagtaagatggcagagcccggtaatgactcctctagagggtatataatgggggcc

SEQ ID NO: 514 - DNA, 133 bp, synthetic construct
  source 1..133: mol_type = other DNA
taagatggcagagcccggtaatagagttaagatggcagagcccggtaatagagttaagatggcagagcccggtaatagagttaagatggcagagcccggtaatgactcctctagagggtatataatgggggcc

SEQ ID NO: 515 - DNA, 136 bp, synthetic construct
  source 1..136: mol_type = other DNA
taagatggcagagcccggtaatagagtgtaagatggcagagcccggtaatagagtgtaagatggcagagcccggtaatagagtgtaagatggcagagcccggtaatgactcctctagagggtatataatgggggcc

SEQ ID NO: 516 - DNA, 139 bp, synthetic construct
  source 1..139: mol_type = other DNA
taagatggcagagcccggtaatagagtgctaagatggcagagcccggtaatagagtgctaagatggcagagcccggtaatagagtgctaagatggcagagcccggtaatgactcctctagagggtatataatgggggcc

SEQ ID NO: 517 - DNA, 142 bp, synthetic construct
  source 1..142: mol_type = other DNA
taagatggcagagcccggtaatagagtgcgtaagatggcagagcccggtaatagagtgcgtaagatggcagagcccggtaatagagtgcgtaagatggcagagcccggtaatgactcctctagagggtatataatgggggcc

SEQ ID NO: 518 - DNA, 145 bp, synthetic construct
  source 1..145: mol_type = other DNA
taagatggcagagcccggtaatagagtgcgataagatggcagagcccggtaatagagtgcgataagatggcagagcccggtaatagagtgcgataagatggcagagcccggtaatgactcctctagagggtatataatgggggcc

SEQ ID NO: 519 - DNA, 148 bp, synthetic construct
  source 1..148: mol_type = other DNA
taagatggcagagcccggtaatagagtgcgagtaagatggcagagcccggtaatagagtgcgagtaagatggcagagcccggtaatagagtgcgagtaagatggcagagcccggtaatgactcctctagagggtatataatgggggcc

SEQ ID NO: 520 - DNA, 119 bp, synthetic construct
  source 1..119: mol_type = other DNA
taagatggcagagcccggtaattaagatggcagagcccggtaattaagatggcagagcccggtaattaagatggcagagcccggtaatgactcactctagagggtatataatgggggcc

SEQ ID NO: 521 - DNA, 122 bp, synthetic construct
  source 1..122: mol_type = other DNA
taagatggcagagcccggtaatataagatggcagagcccggtaatataagatggcagagcccggtaatataagatggcagagcccggtaatgactcactctagagggtatataatgggggcc

SEQ ID NO: 522 - DNA, 125 bp, synthetic construct
  source 1..125: mol_type = other DNA
taagatggcagagcccggtaatagtaagatggcagagcccggtaatagtaagatggcagagcccggtaatagtaagatggcagagcccggtaatgactcactctagagggtatataatgggggcc

SEQ ID NO: 523 - DNA, 128 bp, synthetic construct
  source 1..128: mol_type = other DNA
taagatggcagagcccggtaatagataagatggcagagcccggtaatagataagatggcagagcccggtaatagataagatggcagagcccggtaatgactcactctagagggtatataatgggggcc

SEQ ID NO: 524 - DNA, 131 bp, synthetic construct
  source 1..131: mol_type = other DNA
taagatggcagagcccggtaatagagtaagatggcagagcccggtaatagagtaagatggcagagcccggtaatagagtaagatggcagagcccggtaatgactcactctagagggtatataatgggggcc

SEQ ID NO: 525 - DNA, 134 bp, synthetic construct
  source 1..134: mol_type = other DNA
taagatggcagagcccggtaatagagttaagatggcagagcccggtaatagagttaagatggcagagcccggtaatagagttaagatggcagagcccggtaatgactcactctagagggtatataatgggggcc

SEQ ID NO: 526 - DNA, 137 bp, synthetic construct
  source 1..137: mol_type = other DNA
taagatggcagagcccggtaatagagtgtaagatggcagagcccggtaatagagtgtaagatggcagagcccggtaatagagtgtaagatggcagagcccggtaatgactcactctagagggtatataatgggggcc

SEQ ID NO: 527 - DNA, 140 bp, synthetic construct
  source 1..140: mol_type = other DNA
taagatggcagagcccggtaatagagtgctaagatggcagagcccggtaatagagtgctaagatggcagagcccggtaatagagtgctaagatggcagagcccggtaatgactcactctagagggtatataatgggggcc

SEQ ID NO: 528 - DNA, 143 bp, synthetic construct
  source 1..143: mol_type = other DNA
taagatggcagagcccggtaatagagtgcgtaagatggcagagcccggtaatagagtgcgtaagatggcagagcccggtaatagagtgcgtaagatggcagagcccggtaatgactcactctagagggtatataatgggggcc

SEQ ID NO: 529 - DNA, 146 bp, synthetic construct
  source 1..146: mol_type = other DNA
taagatggcagagcccggtaatagagtgcgataagatggcagagcccggtaatagagtgcgataagatggcagagcccggtaatagagtgcgataagatggcagagcccggtaatgactcactctagagggtatataatgggggcc

SEQ ID NO: 530 - DNA, 149 bp, synthetic construct
  source 1..149: mol_type = other DNA
taagatggcagagcccggtaatagagtgcgagtaagatggcagagcccggtaatagagtgcgagtaagatggcagagcccggtaatagagtgcgagtaagatggcagagcccggtaatgactcactctagagggtatataatgggggcc

SEQ ID NO: 531 - DNA, 120 bp, synthetic construct
  source 1..120: mol_type = other DNA
taagatggcagagcccggtaattaagatggcagagcccggtaattaagatggcagagcccggtaattaagatggcagagcccggtaatgactcatctctagagggtatataatgggggcc

SEQ ID NO: 532 - DNA, 123 bp, synthetic construct
  source 1..123: mol_type = other DNA
taagatggcagagcccggtaatataagatggcagagcccggtaatataagatggcagagcccggtaatataagatggcagagcccggtaatgactcatctctagagggtatataatgggggcc

SEQ ID NO: 533 - DNA, 126 bp, synthetic construct
  source 1..126: mol_type = other DNA
taagatggcagagcccggtaatagtaagatggcagagcccggtaatagtaagatggcagagcccggtaatagtaagatggcagagcccggtaatgactcatctctagagggtatataatgggggcc

SEQ ID NO: 534 - DNA, 129 bp, synthetic construct
  source 1..129: mol_type = other DNA
taagatggcagagcccggtaatagataagatggcagagcccggtaatagataagatggcagagcccggtaatagataagatggcagagcccggtaatgactcatctctagagggtatataatgggggcc

SEQ ID NO: 535 - DNA, 132 bp, synthetic construct
  source 1..132: mol_type = other DNA
taagatggcagagcccggtaatagagtaagatggcagagcccggtaatagagtaagatggcagagcccggtaatagagtaagatggcagagcccggtaatgactcatctctagagggtatataatgggggcc

SEQ ID NO: 536 - DNA, 135 bp, synthetic construct
  source 1..135: mol_type = other DNA
taagatggcagagcccggtaatagagttaagatggcagagcccggtaatagagttaagatggcagagcccggtaatagagttaagatggcagagcccggtaatgactcatctctagagggtatataatgggggcc

SEQ ID NO: 537 - DNA, 138 bp, synthetic construct
  source 1..138: mol_type = other DNA
taagatggcagagcccggtaatagagtgtaagatggcagagcccggtaatagagtgtaagatggcagagcccggtaatagagtgtaagatggcagagcccggtaatgactcatctctagagggtatataatgggggcc

SEQ ID NO: 538 - DNA, 141 bp, synthetic construct
  source 1..141: mol_type = other DNA
taagatggcagagcccggtaatagagtgctaagatggcagagcccggtaatagagtgctaagatggcagagcccggtaatagagtgctaagatggcagagcccggtaatgactcatctctagagggtatataatgggggcc

SEQ ID NO: 539 - DNA, 144 bp, synthetic construct
  source 1..144: mol_type = other DNA
taagatggcagagcccggtaatagagtgcgtaagatggcagagcccggtaatagagtgcgtaagatggcagagcccggtaatagagtgcgtaagatggcagagcccggtaatgactcatctctagagggtatataatgggggcc

SEQ ID NO: 540 - DNA, 147 bp, synthetic construct
  source 1..147: mol_type = other DNA
taagatggcagagcccggtaatagagtgcgataagatggcagagcccggtaatagagtgcgataagatggcagagcccggtaatagagtgcgataagatggcagagcccggtaatgactcatctctagagggtatataatgggggcc

SEQ ID NO: 541 - DNA, 150 bp, synthetic construct
  source 1..150: mol_type = other DNA
taagatggcagagcccggtaatagagtgcgagtaagatggcagagcccggtaatagagtgcgagtaagatggcagagcccggtaatagagtgcgagtaagatggcagagcccggtaatgactcatctctagagggtatataatgggggcc

SEQ ID NO: 542 - DNA, 121 bp, synthetic construct
  source 1..121: mol_type = other DNA
taagatggcagagcccggtaattaagatggcagagcccggtaattaagatggcagagcccggtaattaagatggcagagcccggtaatgactcatactctagagggtatataatgggggcc

SEQ ID NO: 543 - DNA, 124 bp, synthetic construct
  source 1..124: mol_type = other DNA
taagatggcagagcccggtaatataagatggcagagcccggtaatataagatggcagagcccggtaatataagatggcagagcccggtaatgactcatactctagagggtatataatgggggcc

SEQ ID NO: 544 - DNA, 127 bp, synthetic construct
  source 1..127: mol_type = other DNA
taagatggcagagcccggtaatagtaagatggcagagcccggtaatagtaagatggcagagcccggtaatagtaagatggcagagcccggtaatgactcatactctagagggtatataatgggggcc

SEQ ID NO: 545 - DNA, 130 bp, synthetic construct
  source 1..130: mol_type = other DNA
taagatggcagagcccggtaatagataagatggcagagcccggtaatagataagatggcagagcccggtaatagataagatggcagagcccggtaatgactcatactctagagggtatataatgggggcc

SEQ ID NO: 546 - DNA, 133 bp, synthetic construct
  source 1..133: mol_type = other DNA
taagatggcagagcccggtaatagagtaagatggcagagcccggtaatagagtaagatggcagagcccggtaatagagtaagatggcagagcccggtaatgactcatactctagagggtatataatgggggcc

SEQ ID NO: 547 - DNA, 136 bp, synthetic construct
  source 1..136: mol_type = other DNA
taagatggcagagcccggtaatagagttaagatggcagagcccggtaatagagttaagatggcagagcccggtaatagagttaagatggcagagcccggtaatgactcatactctagagggtatataatgggggcc

SEQ ID NO: 548 - DNA, 139 bp, synthetic construct
  source 1..139: mol_type = other DNA
taagatggcagagcccggtaatagagtgtaagatggcagagcccggtaatagagtgtaagatggcagagcccggtaatagagtgtaagatggcagagcccggtaatgactcatactctagagggtatataatgggggcc

SEQ ID NO: 549 - DNA, 142 bp, synthetic construct
  source 1..142: mol_type = other DNA
taagatggcagagcccggtaatagagtgctaagatggcagagcccggtaatagagtgctaagatggcagagcccggtaatagagtgctaagatggcagagcccggtaatgactcatactctagagggtatataatgggggcc

SEQ ID NO: 550 - DNA, 145 bp, synthetic construct
  source 1..145: mol_type = other DNA
taagatggcagagcccggtaatagagtgcgtaagatggcagagcccggtaatagagtgcgtaagatggcagagcccggtaatagagtgcgtaagatggcagagcccggtaatgactcatactctagagggtatataatgggggcc

SEQ ID NO: 551 - DNA, 148 bp, synthetic construct
  source 1..148: mol_type = other DNA
taagatggcagagcccggtaatagagtgcgataagatggcagagcccggtaatagagtgcgataagatggcagagcccggtaatagagtgcgataagatggcagagcccggtaatgactcatactctagagggtatataatgggggcc

SEQ ID NO: 552 - DNA, 151 bp, synthetic construct
  source 1..151: mol_type = other DNA
taagatggcagagcccggtaatagagtgcgagtaagatggcagagcccggtaatagagtgcgagtaagatggcagagcccggtaatagagtgcgagtaagatggcagagcccggtaatgactcatactctagagggtatataatgggggcc

SEQ ID NO: 553 - DNA, 122 bp, synthetic construct
  source 1..122: mol_type = other DNA
taagatggcagagcccggtaattaagatggcagagcccggtaattaagatggcagagcccggtaattaagatggcagagcccggtaatgactcatatctctagagggtatataatgggggcc

SEQ ID NO: 554 - DNA, 125 bp, synthetic construct
  source 1..125: mol_type = other DNA
taagatggcagagcccggtaatataagatggcagagcccggtaatataagatggcagagcccggtaatataagatggcagagcccggtaatgactcatatctctagagggtatataatgggggcc

SEQ ID NO: 555 - DNA, 128 bp, synthetic construct
  source 1..128: mol_type = other DNA
taagatggcagagcccggtaatagtaagatggcagagcccggtaatagtaagatggcagagcccggtaatagtaagatggcagagcccggtaatgactcatatctctagagggtatataatgggggcc

SEQ ID NO: 556 - DNA, 131 bp, synthetic construct
  source 1..131: mol_type = other DNA
taagatggcagagcccggtaatagataagatggcagagcccggtaatagataagatggcagagcccggtaatagataagatggcagagcccggtaatgactcatatctctagagggtatataatgggggcc

SEQ ID NO: 557 - DNA, 134 bp, synthetic construct
  source 1..134: mol_type = other DNA
taagatggcagagcccggtaatagagtaagatggcagagcccggtaatagagtaagatggcagagcccggtaatagagtaagatggcagagcccggtaatgactcatatctctagagggtatataatgggggcc

SEQ ID NO: 558 - DNA, 137 bp, synthetic construct
  source 1..137: mol_type = other DNA
taagatggcagagcccggtaatagagttaagatggcagagcccggtaatagagttaagatggcagagcccggtaatagagttaagatggcagagcccggtaatgactcatatctctagagggtatataatgggggcc

SEQ ID NO: 559 - DNA, 140 bp, synthetic construct
  source 1..140: mol_type = other DNA
taagatggcagagcccggtaatagagtgtaagatggcagagcccggtaatagagtgtaagatggcagagcccggtaatagagtgtaagatggcagagcccggtaatgactcatatctctagagggtatataatgggggcc

SEQ ID NO: 560 - DNA, 143 bp, synthetic construct
  source 1..143: mol_type = other DNA
taagatggcagagcccggtaatagagtgctaagatggcagagcccggtaatagagtgctaagatggcagagcccggtaatagagtgctaagatggcagagcccggtaatgactcatatctctagagggtatataatgggggcc

SEQ ID NO: 561 - DNA, 146 bp, synthetic construct
  source 1..146: mol_type = other DNA
taagatggcagagcccggtaatagagtgcgtaagatggcagagcccggtaatagagtgcgtaagatggcagagcccggtaatagagtgcgtaagatggcagagcccggtaatgactcatatctctagagggtatataatgggggcc

SEQ ID NO: 562 - DNA, 149 bp, synthetic construct
  source 1..149: mol_type = other DNA
taagatggcagagcccggtaatagagtgcgataagatggcagagcccggtaatagagtgcgataagatggcagagcccggtaatagagtgcgataagatggcagagcccggtaatgactcatatctctagagggtatataatgggggcc

SEQ ID NO: 563 - DNA, 152 bp, synthetic construct
  source 1..152: mol_type = other DNA
taagatggcagagcccggtaatagagtgcgagtaagatggcagagcccggtaatagagtgcgagtaagatggcagagcccggtaatagagtgcgagtaagatggcagagcccggtaatgactcatatctctagagggtatataatgggggcc

SEQ ID NO: 564 - DNA, 123 bp, synthetic construct
  source 1..123: mol_type = other DNA
taagatggcagagcccggtaattaagatggcagagcccggtaattaagatggcagagcccggtaattaagatggcagagcccggtaatgactcatatgctctagagggtatataatgggggcc

SEQ ID NO: 565 - DNA, 126 bp, synthetic construct
  source 1..126: mol_type = other DNA
taagatggcagagcccggtaatataagatggcagagcccggtaatataagatggcagagcccggtaatataagatggcagagcccggtaatgactcatatgctctagagggtatataatgggggcc

SEQ ID NO: 566 - DNA, 129 bp, synthetic construct
  source 1..129: mol_type = other DNA
taagatggcagagcccggtaatagtaagatggcagagcccggtaatagtaagatggcagagcccggtaatagtaagatggcagagcccggtaatgactcatatgctctagagggtatataatgggggcc

SEQ ID NO: 567 - DNA, 132 bp, synthetic construct
  source 1..132: mol_type = other DNA
taagatggcagagcccggtaatagataagatggcagagcccggtaatagataagatggcagagcccggtaatagataagatggcagagcccggtaatgactcatatgctctagagggtatataatgggggcc

SEQ ID NO: 568 - DNA, 135 bp, synthetic construct
  source 1..135: mol_type = other DNA
taagatggcagagcccggtaatagagtaagatggcagagcccggtaatagagtaagatggcagagcccggtaatagagtaagatggcagagcccggtaatgactcatatgctctagagggtatataatgggggcc

SEQ ID NO: 569 - DNA, 138 bp, synthetic construct
  source 1..138: mol_type = other DNA
taagatggcagagcccggtaatagagttaagatggcagagcccggtaatagagttaagatggcagagcccggtaatagagttaagatggcagagcccggtaatgactcatatgctctagagggtatataatgggggcc

SEQ ID NO: 570 - DNA, 141 bp, synthetic construct
  source 1..141: mol_type = other DNA
taagatggcagagcccggtaatagagtgtaagatggcagagcccggtaatagagtgtaagatggcagagcccggtaatagagtgtaagatggcagagcccggtaatgactcatatgctctagagggtatataatgggggcc

SEQ ID NO: 571 - DNA, 144 bp, synthetic construct
  source 1..144: mol_type = other DNA
taagatggcagagcccggtaatagagtgctaagatggcagagcccggtaatagagtgctaagatggcagagcccggtaatagagtgctaagatggcagagcccggtaatgactcatatgctctagagggtatataatgggggcc

SEQ ID NO: 572 - DNA, 147 bp, synthetic construct
  source 1..147: mol_type = other DNA
taagatggcagagcccggtaatagagtgcgtaagatggcagagcccggtaatagagtgcgtaagatggcagagcccggtaatagagtgcgtaagatggcagagcccggtaatgactcatatgctctagagggtatataatgggggcc

SEQ ID NO: 573 - DNA, 150 bp, synthetic construct
  source 1..150: mol_type = other DNA
taagatggcagagcccggtaatagagtgcgataagatggcagagcccggtaatagagtgcgataagatggcagagcccggtaatagagtgcgataagatggcagagcccggtaatgactcatatgctctagagggtatataatgggggcc

SEQ ID NO: 574 - DNA, 153 bp, synthetic construct
  source 1..153: mol_type = other DNA
taagatggcagagcccggtaatagagtgcgagtaagatggcagagcccggtaatagagtgcgagtaagatggcagagcccggtaatagagtgcgagtaagatggcagagcccggtaatgactcatatgctctagagggtatataatgggggcc

SEQ ID NO: 575 - DNA, 135 bp, synthetic construct
  source 1..135: mol_type = other DNA
taagatggcagagcccggtaattaagatggcagagcccggtaattaagatggcagagcccggtaattaagatggcagagcccggtaattaagatggcagagcccggtaatctctagagggtatataatgggggcc

SEQ ID NO: 576 - DNA, 139 bp, synthetic construct
  source 1..139: mol_type = other DNA
taagatggcagagcccggtaatataagatggcagagcccggtaatataagatggcagagcccggtaatataagatggcagagcccggtaatataagatggcagagcccggtaatctctagagggtatataatgggggcc

SEQ ID NO: 577 - DNA, 143 bp, synthetic construct
  source 1..143: mol_type = other DNA
taagatggcagagcccggtaatagtaagatggcagagcccggtaatagtaagatggcagagcccggtaatagtaagatggcagagcccggtaatagtaagatggcagagcccggtaatctctagagggtatataatgggggcc

SEQ ID NO: 578 - DNA, 147 bp, synthetic construct
  source 1..147: mol_type = other DNA
taagatggcagagcccggtaatagataagatggcagagcccggtaatagataagatggcagagcccggtaatagataagatggcagagcccggtaatagataagatggcagagcccggtaatctctagagggtatataatgggggcc

SEQ ID NO: 579 - DNA, 151 bp, synthetic construct
  source 1..151: mol_type = other DNA
taagatggcagagcccggtaatagagtaagatggcagagcccggtaatagagtaagatggcagagcccggtaatagagtaagatggcagagcccggtaatagagtaagatggcagagcccggtaatctctagagggtatataatgggggcc

SEQ ID NO: 580 - DNA, 155 bp, synthetic construct
  source 1..155: mol_type = other DNA
taagatggcagagcccggtaatagagttaagatggcagagcccggtaatagagttaagatggcagagcccggtaatagagttaagatggcagagcccggtaatagagttaagatggcagagcccggtaatctctagagggtatataatgggggcc

SEQ ID NO: 581 - DNA, 159 bp, synthetic construct
  source 1..159: mol_type = other DNA
taagatggcagagcccggtaatagagtgtaagatggcagagcccggtaatagagtgtaagatggcagagcccggtaatagagtgtaagatggcagagcccggtaatagagtgtaagatggcagagcccggtaatctctagagggtatataatgggggcc

SEQ ID NO: 582 - DNA, 163 bp, synthetic construct
  source 1..163: mol_type = other DNA
taagatggcagagcccggtaatagagtgctaagatggcagagcccggtaatagagtgctaagatggcagagcccggtaatagagtgctaagatggcagagcccggtaatagagtgctaagatggcagagcccggtaatctctagagggtatataatgggggcc

SEQ ID NO: 583 - DNA, 167 bp, synthetic construct
  source 1..167: mol_type = other DNA
taagatggcagagcccggtaatagagtgcgtaagatggcagagcccggtaatagagtgcgtaagatggcagagcccggtaatagagtgcgtaagatggcagagcccggtaatagagtgcgtaagatggcagagcccggtaatctctagagggtatataatgggggcc

SEQ ID NO: 584 - DNA, 171 bp, synthetic construct
  source 1..171: mol_type = other DNA
taagatggcagagcccggtaatagagtgcgataagatggcagagcccggtaatagagtgcgataagatggcagagcccggtaatagagtgcgataagatggcagagcccggtaatagagtgcgataagatggcagagcccggtaatctctagagggtatataatgggggcc

SEQ ID NO: 585 - DNA, 175 bp, synthetic construct
  source 1..175: mol_type = other DNA
taagatggcagagcccggtaatagagtgcgagtaagatggcagagcccggtaatagagtgcgagtaagatggcagagcccggtaatagagtgcgagtaagatggcagagcccggtaatagagtgcgagtaagatggcagagcccggtaatctctagagggtatataatgggggcc

SEQ ID NO: 586 - DNA, 136 bp, synthetic construct
  source 1..136: mol_type = other DNA
taagatggcagagcccggtaattaagatggcagagcccggtaattaagatggcagagcccggtaattaagatggcagagcccggtaattaagatggcagagcccggtaatgctctagagggtatataatgggggcc

SEQ ID NO: 587 - DNA, 140 bp, synthetic construct
  source 1..140: mol_type = other DNA
taagatggcagagcccggtaatataagatggcagagcccggtaatataagatggcagagcccggtaatataagatggcagagcccggtaatataagatggcagagcccggtaatgctctagagggtatataatgggggcc

SEQ ID NO: 588 - DNA, 144 bp, synthetic construct
  source 1..144: mol_type = other DNA
taagatggcagagcccggtaatagtaagatggcagagcccggtaatagtaagatggcagagcccggtaatagtaagatggcagagcccggtaatagtaagatggcagagcccggtaatgctctagagggtatataatgggggcc

SEQ ID NO: 589 - DNA, 148 bp, synthetic construct
  source 1..148: mol_type = other DNA
taagatggcagagcccggtaatagataagatggcagagcccggtaatagataagatggcagagcccggtaatagataagatggcagagcccggtaatagataagatggcagagcccggtaatgctctagagggtatataatgggggcc

SEQ ID NO: 590 - DNA, 152 bp, synthetic construct
  source 1..152: mol_type = other DNA
taagatggcagagcccggtaatagagtaagatggcagagcccggtaatagagtaagatggcagagcccggtaatagagtaagatggcagagcccggtaatagagtaagatggcagagcccggtaatgctctagagggtatataatgggggcc

SEQ ID NO: 591 - DNA, 156 bp, synthetic construct
  source 1..156: mol_type = other DNA
taagatggcagagcccggtaatagagttaagatggcagagcccggtaatagagttaagatggcagagcccggtaatagagttaagatggcagagcccggtaatagagttaagatggcagagcccggtaatgctctagagggtatataatgggggcc

SEQ ID NO: 592 - DNA, 160 bp, synthetic construct
  source 1..160: mol_type = other DNA
taagatggcagagcccggtaatagagtgtaagatggcagagcccggtaatagagtgtaagatggcagagcccggtaatagagtgtaagatggcagagcccggtaatagagtgtaagatggcagagcccggtaatgctctagagggtatataatgggggcc

SEQ ID NO: 593 - DNA, 164 bp, synthetic construct
  source 1..164: mol_type = other DNA
taagatggcagagcccggtaatagagtgctaagatggcagagcccggtaatagagtgctaagatggcagagcccggtaatagagtgctaagatggcagagcccggtaatagagtgctaagatggcagagcccggtaatgctctagagggtatataatgggggcc

SEQ ID NO: 594 - DNA, 168 bp, synthetic construct
  source 1..168: mol_type = other DNA
taagatggcagagcccggtaatagagtgcgtaagatggcagagcccggtaatagagtgcgtaagatggcagagcccggtaatagagtgcgtaagatggcagagcccggtaatagagtgcgtaagatggcagagcccggtaatgctctagagggtatataatgggggcc

SEQ ID NO: 595 - DNA, 172 bp, synthetic construct
  source 1..172: mol_type = other DNA
taagatggcagagcccggtaatagagtgcgataagatggcagagcccggtaatagagtgcgataagatggcagagcccggtaatagagtgcgataagatggcagagcccggtaatagagtgcgataagatggcagagcccggtaatgctctagagggtatataatgggggcc

SEQ ID NO: 596 - DNA, 176 bp, synthetic construct
  source 1..176: mol_type = other DNA
taagatggcagagcccggtaatagagtgcgagtaagatggcagagcccggtaatagagtgcgagtaagatggcagagcccggtaatagagtgcgagtaagatggcagagcccggtaatagagtgcgagtaagatggcagagcccggtaatgctctagagggtatataatgggggcc

SEQ ID NO: 597 - DNA, 137 bp, synthetic construct
  source 1..137: mol_type = other DNA
taagatggcagagcccggtaattaagatggcagagcccggtaattaagatggcagagcccggtaattaagatggcagagcccggtaattaagatggcagagcccggtaatgactctagagggtatataatgggggcc

SEQ ID NO: 598 - DNA, 141 bp, synthetic construct
  source 1..141: mol_type = other DNA
taagatggcagagcccggtaatataagatggcagagcccggtaatataagatggcagagcccggtaatataagatggcagagcccggtaatataagatggcagagcccggtaatgactctagagggtatataatgggggcc

SEQ ID NO: 599 - DNA, 145 bp, synthetic construct
  source 1..145: mol_type = other DNA
taagatggcagagcccggtaatagtaagatggcagagcccggtaatagtaagatggcagagcccggtaatagtaagatggcagagcccggtaatagtaagatggcagagcccggtaatgactctagagggtatataatgggggcc

SEQ ID NO: 600 - DNA, 149 bp, synthetic construct
  source 1..149: mol_type = other DNA
taagatggcagagcccggtaatagataagatggcagagcccggtaatagataagatggcagagcccggtaatagataagatggcagagcccggtaatagataagatggcagagcccggtaatgactctagagggtatataatgggggcc

SEQ ID NO: 601 - DNA, 153 bp, synthetic construct
  source 1..153: mol_type = other DNA
taagatggcagagcccggtaatagagtaagatggcagagcccggtaatagagtaagatggcagagcccggtaatagagtaagatggcagagcccggtaatagagtaagatggcagagcccggtaatgactctagagggtatataatgggggcc

SEQ ID NO: 602 - DNA, 157 bp, synthetic construct
  source 1..157: mol_type = other DNA
taagatggcagagcccggtaatagagttaagatggcagagcccggtaatagagttaagatggcagagcccggtaatagagttaagatggcagagcccggtaatagagttaagatggcagagcccggtaatgactctagagggtatataatgggggcc

SEQ ID NO: 603 - DNA, 161 bp, synthetic construct
  source 1..161: mol_type = other DNA
taagatggcagagcccggtaatagagtgtaagatggcagagcccggtaatagagtgtaagatggcagagcccggtaatagagtgtaagatggcagagcccggtaatagagtgtaagatggcagagcccggtaatgactctagagggtatataatgggggcc

SEQ ID NO: 604 - DNA, 165 bp, synthetic construct
  source 1..165: mol_type = other DNA
taagatggcagagcccggtaatagagtgctaagatggcagagcccggtaatagagtgctaagatggcagagcccggtaatagagtgctaagatggcagagcccggtaatagagtgctaagatggcagagcccggtaatgactctagagggtatataatgggggcc

SEQ ID NO: 605 - DNA, 169 bp, synthetic construct
  source 1..169: mol_type = other DNA
taagatggcagagcccggtaatagagtgcgtaagatggcagagcccggtaatagagtgcgtaagatggcagagcccggtaatagagtgcgtaagatggcagagcccggtaatagagtgcgtaagatggcagagcccggtaatgactctagagggtatataatgggggcc

SEQ ID NO: 606 - DNA, 173 bp, synthetic construct
  source 1..173: mol_type = other DNA
taagatggcagagcccggtaatagagtgcgataagatggcagagcccggtaatagagtgcgataagatggcagagcccggtaatagagtgcgataagatggcagagcccggtaatagagtgcgataagatggcagagcccggtaatgactctagagggtatataatgggggcc

SEQ ID NO: 607 - DNA, 177 bp, synthetic construct
  source 1..177: mol_type = other DNA
taagatggcagagcccggtaatagagtgcgagtaagatggcagagcccggtaatagagtgcgagtaagatggcagagcccggtaatagagtgcgagtaagatggcagagcccggtaatagagtgcgagtaagatggcagagcccggtaatgactctagagggtatataatgggggcc

SEQ ID NO: 608 - DNA, 138 bp, synthetic construct
  source 1..138: mol_type = other DNA
taagatggcagagcccggtaattaagatggcagagcccggtaattaagatggcagagcccggtaattaagatggcagagcccggtaattaagatggcagagcccggtaatgacctctagagggtatataatgggggcc

SEQ ID NO: 609 - DNA, 142 bp, synthetic construct
  source 1..142: mol_type = other DNA
taagatggcagagcccggtaatataagatggcagagcccggtaatataagatggcagagcccggtaatataagatggcagagcccggtaatataagatggcagagcccggtaatgacctctagagggtatataatgggggcc

SEQ ID NO: 610 - DNA, 146 bp, synthetic construct
  source 1..146: mol_type = other DNA
taagatggcagagcccggtaatagtaagatggcagagcccggtaatagtaagatggcagagcccggtaatagtaagatggcagagcccggtaatagtaagatggcagagcccggtaatgacctctagagggtatataatgggggcc

SEQ ID NO: 611 - DNA, 150 bp, synthetic construct
  source 1..150: mol_type = other DNA
taagatggcagagcccggtaatagataagatggcagagcccggtaatagataagatggcagagcccggtaatagataagatggcagagcccggtaatagataagatggcagagcccggtaatgacctctagagggtatataatgggggcc

SEQ ID NO: 612 - DNA, 154 bp, synthetic construct
  source 1..154: mol_type = other DNA
taagatggcagagcccggtaatagagtaagatggcagagcccggtaatagagtaagatggcagagcccggtaatagagtaagatggcagagcccggtaatagagtaagatggcagagcccggtaatgacctctagagggtatataatgggggcc

SEQ ID NO: 613 - DNA, 158 bp, synthetic construct
  source 1..158: mol_type = other DNA
taagatggcagagcccggtaatagagttaagatggcagagcccggtaatagagttaagatggcagagcccggtaatagagttaagatggcagagcccggtaatagagttaagatggcagagcccggtaatgacctctagagggtatataatgggggcc

SEQ ID NO: 614 - DNA, 162 bp, synthetic construct
  source 1..162: mol_type = other DNA
taagatggcagagcccggtaatagagtgtaagatggcagagcccggtaatagagtgtaagatggcagagcccggtaatagagtgtaagatggcagagcccggtaatagagtgtaagatggcagagcccggtaatgacctctagagggtatataatgggggcc

SEQ ID NO: 615 - DNA, 166 bp, synthetic construct
  source 1..166: mol_type = other DNA
taagatggcagagcccggtaatagagtgctaagatggcagagcccggtaatagagtgctaagatggcagagcccggtaatagagtgctaagatggcagagcccggtaatagagtgctaagatggcagagcccggtaatgacctctagagggtatataatgggggcc

SEQ ID NO: 616 - DNA, 170 bp, synthetic construct
  source 1..170: mol_type = other DNA
taagatggcagagcccggtaatagagtgcgtaagatggcagagcccggtaatagagtgcgtaagatggcagagcccggtaatagagtgcgtaagatggcagagcccggtaatagagtgcgtaagatggcagagcccggtaatgacctctagagggtatataatgggggcc

SEQ ID NO: 617 - DNA, 174 bp, synthetic construct
  source 1..174: mol_type = other DNA
taagatggcagagcccggtaatagagtgcgataagatggcagagcccggtaatagagtgcgataagatggcagagcccggtaatagagtgcgataagatggcagagcccggtaatagagtgcgataagatggcagagcccggtaatgacctctagagggtatataatgggggcc

SEQ ID NO: 618 - DNA, 178 bp, synthetic construct
  source 1..178: mol_type = other DNA
taagatggcagagcccggtaatagagtgcgagtaagatggcagagcccggtaatagagtgcgagtaagatggcagagcccggtaatagagtgcgagtaagatggcagagcccggtaatagagtgcgagtaagatggcagagcccggtaatgacctctagagggtatataatgggggcc

SEQ ID NO: 619 - DNA, 139 bp, synthetic construct
  source 1..139: mol_type = other DNA
taagatggcagagcccggtaattaagatggcagagcccggtaattaagatggcagagcccggtaattaagatggcagagcccggtaattaagatggcagagcccggtaatgactctctagagggtatataatgggggcc

SEQ ID NO: 620 - DNA, 143 bp, synthetic construct
  source 1..143: mol_type = other DNA
taagatggcagagcccggtaatataagatggcagagcccggtaatataagatggcagagcccggtaatataagatggcagagcccggtaatataagatggcagagcccggtaatgactctctagagggtatataatgggggcc

SEQ ID NO: 621 - DNA, 147 bp, synthetic construct
  source 1..147: mol_type = other DNA
taagatggcagagcccggtaatagtaagatggcagagcccggtaatagtaagatggcagagcccggtaatagtaagatggcagagcccggtaatagtaagatggcagagcccggtaatgactctctagagggtatataatgggggcc

SEQ ID NO: 622 - DNA, 151 bp, synthetic construct
  source 1..151: mol_type = other DNA
taagatggcagagcccggtaatagataagatggcagagcccggtaatagataagatggcagagcccggtaatagataagatggcagagcccggtaatagataagatggcagagcccggtaatgactctctagagggtatataatgggggcc

SEQ ID NO: 623 - DNA, 155 bp, synthetic construct
  source 1..155: mol_type = other DNA
taagatggcagagcccggtaatagagtaagatggcagagcccggtaatagagtaagatggcagagcccggtaatagagtaagatggcagagcccggtaatagagtaagatggcagagcccggtaatgactctctagagggtatataatgggggcc

SEQ ID NO: 624 - DNA, 159 bp, synthetic construct
  source 1..159: mol_type = other DNA
taagatggcagagcccggtaatagagttaagatggcagagcccggtaatagagttaagatggcagagcccggtaatagagttaagatggcagagcccggtaatagagttaagatggcagagcccggtaatgactctctagagggtatataatgggggcc

SEQ ID NO: 625 - DNA, 163 bp, synthetic construct
  source 1..163: mol_type = other DNA
taagatggcagagcccggtaatagagtgtaagatggcagagcccggtaatagagtgtaagatggcagagcccggtaatagagtgtaagatggcagagcccggtaatagagtgtaagatggcagagcccggtaatgactctctagagggtatataatgggggcc

SEQ ID NO: 626 - DNA, 167 bp, synthetic construct
  source 1..167: mol_type = other DNA
taagatggcagagcccggtaatagagtgctaagatggcagagcccggtaatagagtgctaagatggcagagcccggtaatagagtgctaagatggcagagcccggtaatagagtgctaagatggcagagcccggtaatgactctctagagggtatataatgggggcc

SEQ ID NO: 627 - DNA, 171 bp, synthetic construct
  source 1..171: mol_type = other DNA
taagatggcagagcccggtaatagagtgcgtaagatggcagagcccggtaatagagtgcgtaagatggcagagcccggtaatagagtgcgtaagatggcagagcccggtaatagagtgcgtaagatggcagagcccggtaatgactctctagagggtatataatgggggcc

SEQ ID NO: 628 - DNA, 175 bp, synthetic construct
  source 1..175: mol_type = other DNA
taagatggcagagcccggtaatagagtgcgataagatggcagagcccggtaatagagtgcgataagatggcagagcccggtaatagagtgcgataagatggcagagcccggtaatagagtgcgataagatggcagagcccggtaatgactctctagagggtatataatgggggcc

SEQ ID NO: 629 - DNA, 179 bp, synthetic construct
  source 1..179: mol_type = other DNA
taagatggcagagcccggtaatagagtgcgagtaagatggcagagcccggtaatagagtgcgagtaagatggcagagcccggtaatagagtgcgagtaagatggcagagcccggtaatagagtgcgagtaagatggcagagcccggtaatgactctctagagggtatataatgggggcc

SEQ ID NO: 630 - DNA, 140 bp, synthetic construct
  source 1..140: mol_type = other DNA
taagatggcagagcccggtaattaagatggcagagcccggtaattaagatggcagagcccggtaattaagatggcagagcccggtaattaagatggcagagcccggtaatgactcctctagagggtatataatgggggcc

SEQ ID NO: 631 - DNA, 144 bp, synthetic construct
  source 1..144: mol_type = other DNA
taagatggcagagcccggtaatataagatggcagagcccggtaatataagatggcagagcccggtaatataagatggcagagcccggtaatataagatggcagagcccggtaatgactcctctagagggtatataatgggggcc

SEQ ID NO: 632 - DNA, 148 bp, synthetic construct
  source 1..148: mol_type = other DNA
taagatggcagagcccggtaatagtaagatggcagagcccggtaatagtaagatggcagagcccggtaatagtaagatggcagagcccggtaatagtaagatggcagagcccggtaatgactcctctagagggtatataatgggggcc

SEQ ID NO: 633 - DNA, 152 bp, synthetic construct
  source 1..152: mol_type = other DNA
taagatggcagagcccggtaatagataagatggcagagcccggtaatagataagatggcagagcccggtaatagataagatggcagagcccggtaatagataagatggcagagcccggtaatgactcctctagagggtatataatgggggcc

SEQ ID NO: 634 - DNA, 156 bp, synthetic construct
  source 1..156: mol_type = other DNA
taagatggcagagcccggtaatagagtaagatggcagagcccggtaatagagtaagatggcagagcccggtaatagagtaagatggcagagcccggtaatagagtaagatggcagagcccggtaatgactcctctagagggtatataatgggggcc

SEQ ID NO: 635 - DNA, 160 bp, synthetic construct
  source 1..160: mol_type = other DNA
taagatggcagagcccggtaatagagttaagatggcagagcccggtaatagagttaagatggcagagcccggtaatagagttaagatggcagagcccggtaatagagttaagatggcagagcccggtaatgactcctctagagggtatataatgggggcc

SEQ ID NO: 636 - DNA, 164 bp, synthetic construct
  source 1..164: mol_type = other DNA
taagatggcagagcccggtaatagagtgtaagatggcagagcccggtaatagagtgtaagatggcagagcccggtaatagagtgtaagatggcagagcccggtaatagagtgtaagatggcagagcccggtaatgactcctctagagggtatataatgggggcc

SEQ ID NO: 637 - DNA, 168 bp, synthetic construct
  source 1..168: mol_type = other DNA
taagatggcagagcccggtaatagagtgctaagatggcagagcccggtaatagagtgctaagatggcagagcccggtaatagagtgctaagatggcagagcccggtaatagagtgctaagatggcagagcccggtaatgactcctctagagggtatataatgggggcc

SEQ ID NO: 638 - DNA, 172 bp, synthetic construct
  source 1..172: mol_type = other DNA
taagatggcagagcccggtaatagagtgcgtaagatggcagagcccggtaatagagtgcgtaagatggcagagcccggtaatagagtgcgtaagatggcagagcccggtaatagagtgcgtaagatggcagagcccggtaatgactcctctagagggtatataatgggggcc

SEQ ID NO: 639 - DNA, 176 bp, synthetic construct
  source 1..176: mol_type = other DNA
taagatggcagagcccggtaatagagtgcgataagatggcagagcccggtaatagagtgcgataagatggcagagcccggtaatagagtgcgataagatggcagagcccggtaatagagtgcgataagatggcagagcccggtaatgactcctctagagggtatataatgggggcc

SEQ ID NO: 640 - DNA, 180 bp, synthetic construct
  source 1..180: mol_type = other DNA
taagatggcagagcccggtaatagagtgcgagtaagatggcagagcccggtaatagagtgcgagtaagatggcagagcccggtaatagagtgcgagtaagatggcagagcccggtaatagagtgcgagtaagatggcagagcccggtaatgactcctctagagggtatataatgggggcc

SEQ ID NO: 641 - DNA, 141 bp, synthetic construct
  source 1..141: mol_type = other DNA
taagatggcagagcccggtaattaagatggcagagcccggtaattaagatggcagagcccggtaattaagatggcagagcccggtaattaagatggcagagcccggtaatgactcactctagagggtatataatgggggcc

SEQ ID NO: 642 - DNA, 145 bp, synthetic construct
  source 1..145: mol_type = other DNA
taagatggcagagcccggtaatataagatggcagagcccggtaatataagatggcagagcccggtaatataagatggcagagcccggtaatataagatggcagagcccggtaatgactcactctagagggtatataatgggggcc

SEQ ID NO: 643 - DNA, 149 bp, synthetic construct
  source 1..149: mol_type = other DNA
taagatggcagagcccggtaatagtaagatggcagagcccggtaatagtaagatggcagagcccggtaatagtaagatggcagagcccggtaatagtaagatggcagagcccggtaatgactcactctagagggtatataatgggggcc

SEQ ID NO: 644 - DNA, 153 bp, synthetic construct
  source 1..153: mol_type = other DNA
taagatggcagagcccggtaatagataagatggcagagcccggtaatagataagatggcagagcccggtaatagataagatggcagagcccggtaatagataagatggcagagcccggtaatgactcactctagagggtatataatgggggcc

SEQ ID NO: 645 - DNA, 157 bp, synthetic construct
  source 1..157: mol_type = other DNA
taagatggcagagcccggtaatagagtaagatggcagagcccggtaatagagtaagatggcagagcccggtaatagagtaagatggcagagcccggtaatagagtaagatggcagagcccggtaatgactcactctagagggtatataatgggggcc

SEQ ID NO: 646 - DNA, 161 bp, synthetic construct
  source 1..161: mol_type = other DNA
taagatggcagagcccggtaatagagttaagatggcagagcccggtaatagagttaagatggcagagcccggtaatagagttaagatggcagagcccggtaatagagttaagatggcagagcccggtaatgactcactctagagggtatataatgggggcc

SEQ ID NO: 647 - DNA, 165 bp, synthetic construct
  source 1..165: mol_type = other DNA
taagatggcagagcccggtaatagagtgtaagatggcagagcccggtaatagagtgtaagatggcagagcccggtaatagagtgtaagatggcagagcccggtaatagagtgtaagatggcagagcccggtaatgactcactctagagggtatataatgggggcc

SEQ ID NO: 648 - DNA, 169 bp, synthetic construct
  source 1..169: mol_type = other DNA
taagatggcagagcccggtaatagagtgctaagatggcagagcccggtaatagagtgctaagatggcagagcccggtaatagagtgctaagatggcagagcccggtaatagagtgctaagatggcagagcccggtaatgactcactctagagggtatataatgggggcc

SEQ ID NO: 649 - DNA, 173 bp, synthetic construct
  source 1..173: mol_type = other DNA
taagatggcagagcccggtaatagagtgcgtaagatggcagagcccggtaatagagtgcgtaagatggcagagcccggtaatagagtgcgtaagatggcagagcccggtaatagagtgcgtaagatggcagagcccggtaatgactcactctagagggtatataatgggggcc

SEQ ID NO: 650 - DNA, 177 bp, synthetic construct
  source 1..177: mol_type = other DNA
taagatggcagagcccggtaatagagtgcgataagatggcagagcccggtaatagagtgcgataagatggcagagcccggtaatagagtgcgataagatggcagagcccggtaatagagtgcgataagatggcagagcccggtaatgactcactctagagggtatataatgggggcc

SEQ ID NO: 651 - DNA, 181 bp, synthetic construct
  source 1..181: mol_type = other DNA
taagatggcagagcccggtaatagagtgcgagtaagatggcagagcccggtaatagagtgcgagtaagatggcagagcccggtaatagagtgcgagtaagatggcagagcccggtaatagagtgcgagtaagatggcagagcccggtaatgactcactctagagggtatataatgggggcc

SEQ ID NO: 652 - DNA, 142 bp, synthetic construct
  source 1..142: mol_type = other DNA
taagatggcagagcccggtaattaagatggcagagcccggtaattaagatggcagagcccggtaattaagatggcagagcccggtaattaagatggcagagcccggtaatgactcatctctagagggtatataatgggggcc

SEQ ID NO: 653 - DNA, 146 bp, synthetic construct
  source 1..146: mol_type = other DNA
taagatggcagagcccggtaatataagatggcagagcccggtaatataagatggcagagcccggtaatataagatggcagagcccggtaatataagatggcagagcccggtaatgactcatctctagagggtatataatgggggcc

SEQ ID NO: 654 - DNA, 150 bp, synthetic construct
  source 1..150: mol_type = other DNA
taagatggcagagcccggtaatagtaagatggcagagcccggtaatagtaagatggcagagcccggtaatagtaagatggcagagcccggtaatagtaagatggcagagcccggtaatgactcatctctagagggtatataatgggggcc

SEQ ID NO: 655 - DNA, 154 bp, synthetic construct
  source 1..154: mol_type = other DNA
taagatggcagagcccggtaatagataagatggcagagcccggtaatagataagatggcagagcccggtaatagataagatggcagagcccggtaatagataagatggcagagcccggtaatgactcatctctagagggtatataatgggggcc

SEQ ID NO: 656 - DNA, 158 bp, synthetic construct
  source 1..158: mol_type = other DNA
taagatggcagagcccggtaatagagtaagatggcagagcccggtaatagagtaagatggcagagcccggtaatagagtaagatggcagagcccggtaatagagtaagatggcagagcccggtaatgactcatctctagagggtatataatgggggcc

SEQ ID NO: 657 - DNA, 162 bp, synthetic construct
  source 1..162: mol_type = other DNA
taagatggcagagcccggtaatagagttaagatggcagagcccggtaatagagttaagatggcagagcccggtaatagagttaagatggcagagcccggtaatagagttaagatggcagagcccggtaatgactcatctctagagggtatataatgggggcc

SEQ ID NO: 658 - DNA, 166 bp, synthetic construct
  source 1..166: mol_type = other DNA
taagatggcagagcccggtaatagagtgtaagatggcagagcccggtaatagagtgtaagatggcagagcccggtaatagagtgtaagatggcagagcccggtaatagagtgtaagatggcagagcccggtaatgactcatctctagagggtatataatgggggcc

SEQ ID NO: 659 - DNA, 170 bp, synthetic construct
  source 1..170: mol_type = other DNA
taagatggcagagcccggtaatagagtgctaagatggcagagcccggtaatagagtgctaagatggcagagcccggtaatagagtgctaagatggcagagcccggtaatagagtgctaagatggcagagcccggtaatgactcatctctagagggtatataatgggggcc

SEQ ID NO: 660 - DNA, 174 bp, synthetic construct
  source 1..174: mol_type = other DNA
taagatggcagagcccggtaatagagtgcgtaagatggcagagcccggtaatagagtgcgtaagatggcagagcccggtaatagagtgcgtaagatggcagagcccggtaatagagtgcgtaagatggcagagcccggtaatgactcatctctagagggtatataatgggggcc

SEQ ID NO: 661 - DNA, 178 bp, synthetic construct
  source 1..178: mol_type = other DNA
taagatggcagagcccggtaatagagtgcgataagatggcagagcccggtaatagagtgcgataagatggcagagcccggtaatagagtgcgataagatggcagagcccggtaatagagtgcgataagatggcagagcccggtaatgactcatctctagagggtatataatgggggcc

SEQ ID NO: 662 - DNA, 182 bp, synthetic construct
  source 1..182: mol_type = other DNA
taagatggcagagcccggtaatagagtgcgagtaagatggcagagcccggtaatagagtgcgagtaagatggcagagcccggtaatagagtgcgagtaagatggcagagcccggtaatagagtgcgagtaagatggcagagcccggtaatgactcatctctagagggtatataatgggggcc

SEQ ID NO: 663 - DNA, 143 bp, synthetic construct
  source 1..143: mol_type = other DNA
taagatggcagagcccggtaattaagatggcagagcccggtaattaagatggcagagcccggtaattaagatggcagagcccggtaattaagatggcagagcccggtaatgactcatactctagagggtatataatgggggcc

SEQ ID NO: 664 - DNA, 147 bp, synthetic construct
  source 1..147: mol_type = other DNA
taagatggcagagcccggtaatataagatggcagagcccggtaatataagatggcagagcccggtaatataagatggcagagcccggtaatataagatggcagagcccggtaatgactcatactctagagggtatataatgggggcc

SEQ ID NO: 665 - DNA, 151 bp, synthetic construct
  source 1..151: mol_type = other DNA
taagatggcagagcccggtaatagtaagatggcagagcccggtaatagtaagatggcagagcccggtaatagtaagatggcagagcccggtaatagtaagatggcagagcccggtaatgactcatactctagagggtatataatgggggcc

SEQ ID NO: 666 - DNA, 155 bp, synthetic construct
  source 1..155: mol_type = other DNA
taagatggcagagcccggtaatagataagatggcagagcccggtaatagataagatggcagagcccggtaatagataagatggcagagcccggtaatagataagatggcagagcccggtaatgactcatactctagagggtatataatgggggcc

SEQ ID NO: 667 - DNA, 159 bp, synthetic construct
  source 1..159: mol_type = other DNA
taagatggcagagcccggtaatagagtaagatggcagagcccggtaatagagtaagatggcagagcccggtaatagagtaagatggcagagcccggtaatagagtaagatggcagagcccggtaatgactcatactctagagggtatataatgggggcc

SEQ ID NO: 668 - DNA, 163 bp, synthetic construct
  source 1..163: mol_type = other DNA
taagatggcagagcccggtaatagagttaagatggcagagcccggtaatagagttaagatggcagagcccggtaatagagttaagatggcagagcccggtaatagagttaagatggcagagcccggtaatgactcatactctagagggtatataatgggggcc

SEQ ID NO: 669 - DNA, 167 bp, synthetic construct
  source 1..167: mol_type = other DNA
taagatggcagagcccggtaatagagtgtaagatggcagagcccggtaatagagtgtaagatggcagagcccggtaatagagtgtaagatggcagagcccggtaatagagtgtaagatggcagagcccggtaatgactcatactctagagggtatataatgggggcc

SEQ ID NO: 670 - DNA, 171 bp, synthetic construct
  source 1..171: mol_type = other DNA
taagatggcagagcccggtaatagagtgctaagatggcagagcccggtaatagagtgctaagatggcagagcccggtaatagagtgctaagatggcagagcccggtaatagagtgctaagatggcagagcccggtaatgactcatactctagagggtatataatgggggcc

SEQ ID NO: 671 - DNA, 175 bp, synthetic construct
  source 1..175: mol_type = other DNA
taagatggcagagcccggtaatagagtgcgtaagatggcagagcccggtaatagagtgcgtaagatggcagagcccggtaatagagtgcgtaagatggcagagcccggtaatagagtgcgtaagatggcagagcccggtaatgactcatactctagagggtatataatgggggcc

SEQ ID NO: 672 - DNA, 179 bp, synthetic construct
  source 1..179: mol_type = other DNA
taagatggcagagcccggtaatagagtgcgataagatggcagagcccggtaatagagtgcgataagatggcagagcccggtaatagagtgcgataagatggcagagcccggtaatagagtgcgataagatggcagagcccggtaatgactcatactctagagggtatataatgggggcc

SEQ ID NO: 673 - DNA, 183 bp, synthetic construct
  source 1..183: mol_type = other DNA
taagatggcagagcccggtaatagagtgcgagtaagatggcagagcccggtaatagagtgcgagtaagatggcagagcccggtaatagagtgcgagtaagatggcagagcccggtaatagagtgcgagtaagatggcagagcccggtaatgactcatactctagagggtatataatgggggcc

SEQ ID NO: 674 - DNA, 144 bp, synthetic construct
  source 1..144: mol_type = other DNA
taagatggcagagcccggtaattaagatggcagagcccggtaattaagatggcagagcccggtaattaagatggcagagcccggtaattaagatggcagagcccggtaatgactcatatctctagagggtatataatgggggcc

SEQ ID NO: 675 - DNA, 148 bp, synthetic construct
  source 1..148: mol_type = other DNA
taagatggcagagcccggtaatataagatggcagagcccggtaatataagatggcagagcccggtaatataagatggcagagcccggtaatataagatggcagagcccggtaatgactcatatctctagagggtatataatgggggcc

SEQ ID NO: 676 - DNA, 152 bp, synthetic construct
  source 1..152: mol_type = other DNA
taagatggcagagcccggtaatagtaagatggcagagcccggtaatagtaagatggcagagcccggtaatagtaagatggcagagcccggtaatagtaagatggcagagcccggtaatgactcatatctctagagggtatataatgggggcc

SEQ ID NO: 677 - DNA, 156 bp, synthetic construct
  source 1..156: mol_type = other DNA
taagatggcagagcccggtaatagataagatggcagagcccggtaatagataagatggcagagcccggtaatagataagatggcagagcccggtaatagataagatggcagagcccggtaatgactcatatctctagagggtatataatgggggcc

SEQ ID NO: 678 - DNA, 160 bp, synthetic construct
  source 1..160: mol_type = other DNA
taagatggcagagcccggtaatagagtaagatggcagagcccggtaatagagtaagatggcagagcccggtaatagagtaagatggcagagcccggtaatagagtaagatggcagagcccggtaatgactcatatctctagagggtatataatgggggcc

SEQ ID NO: 679 - DNA, 164 bp, synthetic construct
  source 1..164: mol_type = other DNA
taagatggcagagcccggtaatagagttaagatggcagagcccggtaatagagttaagatggcagagcccggtaatagagttaagatggcagagcccggtaatagagttaagatggcagagcccggtaatgactcatatctctagagggtatataatgggggcc

SEQ ID NO: 680 - DNA, 168 bp, synthetic construct
  source 1..168: mol_type = other DNA
taagatggcagagcccggtaatagagtgtaagatggcagagcccggtaatagagtgtaagatggcagagcccggtaatagagtgtaagatggcagagcccggtaatagagtgtaagatggcagagcccggtaatgactcatatctctagagggtatataatgggggcc

SEQ ID NO: 681 - DNA, 172 bp, synthetic construct
  source 1..172: mol_type = other DNA
taagatggcagagcccggtaatagagtgctaagatggcagagcccggtaatagagtgctaagatggcagagcccggtaatagagtgctaagatggcagagcccggtaatagagtgctaagatggcagagcccggtaatgactcatatctctagagggtatataatgggggcc

SEQ ID NO: 682 - DNA, 176 bp, synthetic construct
  source 1..176: mol_type = other DNA
taagatggcagagcccggtaatagagtgcgtaagatggcagagcccggtaatagagtgcgtaagatggcagagcccggtaatagagtgcgtaagatggcagagcccggtaatagagtgcgtaagatggcagagcccggtaatgactcatatctctagagggtatataatgggggcc

SEQ ID NO: 683 - DNA, 180 bp, synthetic construct
  source 1..180: mol_type = other DNA
taagatggcagagcccggtaatagagtgcgataagatggcagagcccggtaatagagtgcgataagatggcagagcccggtaatagagtgcgataagatggcagagcccggtaatagagtgcgataagatggcagagcccggtaatgactcatatctctagagggtatataatgggggcc

SEQ ID NO: 684 - DNA, 184 bp, synthetic construct
  source 1..184: mol_type = other DNA
taagatggcagagcccggtaatagagtgcgagtaagatggcagagcccggtaatagagtgcgagtaagatggcagagcccggtaatagagtgcgagtaagatggcagagcccggtaatagagtgcgagtaagatggcagagcccggtaatgactcatatctctagagggtatataatgggggcc

SEQ ID NO: 685 - DNA, 145 bp, synthetic construct
  source 1..145: mol_type = other DNA
taagatggcagagcccggtaattaagatggcagagcccggtaattaagatggcagagcccggtaattaagatggcagagcccggtaattaagatggcagagcccggtaatgactcatatgctctagagggtatataatgggggcc

SEQ ID NO: 686 - DNA, 149 bp, synthetic construct
  source 1..149: mol_type = other DNA
taagatggcagagcccggtaatataagatggcagagcccggtaatataagatggcagagcccggtaatataagatggcagagcccggtaatataagatggcagagcccggtaatgactcatatgctctagagggtatataatgggggcc

SEQ ID NO: 687 - DNA, 153 bp, synthetic construct
  source 1..153: mol_type = other DNA
taagatggcagagcccggtaatagtaagatggcagagcccggtaatagtaagatggcagagcccggtaatagtaagatggcagagcccggtaatagtaagatggcagagcccggtaatgactcatatgctctagagggtatataatgggggcc

SEQ ID NO: 688 - DNA, 157 bp, synthetic construct
  source 1..157: mol_type = other DNA
taagatggcagagcccggtaatagataagatggcagagcccggtaatagataagatggcagagcccggtaatagataagatggcagagcccggtaatagataagatggcagagcccggtaatgactcatatgctctagagggtatataatgggggcc

SEQ ID NO: 689 - DNA, 161 bp, synthetic construct
  source 1..161: mol_type = other DNA
taagatggcagagcccggtaatagagtaagatggcagagcccggtaatagagtaagatggcagagcccggtaatagagtaagatggcagagcccggtaatagagtaagatggcagagcccggtaatgactcatatgctctagagggtatataatgggggcc

SEQ ID NO: 690 - DNA, 165 bp, synthetic construct
  source 1..165: mol_type = other DNA
taagatggcagagcccggtaatagagttaagatggcagagcccggtaatagagttaagatggcagagcccggtaatagagttaagatggcagagcccggtaatagagttaagatggcagagcccggtaatgactcatatgctctagagggtatataatgggggcc

SEQ ID NO: 691 - DNA, 169 bp, synthetic construct
  source 1..169: mol_type = other DNA
taagatggcagagcccggtaatagagtgtaagatggcagagcccggtaatagagtgtaagatggcagagcccggtaatagagtgtaagatggcagagcccggtaatagagtgtaagatggcagagcccggtaatgactcatatgctctagagggtatataatgggggcc

SEQ ID NO: 692 - DNA, 173 bp, synthetic construct
  source 1..173: mol_type = other DNA
taagatggcagagcccggtaatagagtgctaagatggcagagcccggtaatagagtgctaagatggcagagcccggtaatagagtgctaagatggcagagcccggtaatagagtgctaagatggcagagcccggtaatgactcatatgctctagagggtatataatgggggcc

SEQ ID NO: 693 - DNA, 177 bp, synthetic construct
  source 1..177: mol_type = other DNA
taagatggcagagcccggtaatagagtgcgtaagatggcagagcccggtaatagagtgcgtaagatggcagagcccggtaatagagtgcgtaagatggcagagcccggtaatagagtgcgtaagatggcagagcccggtaatgactcatatgctctagagggtatataatgggggcc

SEQ ID NO: 694 - DNA, 181 bp, synthetic construct
  source 1..181: mol_type = other DNA
taagatggcagagcccggtaatagagtgcgataagatggcagagcccggtaatagagtgcgataagatggcagagcccggtaatagagtgcgataagatggcagagcccggtaatagagtgcgataagatggcagagcccggtaatgactcatatgctctagagggtatataatgggggcc

SEQ ID NO: 695 - DNA, 185 bp, synthetic construct
  source 1..185: mol_type = other DNA
taagatggcagagcccggtaatagagtgcgagtaagatggcagagcccggtaatagagtgcgagtaagatggcagagcccggtaatagagtgcgagtaagatggcagagcccggtaatagagtgcgagtaagatggcagagcccggtaatgactcatatgctctagagggtatataatgggggcc

SEQ ID NO: 696 - DNA, 47 bp, synthetic construct
  source 1..47: mol_type = other DNA
attaccgggctctgccatcttactctagagggtatataatgggggcc

SEQ ID NO: 697 - DNA, 48 bp, synthetic construct
  source 1..48: mol_type = other DNA
attaccgggctctgccatcttagctctagagggtatataatgggggcc

SEQ ID NO: 698 - DNA, 49 bp, synthetic construct
  source 1..49: mol_type = other DNA
attaccgggctctgccatcttagactctagagggtatataatgggggcc

SEQ ID NO: 699 - DNA, 50 bp, synthetic construct
  source 1..50: mol_type = other DNA
attaccgggctctgccatcttagacctctagagggtatataatgggggcc

SEQ ID NO: 700 - DNA, 51 bp, synthetic construct
  source 1..51: mol_type = other DNA
attaccgggctctgccatcttagactctctagagggtatataatgggggcc

SEQ ID NO: 701 - DNA, 52 bp, synthetic construct
  source 1..52: mol_type = other DNA
attaccgggctctgccatcttagactcctctagagggtatataatgggggcc

SEQ ID NO: 702 - DNA, 53 bp, synthetic construct
  source 1..53: mol_type = other DNA
attaccgggctctgccatcttagactcactctagagggtatataatgggggcc

SEQ ID NO: 703 - DNA, 54 bp, synthetic construct
  source 1..54: mol_type = other DNA
attaccgggctctgccatcttagactcatctctagagggtatataatgggggcc

SEQ ID NO: 704 - DNA, 55 bp, synthetic construct
  source 1..55: mol_type = other DNA
attaccgggctctgccatcttagactcatactctagagggtatataatgggggcc

SEQ ID NO: 705 - DNA, 56 bp, synthetic construct
  source 1..56: mol_type = other DNA
attaccgggctctgccatcttagactcatatctctagagggtatataatgggggcc

SEQ ID NO: 706 - DNA, 57 bp, synthetic construct
  source 1..57: mol_type = other DNA
attaccgggctctgccatcttagactcatatgctctagagggtatataatgggggcc

SEQ ID NO: 707 - DNA, 69 bp, synthetic construct
  source 1..69: mol_type = other DNA
attaccgggctctgccatcttaattaccgggctctgccatcttactctagagggtatataatgggggcc

SEQ ID NO: 708 - DNA, 70 bp, synthetic construct
  source 1..70: mol_type = other DNA
attaccgggctctgccatcttaaattaccgggctctgccatcttactctagagggtatataatgggggcc

SEQ ID NO: 709 - DNA, 71 bp, synthetic construct
  source 1..71: mol_type = other DNA
attaccgggctctgccatcttaagattaccgggctctgccatcttactctagagggtatataatgggggcc

SEQ ID NO: 710 - DNA, 72 bp, synthetic construct
  source 1..72: mol_type = other DNA
attaccgggctctgccatcttaagaattaccgggctctgccatcttactctagagggtatataatgggggcc

SEQ ID NO: 711 - DNA, 73 bp, synthetic construct
  source 1..73: mol_type = other DNA
attaccgggctctgccatcttaagagattaccgggctctgccatcttactctagagggtatataatgggggcc

SEQ ID NO: 712 - DNA, 74 bp, synthetic construct
  source 1..74: mol_type = other DNA
attaccgggctctgccatcttaagagtattaccgggctctgccatcttactctagagggtatataatgggggcc

SEQ ID NO: 713 - DNA, 75 bp, synthetic construct
  source 1..75: mol_type = other DNA
attaccgggctctgccatcttaagagtgattaccgggctctgccatcttactctagagggtatataatgggggcc

SEQ ID NO: 714 - DNA, 76 bp, synthetic construct
  source 1..76: mol_type = other DNA
attaccgggctctgccatcttaagagtgcattaccgggctctgccatcttactctagagggtatataatgggggcc

SEQ ID NO: 715 - DNA, 77 bp, synthetic construct
  source 1..77: mol_type = other DNA
attaccgggctctgccatcttaagagtgcgattaccgggctctgccatcttactctagagggtatataatgggggcc

SEQ ID NO: 716 - DNA, 78 bp, synthetic construct
  source 1..78: mol_type = other DNA
attaccgggctctgccatcttaagagtgcgaattaccgggctctgccatcttactctagagggtatataatgggggcc

SEQ ID NO: 717 - DNA, 79 bp, synthetic construct
  source 1..79: mol_type = other DNA
attaccgggctctgccatcttaagagtgcgagattaccgggctctgccatcttactctagagggtatataatgggggcc

SEQ ID NO: 718 - DNA, 70 bp, synthetic construct
  source 1..70: mol_type = other DNA
attaccgggctctgccatcttaattaccgggctctgccatcttagctctagagggtatataatgggggcc

SEQ ID NO: 719 - DNA, 71 bp, synthetic construct
  source 1..71: mol_type = other DNA
attaccgggctctgccatcttaaattaccgggctctgccatcttagctctagagggtatataatgggggcc

SEQ ID NO: 720 - DNA, 72 bp, synthetic construct
  source 1..72: mol_type = other DNA
attaccgggctctgccatcttaagattaccgggctctgccatcttagctctagagggtatataatgggggcc

SEQ ID NO: 721 - DNA, 73 bp, synthetic construct
  source 1..73: mol_type = other DNA
attaccgggctctgccatcttaagaattaccgggctctgccatcttagctctagagggtatataatgggggcc

SEQ ID NO: 722 - DNA, 74 bp, synthetic construct
  source 1..74: mol_type = other DNA
attaccgggctctgccatcttaagagattaccgggctctgccatcttagctctagagggtatataatgggggcc

SEQ ID NO: 723 - DNA, 75 bp, synthetic construct
  source 1..75: mol_type = other DNA
attaccgggctctgccatcttaagagtattaccgggctctgccatcttagctctagagggtatataatgggggcc

SEQ ID NO: 724 - DNA, 76 bp, synthetic construct
  source 1..76: mol_type = other DNA
attaccgggctctgccatcttaagagtgattaccgggctctgccatcttagctctagagggtatataatgggggcc

SEQ ID NO: 725 - DNA, 77 bp, synthetic construct
  source 1..77: mol_type = other DNA
attaccgggctctgccatcttaagagtgcattaccgggctctgccatcttagctctagagggtatataatgggggcc

SEQ ID NO: 726 - DNA, 78 bp, synthetic construct
  source 1..78: mol_type = other DNA
attaccgggctctgccatcttaagagtgcgattaccgggctctgccatcttagctctagagggtatataatgggggcc

SEQ ID NO: 727 - DNA, 79 bp, synthetic construct
  source 1..79: mol_type = other DNA
attaccgggctctgccatcttaagagtgcgaattaccgggctctgccatcttagctctagagggtatataatgggggcc

SEQ ID NO: 728 - DNA, 80 bp, synthetic construct
  source 1..80: mol_type = other DNA
attaccgggctctgccatcttaagagtgcgagattaccgggctctgccatcttagctctagagggtatataatgggggcc

SEQ ID NO: 729 - DNA, 71 bp, synthetic construct
  source 1..71: mol_type = other DNA
attaccgggctctgccatcttaattaccgggctctgccatcttagactctagagggtatataatgggggcc

SEQ ID NO: 730 - DNA, 72 bp, synthetic construct
  source 1..72: mol_type = other DNA
attaccgggctctgccatcttaaattaccgggctctgccatcttagactctagagggtatataatgggggcc

SEQ ID NO: 731 - DNA, 73 bp, synthetic construct
  source 1..73: mol_type = other DNA
attaccgggctctgccatcttaagattaccgggctctgccatcttagactctagagggtatataatgggggcc

SEQ ID NO: 732 - DNA, 74 bp, synthetic construct
  source 1..74: mol_type = other DNA
attaccgggctctgccatcttaagaattaccgggctctgccatcttagactctagagggtatataatgggggcc

SEQ ID NO: 733 - DNA, 75 bp, synthetic construct
  source 1..75: mol_type = other DNA
attaccgggctctgccatcttaagagattaccgggctctgccatcttagactctagagggtatataatgggggcc

SEQ ID NO: 734 - DNA, 76 bp, synthetic construct
  source 1..76: mol_type = other DNA
attaccgggctctgccatcttaagagtattaccgggctctgccatcttagactctagagggtatataatgggggcc

SEQ ID NO: 735 - DNA, 77 bp, synthetic construct
  source 1..77: mol_type = other DNA
attaccgggctctgccatcttaagagtgattaccgggctctgccatcttagactctagagggtatataatgggggcc

SEQ ID NO: 736 - DNA, 78 bp, synthetic construct
  source 1..78: mol_type = other DNA
attaccgggctctgccatcttaagagtgcattaccgggctctgccatcttagactctagagggtatataatgggggcc

SEQ ID NO: 737 - DNA, 79 bp, synthetic construct
  source 1..79: mol_type = other DNA
attaccgggctctgccatcttaagagtgcgattaccgggctctgccatcttagactctagagggtatataatgggggcc

SEQ ID NO: 738 - DNA, 80 bp, synthetic construct
  source 1..80: mol_type = other DNA
attaccgggctctgccatcttaagagtgcgaattaccgggctctgccatcttagactctagagggtatataatgggggcc

SEQ ID NO: 739 - DNA, 81 bp, synthetic construct
  source 1..81: mol_type = other DNA
attaccgggctctgccatcttaagagtgcgagattaccgggctctgccatcttagactctagagggtatataatgggggcc

SEQ ID NO: 740 - DNA, 72 bp, synthetic construct
  source 1..72: mol_type = other DNA
attaccgggctctgccatcttaattaccgggctctgccatcttagacctctagagggtatataatgggggcc

SEQ ID NO: 741 - DNA, 73 bp, synthetic construct
  source 1..73: mol_type = other DNA
attaccgggctctgccatcttaaattaccgggctctgccatcttagacctctagagggtatataatgggggcc

SEQ ID NO: 742 - DNA, 74 bp, synthetic construct
  source 1..74: mol_type = other DNA
attaccgggctctgccatcttaagattaccgggctctgccatcttagacctctagagggtatataatgggggcc

SEQ ID NO: 743 - DNA, 75 bp, synthetic construct
  source 1..75: mol_type = other DNA
attaccgggctctgccatcttaagaattaccgggctctgccatcttagacctctagagggtatataatgggggcc

SEQ ID NO: 744 - DNA, 76 bp, synthetic construct
  source 1..76: mol_type = other DNA
attaccgggctctgccatcttaagagattaccgggctctgccatcttagacctctagagggtatataatgggggcc

SEQ ID NO: 745 - DNA, 77 bp, synthetic construct
  source 1..77: mol_type = other DNA
attaccgggctctgccatcttaagagtattaccgggctctgccatcttagacctctagagggtatataatgggggcc

SEQ ID NO: 746 - DNA, 78 bp, synthetic construct
  source 1..78: mol_type = other DNA
attaccgggctctgccatcttaagagtgattaccgggctctgccatcttagacctctagagggtatataatgggggcc

SEQ ID NO: 747 - DNA, 79 bp, synthetic construct
  source 1..79: mol_type = other DNA
attaccgggctctgccatcttaagagtgcattaccgggctctgccatcttagacctctagagggtatataatgggggcc

SEQ ID NO: 748 - DNA, 80 bp, synthetic construct
  source 1..80: mol_type = other DNA
attaccgggctctgccatcttaagagtgcgattaccgggctctgccatcttagacctctagagggtatataatgggggcc

SEQ ID NO: 749 - DNA, 81 bp, synthetic construct
  source 1..81: mol_type = other DNA
attaccgggctctgccatcttaagagtgcgaattaccgggctctgccatcttagacctctagagggtatataatgggggcc

SEQ ID NO: 750 - DNA, 82 bp, synthetic construct
  source 1..82: mol_type = other DNA
attaccgggctctgccatcttaagagtgcgagattaccgggctctgccatcttagacctctagagggtatataatgggggcc

SEQ ID NO: 751 - DNA, 73 bp, synthetic construct
  source 1..73: mol_type = other DNA
attaccgggctctgccatcttaattaccgggctctgccatcttagactctctagagggtatataatgggggcc

SEQ ID NO: 752 - DNA, 74 bp, synthetic construct
  source 1..74: mol_type = other DNA
attaccgggctctgccatcttaaattaccgggctctgccatcttagactctctagagggtatataatgggggcc

SEQ ID NO: 753 - DNA, 75 bp, synthetic construct
  source 1..75: mol_type = other DNA
attaccgggctctgccatcttaagattaccgggctctgccatcttagactctctagagggtatataatgggggcc

SEQ ID NO: 754 - DNA, 76 bp, synthetic construct
  source 1..76: mol_type = other DNA
attaccgggctctgccatcttaagaattaccgggctctgccatcttagactctctagagggtatataatgggggcc

SEQ ID NO: 755 - DNA, 77 bp, synthetic construct
  source 1..77: mol_type = other DNA
attaccgggctctgccatcttaagagattaccgggctctgccatcttagactctctagagggtatataatgggggcc

SEQ ID NO: 756 - DNA, 78 bp, synthetic construct
  source 1..78: mol_type = other DNA
attaccgggctctgccatcttaagagtattaccgggctctgccatcttagactctctagagggtatataatgggggcc

SEQ ID NO: 757 - DNA, 79 bp, synthetic construct
  source 1..79: mol_type = other DNA
attaccgggctctgccatcttaagagtgattaccgggctctgccatcttagactctctagagggtatataatgggggcc

SEQ ID NO: 758 - DNA, 80 bp, synthetic construct
  source 1..80: mol_type = other DNA
attaccgggctctgccatcttaagagtgcattaccgggctctgccatcttagactctctagagggtatataatgggggcc

SEQ ID NO: 759 - DNA, 81 bp, synthetic construct
  source 1..81: mol_type = other DNA
attaccgggctctgccatcttaagagtgcgattaccgggctctgccatcttagactctctagagggtatataatgggggcc

SEQ ID NO: 760 - DNA, 82 bp, synthetic construct
  source 1..82: mol_type = other DNA
attaccgggctctgccatcttaagagtgcgaattaccgggctctgccatcttagactctctagagggtatataatgggggcc

SEQ ID NO: 761 - DNA, 83 bp, synthetic construct
  source 1..83: mol_type = other DNA
attaccgggctctgccatcttaagagtgcgagattaccgggctctgccatcttagactctctagagggtatataatgggggcc

SEQ ID NO: 762 - DNA, 74 bp, synthetic construct
  source 1..74: mol_type = other DNA
attaccgggctctgccatcttaattaccgggctctgccatcttagactcctctagagggtatataatgggggcc

SEQ ID NO: 763 - DNA, 75 bp, synthetic construct
  source 1..75: mol_type = other DNA
attaccgggctctgccatcttaaattaccgggctctgccatcttagactcctctagagggtatataatgggggcc

SEQ ID NO: 764 - DNA, 76 bp, synthetic construct
  source 1..76: mol_type = other DNA
attaccgggctctgccatcttaagattaccgggctctgccatcttagactcctctagagggtatataatgggggcc

SEQ ID NO: 765 - DNA, 77 bp, synthetic construct
  source 1..77: mol_type = other DNA
attaccgggctctgccatcttaagaattaccgggctctgccatcttagactcctctagagggtatataatgggggcc

SEQ ID NO: 766 - DNA, 78 bp, synthetic construct
  source 1..78: mol_type = other DNA
attaccgggctctgccatcttaagagattaccgggctctgccatcttagactcctctagagggtatataatgggggcc

SEQ ID NO: 767 - DNA, 79 bp, synthetic construct
  source 1..79: mol_type = other DNA
attaccgggctctgccatcttaagagtattaccgggctctgccatcttagactcctctagagggtatataatgggggcc

SEQ ID NO: 768 - DNA, 80 bp, synthetic construct
  source 1..80: mol_type = other DNA
attaccgggctctgccatcttaagagtgattaccgggctctgccatcttagactcctctagagggtatataatgggggcc

SEQ ID NO: 769 - DNA, 81 bp, synthetic construct
  source 1..81: mol_type = other DNA
attaccgggctctgccatcttaagagtgcattaccgggctctgccatcttagactcctctagagggtatataatgggggcc

SEQ ID NO: 770 - DNA, 82 bp, synthetic construct
  source 1..82: mol_type = other DNA
attaccgggctctgccatcttaagagtgcgattaccgggctctgccatcttagactcctctagagggtatataatgggggcc

SEQ ID NO: 771 - DNA, 83 bp, synthetic construct
  source 1..83: mol_type = other DNA
attaccgggctctgccatcttaagagtgcgaattaccgggctctgccatcttagactcctctagagggtatataatgggggcc

SEQ ID NO: 772 - DNA, 84 bp, synthetic construct
  source 1..84: mol_type = other DNA
attaccgggctctgccatcttaagagtgcgagattaccgggctctgccatcttagactcctctagagggtatataatgggggcc

SEQ ID NO: 773 - DNA, 75 bp, synthetic construct
  source 1..75: mol_type = other DNA
attaccgggctctgccatcttaattaccgggctctgccatcttagactcactctagagggtatataatgggggcc

SEQ ID NO: 774 - DNA, 76 bp, synthetic construct
  source 1..76: mol_type = other DNA
attaccgggctctgccatcttaaattaccgggctctgccatcttagactcactctagagggtatataatgggggcc

SEQ ID NO: 775 - DNA, 77 bp, synthetic construct
  source 1..77: mol_type = other DNA
attaccgggctctgccatcttaagattaccgggctctgccatcttagactcactctagagggtatataatgggggcc

SEQ ID NO: 776 - DNA, 78 bp, synthetic construct
  source 1..78: mol_type = other DNA
attaccgggctctgccatcttaagaattaccgggctctgccatcttagactcactctagagggtatataatgggggcc

SEQ ID NO: 777 - DNA, 79 bp, synthetic construct
  source 1..79: mol_type = other DNA
attaccgggctctgccatcttaagagattaccgggctctgccatcttagactcactctagagggtatataatgggggcc

SEQ ID NO: 778 - DNA, 80 bp, synthetic construct
  source 1..80: mol_type = other DNA
attaccgggctctgccatcttaagagtattaccgggctctgccatcttagactcactctagagggtatataatgggggcc

SEQ ID NO: 779 - DNA, 81 bp, synthetic construct
  source 1..81: mol_type = other DNA
attaccgggctctgccatcttaagagtgattaccgggctctgccatcttagactcactctagagggtatataatgggggcc

SEQ ID NO: 780 - DNA, 82 bp, synthetic construct
  source 1..82: mol_type = other DNA
attaccgggctctgccatcttaagagtgcattaccgggctctgccatcttagactcactctagagggtatataatgggggcc

SEQ ID NO: 781 - DNA, 83 bp, synthetic construct
  source 1..83: mol_type = other DNA
attaccgggctctgccatcttaagagtgcgattaccgggctctgccatcttagactcactctagagggtatataatgggggcc

SEQ ID NO: 782 - DNA, 84 bp, synthetic construct
  source 1..84: mol_type = other DNA
attaccgggctctgccatcttaagagtgcgaattaccgggctctgccatcttagactcactctagagggtatataatgggggcc

SEQ ID NO: 783 - DNA, 85 bp, synthetic construct
  source 1..85: mol_type = other DNA
attaccgggctctgccatcttaagagtgcgagattaccgggctctgccatcttagactcactctagagggtatataatgggggcc

SEQ ID NO: 784 - DNA, 76 bp, synthetic construct
  source 1..76: mol_type = other DNA
attaccgggctctgccatcttaattaccgggctctgccatcttagactcatctctagagggtatataatgggggcc

SEQ ID NO: 785 - DNA, 77 bp, synthetic construct
  source 1..77: mol_type = other DNA
attaccgggctctgccatcttaaattaccgggctctgccatcttagactcatctctagagggtatataatgggggcc

SEQ ID NO: 786 - DNA, 78 bp, synthetic construct
  source 1..78: mol_type = other DNA
attaccgggctctgccatcttaagattaccgggctctgccatcttagactcatctctagagggtatataatgggggcc

SEQ ID NO: 787 - DNA, 79 bp, synthetic construct
  source 1..79: mol_type = other DNA
attaccgggctctgccatcttaagaattaccgggctctgccatcttagactcatctctagagggtatataatgggggcc

SEQ ID NO: 788 - DNA, 80 bp, synthetic construct
  source 1..80: mol_type = other DNA
attaccgggctctgccatcttaagagattaccgggctctgccatcttagactcatctctagagggtatataatgggggcc

SEQ ID NO: 789 - DNA, 81 bp, synthetic construct
  source 1..81: mol_type = other DNA
attaccgggctctgccatcttaagagtattaccgggctctgccatcttagactcatctctagagggtatataatgggggcc

SEQ ID NO: 790 - DNA, 82 bp, synthetic construct
  source 1..82: mol_type = other DNA
attaccgggctctgccatcttaagagtgattaccgggctctgccatcttagactcatctctagagggtatataatgggggcc

SEQ ID NO: 791 - DNA, 83 bp, synthetic construct
  source 1..83: mol_type = other DNA
attaccgggctctgccatcttaagagtgcattaccgggctctgccatcttagactcatctctagagggtatataatgggggcc

SEQ ID NO: 792 - DNA, 84 bp, synthetic construct
  source 1..84: mol_type = other DNA
attaccgggctctgccatcttaagagtgcgattaccgggctctgccatcttagactcatctctagagggtatataatgggggcc

SEQ ID NO: 793 - DNA, 85 bp, synthetic construct
  source 1..85: mol_type = other DNA
attaccgggctctgccatcttaagagtgcgaattaccgggctctgccatcttagactcatctctagagggtatataatgggggcc

SEQ ID NO: 794 - DNA, 86 bp, synthetic construct
  source 1..86: mol_type = other DNA
attaccgggctctgccatcttaagagtgcgagattaccgggctctgccatcttagactcatctctagagggtatataatgggggcc

SEQ ID NO: 795 - DNA, 77 bp, synthetic construct
  source 1..77: mol_type = other DNA
attaccgggctctgccatcttaattaccgggctctgccatcttagactcatactctagagggtatataatgggggcc

SEQ ID NO: 796 - DNA, 78 bp, synthetic construct
  source 1..78: mol_type = other DNA
attaccgggctctgccatcttaaattaccgggctctgccatcttagactcatactctagagggtatataatgggggcc

SEQ ID NO: 797 - DNA, 79 bp, synthetic construct
  source 1..79: mol_type = other DNA
attaccgggctctgccatcttaagattaccgggctctgccatcttagactcatactctagagggtatataatgggggcc

SEQ ID NO: 798 - DNA, 80 bp, synthetic construct
  source 1..80: mol_type = other DNA
attaccgggctctgccatcttaagaattaccgggctctgccatcttagactcatactctagagggtatataatgggggcc

SEQ ID NO: 799 - DNA, 81 bp, synthetic construct
  source 1..81: mol_type = other DNA
attaccgggctctgccatcttaagagattaccgggctctgccatcttagactcatactctagagggtatataatgggggcc

SEQ ID NO: 800 - DNA, 82 bp, synthetic construct
  source 1..82: mol_type = other DNA
attaccgggctctgccatcttaagagtattaccgggctctgccatcttagactcatactctagagggtatataatgggggcc

SEQ ID NO: 801 - DNA, 83 bp, synthetic construct
  source 1..83: mol_type = other DNA
attaccgggctctgccatcttaagagtgattaccgggctctgccatcttagactcatactctagagggtatataatgggggcc

SEQ ID NO: 802 - DNA, 84 bp, synthetic construct
  source 1..84: mol_type = other DNA
attaccgggctctgccatcttaagagtgcattaccgggctctgccatcttagactcatactctagagggtatataatgggggcc

SEQ ID NO: 803 - DNA, 85 bp, synthetic construct
  source 1..85: mol_type = other DNA
attaccgggctctgccatcttaagagtgcgattaccgggctctgccatcttagactcatactctagagggtatataatgggggcc

SEQ ID NO: 804 - DNA, 86 bp, synthetic construct
  source 1..86: mol_type = other DNA
attaccgggctctgccatcttaagagtgcgaattaccgggctctgccatcttagactcatactctagagggtatataatgggggcc

SEQ ID NO: 805 - DNA, 87 bp, synthetic construct
  source 1..87: mol_type = other DNA
attaccgggctctgccatcttaagagtgcgagattaccgggctctgccatcttagactcatactctagagggtatataatgggggcc

SEQ ID NO: 806 - DNA, 78 bp, synthetic construct
  source 1..78: mol_type = other DNA
attaccgggctctgccatcttaattaccgggctctgccatcttagactcatatctctagagggtatataatgggggcc

SEQ ID NO: 807 - DNA, 79 bp, synthetic construct
  source 1..79: mol_type = other DNA
attaccgggctctgccatcttaaattaccgggctctgccatcttagactcatatctctagagggtatataatgggggcc

SEQ ID NO: 808 - DNA, 80 bp, synthetic construct
  source 1..80: mol_type = other DNA
attaccgggctctgccatcttaagattaccgggctctgccatcttagactcatatctctagagggtatataatgggggcc

SEQ ID NO: 809 - DNA, 81 bp, synthetic construct
  source 1..81: mol_type = other DNA
attaccgggctctgccatcttaagaattaccgggctctgccatcttagactcatatctctagagggtatataatgggggcc

SEQ ID NO: 810 - DNA, 82 bp, synthetic construct
  source 1..82: mol_type = other DNA
attaccgggctctgccatcttaagagattaccgggctctgccatcttagactcatatctctagagggtatataatgggggcc

SEQ ID NO: 811 - DNA, 83 bp, synthetic construct
  source 1..83: mol_type = other DNA
attaccgggctctgccatcttaagagtattaccgggctctgccatcttagactcatatctctagagggtatataatgggggcc

SEQ ID NO: 812 - DNA, 84 bp, synthetic construct
  source 1..84: mol_type = other DNA
attaccgggctctgccatcttaagagtgattaccgggctctgccatcttagactcatatctctagagggtatataatgggggcc

SEQ ID NO: 813 - DNA, 85 bp, synthetic construct
  source 1..85: mol_type = other DNA
attaccgggctctgccatcttaagagtgcattaccgggctctgccatcttagactcatatctctagagggtatataatgggggcc

SEQ ID NO: 814 - DNA, 86 bp, synthetic construct
  source 1..86: mol_type = other DNA
attaccgggctctgccatcttaagagtgcgattaccgggctctgccatcttagactcatatctctagagggtatataatgggggcc

SEQ ID NO: 815 - DNA, 87 bp, synthetic construct
  source 1..87: mol_type = other DNA
attaccgggctctgccatcttaagagtgcgaattaccgggctctgccatcttagactcatatctctagagggtatataatgggggcc

SEQ ID NO: 816 - DNA, 88 bp, synthetic construct
  source 1..88: mol_type = other DNA
attaccgggctctgccatcttaagagtgcgagattaccgggctctgccatcttagactcatatctctagagggtatataatgggggcc

SEQ ID NO: 817 - DNA, 79 bp, synthetic construct
  source 1..79: mol_type = other DNA
attaccgggctctgccatcttaattaccgggctctgccatcttagactcatatgctctagagggtatataatgggggcc

SEQ ID NO: 818 - DNA, 80 bp, synthetic construct
  source 1..80: mol_type = other DNA
attaccgggctctgccatcttaaattaccgggctctgccatcttagactcatatgctctagagggtatataatgggggcc

SEQ ID NO: 819 - DNA, 81 bp, synthetic construct
  source 1..81: mol_type = other DNA
attaccgggctctgccatcttaagattaccgggctctgccatcttagactcatatgctctagagggtatataatgggggcc

SEQ ID NO: 820 - DNA, 82 bp, synthetic construct
  source 1..82: mol_type = other DNA
attaccgggctctgccatcttaagaattaccgggctctgccatcttagactcatatgctctagagggtatataatgggggcc

SEQ ID NO: 821 - DNA, 83 bp, synthetic construct
  source 1..83: mol_type = other DNA
attaccgggctctgccatcttaagagattaccgggctctgccatcttagactcatatgctctagagggtatataatgggggcc

SEQ ID NO: 822 - DNA, 84 bp, synthetic construct
  source 1..84: mol_type = other DNA
attaccgggctctgccatcttaagagtattaccgggctctgccatcttagactcatatgctctagagggtatataatgggggcc

SEQ ID NO: 823 - DNA, 85 bp, synthetic construct
  source 1..85: mol_type = other DNA
attaccgggctctgccatcttaagagtgattaccgggctctgccatcttagactcatatgctctagagggtatataatgggggcc

SEQ ID NO: 824 - DNA, 86 bp, synthetic construct
  source 1..86: mol_type = other DNA
attaccgggctctgccatcttaagagtgcattaccgggctctgccatcttagactcatatgctctagagggtatataatgggggcc

SEQ ID NO: 825 - DNA, 87 bp, synthetic construct
  source 1..87: mol_type = other DNA
attaccgggctctgccatcttaagagtgcgattaccgggctctgccatcttagactcatatgctctagagggtatataatgggggcc

SEQ ID NO: 826 - DNA, 88 bp, synthetic construct
  source 1..88: mol_type = other DNA
attaccgggctctgccatcttaagagtgcgaattaccgggctctgccatcttagactcatatgctctagagggtatataatgggggcc

SEQ ID NO: 827 - DNA, 89 bp, synthetic construct
  source 1..89: mol_type = other DNA
attaccgggctctgccatcttaagagtgcgagattaccgggctctgccatcttagactcatatgctctagagggtatataatgggggcc

SEQ ID NO: 828 - DNA, 91 bp, synthetic construct
  source 1..91: mol_type = other DNA
attaccgggctctgccatcttaattaccgggctctgccatcttaattaccgggctctgccatcttactctagagggtatataatgggggcc

SEQ ID NO: 829 - DNA, 93 bp, synthetic construct
  source 1..93: mol_type = other DNA
attaccgggctctgccatcttaaattaccgggctctgccatcttaaattaccgggctctgccatcttactctagagggtatataatgggggcc

SEQ ID NO: 830 - DNA, 95 bp, synthetic construct
  source 1..95: mol_type = other DNA
attaccgggctctgccatcttaagattaccgggctctgccatcttaagattaccgggctctgccatcttactctagagggtatataatgggggcc

SEQ ID NO: 831 - DNA, 97 bp, synthetic construct
  source 1..97: mol_type = other DNA
attaccgggctctgccatcttaagaattaccgggctctgccatcttaagaattaccgggctctgccatcttactctagagggtatataatgggggcc

SEQ ID NO: 832 - DNA, 99 bp, synthetic construct
  source 1..99: mol_type = other DNA
attaccgggctctgccatcttaagagattaccgggctctgccatcttaagagattaccgggctctgccatcttactctagagggtatataatgggggcc

SEQ ID NO: 833 - DNA, 101 bp, synthetic construct
  source 1..101: mol_type = other DNA
attaccgggctctgccatcttaagagtattaccgggctctgccatcttaagagtattaccgggctctgccatcttactctagagggtatataatgggggcc

SEQ ID NO: 834 - DNA, 103 bp, synthetic construct
  source 1..103: mol_type = other DNA
attaccgggctctgccatcttaagagtgattaccgggctctgccatcttaagagtgattaccgggctctgccatcttactctagagggtatataatgggggcc

SEQ ID NO: 835 - DNA, 105 bp, synthetic construct
  source 1..105: mol_type = other DNA
attaccgggctctgccatcttaagagtgcattaccgggctctgccatcttaagagtgcattaccgggctctgccatcttactctagagggtatataatgggggcc

SEQ ID NO: 836 - DNA, 107 bp, synthetic construct
  source 1..107: mol_type = other DNA
attaccgggctctgccatcttaagagtgcgattaccgggctctgccatcttaagagtgcgattaccgggctctgccatcttactctagagggtatataatgggggcc

SEQ ID NO: 837 - DNA, 109 bp, synthetic construct
  source 1..109: mol_type = other DNA
attaccgggctctgccatcttaagagtgcgaattaccgggctctgccatcttaagagtgcgaattaccgggctctgccatcttactctagagggtatataatgggggcc

SEQ ID NO: 838 - DNA, 111 bp, synthetic construct
  source 1..111: mol_type = other DNA
attaccgggctctgccatcttaagagtgcgagattaccgggctctgccatcttaagagtgcgagattaccgggctctgccatcttactctagagggtatataatgggggcc

SEQ ID NO: 839 - DNA, 92 bp, synthetic construct
  source 1..92: mol_type = other DNA
attaccgggctctgccatcttaattaccgggctctgccatcttaattaccgggctctgccatcttagctctagagggtatataatgggggcc

SEQ ID NO: 840 - DNA, 94 bp, synthetic construct
  source 1..94: mol_type = other DNA
attaccgggctctgccatcttaaattaccgggctctgccatcttaaattaccgggctctgccatcttagctctagagggtatataatgggggcc

SEQ ID NO: 841 - DNA, 96 bp, synthetic construct
  source 1..96: mol_type = other DNA
attaccgggctctgccatcttaagattaccgggctctgccatcttaagattaccgggctctgccatcttagctctagagggtatataatgggggcc

SEQ ID NO: 842 - DNA, 98 bp, synthetic construct
  source 1..98: mol_type = other DNA
attaccgggctctgccatcttaagaattaccgggctctgccatcttaagaattaccgggctctgccatcttagctctagagggtatataatgggggcc

SEQ ID NO: 843 - DNA, 100 bp, synthetic construct
  source 1..100: mol_type = other DNA
attaccgggctctgccatcttaagagattaccgggctctgccatcttaagagattaccgggctctgccatcttagctctagagggtatataatgggggcc

SEQ ID NO: 844 - DNA, 102 bp, synthetic construct
  source 1..102: mol_type = other DNA
attaccgggctctgccatcttaagagtattaccgggctctgccatcttaagagtattaccgggctctgccatcttagctctagagggtatataatgggggcc

SEQ ID NO: 845 - DNA, 104 bp, synthetic construct
  source 1..104: mol_type = other DNA
attaccgggctctgccatcttaagagtgattaccgggctctgccatcttaagagtgattaccgggctctgccatcttagctctagagggtatataatgggggcc

SEQ ID NO: 846 - DNA, 106 bp, synthetic construct
  source 1..106: mol_type = other DNA
attaccgggctctgccatcttaagagtgcattaccgggctctgccatcttaagagtgcattaccgggctctgccatcttagctctagagggtatataatgggggcc

SEQ ID NO: 847 - DNA, 108 bp, synthetic construct
  source 1..108: mol_type = other DNA
attaccgggctctgccatcttaagagtgcgattaccgggctctgccatcttaagagtgcgattaccgggctctgccatcttagctctagagggtatataatgggggcc

SEQ ID NO: 848 - DNA, 110 bp, synthetic construct
  source 1..110: mol_type = other DNA
attaccgggctctgccatcttaagagtgcgaattaccgggctctgccatcttaagagtgcgaattaccgggctctgccatcttagctctagagggtatataatgggggcc

SEQ ID NO: 849 - DNA, 112 bp, synthetic construct
  source 1..112: mol_type = other DNA
attaccgggctctgccatcttaagagtgcgagattaccgggctctgccatcttaagagtgcgagattaccgggctctgccatcttagctctagagggtatataatgggggcc

SEQ ID NO: 850 - DNA, 93 bp, synthetic construct
  source 1..93: mol_type = other DNA
attaccgggctctgccatcttaattaccgggctctgccatcttaattaccgggctctgccatcttagactctagagggtatataatgggggcc

SEQ ID NO: 851 - DNA, 95 bp, synthetic construct
  source 1..95: mol_type = other DNA
attaccgggctctgccatcttaaattaccgggctctgccatcttaaattaccgggctctgccatcttagactctagagggtatataatgggggcc

SEQ ID NO: 852 - DNA, 97 bp, synthetic construct
  source 1..97: mol_type = other DNA
attaccgggctctgccatcttaagattaccgggctctgccatcttaagattaccgggctctgccatcttagactctagagggtatataatgggggcc

SEQ ID NO: 853 - DNA, 99 bp, synthetic construct
  source 1..99: mol_type = other DNA
attaccgggctctgccatcttaagaattaccgggctctgccatcttaagaattaccgggctctgccatcttagactctagagggtatataatgggggcc

SEQ ID NO: 854 - DNA, 101 bp, synthetic construct
  source 1..101: mol_type = other DNA
attaccgggctctgccatcttaagagattaccgggctctgccatcttaagagattaccgggctctgccatcttagactctagagggtatataatgggggcc

SEQ ID NO: 855 - DNA, 103 bp, synthetic construct
  source 1..103: mol_type = other DNA
attaccgggctctgccatcttaagagtattaccgggctctgccatcttaagagtattaccgggctctgccatcttagactctagagggtatataatgggggcc

SEQ ID NO: 856 - DNA, 105 bp, synthetic construct
  source 1..105: mol_type = other DNA
attaccgggctctgccatcttaagagtgattaccgggctctgccatcttaagagtgattaccgggctctgccatcttagactctagagggtatataatgggggcc

SEQ ID NO: 857 - DNA, 107 bp, synthetic construct
  source 1..107: mol_type = other DNA
attaccgggctctgccatcttaagagtgcattaccgggctctgccatcttaagagtgcattaccgggctctgccatcttagactctagagggtatataatgggggcc

SEQ ID NO: 858 - DNA, 109 bp, synthetic construct
  source 1..109: mol_type = other DNA
attaccgggctctgccatcttaagagtgcgattaccgggctctgccatcttaagagtgcgattaccgggctctgccatcttagactctagagggtatataatgggggcc

SEQ ID NO: 859 - DNA, 111 bp, synthetic construct
  source 1..111: mol_type = other DNA
attaccgggctctgccatcttaagagtgcgaattaccgggctctgccatcttaagagtgcgaattaccgggctctgccatcttagactctagagggtatataatgggggcc

SEQ ID NO: 860 - DNA, 113 bp, synthetic construct
  source 1..113: mol_type = other DNA
attaccgggctctgccatcttaagagtgcgagattaccgggctctgccatcttaagagtgcgagattaccgggctctgccatcttagactctagagggtatataatgggggcc

SEQ ID NO: 861 - DNA, 94 bp, synthetic construct
  source 1..94: mol_type = other DNA
attaccgggctctgccatcttaattaccgggctctgccatcttaattaccgggctctgccatcttagacctctagagggtatataatgggggcc

SEQ ID NO: 862 - DNA, 96 bp, synthetic construct
  source 1..96: mol_type = other DNA
attaccgggctctgccatcttaaattaccgggctctgccatcttaaattaccgggctctgccatcttagacctctagagggtatataatgggggcc

SEQ ID NO: 863 - DNA, 98 bp, synthetic construct
  source 1..98: mol_type = other DNA
attaccgggctctgccatcttaagattaccgggctctgccatcttaagattaccgggctctgccatcttagacctctagagggtatataatgggggcc

SEQ ID NO: 864 - DNA, 100 bp, synthetic construct
  source 1..100: mol_type = other DNA
attaccgggctctgccatcttaagaattaccgggctctgccatcttaagaattaccgggctctgccatcttagacctctagagggtatataatgggggcc

SEQ ID NO: 865 - DNA, 102 bp, synthetic construct
  source 1..102: mol_type = other DNA
attaccgggctctgccatcttaagagattaccgggctctgccatcttaagagattaccgggctctgccatcttagacctctagagggtatataatgggggcc

SEQ ID NO: 866 - DNA, 104 bp, synthetic construct
  source 1..104: mol_type = other DNA
attaccgggctctgccatcttaagagtattaccgggctctgccatcttaagagtattaccgggctctgccatcttagacctctagagggtatataatgggggcc

SEQ ID NO: 867 - DNA, 106 bp, synthetic construct
  source 1..106: mol_type = other DNA
attaccgggctctgccatcttaagagtgattaccgggctctgccatcttaagagtgattaccgggctctgccatcttagacctctagagggtatataatgggggcc

SEQ ID NO: 868 - DNA, 108 bp, synthetic construct
  source 1..108: mol_type = other DNA
attaccgggctctgccatcttaagagtgcattaccgggctctgccatcttaagagtgcattaccgggctctgccatcttagacctctagagggtatataatgggggcc

SEQ ID NO: 869 - DNA, 110 bp, synthetic construct
  source 1..110: mol_type = other DNA
attaccgggctctgccatcttaagagtgcgattaccgggctctgccatcttaagagtgcgattaccgggctctgccatcttagacctctagagggtatataatgggggcc

SEQ ID NO: 870 - DNA, 112 bp, synthetic construct
  source 1..112: mol_type = other DNA
attaccgggctctgccatcttaagagtgcgaattaccgggctctgccatcttaagagtgcgaattaccgggctctgccatcttagacctctagagggtatataatgggggcc

SEQ ID NO: 871 - DNA, 114 bp, synthetic construct
  source 1..114: mol_type = other DNA
attaccgggctctgccatcttaagagtgcgagattaccgggctctgccatcttaagagtgcgagattaccgggctctgccatcttagacctctagagggtatataatgggggcc

SEQ ID NO: 872 - DNA, 95 bp, synthetic construct
  source 1..95: mol_type = other DNA
attaccgggctctgccatcttaattaccgggctctgccatcttaattaccgggctctgccatcttagactctctagagggtatataatgggggcc

SEQ ID NO: 873 - DNA, 97 bp, synthetic construct
  source 1..97: mol_type = other DNA
attaccgggctctgccatcttaaattaccgggctctgccatcttaaattaccgggctctgccatcttagactctctagagggtatataatgggggcc

SEQ ID NO: 874 - DNA, 99 bp, synthetic construct
  source 1..99: mol_type = other DNA
attaccgggctctgccatcttaagattaccgggctctgccatcttaagattaccgggctctgccatcttagactctctagagggtatataatgggggcc

SEQ ID NO: 875 - DNA, 101 bp, synthetic construct
  source 1..101: mol_type = other DNA
attaccgggctctgccatcttaagaattaccgggctctgccatcttaagaattaccgggctctgccatcttagactctctagagggtatataatgggggcc

SEQ ID NO: 876 - DNA, 103 bp, synthetic construct
  source 1..103: mol_type = other DNA
attaccgggctctgccatcttaagagattaccgggctctgccatcttaagagattaccgggctctgccatcttagactctctagagggtatataatgggggcc

SEQ ID NO: 877 - DNA, 105 bp, synthetic construct
  source 1..105: mol_type = other DNA
attaccgggctctgccatcttaagagtattaccgggctctgccatcttaagagtattaccgggctctgccatcttagactctctagagggtatataatgggggcc

SEQ ID NO: 878 - DNA, 107 bp, synthetic construct
  source 1..107: mol_type = other DNA
attaccgggctctgccatcttaagagtgattaccgggctctgccatcttaagagtgattaccgggctctgccatcttagactctctagagggtatataatgggggcc

SEQ ID NO: 879 - DNA, 109 bp, synthetic construct
  source 1..109: mol_type = other DNA
attaccgggctctgccatcttaagagtgcattaccgggctctgccatcttaagagtgcattaccgggctctgccatcttagactctctagagggtatataatgggggcc

SEQ ID NO: 880 - DNA, 111 bp, synthetic construct
  source 1..111: mol_type = other DNA
attaccgggctctgccatcttaagagtgcgattaccgggctctgccatcttaagagtgcgattaccgggctctgccatcttagactctctagagggtatataatgggggcc

SEQ ID NO: 881 - DNA, 113 bp, synthetic construct
  source 1..113: mol_type = other DNA
attaccgggctctgccatcttaagagtgcgaattaccgggctctgccatcttaagagtgcgaattaccgggctctgccatcttagactctctagagggtatataatgggggcc

SEQ ID NO: 882 - DNA, 115 bp, synthetic construct
  source 1..115: mol_type = other DNA
attaccgggctctgccatcttaagagtgcgagattaccgggctctgccatcttaagagtgcgagattaccgggctctgccatcttagactctctagagggtatataatgggggcc

SEQ ID NO: 883 - DNA, 96 bp, synthetic construct
  source 1..96: mol_type = other DNA
attaccgggctctgccatcttaattaccgggctctgccatcttaattaccgggctctgccatcttagactcctctagagggtatataatgggggcc

SEQ ID NO: 884 - DNA, 98 bp, synthetic construct
  source 1..98: mol_type = other DNA
attaccgggctctgccatcttaaattaccgggctctgccatcttaaattaccgggctctgccatcttagactcctctagagggtatataatgggggcc

SEQ ID NO: 885 - DNA, 100 bp, synthetic construct
  source 1..100: mol_type = other DNA
attaccgggctctgccatcttaagattaccgggctctgccatcttaagattaccgggctctgccatcttagactcctctagagggtatataatgggggcc

SEQ ID NO: 886 - DNA, 102 bp, synthetic construct
  source 1..102: mol_type = other DNA
attaccgggctctgccatcttaagaattaccgggctctgccatcttaagaattaccgggctctgccatcttagactcctctagagggtatataatgggggcc

SEQ ID NO: 887 - DNA, 104 bp, synthetic construct
  source 1..104: mol_type = other DNA
attaccgggctctgccatcttaagagattaccgggctctgccatcttaagagattaccgggctctgccatcttagactcctctagagggtatataatgggggcc

SEQ ID NO: 888 - DNA, 106 bp, synthetic construct
  source 1..106: mol_type = other DNA
attaccgggctctgccatcttaagagtattaccgggctctgccatcttaagagtattaccgggctctgccatcttagactcctctagagggtatataatgggggcc

SEQ ID NO: 889 - DNA, 108 bp, synthetic construct
  source 1..108: mol_type = other DNA
attaccgggctctgccatcttaagagtgattaccgggctctgccatcttaagagtgattaccgggctctgccatcttagactcctctagagggtatataatgggggcc

SEQ ID NO: 890 - DNA, 110 bp, synthetic construct
  source 1..110: mol_type = other DNA
attaccgggctctgccatcttaagagtgcattaccgggctctgccatcttaagagtgcattaccgggctctgccatcttagactcctctagagggtatataatgggggcc

SEQ ID NO: 891 - DNA, 112 bp, synthetic construct
  source 1..112: mol_type = other DNA
attaccgggctctgccatcttaagagtgcgattaccgggctctgccatcttaagagtgcgattaccgggctctgccatcttagactcctctagagggtatataatgggggcc

SEQ ID NO: 892 - DNA, 114 bp, synthetic construct
  source 1..114: mol_type = other DNA
attaccgggctctgccatcttaagagtgcgaattaccgggctctgccatcttaagagtgcgaattaccgggctctgccatcttagactcctctagagggtatataatgggggcc

SEQ ID NO: 893 - DNA, 116 bp, synthetic construct
  source 1..116: mol_type = other DNA
attaccgggctctgccatcttaagagtgcgagattaccgggctctgccatcttaagagtgcgagattaccgggctctgccatcttagactcctctagagggtatataatgggggcc

SEQ ID NO: 894 - DNA, 97 bp, synthetic construct
  source 1..97: mol_type = other DNA
attaccgggctctgccatcttaattaccgggctctgccatcttaattaccgggctctgccatcttagactcactctagagggtatataatgggggcc

SEQ ID NO: 895 - DNA, 99 bp, synthetic construct
  source 1..99: mol_type = other DNA
attaccgggctctgccatcttaaattaccgggctctgccatcttaaattaccgggctctgccatcttagactcactctagagggtatataatgggggcc

SEQ ID NO: 896 - DNA, 101 bp, synthetic construct
  source 1..101: mol_type = other DNA
attaccgggctctgccatcttaagattaccgggctctgccatcttaagattaccgggctctgccatcttagactcactctagagggtatataatgggggcc

SEQ ID NO: 897 - DNA, 103 bp, synthetic construct
  source 1..103: mol_type = other DNA
attaccgggctctgccatcttaagaattaccgggctctgccatcttaagaattaccgggctctgccatcttagactcactctagagggtatataatgggggcc

SEQ ID NO: 898 - DNA, 105 bp, synthetic construct
  source 1..105: mol_type = other DNA
attaccgggctctgccatcttaagagattaccgggctctgccatcttaagagattaccgggctctgccatcttagactcactctagagggtatataatgggggcc

SEQ ID NO: 899 - DNA, 107 bp, synthetic construct
  source 1..107: mol_type = other DNA
attaccgggctctgccatcttaagagtattaccgggctctgccatcttaagagtattaccgggctctgccatcttagactcactctagagggtatataatgggggcc

SEQ ID NO: 900 - DNA, 109 bp, synthetic construct
  source 1..109: mol_type = other DNA
attaccgggctctgccatcttaagagtgattaccgggctctgccatcttaagagtgattaccgggctctgccatcttagactcactctagagggtatataatgggggcc

SEQ ID NO: 901 - DNA, 111 bp, synthetic construct
  source 1..111: mol_type = other DNA
attaccgggctctgccatcttaagagtgcattaccgggctctgccatcttaagagtgcattaccgggctctgccatcttagactcactctagagggtatataatgggggcc

SEQ ID NO: 902 - DNA, 113 bp, synthetic construct
  source 1..113: mol_type = other DNA
attaccgggctctgccatcttaagagtgcgattaccgggctctgccatcttaagagtgcgattaccgggctctgccatcttagactcactctagagggtatataatgggggcc

SEQ ID NO: 903 - DNA, 115 bp, synthetic construct
  source 1..115: mol_type = other DNA
attaccgggctctgccatcttaagagtgcgaattaccgggctctgccatcttaagagtgcgaattaccgggctctgccatcttagactcactctagagggtatataatgggggcc

SEQ ID NO: 904 - DNA, 117 bp, synthetic construct
  source 1..117: mol_type = other DNA
attaccgggctctgccatcttaagagtgcgagattaccgggctctgccatcttaagagtgcgagattaccgggctctgccatcttagactcactctagagggtatataatgggggcc

SEQ ID NO: 905 - DNA, 98 bp, synthetic construct
  source 1..98: mol_type = other DNA
attaccgggctctgccatcttaattaccgggctctgccatcttaattaccgggctctgccatcttagactcatctctagagggtatataatgggggcc

SEQ ID NO: 906 - DNA, 100 bp, synthetic construct
  source 1..100: mol_type = other DNA
attaccgggctctgccatcttaaattaccgggctctgccatcttaaattaccgggctctgccatcttagactcatctctagagggtatataatgggggcc

SEQ ID NO: 907 - DNA, 102 bp, synthetic construct
  source 1..102: mol_type = other DNA
attaccgggctctgccatcttaagattaccgggctctgccatcttaagattaccgggctctgccatcttagactcatctctagagggtatataatgggggcc

SEQ ID NO: 908 - DNA, 104 bp, synthetic construct
  source 1..104: mol_type = other DNA
attaccgggctctgccatcttaagaattaccgggctctgccatcttaagaattaccgggctctgccatcttagactcatctctagagggtatataatgggggcc

SEQ ID NO: 909 - DNA, 106 bp, synthetic construct
  source 1..106: mol_type = other DNA
attaccgggctctgccatcttaagagattaccgggctctgccatcttaagagattaccgggctctgccatcttagactcatctctagagggtatataatgggggcc

SEQ ID NO: 910 - DNA, 108 bp, synthetic construct
  source 1..108: mol_type = other DNA
attaccgggctctgccatcttaagagtattaccgggctctgccatcttaagagtattaccgggctctgccatcttagactcatctctagagggtatataatgggggcc

SEQ ID NO: 911 - DNA, 110 bp, synthetic construct
  source 1..110: mol_type = other DNA
attaccgggctctgccatcttaagagtgattaccgggctctgccatcttaagagtgattaccgggctctgccatcttagactcatctctagagggtatataatgggggcc

SEQ ID NO: 912 - DNA, 112 bp, synthetic construct
  source 1..112: mol_type = other DNA
attaccgggctctgccatcttaagagtgcattaccgggctctgccatcttaagagtgcattaccgggctctgccatcttagactcatctctagagggtatataatgggggcc

SEQ ID NO: 913 - DNA, 114 bp, synthetic construct
  source 1..114: mol_type = other DNA
attaccgggctctgccatcttaagagtgcgattaccgggctctgccatcttaagagtgcgattaccgggctctgccatcttagactcatctctagagggtatataatgggggcc

SEQ ID NO: 914 - DNA, 116 bp, synthetic construct
  source 1..116: mol_type = other DNA
attaccgggctctgccatcttaagagtgcgaattaccgggctctgccatcttaagagtgcgaattaccgggctctgccatcttagactcatctctagagggtatataatgggggcc

SEQ ID NO: 915 - DNA, 118 bp, synthetic construct
  source 1..118: mol_type = other DNA
attaccgggctctgccatcttaagagtgcgagattaccgggctctgccatcttaagagtgcgagattaccgggctctgccatcttagactcatctctagagggtatataatgggggcc

SEQ ID NO: 916 - DNA, 99 bp, synthetic construct
  source 1..99: mol_type = other DNA
attaccgggctctgccatcttaattaccgggctctgccatcttaattaccgggctctgccatcttagactcatactctagagggtatataatgggggcc

SEQ ID NO: 917 - DNA, 101 bp, synthetic construct
  source 1..101: mol_type = other DNA
attaccgggctctgccatcttaaattaccgggctctgccatcttaaattaccgggctctgccatcttagactcatactctagagggtatataatgggggcc

SEQ ID NO: 918 - DNA, 103 bp, synthetic construct
  source 1..103: mol_type = other DNA
attaccgggctctgccatcttaagattaccgggctctgccatcttaagattaccgggctctgccatcttagactcatactctagagggtatataatgggggcc

SEQ ID NO: 919 - DNA, 105 bp, synthetic construct
  source 1..105: mol_type = other DNA
attaccgggctctgccatcttaagaattaccgggctctgccatcttaagaattaccgggctctgccatcttagactcatactctagagggtatataatgggggcc

SEQ ID NO: 920 - DNA, 107 bp, synthetic construct
  source 1..107: mol_type = other DNA
attaccgggctctgccatcttaagagattaccgggctctgccatcttaagagattaccgggctctgccatcttagactcatactctagagggtatataatgggggcc

SEQ ID NO: 921 - DNA, 109 bp, synthetic construct
  source 1..109: mol_type = other DNA
attaccgggctctgccatcttaagagtattaccgggctctgccatcttaagagtattaccgggctctgccatcttagactcatactctagagggtatataatgggggcc

SEQ ID NO: 922 - DNA, 111 bp, synthetic construct
  source 1..111: mol_type = other DNA
attaccgggctctgccatcttaagagtgattaccgggctctgccatcttaagagtgattaccgggctctgccatcttagactcatactctagagggtatataatgggggcc

SEQ ID NO: 923 - DNA, 113 bp, synthetic construct
  source 1..113: mol_type = other DNA
attaccgggctctgccatcttaagagtgcattaccgggctctgccatcttaagagtgcattaccgggctctgccatcttagactcatactctagagggtatataatgggggcc

SEQ ID NO: 924 - DNA, 115 bp, synthetic construct
  source 1..115: mol_type = other DNA
attaccgggctctgccatcttaagagtgcgattaccgggctctgccatcttaagagtgcgattaccgggctctgccatcttagactcatactctagagggtatataatgggggcc

SEQ ID NO: 925 - DNA, 117 bp, synthetic construct
  source 1..117: mol_type = other DNA
attaccgggctctgccatcttaagagtgcgaattaccgggctctgccatcttaagagtgcgaattaccgggctctgccatcttagactcatactctagagggtatataatgggggcc

SEQ ID NO: 926 - DNA, 119 bp, synthetic construct
  source 1..119: mol_type = other DNA
attaccgggctctgccatcttaagagtgcgagattaccgggctctgccatcttaagagtgcgagattaccgggctctgccatcttagactcatactctagagggtatataatgggggcc

SEQ ID NO: 927 - DNA, 100 bp, synthetic construct
  source 1..100: mol_type = other DNA
attaccgggctctgccatcttaattaccgggctctgccatcttaattaccgggctctgccatcttagactcatatctctagagggtatataatgggggcc

SEQ ID NO: 928 - DNA, 102 bp, synthetic construct
  source 1..102: mol_type = other DNA
attaccgggctctgccatcttaaattaccgggctctgccatcttaaattaccgggctctgccatcttagactcatatctctagagggtatataatgggggcc

SEQ ID NO: 929 - DNA, 104 bp, synthetic construct
  source 1..104: mol_type = other DNA
attaccgggctctgccatcttaagattaccgggctctgccatcttaagattaccgggctctgccatcttagactcatatctctagagggtatataatgggggcc

SEQ ID NO: 930 - DNA, 106 bp, synthetic construct
  source 1..106: mol_type = other DNA
attaccgggctctgccatcttaagaattaccgggctctgccatcttaagaattaccgggctctgccatcttagactcatatctctagagggtatataatgggggcc

SEQ ID NO: 931 - DNA, 108 bp, synthetic construct
  source 1..108: mol_type = other DNA
attaccgggctctgccatcttaagagattaccgggctctgccatcttaagagattaccgggctctgccatcttagactcatatctctagagggtatataatgggggcc

SEQ ID NO: 932 - DNA, 110 bp, synthetic construct
  source 1..110: mol_type = other DNA
attaccgggctctgccatcttaagagtattaccgggctctgccatcttaagagtattaccgggctctgccatcttagactcatatctctagagggtatataatgggggcc

SEQ ID NO: 933 - DNA, 112 bp, synthetic construct
  source 1..112: mol_type = other DNA
attaccgggctctgccatcttaagagtgattaccgggctctgccatcttaagagtgattaccgggctctgccatcttagactcatatctctagagggtatataatgggggcc

SEQ ID NO: 934 - DNA, 114 bp, synthetic construct
  source 1..114: mol_type = other DNA
attaccgggctctgccatcttaagagtgcattaccgggctctgccatcttaagagtgcattaccgggctctgccatcttagactcatatctctagagggtatataatgggggcc

SEQ ID NO: 935 - DNA, 116 bp, synthetic construct
  source 1..116: mol_type = other DNA
attaccgggctctgccatcttaagagtgcgattaccgggctctgccatcttaagagtgcgattaccgggctctgccatcttagactcatatctctagagggtatataatgggggcc

SEQ ID NO: 936 - DNA, 118 bp, synthetic construct
  source 1..118: mol_type = other DNA
attaccgggctctgccatcttaagagtgcgaattaccgggctctgccatcttaagagtgcgaattaccgggctctgccatcttagactcatatctctagagggtatataatgggggcc

SEQ ID NO: 937 - DNA, 120 bp, synthetic construct
  source 1..120: mol_type = other DNA
attaccgggctctgccatcttaagagtgcgagattaccgggctctgccatcttaagagtgcgagattaccgggctctgccatcttagactcatatctctagagggtatataatgggggcc

SEQ ID NO: 938 - DNA, 101 bp, synthetic construct
  source 1..101: mol_type = other DNA
attaccgggctctgccatcttaattaccgggctctgccatcttaattaccgggctctgccatcttagactcatatgctctagagggtatataatgggggcc

SEQ ID NO: 939 - DNA, 103 bp, synthetic construct
  source 1..103: mol_type = other DNA
attaccgggctctgccatcttaaattaccgggctctgccatcttaaattaccgggctctgccatcttagactcatatgctctagagggtatataatgggggcc

SEQ ID NO: 940 - DNA, 105 bp, synthetic construct
  source 1..105: mol_type = other DNA
attaccgggctctgccatcttaagattaccgggctctgccatcttaagattaccgggctctgccatcttagactcatatgctctagagggtatataatgggggcc

SEQ ID NO: 941 - DNA, 107 bp, synthetic construct
  source 1..107: mol_type = other DNA
attaccgggctctgccatcttaagaattaccgggctctgccatcttaagaattaccgggctctgccatcttagactcatatgctctagagggtatataatgggggcc

SEQ ID NO: 942 - DNA, 109 bp, synthetic construct
  source 1..109: mol_type = other DNA
attaccgggctctgccatcttaagagattaccgggctctgccatcttaagagattaccgggctctgccatcttagactcatatgctctagagggtatataatgggggcc

SEQ ID NO: 943 - DNA, 111 bp, synthetic construct
  source 1..111: mol_type = other DNA
attaccgggctctgccatcttaagagtattaccgggctctgccatcttaagagtattaccgggctctgccatcttagactcatatgctctagagggtatataatgggggcc

SEQ ID NO: 944 - DNA, 113 bp, synthetic construct
  source 1..113: mol_type = other DNA
attaccgggctctgccatcttaagagtgattaccgggctctgccatcttaagagtgattaccgggctctgccatcttagactcatatgctctagagggtatataatgggggcc

SEQ ID NO: 945 - DNA, 115 bp, synthetic construct
  source 1..115: mol_type = other DNA
attaccgggctctgccatcttaagagtgcattaccgggctctgccatcttaagagtgcattaccgggctctgccatcttagactcatatgctctagagggtatataatgggggcc

SEQ ID NO: 946 - DNA, 117 bp, synthetic construct
  source 1..117: mol_type = other DNA
attaccgggctctgccatcttaagagtgcgattaccgggctctgccatcttaagagtgcgattaccgggctctgccatcttagactcatatgctctagagggtatataatgggggcc

SEQ ID NO: 947 - DNA, 119 bp, synthetic construct
  source 1..119: mol_type = other DNA
attaccgggctctgccatcttaagagtgcgaattaccgggctctgccatcttaagagtgcgaattaccgggctctgccatcttagactcatatgctctagagggtatataatgggggcc

SEQ ID NO: 948 - DNA, 121 bp, synthetic construct
  source 1..121: mol_type = other DNA
attaccgggctctgccatcttaagagtgcgagattaccgggctctgccatcttaagagtgcgagattaccgggctctgccatcttagactcatatgctctagagggtatataatgggggcc

SEQ ID NO: 949 - DNA, 113 bp, synthetic construct
  source 1..113: mol_type = other DNA
attaccgggctctgccatcttaattaccgggctctgccatcttaattaccgggctctgccatcttaattaccgggctctgccatcttactctagagggtatataatgggggcc

SEQ ID NO: 950 - DNA, 116 bp, synthetic construct
  source 1..116: mol_type = other DNA
attaccgggctctgccatcttaaattaccgggctctgccatcttaaattaccgggctctgccatcttaaattaccgggctctgccatcttactctagagggtatataatgggggcc

SEQ ID NO: 951 - DNA, 119 bp, synthetic construct
  source 1..119: mol_type = other DNA
attaccgggctctgccatcttaagattaccgggctctgccatcttaagattaccgggctctgccatcttaagattaccgggctctgccatcttactctagagggtatataatgggggcc

SEQ ID NO: 952 - DNA, 122 bp, synthetic construct
  source 1..122: mol_type = other DNA
attaccgggctctgccatcttaagaattaccgggctctgccatcttaagaattaccgggctctgccatcttaagaattaccgggctctgccatcttactctagagggtatataatgggggcc

SEQ ID NO: 953 - DNA, 125 bp, synthetic construct
  source 1..125: mol_type = other DNA
attaccgggctctgccatcttaagagattaccgggctctgccatcttaagagattaccgggctctgccatcttaagagattaccgggctctgccatcttactctagagggtatataatgggggcc

SEQ ID NO: 954 - DNA, 128 bp, synthetic construct
  source 1..128: mol_type = other DNA
attaccgggctctgccatcttaagagtattaccgggctctgccatcttaagagtattaccgggctctgccatcttaagagtattaccgggctctgccatcttactctagagggtatataatgggggcc

SEQ ID NO: 955 - DNA, 131 bp, synthetic construct
  source 1..131: mol_type = other DNA
attaccgggctctgccatcttaagagtgattaccgggctctgccatcttaagagtgattaccgggctctgccatcttaagagtgattaccgggctctgccatcttactctagagggtatataatgggggcc

SEQ ID NO: 956 - DNA, 134 bp, synthetic construct
  source 1..134: mol_type = other DNA
attaccgggctctgccatcttaagagtgcattaccgggctctgccatcttaagagtgcattaccgggctctgccatcttaagagtgcattaccgggctctgccatcttactctagagggtatataatgggggcc

SEQ ID NO: 957 - DNA, 137 bp, synthetic construct
  source 1..137: mol_type = other DNA
attaccgggctctgccatcttaagagtgcgattaccgggctctgccatcttaagagtgcgattaccgggctctgccatcttaagagtgcgattaccgggctctgccatcttactctagagggtatataatgggggcc

SEQ ID NO: 958 - DNA, 140 bp, synthetic construct
  source 1..140: mol_type = other DNA
attaccgggctctgccatcttaagagtgcgaattaccgggctctgccatcttaagagtgcgaattaccgggctctgccatcttaagagtgcgaattaccgggctctgccatcttactctagagggtatataatgggggcc

SEQ ID NO: 959 - DNA, 143 bp, synthetic construct
  source 1..143: mol_type = other DNA
attaccgggctctgccatcttaagagtgcgagattaccgggctctgccatcttaagagtgcgagattaccgggctctgccatcttaagagtgcgagattaccgggctctgccatcttactctagagggtatataatgggggcc

SEQ ID NO: 960 - DNA, 114 bp, synthetic construct
  source 1..114: mol_type = other DNA
attaccgggctctgccatcttaattaccgggctctgccatcttaattaccgggctctgccatcttaattaccgggctctgccatcttagctctagagggtatataatgggggcc

SEQ ID NO: 961 - DNA, 117 bp, synthetic construct
  source 1..117: mol_type = other DNA
attaccgggctctgccatcttaaattaccgggctctgccatcttaaattaccgggctctgccatcttaaattaccgggctctgccatcttagctctagagggtatataatgggggcc

SEQ ID NO: 962 - DNA, 120 bp, synthetic construct
  source 1..120: mol_type = other DNA
attaccgggctctgccatcttaagattaccgggctctgccatcttaagattaccgggctctgccatcttaagattaccgggctctgccatcttagctctagagggtatataatgggggcc

SEQ ID NO: 963 - DNA, 123 bp, synthetic construct
  source 1..123: mol_type = other DNA
attaccgggctctgccatcttaagaattaccgggctctgccatcttaagaattaccgggctctgccatcttaagaattaccgggctctgccatcttagctctagagggtatataatgggggcc

SEQ ID NO: 964 - DNA, 126 bp, synthetic construct
  source 1..126: mol_type = other DNA
attaccgggctctgccatcttaagagattaccgggctctgccatcttaagagattaccgggctctgccatcttaagagattaccgggctctgccatcttagctctagagggtatataatgggggcc

SEQ ID NO: 965 - DNA, 129 bp, synthetic construct
  source 1..129: mol_type = other DNA
attaccgggctctgccatcttaagagtattaccgggctctgccatcttaagagtattaccgggctctgccatcttaagagtattaccgggctctgccatcttagctctagagggtatataatgggggcc

SEQ ID NO: 966 - DNA, 132 bp, synthetic construct
  source 1..132: mol_type = other DNA
attaccgggctctgccatcttaagagtgattaccgggctctgccatcttaagagtgattaccgggctctgccatcttaagagtgattaccgggctctgccatcttagctctagagggtatataatgggggcc

SEQ ID NO: 967 - DNA, 135 bp, synthetic construct
  source 1..135: mol_type = other DNA
attaccgggctctgccatcttaagagtgcattaccgggctctgccatcttaagagtgcattaccgggctctgccatcttaagagtgcattaccgggctctgccatcttagctctagagggtatataatgggggcc

SEQ ID NO: 968 - DNA, 138 bp, synthetic construct
  source 1..138: mol_type = other DNA
attaccgggctctgccatcttaagagtgcgattaccgggctctgccatcttaagagtgcgattaccgggctctgccatcttaagagtgcgattaccgggctctgccatcttagctctagagggtatataatgggggcc

SEQ ID NO: 969 - DNA, 141 bp, synthetic construct
  source 1..141: mol_type = other DNA
attaccgggctctgccatcttaagagtgcgaattaccgggctctgccatcttaagagtgcgaattaccgggctctgccatcttaagagtgcgaattaccgggctctgccatcttagctctagagggtatataatgggggcc

SEQ ID NO: 970 - DNA, 144 bp, synthetic construct
  source 1..144: mol_type = other DNA
attaccgggctctgccatcttaagagtgcgagattaccgggctctgccatcttaagagtgcgagattaccgggctctgccatcttaagagtgcgagattaccgggctctgccatcttagctctagagggtatataatgggggcc

SEQ ID NO: 971 - DNA, 115 bp, synthetic construct
  source 1..115: mol_type = other DNA
attaccgggctctgccatcttaattaccgggctctgccatcttaattaccgggctctgccatcttaattaccgggctctgccatcttagactctagagggtatataatgggggcc

SEQ ID NO: 972 - DNA, 118 bp, synthetic construct
  source 1..118: mol_type = other DNA
attaccgggctctgccatcttaaattaccgggctctgccatcttaaattaccgggctctgccatcttaaattaccgggctctgccatcttagactctagagggtatataatgggggcc

SEQ ID NO: 973 - DNA, 121 bp, synthetic construct
  source 1..121: mol_type = other DNA
attaccgggctctgccatcttaagattaccgggctctgccatcttaagattaccgggctctgccatcttaagattaccgggctctgccatcttagactctagagggtatataatgggggcc

SEQ ID NO: 974 - DNA, 124 bp, synthetic construct
  source 1..124: mol_type = other DNA
attaccgggctctgccatcttaagaattaccgggctctgccatcttaagaattaccgggctctgccatcttaagaattaccgggctctgccatcttagactctagagggtatataatgggggcc

SEQ ID NO: 975 - DNA, 127 bp, synthetic construct
  source 1..127: mol_type = other DNA
attaccgggctctgccatcttaagagattaccgggctctgccatcttaagagattaccgggctctgccatcttaagagattaccgggctctgccatcttagactctagagggtatataatgggggcc

SEQ ID NO: 976 - DNA, 130 bp, synthetic construct
  source 1..130: mol_type = other DNA
attaccgggctctgccatcttaagagtattaccgggctctgccatcttaagagtattaccgggctctgccatcttaagagtattaccgggctctgccatcttagactctagagggtatataatgggggcc

SEQ ID NO: 977 - DNA, 133 bp, synthetic construct
  source 1..133: mol_type = other DNA
attaccgggctctgccatcttaagagtgattaccgggctctgccatcttaagagtgattaccgggctctgccatcttaagagtgattaccgggctctgccatcttagactctagagggtatataatgggggcc

SEQ ID NO: 978 - DNA, 136 bp, synthetic construct
  source 1..136: mol_type = other DNA
attaccgggctctgccatcttaagagtgcattaccgggctctgccatcttaagagtgcattaccgggctctgccatcttaagagtgcattaccgggctctgccatcttagactctagagggtatataatgggggcc

SEQ ID NO: 979 - DNA, 139 bp, synthetic construct
  source 1..139: mol_type = other DNA
attaccgggctctgccatcttaagagtgcgattaccgggctctgccatcttaagagtgcgattaccgggctctgccatcttaagagtgcgattaccgggctctgccatcttagactctagagggtatataatgggggcc

SEQ ID NO: 980 - DNA, 142 bp, synthetic construct
  source 1..142: mol_type = other DNA
attaccgggctctgccatcttaagagtgcgaattaccgggctctgccatcttaagagtgcgaattaccgggctctgccatcttaagagtgcgaattaccgggctctgccatcttagactctagagggtatataatgggggcc

SEQ ID NO: 981 - DNA, 145 bp, synthetic construct
  source 1..145: mol_type = other DNA
attaccgggctctgccatcttaagagtgcgagattaccgggctctgccatcttaagagtgcgagattaccgggctctgccatcttaagagtgcgagattaccgggctctgccatcttagactctagagggtatataatgggggcc

SEQ ID NO: 982 - DNA, 116 bp, synthetic construct
  source 1..116: mol_type = other DNA
attaccgggctctgccatcttaattaccgggctctgccatcttaattaccgggctctgccatcttaattaccgggctctgccatcttagacctctagagggtatataatgggggcc

SEQ ID NO: 983 - DNA, 119 bp, synthetic construct
  source 1..119: mol_type = other DNA
attaccgggctctgccatcttaaattaccgggctctgccatcttaaattaccgggctctgccatcttaaattaccgggctctgccatcttagacctctagagggtatataatgggggcc

SEQ ID NO: 984 - DNA, 122 bp, synthetic construct
  source 1..122: mol_type = other DNA
attaccgggctctgccatcttaagattaccgggctctgccatcttaagattaccgggctctgccatcttaagattaccgggctctgccatcttagacctctagagggtatataatgggggcc

SEQ ID NO: 985 - DNA, 125 bp, synthetic construct
  source 1..125: mol_type = other DNA
attaccgggctctgccatcttaagaattaccgggctctgccatcttaagaattaccgggctctgccatcttaagaattaccgggctctgccatcttagacctctagagggtatataatgggggcc

SEQ ID NO: 986 - DNA, 128 bp, synthetic construct
  source 1..128: mol_type = other DNA
attaccgggctctgccatcttaagagattaccgggctctgccatcttaagagattaccgggctctgccatcttaagagattaccgggctctgccatcttagacctctagagggtatataatgggggcc

SEQ ID NO: 987 - DNA, 131 bp, synthetic construct
  source 1..131: mol_type = other DNA
attaccgggctctgccatcttaagagtattaccgggctctgccatcttaagagtattaccgggctctgccatcttaagagtattaccgggctctgccatcttagacctctagagggtatataatgggggcc

SEQ ID NO: 988 - DNA, 134 bp, synthetic construct
  source 1..134: mol_type = other DNA
attaccgggctctgccatcttaagagtgattaccgggctctgccatcttaagagtgattaccgggctctgccatcttaagagtgattaccgggctctgccatcttagacctctagagggtatataatgggggcc

SEQ ID NO: 989 - DNA, 137 bp, synthetic construct
  source 1..137: mol_type = other DNA
attaccgggctctgccatcttaagagtgcattaccgggctctgccatcttaagagtgcattaccgggctctgccatcttaagagtgcattaccgggctctgccatcttagacctctagagggtatataatgggggcc

SEQ ID NO: 990 - DNA, 140 bp, synthetic construct
  source 1..140: mol_type = other DNA
attaccgggctctgccatcttaagagtgcgattaccgggctctgccatcttaagagtgcgattaccgggctctgccatcttaagagtgcgattaccgggctctgccatcttagacctctagagggtatataatgggggcc

SEQ ID NO: 991 - DNA, 143 bp, synthetic construct
  source 1..143: mol_type = other DNA
attaccgggctctgccatcttaagagtgcgaattaccgggctctgccatcttaagagtgcgaattaccgggctctgccatcttaagagtgcgaattaccgggctctgccatcttagacctctagagggtatataatgggggcc

SEQ ID NO: 992 - DNA, 146 bp, synthetic construct
  source 1..146: mol_type = other DNA
attaccgggctctgccatcttaagagtgcgagattaccgggctctgccatcttaagagtgcgagattaccgggctctgccatcttaagagtgcgagattaccgggctctgccatcttagacctctagagggtatataatgggggcc

SEQ ID NO: 993 - DNA, 117 bp, synthetic construct
  source 1..117: mol_type = other DNA
attaccgggctctgccatcttaattaccgggctctgccatcttaattaccgggctctgccatcttaattaccgggctctgccatcttagactctctagagggtatataatgggggcc

SEQ ID NO: 994 - DNA, 120 bp, synthetic construct
  source 1..120: mol_type = other DNA
attaccgggctctgccatcttaaattaccgggctctgccatcttaaattaccgggctctgccatcttaaattaccgggctctgccatcttagactctctagagggtatataatgggggcc

SEQ ID NO: 995 - DNA, 123 bp, synthetic construct
  source 1..123: mol_type = other DNA
attaccgggctctgccatcttaagattaccgggctctgccatcttaagattaccgggctctgccatcttaagattaccgggctctgccatcttagactctctagagggtatataatgggggcc

SEQ ID NO: 996 - DNA, 126 bp, synthetic construct
  source 1..126: mol_type = other DNA
attaccgggctctgccatcttaagaattaccgggctctgccatcttaagaattaccgggctctgccatcttaagaattaccgggctctgccatcttagactctctagagggtatataatgggggcc

SEQ ID NO: 997 - DNA, 129 bp, synthetic construct
  source 1..129: mol_type = other DNA
attaccgggctctgccatcttaagagattaccgggctctgccatcttaagagattaccgggctctgccatcttaagagattaccgggctctgccatcttagactctctagagggtatataatgggggcc

SEQ ID NO: 998 - DNA, 132 bp, synthetic construct
  source 1..132: mol_type = other DNA
attaccgggctctgccatcttaagagtattaccgggctctgccatcttaagagtattaccgggctctgccatcttaagagtattaccgggctctgccatcttagactctctagagggtatataatgggggcc

SEQ ID NO: 999 - DNA, 135 bp, synthetic construct
  source 1..135: mol_type = other DNA
attaccgggctctgccatcttaagagtgattaccgggctctgccatcttaagagtgattaccgggctctgccatcttaagagtgattaccgggctctgccatcttagactctctagagggtatataatgggggcc

SEQ ID NO: 1000 - DNA, 138 bp, synthetic construct
  source 1..138: mol_type = other DNA
attaccgggctctgccatcttaagagtgcattaccgggctctgccatcttaagagtgcattaccgggctctgccatcttaagagtgcattaccgggctctgccatcttagactctctagagggtatataatgggggcc

SEQ ID NO: 1001 - DNA, 141 bp, synthetic construct
  source 1..141: mol_type = other DNA
attaccgggctctgccatcttaagagtgcgattaccgggctctgccatcttaagagtgcgattaccgggctctgccatcttaagagtgcgattaccgggctctgccatcttagactctctagagggtatataatgggggcc

SEQ ID NO: 1002 - DNA, 144 bp, synthetic construct
  source 1..144: mol_type = other DNA
attaccgggctctgccatcttaagagtgcgaattaccgggctctgccatcttaagagtgcgaattaccgggctctgccatcttaagagtgcgaattaccgggctctgccatcttagactctctagagggtatataatgggggcc

SEQ ID NO: 1003 - DNA, 147 bp, synthetic construct
  source 1..147: mol_type = other DNA
attaccgggctctgccatcttaagagtgcgagattaccgggctctgccatcttaagagtgcgagattaccgggctctgccatcttaagagtgcgagattaccgggctctgccatcttagactctctagagggtatataatgggggcc

SEQ ID NO: 1004 - DNA, 118 bp, synthetic construct
  source 1..118: mol_type = other DNA
attaccgggctctgccatcttaattaccgggctctgccatcttaattaccgggctctgccatcttaattaccgggctctgccatcttagactcctctagagggtatataatgggggcc

SEQ ID NO: 1005 - DNA, 121 bp, synthetic construct
  source 1..121: mol_type = other DNA
attaccgggctctgccatcttaaattaccgggctctgccatcttaaattaccgggctctgccatcttaaattaccgggctctgccatcttagactcctctagagggtatataatgggggcc

SEQ ID NO: 1006 - DNA, 124 bp, synthetic construct
  source 1..124: mol_type = other DNA
attaccgggctctgccatcttaagattaccgggctctgccatcttaagattaccgggctctgccatcttaagattaccgggctctgccatcttagactcctctagagggtatataatgggggcc

SEQ ID NO: 1007 - DNA, 127 bp, synthetic construct
  source 1..127: mol_type = other DNA
attaccgggctctgccatcttaagaattaccgggctctgccatcttaagaattaccgggctctgccatcttaagaattaccgggctctgccatcttagactcctctagagggtatataatgggggcc

SEQ ID NO: 1008 - DNA, 130 bp, synthetic construct
  source 1..130: mol_type = other DNA
attaccgggctctgccatcttaagagattaccgggctctgccatcttaagagattaccgggctctgccatcttaagagattaccgggctctgccatcttagactcctctagagggtatataatgggggcc

SEQ ID NO: 1009 - DNA, 133 bp, synthetic construct
  source 1..133: mol_type = other DNA
attaccgggctctgccatcttaagagtattaccgggctctgccatcttaagagtattaccgggctctgccatcttaagagtattaccgggctctgccatcttagactcctctagagggtatataatgggggcc

SEQ ID NO: 1010 - DNA, 136 bp, synthetic construct
  source 1..136: mol_type = other DNA
attaccgggctctgccatcttaagagtgattaccgggctctgccatcttaagagtgattaccgggctctgccatcttaagagtgattaccgggctctgccatcttagactcctctagagggtatataatgggggcc

SEQ ID NO: 1011 - DNA, 139 bp, synthetic construct
  source 1..139: mol_type = other DNA
attaccgggctctgccatcttaagagtgcattaccgggctctgccatcttaagagtgcattaccgggctctgccatcttaagagtgcattaccgggctctgccatcttagactcctctagagggtatataatgggggcc

SEQ ID NO: 1012 - DNA, 142 bp, synthetic construct
  source 1..142: mol_type = other DNA
attaccgggctctgccatcttaagagtgcgattaccgggctctgccatcttaagagtgcgattaccgggctctgccatcttaagagtgcgattaccgggctctgccatcttagactcctctagagggtatataatgggggcc

SEQ ID NO: 1013 - DNA, 145 bp, synthetic construct
  source 1..145: mol_type = other DNA
attaccgggctctgccatcttaagagtgcgaattaccgggctctgccatcttaagagtgcgaattaccgggctctgccatcttaagagtgcgaattaccgggctctgccatcttagactcctctagagggtatataatgggggcc

SEQ ID NO: 1014 - DNA, 148 bp, synthetic construct
  source 1..148: mol_type = other DNA
attaccgggctctgccatcttaagagtgcgagattaccgggctctgccatcttaagagtgcgagattaccgggctctgccatcttaagagtgcgagattaccgggctctgccatcttagactcctctagagggtatataatgggggcc

SEQ ID NO: 1015 - DNA, 119 bp, synthetic construct
  source 1..119: mol_type = other DNA
attaccgggctctgccatcttaattaccgggctctgccatcttaattaccgggctctgccatcttaattaccgggctctgccatcttagactcactctagagggtatataatgggggcc

SEQ ID NO: 1016 - DNA, 122 bp, synthetic construct
  source 1..122: mol_type = other DNA
attaccgggctctgccatcttaaattaccgggctctgccatcttaaattaccgggctctgccatcttaaattaccgggctctgccatcttagactcactctagagggtatataatgggggcc

SEQ ID NO: 1017 - DNA, 125 bp, synthetic construct
  source 1..125: mol_type = other DNA
attaccgggctctgccatcttaagattaccgggctctgccatcttaagattaccgggctctgccatcttaagattaccgggctctgccatcttagactcactctagagggtatataatgggggcc

SEQ ID NO: 1018 - DNA, 128 bp, synthetic construct
  source 1..128: mol_type = other DNA
attaccgggctctgccatcttaagaattaccgggctctgccatcttaagaattaccgggctctgccatcttaagaattaccgggctctgccatcttagactcactctagagggtatataatgggggcc

SEQ ID NO: 1019 - DNA, 131 bp, synthetic construct
  source 1..131: mol_type = other DNA
attaccgggctctgccatcttaagagattaccgggctctgccatcttaagagattaccgggctctgccatcttaagagattaccgggctctgccatcttagactcactctagagggtatataatgggggcc

SEQ ID NO: 1020 - DNA, 134 bp, synthetic construct
  source 1..134: mol_type = other DNA
attaccgggctctgccatcttaagagtattaccgggctctgccatcttaagagtattaccgggctctgccatcttaagagtattaccgggctctgccatcttagactcactctagagggtatataatgggggcc

SEQ ID NO: 1021 - DNA, 137 bp, synthetic construct
  source 1..137: mol_type = other DNA
attaccgggctctgccatcttaagagtgattaccgggctctgccatcttaagagtgattaccgggctctgccatcttaagagtgattaccgggctctgccatcttagactcactctagagggtatataatgggggcc

SEQ ID NO: 1022 - DNA, 140 bp, synthetic construct
  source 1..140: mol_type = other DNA
attaccgggctctgccatcttaagagtgcattaccgggctctgccatcttaagagtgcattaccgggctctgccatcttaagagtgcattaccgggctctgccatcttagactcactctagagggtatataatgggggcc

SEQ ID NO: 1023 - DNA, 143 bp, synthetic construct
  source 1..143: mol_type = other DNA
attaccgggctctgccatcttaagagtgcgattaccgggctctgccatcttaagagtgcgattaccgggctctgccatcttaagagtgcgattaccgggctctgccatcttagactcactctagagggtatataatgggggcc

SEQ ID NO: 1024 - DNA, 146 bp, synthetic construct
  source 1..146: mol_type = other DNA
attaccgggctctgccatcttaagagtgcgaattaccgggctctgccatcttaagagtgcgaattaccgggctctgccatcttaagagtgcgaattaccgggctctgccatcttagactcactctagagggtatataatgggggcc

SEQ ID NO: 1025 - DNA, 149 bp, synthetic construct
  source 1..149: mol_type = other DNA
attaccgggctctgccatcttaagagtgcgagattaccgggctctgccatcttaagagtgcgagattaccgggctctgccatcttaagagtgcgagattaccgggctctgccatcttagactcactctagagggtatataatgggggcc

SEQ ID NO: 1026 - DNA, 120 bp, synthetic construct
  source 1..120: mol_type = other DNA
attaccgggctctgccatcttaattaccgggctctgccatcttaattaccgggctctgccatcttaattaccgggctctgccatcttagactcatctctagagggtatataatgggggcc

SEQ ID NO: 1027 - DNA, 123 bp, synthetic construct
  source 1..123: mol_type = other DNA
attaccgggctctgccatcttaaattaccgggctctgccatcttaaattaccgggctctgccatcttaaattaccgggctctgccatcttagactcatctctagagggtatataatgggggcc

SEQ ID NO: 1028 - DNA, 126 bp, synthetic construct
  source 1..126: mol_type = other DNA
attaccgggctctgccatcttaagattaccgggctctgccatcttaagattaccgggctctgccatcttaagattaccgggctctgccatcttagactcatctctagagggtatataatgggggcc

SEQ ID NO: 1029 - DNA, 129 bp, synthetic construct
  source 1..129: mol_type = other DNA
attaccgggctctgccatcttaagaattaccgggctctgccatcttaagaattaccgggctctgccatcttaagaattaccgggctctgccatcttagactcatctctagagggtatataatgggggcc

SEQ ID NO: 1030 - DNA, 132 bp, synthetic construct
  source 1..132: mol_type = other DNA
attaccgggctctgccatcttaagagattaccgggctctgccatcttaagagattaccgggctctgccatcttaagagattaccgggctctgccatcttagactcatctctagagggtatataatgggggcc

SEQ ID NO: 1031 - DNA, 135 bp, synthetic construct
  source 1..135: mol_type = other DNA
attaccgggctctgccatcttaagagtattaccgggctctgccatcttaagagtattaccgggctctgccatcttaagagtattaccgggctctgccatcttagactcatctctagagggtatataatgggggcc

SEQ ID NO: 1032 - DNA, 138 bp, synthetic construct
  source 1..138: mol_type = other DNA
attaccgggctctgccatcttaagagtgattaccgggctctgccatcttaagagtgattaccgggctctgccatcttaagagtgattaccgggctctgccatcttagactcatctctagagggtatataatgggggcc

SEQ ID NO: 1033 - DNA, 141 bp, synthetic construct
  source 1..141: mol_type = other DNA
attaccgggctctgccatcttaagagtgcattaccgggctctgccatcttaagagtgcattaccgggctctgccatcttaagagtgcattaccgggctctgccatcttagactcatctctagagggtatataatgggggcc

SEQ ID NO: 1034 - DNA, 144 bp, synthetic construct
  source 1..144: mol_type = other DNA
attaccgggctctgccatcttaagagtgcgattaccgggctctgccatcttaagagtgcgattaccgggctctgccatcttaagagtgcgattaccgggctctgccatcttagactcatctctagagggtatataatgggggcc

SEQ ID NO: 1035 - DNA, 147 bp, synthetic construct
  source 1..147: mol_type = other DNA
attaccgggctctgccatcttaagagtgcgaattaccgggctctgccatcttaagagtgcgaattaccgggctctgccatcttaagagtgcgaattaccgggctctgccatcttagactcatctctagagggtatataatgggggcc

SEQ ID NO: 1036 - DNA, 150 bp, synthetic construct
  source 1..150: mol_type = other DNA
attaccgggctctgccatcttaagagtgcgagattaccgggctctgccatcttaagagtgcgagattaccgggctctgccatcttaagagtgcgagattaccgggctctgccatcttagactcatctctagagggtatataatgggggcc

SEQ ID NO: 1037 - DNA, 121 bp, synthetic construct
  source 1..121: mol_type = other DNA
attaccgggctctgccatcttaattaccgggctctgccatcttaattaccgggctctgccatcttaattaccgggctctgccatcttagactcatactctagagggtatataatgggggcc

SEQ ID NO: 1038 - DNA, 124 bp, synthetic construct
  source 1..124: mol_type = other DNA
attaccgggctctgccatcttaaattaccgggctctgccatcttaaattaccgggctctgccatcttaaattaccgggctctgccatcttagactcatactctagagggtatataatgggggcc

SEQ ID NO: 1039 - DNA, 127 bp, synthetic construct
  source 1..127: mol_type = other DNA
attaccgggctctgccatcttaagattaccgggctctgccatcttaagattaccgggctctgccatcttaagattaccgggctctgccatcttagactcatactctagagggtatataatgggggcc

SEQ ID NO: 1040 - DNA, 130 bp, synthetic construct
  source 1..130: mol_type = other DNA
attaccgggctctgccatcttaagaattaccgggctctgccatcttaagaattaccgggctctgccatcttaagaattaccgggctctgccatcttagactcatactctagagggtatataatgggggcc

SEQ ID NO: 1041 - DNA, 133 bp, synthetic construct
  source 1..133: mol_type = other DNA
attaccgggctctgccatcttaagagattaccgggctctgccatcttaagagattaccgggctctgccatcttaagagattaccgggctctgccatcttagactcatactctagagggtatataatgggggcc

SEQ ID NO: 1042 - DNA, 136 bp, synthetic construct
  source 1..136: mol_type = other DNA
attaccgggctctgccatcttaagagtattaccgggctctgccatcttaagagtattaccgggctctgccatcttaagagtattaccgggctctgccatcttagactcatactctagagggtatataatgggggcc

SEQ ID NO: 1043 - DNA, 139 bp, synthetic construct
  source 1..139: mol_type = other DNA
attaccgggctctgccatcttaagagtgattaccgggctctgccatcttaagagtgattaccgggctctgccatcttaagagtgattaccgggctctgccatcttagactcatactctagagggtatataatgggggcc

SEQ ID NO: 1044 - DNA, 142 bp, synthetic construct
  source 1..142: mol_type = other DNA
attaccgggctctgccatcttaagagtgcattaccgggctctgccatcttaagagtgcattaccgggctctgccatcttaagagtgcattaccgggctctgccatcttagactcatactctagagggtatataatgggggcc

SEQ ID NO: 1045 - DNA, 145 bp, synthetic construct
  source 1..145: mol_type = other DNA
attaccgggctctgccatcttaagagtgcgattaccgggctctgccatcttaagagtgcgattaccgggctctgccatcttaagagtgcgattaccgggctctgccatcttagactcatactctagagggtatataatgggggcc

SEQ ID NO: 1046 - DNA, 148 bp, synthetic construct
  source 1..148: mol_type = other DNA
attaccgggctctgccatcttaagagtgcgaattaccgggctctgccatcttaagagtgcgaattaccgggctctgccatcttaagagtgcgaattaccgggctctgccatcttagactcatactctagagggtatataatgggggcc

SEQ ID NO: 1047 - DNA, 151 bp, synthetic construct
  source 1..151: mol_type = other DNA
attaccgggctctgccatcttaagagtgcgagattaccgggctctgccatcttaagagtgcgagattaccgggctctgccatcttaagagtgcgagattaccgggctctgccatcttagactcatactctagagggtatataatgggggcc

SEQ ID NO: 1048 - DNA, 122 bp, synthetic construct
  source 1..122: mol_type = other DNA
attaccgggctctgccatcttaattaccgggctctgccatcttaattaccgggctctgccatcttaattaccgggctctgccatcttagactcatatctctagagggtatataatgggggcc

SEQ ID NO: 1049 - DNA, 125 bp, synthetic construct
  source 1..125: mol_type = other DNA
attaccgggctctgccatcttaaattaccgggctctgccatcttaaattaccgggctctgccatcttaaattaccgggctctgccatcttagactcatatctctagagggtatataatgggggcc

SEQ ID NO: 1050 - DNA, 128 bp, synthetic construct
  source 1..128: mol_type = other DNA
attaccgggctctgccatcttaagattaccgggctctgccatcttaagattaccgggctctgccatcttaagattaccgggctctgccatcttagactcatatctctagagggtatataatgggggcc

SEQ ID NO: 1051 - DNA, 131 bp, synthetic construct
  source 1..131: mol_type = other DNA
attaccgggctctgccatcttaagaattaccgggctctgccatcttaagaattaccgggctctgccatcttaagaattaccgggctctgccatcttagactcatatctctagagggtatataatgggggcc

SEQ ID NO: 1052 - DNA, 134 bp, synthetic construct
  source 1..134: mol_type = other DNA
attaccgggctctgccatcttaagagattaccgggctctgccatcttaagagattaccgggctctgccatcttaagagattaccgggctctgccatcttagactcatatctctagagggtatataatgggggcc

SEQ ID NO: 1053 - DNA, 137 bp, synthetic construct
  source 1..137: mol_type = other DNA
attaccgggctctgccatcttaagagtattaccgggctctgccatcttaagagtattaccgggctctgccatcttaagagtattaccgggctctgccatcttagactcatatctctagagggtatataatgggggcc

SEQ ID NO: 1054 - DNA, 140 bp, synthetic construct
  source 1..140: mol_type = other DNA
attaccgggctctgccatcttaagagtgattaccgggctctgccatcttaagagtgattaccgggctctgccatcttaagagtgattaccgggctctgccatcttagactcatatctctagagggtatataatgggggcc

SEQ ID NO: 1055 - DNA, 143 bp, synthetic construct
  source 1..143: mol_type = other DNA
attaccgggctctgccatcttaagagtgcattaccgggctctgccatcttaagagtgcattaccgggctctgccatcttaagagtgcattaccgggctctgccatcttagactcatatctctagagggtatataatgggggcc

SEQ ID NO: 1056 - DNA, 146 bp, synthetic construct
  source 1..146: mol_type = other DNA
attaccgggctctgccatcttaagagtgcgattaccgggctctgccatcttaagagtgcgattaccgggctctgccatcttaagagtgcgattaccgggctctgccatcttagactcatatctctagagggtatataatgggggcc

SEQ ID NO: 1057 - DNA, 149 bp, synthetic construct
  source 1..149: mol_type = other DNA
attaccgggctctgccatcttaagagtgcgaattaccgggctctgccatcttaagagtgcgaattaccgggctctgccatcttaagagtgcgaattaccgggctctgccatcttagactcatatctctagagggtatataatgggggcc

SEQ ID NO: 1058 - DNA, 152 bp, synthetic construct
  source 1..152: mol_type = other DNA
attaccgggctctgccatcttaagagtgcgagattaccgggctctgccatcttaagagtgcgagattaccgggctctgccatcttaagagtgcgagattaccgggctctgccatcttagactcatatctctagagggtatataatgggggcc

SEQ ID NO: 1059 - DNA, 123 bp, synthetic construct
  source 1..123: mol_type = other DNA
attaccgggctctgccatcttaattaccgggctctgccatcttaattaccgggctctgccatcttaattaccgggctctgccatcttagactcatatgctctagagggtatataatgggggcc

SEQ ID NO: 1060 - DNA, 126 bp, synthetic construct
  source 1..126: mol_type = other DNA
attaccgggctctgccatcttaaattaccgggctctgccatcttaaattaccgggctctgccatcttaaattaccgggctctgccatcttagactcatatgctctagagggtatataatgggggcc

SEQ ID NO: 1061 - DNA, 129 bp, synthetic construct
  source 1..129: mol_type = other DNA
attaccgggctctgccatcttaagattaccgggctctgccatcttaagattaccgggctctgccatcttaagattaccgggctctgccatcttagactcatatgctctagagggtatataatgggggcc

SEQ ID NO: 1062 - DNA, 132 bp, synthetic construct
  source 1..132: mol_type = other DNA
attaccgggctctgccatcttaagaattaccgggctctgccatcttaagaattaccgggctctgccatcttaagaattaccgggctctgccatcttagactcatatgctctagagggtatataatgggggcc

SEQ ID NO: 1063 - DNA, 135 bp, synthetic construct
  source 1..135: mol_type = other DNA
attaccgggctctgccatcttaagagattaccgggctctgccatcttaagagattaccgggctctgccatcttaagagattaccgggctctgccatcttagactcatatgctctagagggtatataatgggggcc

SEQ ID NO: 1064 - DNA, 138 bp, synthetic construct
  source 1..138: mol_type = other DNA
attaccgggctctgccatcttaagagtattaccgggctctgccatcttaagagtattaccgggctctgccatcttaagagtattaccgggctctgccatcttagactcatatgctctagagggtatataatgggggcc

SEQ ID NO: 1065 - DNA, 141 bp, synthetic construct
  source 1..141: mol_type = other DNA
attaccgggctctgccatcttaagagtgattaccgggctctgccatcttaagagtgattaccgggctctgccatcttaagagtgattaccgggctctgccatcttagactcatatgctctagagggtatataatgggggcc

SEQ ID NO: 1066 - DNA, 144 bp, synthetic construct
  source 1..144: mol_type = other DNA
attaccgggctctgccatcttaagagtgcattaccgggctctgccatcttaagagtgcattaccgggctctgccatcttaagagtgcattaccgggctctgccatcttagactcatatgctctagagggtatataatgggggcc

SEQ ID NO: 1067 - DNA, 147 bp, synthetic construct
  source 1..147: mol_type = other DNA
attaccgggctctgccatcttaagagtgcgattaccgggctctgccatcttaagagtgcgattaccgggctctgccatcttaagagtgcgattaccgggctctgccatcttagactcatatgctctagagggtatataatgggggcc

SEQ ID NO: 1068 - DNA, 150 bp, synthetic construct
  source 1..150: mol_type = other DNA
attaccgggctctgccatcttaagagtgcgaattaccgggctctgccatcttaagagtgcgaattaccgggctctgccatcttaagagtgcgaattaccgggctctgccatcttagactcatatgctctagagggtatataatgggggcc

SEQ ID NO: 1069 - DNA, 153 bp, synthetic construct
  source 1..153: mol_type = other DNA
attaccgggctctgccatcttaagagtgcgagattaccgggctctgccatcttaagagtgcgagattaccgggctctgccatcttaagagtgcgagattaccgggctctgccatcttagactcatatgctctagagggtatataatgggggcc

SEQ ID NO: 1070 - DNA, 135 bp, synthetic construct
  source 1..135: mol_type = other DNA
attaccgggctctgccatcttaattaccgggctctgccatcttaattaccgggctctgccatcttaattaccgggctctgccatcttaattaccgggctctgccatcttactctagagggtatataatgggggcc

SEQ ID NO: 1071 - DNA, 139 bp, synthetic construct
  source 1..139: mol_type = other DNA
attaccgggctctgccatcttaaattaccgggctctgccatcttaaattaccgggctctgccatcttaaattaccgggctctgccatcttaaattaccgggctctgccatcttactctagagggtatataatgggggcc

SEQ ID NO: 1072 - DNA, 143 bp, synthetic construct
  source 1..143: mol_type = other DNA
attaccgggctctgccatcttaagattaccgggctctgccatcttaagattaccgggctctgccatcttaagattaccgggctctgccatcttaagattaccgggctctgccatcttactctagagggtatataatgggggcc

SEQ ID NO: 1073 - DNA, 147 bp, synthetic construct
  source 1..147: mol_type = other DNA
attaccgggctctgccatcttaagaattaccgggctctgccatcttaagaattaccgggctctgccatcttaagaattaccgggctctgccatcttaagaattaccgggctctgccatcttactctagagggtatataatgggggcc

SEQ ID NO: 1074 - DNA, 151 bp, synthetic construct
  source 1..151: mol_type = other DNA
attaccgggctctgccatcttaagagattaccgggctctgccatcttaagagattaccgggctctgccatcttaagagattaccgggctctgccatcttaagagattaccgggctctgccatcttactctagagggtatataatgggggcc

SEQ ID NO: 1075 - DNA, 155 bp, synthetic construct
  source 1..155: mol_type = other DNA
attaccgggctctgccatcttaagagtattaccgggctctgccatcttaagagtattaccgggctctgccatcttaagagtattaccgggctctgccatcttaagagtattaccgggctctgccatcttactctagagggtatataatgggggcc

SEQ ID NO: 1076 - DNA, 159 bp, synthetic construct
  source 1..159: mol_type = other DNA
attaccgggctctgccatcttaagagtgattaccgggctctgccatcttaagagtgattaccgggctctgccatcttaagagtgattaccgggctctgccatcttaagagtgattaccgggctctgccatcttactctagagggtatataatgggggcc

SEQ ID NO: 1077 - DNA, 163 bp, synthetic construct
  source 1..163: mol_type = other DNA
attaccgggctctgccatcttaagagtgcattaccgggctctgccatcttaagagtgcattaccgggctctgccatcttaagagtgcattaccgggctctgccatcttaagagtgcattaccgggctctgccatcttactctagagggtatataatgggggcc

SEQ ID NO: 1078 - DNA, 167 bp, synthetic construct
  source 1..167: mol_type = other DNA
attaccgggctctgccatcttaagagtgcgattaccgggctctgccatcttaagagtgcgattaccgggctctgccatcttaagagtgcgattaccgggctctgccatcttaagagtgcgattaccgggctctgccatcttactctagagggtatataatgggggcc

SEQ ID NO: 1079 - DNA, 171 bp, synthetic construct
  source 1..171: mol_type = other DNA
attaccgggctctgccatcttaagagtgcgaattaccgggctctgccatcttaagagtgcgaattaccgggctctgccatcttaagagtgcgaattaccgggctctgccatcttaagagtgcgaattaccgggctctgccatcttactctagagggtatataatgggggcc

SEQ ID NO: 1080 - DNA, 175 bp, synthetic construct
  source 1..175: mol_type = other DNA
attaccgggctctgccatcttaagagtgcgagattaccgggctctgccatcttaagagtgcgagattaccgggctctgccatcttaagagtgcgagattaccgggctctgccatcttaagagtgcgagattaccgggctctgccatcttactctagagggtatataatgggggcc

SEQ ID NO: 1081 - DNA, 136 bp, synthetic construct
  source 1..136: mol_type = other DNA
attaccgggctctgccatcttaattaccgggctctgccatcttaattaccgggctctgccatcttaattaccgggctctgccatcttaattaccgggctctgccatcttagctctagagggtatataatgggggcc

SEQ ID NO: 1082 - DNA, 140 bp, synthetic construct
  source 1..140: mol_type = other DNA
attaccgggctctgccatcttaaattaccgggctctgccatcttaaattaccgggctctgccatcttaaattaccgggctctgccatcttaaattaccgggctctgccatcttagctctagagggtatataatgggggcc

SEQ ID NO: 1083 - DNA, 144 bp, synthetic construct
  source 1..144: mol_type = other DNA
attaccgggctctgccatcttaagattaccgggctctgccatcttaagattaccgggctctgccatcttaagattaccgggctctgccatcttaagattaccgggctctgccatcttagctctagagggtatataatgggggcc

SEQ ID NO: 1084 - DNA, 148 bp, synthetic construct
  source 1..148: mol_type = other DNA
attaccgggctctgccatcttaagaattaccgggctctgccatcttaagaattaccgggctctgccatcttaagaattaccgggctctgccatcttaagaattaccgggctctgccatcttagctctagagggtatataatgggggcc

SEQ ID NO: 1085 - DNA, 152 bp, synthetic construct
  source 1..152: mol_type = other DNA
attaccgggctctgccatcttaagagattaccgggctctgccatcttaagagattaccgggctctgccatcttaagagattaccgggctctgccatcttaagagattaccgggctctgccatcttagctctagagggtatataatgggggcc

SEQ ID NO: 1086 - DNA, 156 bp, synthetic construct
  source 1..156: mol_type = other DNA
attaccgggctctgccatcttaagagtattaccgggctctgccatcttaagagtattaccgggctctgccatcttaagagtattaccgggctctgccatcttaagagtattaccgggctctgccatcttagctctagagggtatataatgggggcc

SEQ ID NO: 1087 - DNA, 160 bp, synthetic construct
  source 1..160: mol_type = other DNA
attaccgggctctgccatcttaagagtgattaccgggctctgccatcttaagagtgattaccgggctctgccatcttaagagtgattaccgggctctgccatcttaagagtgattaccgggctctgccatcttagctctagagggtatataatgggggcc

SEQ ID NO: 1088 - DNA, 164 bp, synthetic construct
  source 1..164: mol_type = other DNA
attaccgggctctgccatcttaagagtgcattaccgggctctgccatcttaagagtgcattaccgggctctgccatcttaagagtgcattaccgggctctgccatcttaagagtgcattaccgggctctgccatcttagctctagagggtatataatgggggcc

SEQ ID NO: 1089 - DNA, 168 bp, synthetic construct
  source 1..168: mol_type = other DNA
attaccgggctctgccatcttaagagtgcgattaccgggctctgccatcttaagagtgcgattaccgggctctgccatcttaagagtgcgattaccgggctctgccatcttaagagtgcgattaccgggctctgccatcttagctctagagggtatataatgggggcc

SEQ ID NO: 1090 - DNA, 172 bp, synthetic construct
  source 1..172: mol_type = other DNA
attaccgggctctgccatcttaagagtgcgaattaccgggctctgccatcttaagagtgcgaattaccgggctctgccatcttaagagtgcgaattaccgggctctgccatcttaagagtgcgaattaccgggctctgccatcttagctctagagggtatataatgggggcc

SEQ ID NO: 1091 - DNA, 176 bp, synthetic construct
  source 1..176: mol_type = other DNA
attaccgggctctgccatcttaagagtgcgagattaccgggctctgccatcttaagagtgcgagattaccgggctctgccatcttaagagtgcgagattaccgggctctgccatcttaagagtgcgagattaccgggctctgccatcttagctctagagggtatataatgggggcc

SEQ ID NO: 1092 - DNA, 137 bp, synthetic construct
  source 1..137: mol_type = other DNA
attaccgggctctgccatcttaattaccgggctctgccatcttaattaccgggctctgccatcttaattaccgggctctgccatcttaattaccgggctctgccatcttagactctagagggtatataatgggggcc

SEQ ID NO: 1093 - DNA, 141 bp, synthetic construct
  source 1..141: mol_type = other DNA
attaccgggctctgccatcttaaattaccgggctctgccatcttaaattaccgggctctgccatcttaaattaccgggctctgccatcttaaattaccgggctctgccatcttagactctagagggtatataatgggggcc

SEQ ID NO: 1094 - DNA, 145 bp, synthetic construct
  source 1..145: mol_type = other DNA
attaccgggctctgccatcttaagattaccgggctctgccatcttaagattaccgggctctgccatcttaagattaccgggctctgccatcttaagattaccgggctctgccatcttagactctagagggtatataatgggggcc

SEQ ID NO: 1095 - DNA, 149 bp, synthetic construct
  source 1..149: mol_type = other DNA
attaccgggctctgccatcttaagaattaccgggctctgccatcttaagaattaccgggctctgccatcttaagaattaccgggctctgccatcttaagaattaccgggctctgccatcttagactctagagggtatataatgggggcc

SEQ ID NO: 1096 - DNA, 153 bp, synthetic construct
  source 1..153: mol_type = other DNA
attaccgggctctgccatcttaagagattaccgggctctgccatcttaagagattaccgggctctgccatcttaagagattaccgggctctgccatcttaagagattaccgggctctgccatcttagactctagagggtatataatgggggcc

SEQ ID NO: 1097 - DNA, 157 bp, synthetic construct
  source 1..157: mol_type = other DNA
attaccgggctctgccatcttaagagtattaccgggctctgccatcttaagagtattaccgggctctgccatcttaagagtattaccgggctctgccatcttaagagtattaccgggctctgccatcttagactctagagggtatataatgggggcc

SEQ ID NO: 1098 - DNA, 161 bp, synthetic construct
  source 1..161: mol_type = other DNA
attaccgggctctgccatcttaagagtgattaccgggctctgccatcttaagagtgattaccgggctctgccatcttaagagtgattaccgggctctgccatcttaagagtgattaccgggctctgccatcttagactctagagggtatataatgggggcc

SEQ ID NO: 1099 - DNA, 165 bp, synthetic construct
  source 1..165: mol_type = other DNA
attaccgggctctgccatcttaagagtgcattaccgggctctgccatcttaagagtgcattaccgggctctgccatcttaagagtgcattaccgggctctgccatcttaagagtgcattaccgggctctgccatcttagactctagagggtatataatgggggcc

SEQ ID NO: 1100 - DNA, 169 bp, synthetic construct
  source 1..169: mol_type = other DNA
attaccgggctctgccatcttaagagtgcgattaccgggctctgccatcttaagagtgcgattaccgggctctgccatcttaagagtgcgattaccgggctctgccatcttaagagtgcgattaccgggctctgccatcttagactctagagggtatataatgggggcc

SEQ ID NO: 1101 - DNA, 173 bp, synthetic construct
  source 1..173: mol_type = other DNA
attaccgggctctgccatcttaagagtgcgaattaccgggctctgccatcttaagagtgcgaattaccgggctctgccatcttaagagtgcgaattaccgggctctgccatcttaagagtgcgaattaccgggctctgccatcttagactctagagggtatataatgggggcc

SEQ ID NO: 1102 - DNA, 177 bp, synthetic construct
  source 1..177: mol_type = other DNA
attaccgggctctgccatcttaagagtgcgagattaccgggctctgccatcttaagagtgcgagattaccgggctctgccatcttaagagtgcgagattaccgggctctgccatcttaagagtgcgagattaccgggctctgccatcttagactctagagggtatataatgggggcc

SEQ ID NO: 1103 - DNA, 138 bp, synthetic construct
  source 1..138: mol_type = other DNA
attaccgggctctgccatcttaattaccgggctctgccatcttaattaccgggctctgccatcttaattaccgggctctgccatcttaattaccgggctctgccatcttagacctctagagggtatataatgggggcc

SEQ ID NO: 1104 - DNA, 142 bp, synthetic construct
  source 1..142: mol_type = other DNA
attaccgggctctgccatcttaaattaccgggctctgccatcttaaattaccgggctctgccatcttaaattaccgggctctgccatcttaaattaccgggctctgccatcttagacctctagagggtatataatgggggcc

SEQ ID NO: 1105 - DNA, 146 bp, synthetic construct
  source 1..146: mol_type = other DNA
attaccgggctctgccatcttaagattaccgggctctgccatcttaagattaccgggctctgccatcttaagattaccgggctctgccatcttaagattaccgggctctgccatcttagacctctagagggtatataatgggggcc

SEQ ID NO: 1106 - DNA, 150 bp, synthetic construct
  source 1..150: mol_type = other DNA
attaccgggctctgccatcttaagaattaccgggctctgccatcttaagaattaccgggctctgccatcttaagaattaccgggctctgccatcttaagaattaccgggctctgccatcttagacctctagagggtatataatgggggcc

SEQ ID NO: 1107 - DNA, 154 bp, synthetic construct
  source 1..154: mol_type = other DNA
attaccgggctctgccatcttaagagattaccgggctctgccatcttaagagattaccgggctctgccatcttaagagattaccgggctctgccatcttaagagattaccgggctctgccatcttagacctctagagggtatataatgggggcc

SEQ ID NO: 1108 - DNA, 158 bp, synthetic construct
  source 1..158: mol_type = other DNA
attaccgggctctgccatcttaagagtattaccgggctctgccatcttaagagtattaccgggctctgccatcttaagagtattaccgggctctgccatcttaagagtattaccgggctctgccatcttagacctctagagggtatataatgggggcc

SEQ ID NO: 1109 - DNA, 162 bp, synthetic construct
  source 1..162: mol_type = other DNA
attaccgggctctgccatcttaagagtgattaccgggctctgccatcttaagagtgattaccgggctctgccatcttaagagtgattaccgggctctgccatcttaagagtgattaccgggctctgccatcttagacctctagagggtatataatgggggcc

SEQ ID NO: 1110 - DNA, 166 bp, synthetic construct
  source 1..166: mol_type = other DNA
attaccgggctctgccatcttaagagtgcattaccgggctctgccatcttaagagtgcattaccgggctctgccatcttaagagtgcattaccgggctctgccatcttaagagtgcattaccgggctctgccatcttagacctctagagggtatataatgggggcc

SEQ ID NO: 1111 - DNA, 170 bp, synthetic construct
  source 1..170: mol_type = other DNA
attaccgggctctgccatcttaagagtgcgattaccgggctctgccatcttaagagtgcgattaccgggctctgccatcttaagagtgcgattaccgggctctgccatcttaagagtgcgattaccgggctctgccatcttagacctctagagggtatataatgggggcc

SEQ ID NO: 1112 - DNA, 174 bp, synthetic construct
  source 1..174: mol_type = other DNA
attaccgggctctgccatcttaagagtgcgaattaccgggctctgccatcttaagagtgcgaattaccgggctctgccatcttaagagtgcgaattaccgggctctgccatcttaagagtgcgaattaccgggctctgccatcttagacctctagagggtatataatgggggcc

SEQ ID NO: 1113 - DNA, 178 bp, synthetic construct
  source 1..178: mol_type = other DNA
attaccgggctctgccatcttaagagtgcgagattaccgggctctgccatcttaagagtgcgagattaccgggctctgccatcttaagagtgcgagattaccgggctctgccatcttaagagtgcgagattaccgggctctgccatcttagacctctagagggtatataatgggggcc

SEQ ID NO: 1114 - DNA, 139 bp, synthetic construct
  source 1..139: mol_type = other DNA
attaccgggctctgccatcttaattaccgggctctgccatcttaattaccgggctctgccatcttaattaccgggctctgccatcttaattaccgggctctgccatcttagactctctagagggtatataatgggggcc

SEQ ID NO: 1115 - DNA, 143 bp, synthetic construct
  source 1..143: mol_type = other DNA
attaccgggctctgccatcttaaattaccgggctctgccatcttaaattaccgggctctgccatcttaaattaccgggctctgccatcttaaattaccgggctctgccatcttagactctctagagggtatataatgggggcc

SEQ ID NO: 1116 - DNA, 147 bp, synthetic construct
  source 1..147: mol_type = other DNA
attaccgggctctgccatcttaagattaccgggctctgccatcttaagattaccgggctctgccatcttaagattaccgggctctgccatcttaagattaccgggctctgccatcttagactctctagagggtatataatgggggcc

SEQ ID NO: 1117 - DNA, 151 bp, synthetic construct
  source 1..151: mol_type = other DNA
attaccgggctctgccatcttaagaattaccgggctctgccatcttaagaattaccgggctctgccatcttaagaattaccgggctctgccatcttaagaattaccgggctctgccatcttagactctctagagggtatataatgggggcc

SEQ ID NO: 1118 - DNA, 155 bp, synthetic construct
  source 1..155: mol_type = other DNA
attaccgggctctgccatcttaagagattaccgggctctgccatcttaagagattaccgggctctgccatcttaagagattaccgggctctgccatcttaagagattaccgggctctgccatcttagactctctagagggtatataatgggggcc

SEQ ID NO: 1119 - DNA, 159 bp, synthetic construct
  source 1..159: mol_type = other DNA
attaccgggctctgccatcttaagagtattaccgggctctgccatcttaagagtattaccgggctctgccatcttaagagtattaccgggctctgccatcttaagagtattaccgggctctgccatcttagactctctagagggtatataatgggggcc

SEQ ID NO: 1120 - DNA, 163 bp, synthetic construct
  source 1..163: mol_type = other DNA
attaccgggctctgccatcttaagagtgattaccgggctctgccatcttaagagtgattaccgggctctgccatcttaagagtgattaccgggctctgccatcttaagagtgattaccgggctctgccatcttagactctctagagggtatataatgggggcc

SEQ ID NO: 1121 - DNA, 167 bp, synthetic construct
  source 1..167: mol_type = other DNA
attaccgggctctgccatcttaagagtgcattaccgggctctgccatcttaagagtgcattaccgggctctgccatcttaagagtgcattaccgggctctgccatcttaagagtgcattaccgggctctgccatcttagactctctagagggtatataatgggggcc

SEQ ID NO: 1122 - DNA, 171 bp, synthetic construct
  source 1..171: mol_type = other DNA
attaccgggctctgccatcttaagagtgcgattaccgggctctgccatcttaagagtgcgattaccgggctctgccatcttaagagtgcgattaccgggctctgccatcttaagagtgcgattaccgggctctgccatcttagactctctagagggtatataatgggggcc

SEQ ID NO: 1123 - DNA, 175 bp, synthetic construct
  source 1..175: mol_type = other DNA
attaccgggctctgccatcttaagagtgcgaattaccgggctctgccatcttaagagtgcgaattaccgggctctgccatcttaagagtgcgaattaccgggctctgccatcttaagagtgcgaattaccgggctctgccatcttagactctctagagggtatataatgggggcc

SEQ ID NO: 1124 - DNA, 179 bp, synthetic construct
  source 1..179: mol_type = other DNA
attaccgggctctgccatcttaagagtgcgagattaccgggctctgccatcttaagagtgcgagattaccgggctctgccatcttaagagtgcgagattaccgggctctgccatcttaagagtgcgagattaccgggctctgccatcttagactctctagagggtatataatgggggcc

SEQ ID NO: 1125 - DNA, 140 bp, synthetic construct
  source 1..140: mol_type = other DNA
attaccgggctctgccatcttaattaccgggctctgccatcttaattaccgggctctgccatcttaattaccgggctctgccatcttaattaccgggctctgccatcttagactcctctagagggtatataatgggggcc

SEQ ID NO: 1126 - DNA, 144 bp, synthetic construct
  source 1..144: mol_type = other DNA
attaccgggctctgccatcttaaattaccgggctctgccatcttaaattaccgggctctgccatcttaaattaccgggctctgccatcttaaattaccgggctctgccatcttagactcctctagagggtatataatgggggcc

SEQ ID NO: 1127 - DNA, 148 bp, synthetic construct
  source 1..148: mol_type = other DNA
attaccgggctctgccatcttaagattaccgggctctgccatcttaagattaccgggctctgccatcttaagattaccgggctctgccatcttaagattaccgggctctgccatcttagactcctctagagggtatataatgggggcc

SEQ ID NO: 1128 - DNA, 152 bp, synthetic construct
  source 1..152: mol_type = other DNA
attaccgggctctgccatcttaagaattaccgggctctgccatcttaagaattaccgggctctgccatcttaagaattaccgggctctgccatcttaagaattaccgggctctgccatcttagactcctctagagggtatataatgggggcc

SEQ ID NO: 1129 - DNA, 156 bp, synthetic construct
  source 1..156: mol_type = other DNA
attaccgggctctgccatcttaagagattaccgggctctgccatcttaagagattaccgggctctgccatcttaagagattaccgggctctgccatcttaagagattaccgggctctgccatcttagactcctctagagggtatataatgggggcc

SEQ ID NO: 1130 - DNA, 160 bp, synthetic construct
  source 1..160: mol_type = other DNA
attaccgggctctgccatcttaagagtattaccgggctctgccatcttaagagtattaccgggctctgccatcttaagagtattaccgggctctgccatcttaagagtattaccgggctctgccatcttagactcctctagagggtatataatgggggcc

SEQ ID NO: 1131 - DNA, 164 bp, synthetic construct
  source 1..164: mol_type = other DNA
attaccgggctctgccatcttaagagtgattaccgggctctgccatcttaagagtgattaccgggctctgccatcttaagagtgattaccgggctctgccatcttaagagtgattaccgggctctgccatcttagactcctctagagggtatataatgggggcc

SEQ ID NO: 1132 - DNA, 168 bp, synthetic construct
  source 1..168: mol_type = other DNA
attaccgggctctgccatcttaagagtgcattaccgggctctgccatcttaagagtgcattaccgggctctgccatcttaagagtgcattaccgggctctgccatcttaagagtgcattaccgggctctgccatcttagactcctctagagggtatataatgggggcc

SEQ ID NO: 1133 - DNA, 172 bp, synthetic construct
  source 1..172: mol_type = other DNA
attaccgggctctgccatcttaagagtgcgattaccgggctctgccatcttaagagtgcgattaccgggctctgccatcttaagagtgcgattaccgggctctgccatcttaagagtgcgattaccgggctctgccatcttagactcctctagagggtatataatgggggcc

SEQ ID NO: 1134 - DNA, 176 bp, synthetic construct
  source 1..176: mol_type = other DNA
attaccgggctctgccatcttaagagtgcgaattaccgggctctgccatcttaagagtgcgaattaccgggctctgccatcttaagagtgcgaattaccgggctctgccatcttaagagtgcgaattaccgggctctgccatcttagactcctctagagggtatataatgggggcc

SEQ ID NO: 1135 - DNA, 180 bp, synthetic construct
  source 1..180: mol_type = other DNA
attaccgggctctgccatcttaagagtgcgagattaccgggctctgccatcttaagagtgcgagattaccgggctctgccatcttaagagtgcgagattaccgggctctgccatcttaagagtgcgagattaccgggctctgccatcttagactcctctagagggtatataatgggggcc

SEQ ID NO: 1136 - DNA, 141 bp, synthetic construct
  source 1..141: mol_type = other DNA
attaccgggctctgccatcttaattaccgggctctgccatcttaattaccgggctctgccatcttaattaccgggctctgccatcttaattaccgggctctgccatcttagactcactctagagggtatataatgggggcc

SEQ ID NO: 1137 - DNA, 145 bp, synthetic construct
  source 1..145: mol_type = other DNA
attaccgggctctgccatcttaaattaccgggctctgccatcttaaattaccgggctctgccatcttaaattaccgggctctgccatcttaaattaccgggctctgccatcttagactcactctagagggtatataatgggggcc

SEQ ID NO: 1138 - DNA, 149 bp, synthetic construct
  source 1..149: mol_type = other DNA
attaccgggctctgccatcttaagattaccgggctctgccatcttaagattaccgggctctgccatcttaagattaccgggctctgccatcttaagattaccgggctctgccatcttagactcactctagagggtatataatgggggcc

SEQ ID NO: 1139 - DNA, 153 bp, synthetic construct
  source 1..153: mol_type = other DNA
attaccgggctctgccatcttaagaattaccgggctctgccatcttaagaattaccgggctctgccatcttaagaattaccgggctctgccatcttaagaattaccgggctctgccatcttagactcactctagagggtatataatgggggcc

SEQ ID NO: 1140 - DNA, 157 bp, synthetic construct
  source 1..157: mol_type = other DNA
attaccgggctctgccatcttaagagattaccgggctctgccatcttaagagattaccgggctctgccatcttaagagattaccgggctctgccatcttaagagattaccgggctctgccatcttagactcactctagagggtatataatgggggcc

SEQ ID NO: 1141 - DNA, 161 bp, synthetic construct
  source 1..161: mol_type = other DNA
attaccgggctctgccatcttaagagtattaccgggctctgccatcttaagagtattaccgggctctgccatcttaagagtattaccgggctctgccatcttaagagtattaccgggctctgccatcttagactcactctagagggtatataatgggggcc

SEQ ID NO: 1142 - DNA, 165 bp, synthetic construct
  source 1..165: mol_type = other DNA
attaccgggctctgccatcttaagagtgattaccgggctctgccatcttaagagtgattaccgggctctgccatcttaagagtgattaccgggctctgccatcttaagagtgattaccgggctctgccatcttagactcactctagagggtatataatgggggcc

SEQ ID NO: 1143 - DNA, 169 bp, synthetic construct
  source 1..169: mol_type = other DNA
attaccgggctctgccatcttaagagtgcattaccgggctctgccatcttaagagtgcattaccgggctctgccatcttaagagtgcattaccgggctctgccatcttaagagtgcattaccgggctctgccatcttagactcactctagagggtatataatgggggcc

SEQ ID NO: 1144 - DNA, 173 bp, synthetic construct
  source 1..173: mol_type = other DNA
attaccgggctctgccatcttaagagtgcgattaccgggctctgccatcttaagagtgcgattaccgggctctgccatcttaagagtgcgattaccgggctctgccatcttaagagtgcgattaccgggctctgccatcttagactcactctagagggtatataatgggggcc

SEQ ID NO: 1145 - DNA, 177 bp, synthetic construct
  source 1..177: mol_type = other DNA
attaccgggctctgccatcttaagagtgcgaattaccgggctctgccatcttaagagtgcgaattaccgggctctgccatcttaagagtgcgaattaccgggctctgccatcttaagagtgcgaattaccgggctctgccatcttagactcactctagagggtatataatgggggcc

SEQ ID NO: 1146 - DNA, 181 bp, synthetic construct
  source 1..181: mol_type = other DNA
attaccgggctctgccatcttaagagtgcgagattaccgggctctgccatcttaagagtgcgagattaccgggctctgccatcttaagagtgcgagattaccgggctctgccatcttaagagtgcgagattaccgggctctgccatcttagactcactctagagggtatataatgggggcc

SEQ ID NO: 1147 - DNA, 142 bp, synthetic construct
  source 1..142: mol_type = other DNA
attaccgggctctgccatcttaattaccgggctctgccatcttaattaccgggctctgccatcttaattaccgggctctgccatcttaattaccgggctctgccatcttagactcatctctagagggtatataatgggggcc

SEQ ID NO: 1148 - DNA, 146 bp, synthetic construct
  source 1..146: mol_type = other DNA
attaccgggctctgccatcttaaattaccgggctctgccatcttaaattaccgggctctgccatcttaaattaccgggctctgccatcttaaattaccgggctctgccatcttagactcatctctagagggtatataatgggggcc

SEQ ID NO: 1149 - DNA, 150 bp, synthetic construct
  source 1..150: mol_type = other DNA
attaccgggctctgccatcttaagattaccgggctctgccatcttaagattaccgggctctgccatcttaagattaccgggctctgccatcttaagattaccgggctctgccatcttagactcatctctagagggtatataatgggggcc

SEQ ID NO: 1150 - DNA, 154 bp, synthetic construct
  source 1..154: mol_type = other DNA
attaccgggctctgccatcttaagaattaccgggctctgccatcttaagaattaccgggctctgccatcttaagaattaccgggctctgccatcttaagaattaccgggctctgccatcttagactcatctctagagggtatataatgggggcc

SEQ ID NO: 1151 - DNA, 158 bp, synthetic construct
  source 1..158: mol_type = other DNA
attaccgggctctgccatcttaagagattaccgggctctgccatcttaagagattaccgggctctgccatcttaagagattaccgggctctgccatcttaagagattaccgggctctgccatcttagactcatctctagagggtatataatgggggcc

SEQ ID NO: 1152 - DNA, 162 bp, synthetic construct
  source 1..162: mol_type = other DNA
attaccgggctctgccatcttaagagtattaccgggctctgccatcttaagagtattaccgggctctgccatcttaagagtattaccgggctctgccatcttaagagtattaccgggctctgccatcttagactcatctctagagggtatataatgggggcc

SEQ ID NO: 1153 - DNA, 166 bp, synthetic construct
  source 1..166: mol_type = other DNA
attaccgggctctgccatcttaagagtgattaccgggctctgccatcttaagagtgattaccgggctctgccatcttaagagtgattaccgggctctgccatcttaagagtgattaccgggctctgccatcttagactcatctctagagggtatataatgggggcc

SEQ ID NO: 1154 - DNA, 170 bp, synthetic construct
  source 1..170: mol_type = other DNA
attaccgggctctgccatcttaagagtgcattaccgggctctgccatcttaagagtgcattaccgggctctgccatcttaagagtgcattaccgggctctgccatcttaagagtgcattaccgggctctgccatcttagactcatctctagagggtatataatgggggcc

SEQ ID NO: 1155 - DNA, 174 bp, synthetic construct
  source 1..174: mol_type = other DNA
attaccgggctctgccatcttaagagtgcgattaccgggctctgccatcttaagagtgcgattaccgggctctgccatcttaagagtgcgattaccgggctctgccatcttaagagtgcgattaccgggctctgccatcttagactcatctctagagggtatataatgggggcc

SEQ ID NO: 1156 - DNA, 178 bp, synthetic construct
  source 1..178: mol_type = other DNA
attaccgggctctgccatcttaagagtgcgaattaccgggctctgccatcttaagagtgcgaattaccgggctctgccatcttaagagtgcgaattaccgggctctgccatcttaagagtgcgaattaccgggctctgccatcttagactcatctctagagggtatataatgggggcc

SEQ ID NO: 1157 - DNA, 182 bp, synthetic construct
  source 1..182: mol_type = other DNA
attaccgggctctgccatcttaagagtgcgagattaccgggctctgccatcttaagagtgcgagattaccgggctctgccatcttaagagtgcgagattaccgggctctgccatcttaagagtgcgagattaccgggctctgccatcttagactcatctctagagggtatataatgggggcc

SEQ ID NO: 1158 - DNA, 143 bp, synthetic construct
  source 1..143: mol_type = other DNA
attaccgggctctgccatcttaattaccgggctctgccatcttaattaccgggctctgccatcttaattaccgggctctgccatcttaattaccgggctctgccatcttagactcatactctagagggtatataatgggggcc

SEQ ID NO: 1159 - DNA, 147 bp, synthetic construct
  source 1..147: mol_type = other DNA
attaccgggctctgccatcttaaattaccgggctctgccatcttaaattaccgggctctgccatcttaaattaccgggctctgccatcttaaattaccgggctctgccatcttagactcatactctagagggtatataatgggggcc

SEQ ID NO: 1160 - DNA, 151 bp, synthetic construct
  source 1..151: mol_type = other DNA
attaccgggctctgccatcttaagattaccgggctctgccatcttaagattaccgggctctgccatcttaagattaccgggctctgccatcttaagattaccgggctctgccatcttagactcatactctagagggtatataatgggggcc

SEQ ID NO: 1161 - DNA, 155 bp, synthetic construct
  source 1..155: mol_type = other DNA
attaccgggctctgccatcttaagaattaccgggctctgccatcttaagaattaccgggctctgccatcttaagaattaccgggctctgccatcttaagaattaccgggctctgccatcttagactcatactctagagggtatataatgggggcc

SEQ ID NO: 1162 - DNA, 159 bp, synthetic construct
  source 1..159: mol_type = other DNA
attaccgggctctgccatcttaagagattaccgggctctgccatcttaagagattaccgggctctgccatcttaagagattaccgggctctgccatcttaagagattaccgggctctgccatcttagactcatactctagagggtatataatgggggcc

SEQ ID NO: 1163 - DNA, 163 bp, synthetic construct
  source 1..163: mol_type = other DNA
attaccgggctctgccatcttaagagtattaccgggctctgccatcttaagagtattaccgggctctgccatcttaagagtattaccgggctctgccatcttaagagtattaccgggctctgccatcttagactcatactctagagggtatataatgggggcc

SEQ ID NO: 1164 - DNA, 167 bp, synthetic construct
  source 1..167: mol_type = other DNA
attaccgggctctgccatcttaagagtgattaccgggctctgccatcttaagagtgattaccgggctctgccatcttaagagtgattaccgggctctgccatcttaagagtgattaccgggctctgccatcttagactcatactctagagggtatataatgggggcc

SEQ ID NO: 1165 - DNA, 171 bp, synthetic construct
  source 1..171: mol_type = other DNA
attaccgggctctgccatcttaagagtgcattaccgggctctgccatcttaagagtgcattaccgggctctgccatcttaagagtgcattaccgggctctgccatcttaagagtgcattaccgggctctgccatcttagactcatactctagagggtatataatgggggcc

SEQ ID NO: 1166 - DNA, 175 bp, synthetic construct
  source 1..175: mol_type = other DNA
attaccgggctctgccatcttaagagtgcgattaccgggctctgccatcttaagagtgcgattaccgggctctgccatcttaagagtgcgattaccgggctctgccatcttaagagtgcgattaccgggctctgccatcttagactcatactctagagggtatataatgggggcc

SEQ ID NO: 1167 - DNA, 179 bp, synthetic construct
  source 1..179: mol_type = other DNA
attaccgggctctgccatcttaagagtgcgaattaccgggctctgccatcttaagagtgcgaattaccgggctctgccatcttaagagtgcgaattaccgggctctgccatcttaagagtgcgaattaccgggctctgccatcttagactcatactctagagggtatataatgggggcc

SEQ ID NO: 1168 - DNA, 183 bp, synthetic construct
  source 1..183: mol_type = other DNA
attaccgggctctgccatcttaagagtgcgagattaccgggctctgccatcttaagagtgcgagattaccgggctctgccatcttaagagtgcgagattaccgggctctgccatcttaagagtgcgagattaccgggctctgccatcttagactcatactctagagggtatataatgggggcc

SEQ ID NO: 1169 - DNA, 144 bp, synthetic construct
  source 1..144: mol_type = other DNA
attaccgggctctgccatcttaattaccgggctctgccatcttaattaccgggctctgccatcttaattaccgggctctgccatcttaattaccgggctctgccatcttagactcatatctctagagggtatataatgggggcc

SEQ ID NO: 1170 - DNA, 148 bp, synthetic construct
  source 1..148: mol_type = other DNA
attaccgggctctgccatcttaaattaccgggctctgccatcttaaattaccgggctctgccatcttaaattaccgggctctgccatcttaaattaccgggctctgccatcttagactcatatctctagagggtatataatgggggcc

SEQ ID NO: 1171 - DNA, 152 bp, synthetic construct
  source 1..152: mol_type = other DNA
attaccgggctctgccatcttaagattaccgggctctgccatcttaagattaccgggctctgccatcttaagattaccgggctctgccatcttaagattaccgggctctgccatcttagactcatatctctagagggtatataatgggggcc

SEQ ID NO: 1172 - DNA, 156 bp, synthetic construct
  source 1..156: mol_type = other DNA
attaccgggctctgccatcttaagaattaccgggctctgccatcttaagaattaccgggctctgccatcttaagaattaccgggctctgccatcttaagaattaccgggctctgccatcttagactcatatctctagagggtatataatgggggcc

SEQ ID NO: 1173 - DNA, 160 bp, synthetic construct
  source 1..160: mol_type = other DNA
attaccgggctctgccatcttaagagattaccgggctctgccatcttaagagattaccgggctctgccatcttaagagattaccgggctctgccatcttaagagattaccgggctctgccatcttagactcatatctctagagggtatataatgggggcc

SEQ ID NO: 1174 - DNA, 164 bp, synthetic construct
  source 1..164: mol_type = other DNA
attaccgggctctgccatcttaagagtattaccgggctctgccatcttaagagtattaccgggctctgccatcttaagagtattaccgggctctgccatcttaagagtattaccgggctctgccatcttagactcatatctctagagggtatataatgggggcc

SEQ ID NO: 1175 - DNA, 168 bp, synthetic construct
  source 1..168: mol_type = other DNA
attaccgggctctgccatcttaagagtgattaccgggctctgccatcttaagagtgattaccgggctctgccatcttaagagtgattaccgggctctgccatcttaagagtgattaccgggctctgccatcttagactcatatctctagagggtatataatgggggcc

SEQ ID NO: 1176 - DNA, 172 bp, synthetic construct
  source 1..172: mol_type = other DNA
attaccgggctctgccatcttaagagtgcattaccgggctctgccatcttaagagtgcattaccgggctctgccatcttaagagtgcattaccgggctctgccatcttaagagtgcattaccgggctctgccatcttagactcatatctctagagggtatataatgggggcc

SEQ ID NO: 1177 - DNA, 176 bp, synthetic construct
  source 1..176: mol_type = other DNA
attaccgggctctgccatcttaagagtgcgattaccgggctctgccatcttaagagtgcgattaccgggctctgccatcttaagagtgcgattaccgggctctgccatcttaagagtgcgattaccgggctctgccatcttagactcatatctctagagggtatataatgggggcc

SEQ ID NO: 1178 - DNA, 180 bp, synthetic construct
  source 1..180: mol_type = other DNA
attaccgggctctgccatcttaagagtgcgaattaccgggctctgccatcttaagagtgcgaattaccgggctctgccatcttaagagtgcgaattaccgggctctgccatcttaagagtgcgaattaccgggctctgccatcttagactcatatctctagagggtatataatgggggcc

SEQ ID NO: 1179 - DNA, 184 bp, synthetic construct
  source 1..184: mol_type = other DNA
attaccgggctctgccatcttaagagtgcgagattaccgggctctgccatcttaagagtgcgagattaccgggctctgccatcttaagagtgcgagattaccgggctctgccatcttaagagtgcgagattaccgggctctgccatcttagactcatatctctagagggtatataatgggggcc

SEQ ID NO: 1180 - DNA, 145 bp, synthetic construct
  source 1..145: mol_type = other DNA
attaccgggctctgccatcttaattaccgggctctgccatcttaattaccgggctctgccatcttaattaccgggctctgccatcttaattaccgggctctgccatcttagactcatatgctctagagggtatataatgggggcc

SEQ ID NO: 1181 - DNA, 149 bp, synthetic construct
  source 1..149: mol_type = other DNA
attaccgggctctgccatcttaaattaccgggctctgccatcttaaattaccgggctctgccatcttaaattaccgggctctgccatcttaaattaccgggctctgccatcttagactcatatgctctagagggtatataatgggggcc

SEQ ID NO: 1182 - DNA, 153 bp, synthetic construct
  source 1..153: mol_type = other DNA
attaccgggctctgccatcttaagattaccgggctctgccatcttaagattaccgggctctgccatcttaagattaccgggctctgccatcttaagattaccgggctctgccatcttagactcatatgctctagagggtatataatgggggcc

SEQ ID NO: 1183 - DNA, 157 bp, synthetic construct
  source 1..157: mol_type = other DNA
attaccgggctctgccatcttaagaattaccgggctctgccatcttaagaattaccgggctctgccatcttaagaattaccgggctctgccatcttaagaattaccgggctctgccatcttagactcatatgctctagagggtatataatgggggcc

SEQ ID NO: 1184 - DNA, 161 bp, synthetic construct
  source 1..161: mol_type = other DNA
attaccgggctctgccatcttaagagattaccgggctctgccatcttaagagattaccgggctctgccatcttaagagattaccgggctctgccatcttaagagattaccgggctctgccatcttagactcatatgctctagagggtatataatgggggcc

SEQ ID NO: 1185 - DNA, 165 bp, synthetic construct
  source 1..165: mol_type = other DNA
attaccgggctctgccatcttaagagtattaccgggctctgccatcttaagagtattaccgggctctgccatcttaagagtattaccgggctctgccatcttaagagtattaccgggctctgccatcttagactcatatgctctagagggtatataatgggggcc

SEQ ID NO: 1186 - DNA, 169 bp, synthetic construct
  source 1..169: mol_type = other DNA
attaccgggctctgccatcttaagagtgattaccgggctctgccatcttaagagtgattaccgggctctgccatcttaagagtgattaccgggctctgccatcttaagagtgattaccgggctctgccatcttagactcatatgctctagagggtatataatgggggcc

SEQ ID NO: 1187 - DNA, 173 bp, synthetic construct
  source 1..173: mol_type = other DNA
attaccgggctctgccatcttaagagtgcattaccgggctctgccatcttaagagtgcattaccgggctctgccatcttaagagtgcattaccgggctctgccatcttaagagtgcattaccgggctctgccatcttagactcatatgctctagagggtatataatgggggcc

SEQ ID NO: 1188 - DNA, 177 bp, synthetic construct
  source 1..177: mol_type = other DNA
attaccgggctctgccatcttaagagtgcgattaccgggctctgccatcttaagagtgcgattaccgggctctgccatcttaagagtgcgattaccgggctctgccatcttaagagtgcgattaccgggctctgccatcttagactcatatgctctagagggtatataatgggggcc

SEQ ID NO: 1189 - DNA, 181 bp, synthetic construct
  source 1..181: mol_type = other DNA
attaccgggctctgccatcttaagagtgcgaattaccgggctctgccatcttaagagtgcgaattaccgggctctgccatcttaagagtgcgaattaccgggctctgccatcttaagagtgcgaattaccgggctctgccatcttagactcatatgctctagagggtatataatgggggcc

SEQ ID NO: 1190 - DNA, 185 bp, synthetic construct
  source 1..185: mol_type = other DNA
attaccgggctctgccatcttaagagtgcgagattaccgggctctgccatcttaagagtgcgagattaccgggctctgccatcttaagagtgcgagattaccgggctctgccatcttaagagtgcgagattaccgggctctgccatcttagactcatatgctctagagggtatataatgggggcc

(19) United States
(12) Patent Application Publication
(10) Pub. No.: US 2026/0200999 A1
(43) Pub. Date: Jul. 16, 2026

(54) SYNTHETIC TRANSCRIPTION FACTORS

(71) Applicant: ETH ZURICH, Zurich (CH)

(72) Inventors: David Schweingruber, Zurich (CH); Elena BOSCHET, Zurich (CH); Jan NELIS, Zurich (CH); Yaakov BENENSON, Zurich (CH); Cheyenne RECHSTEINER, Zurich (CH)

(21) Appl. No.: 19/137,447

(22) PCT Filed: Dec. 21, 2023
(86) PCT No.: PCT/EP2023/087354

§ 371 Date: Jun. 10, 2025

(30) Foreign Application Priority Data
Dec. 23, 2022 (EP) 22216667.0
Aug. 24, 2023 (EP) 23193246.8
Aug. 25, 2023 (EP) 23193528.9

(51) Int. Cl. C07K 14/47; A61K 38/00; A61K 40/11; A61K 40/31; A61K 48/00; C12N 15/10

(52) U.S. Cl. CPC C07K 14/4702; A61K 40/11; A61K 40/31; C12N 15/1093; A61K 38/00; A61K 48/00; C07K 2319/09; C07K 2319/80

(57) ABSTRACT

The present invention is generally in the fields of synthetic biology, gene therapy, and cell therapy. The invention relates, inter alia, to a synthetic transcription factor comprising a DNA binding domain derived from a mitochondrial DNA binding protein (e.g. MTERF1) and a transcriptional modulation domain derived from one or more other proteins; a nucleic acid or combination of nucleic acids encoding the inventive synthetic transcription factor; a DNA construct comprising a MTERF1 binding site and a minimal promoter; a system comprising the inventive synthetic transcription factor and the inventive DNA construct; and uses thereof, e.g. medical uses.

The present invention is generally in the fields of synthetic biology, gene therapy, and cell therapy. The invention relates, inter alia, to a synthetic transcription factor comprising a DNA binding domain derived from a mitochondrial DNA binding protein (e.g. MTERF1) and a transcriptional modulation domain derived from one or more other proteins; a nucleic acid or combination of nucleic acids encoding the inventive synthetic transcription factor; a DNA construct comprising a MTERF1 binding site and a minimal promoter; a system comprising the inventive synthetic transcription factor and the inventive DNA construct; and uses thereof, e.g. medical uses. Furthermore, the invention relates to a library of DNA constructs and its use in a method of optimizing a promoter for binding to a transcription factor.

In gene and cell therapies (GCT), man-made genetic sequences are central to a therapeutic formulation. In gene therapy, a delivery vector harboring the therapeutic genetic sequence (“payload”) is directly administered to the patient, delivering the therapeutic payload to multiple cells in the patient's body. In cell therapy, the payload is introduced ex vivo into cells, e.g., patient-derived cells, which are then (re) infused into the patient [1].

A therapeutic transgene encoded on the genetic payload of a GCT is typically driven by a constitutive or a tissue-specific promoter. However, the repertoire of naturally occurring promoters for specific expression of transgenes is limited [2]. Alternatively, tissue- or cell type-specificity can be achieved with the help of an engineered genetic network (likewise fully encoded on a genetic payload) that processes multiple cellular inputs in a programmable logical fashion (i.e., a “biocomputing circuit”). For example, a cancer cell classifier circuit was engineered to restrict the expression of a pro-apoptotic gene to cancer cells while sparing healthy ones [3]. In the context of a chimeric antigen receptor (CAR) T-cell therapy, an “AND”-gate was built resulting in increased specificity towards cancer cells co-expressing two defined antigens on their surface [4]. Such circuits may lead to safer and more efficacious GCTs. However, the circuits typically encode protein components (“auxiliary proteins”) in addition to the therapeutic transgene. These protein components help to execute the logical control process for precise activation of a therapeutic transgene when multiple conditions are met in the target cell.

Auxiliary proteins, e.g., transcription factors, may elevate toxicity and reduce efficacy of biocomputing-based therapies in human patients, in particular if these proteins are of non-human origin. The use of auxiliary proteins from non-human origin is driven by the so-called “orthogonality requirement”: these proteins should not interfere with endogenous gene regulation in the human cell, and they, or the processes they control, should not be modifiable by, or interfered with, the endogenous human factors. There is, a priori, the concern that a fully-human protein employed as an auxiliary protein in a gene therapy payload will engage with the endogenous components of the human cell and result in side effects and toxicity. In order to achieve orthogonality of an auxiliary protein, in particular when the protein is a transcription factor, the prior art has resorted to non-human DNA binding domains (DBDs) that do not have binding sites in the regulatory sequences of human genes. Non-human DBDs are often derived from prokaryotes, and they are further fused to a transactivation domain of viral or human origin [5]. While fulfilling the orthogonality requirement, it has been found in animal models and in human clinical trials that non-human proteins may elicit an immune response and lead to the elimination of cells expressing such proteins [6] [7]. This results in reduced therapeutic efficacy of GCT product. Thus, the requirement for orthogonality points toward the use of proteins of non-human origin while the requirement for low immunogenicity suggests the opposite, i.e., the use of human or mostly-human proteins, which in turn may lead to reduced orthogonality. Therefore, reconciling these two opposite requirements is challenging.

There are two current approaches to deal with immunogenicity of transgenes in general, and of proteins of non-human origin in particular.

The first approach aims to eliminate an immune response while accepting the potentially immunogenic protein sequence. Often this entails systemic pharmacological suppression of the immune system. However, this strategy may increase a patient's risk to infection. Other strategies take inspiration from or copy viral strategies against host-defenses and include the prevention of proteasomal degradation and epitope production [9], downregulation of MHC class I [10], antibody-degrading surface enzymes [11], and combinations thereof. Among the drawbacks of these strategies are difficulties in fine-tuning and reliability, and typically large DNA cargoes in cases where immune-evasive viral proteins are encoded on the delivery vectors. A more targeted approach introduces antigen presenting cell (APC)-specific miRNA target sites in the 5 or 3′ UTR of the transgene [12]. This prevents transgene expression in APCs, a central step in the induction of the immune response. While shown to be effective in certain conditions, this approach fails in others, hindering application for a range of diseases [13].

For certain delivery vectors, such as AAV, it was shown that inhibiting AAV interaction with TLR9 in dendritic cells reduces the mounting of adaptive immunity against viral capsid and transgene products [14].

Another approach is to try and reduce the immunogenicity of the protein itself by means of modifications to, or a smart choice of, the protein sequence. One strategy is to “humanize” the transgenes, that is, to replace non-human protein domains with human-derived domains that are not recognized as foreign/non-self by the immune system. For synthetic transcription factors, this strategy is applicable to the transactivation domain because those domains are generally not expected not lead to cross-talk with endogenous processes on their own. However, it is very difficult to apply the strategy to the DNA-binding domain (DBD) because a DBD of human origin does entail the risk of interference with endogenous gene expression. In one previously-proposed solution for “humanizing” DBDs, Zinc finger (ZNF) protein domains derived from human proteins, each domain having DNA binding specificity of 2 to 3 DNA base pairs, were fused to each other to recognize longer DNA sequences not found naturally in the human genome, thereby fulfilling the orthogonality criterion [15] [16]. Fusion of several ZNF domains, however, yields multiple domain junction regions of non-human sequence that may themselves be immunogenic. Accordingly, such artificial DBDs are in large parts non-human. Particularly considering a large patient pool with very diverse MHC, TCR, and antibody repertoires, immune responses against junction-derived peptides are still likely to occur. Another way to humanize the DBD of a synthetic transcription factor (TF) is to make use of naturally occurring human DBDs. This reduces the number of novel (non-human) junctions to one, namely the junction between a human-derived DBD and a human-derived TAD, thereby minimizing the immunogenic potential. Typically, one employs a DBD of a human TF that is not expressed in the cell type in which the synthetic TF is expressed [17]. However, there is the risk that chimeric proteins built according to this strategy bind to their cognate response elements in the human genome and therefore lead to unwanted expression of endogenous human genes, violating the requirement for orthogonality.

Accordingly, there is still a need for improved means and methods for regulating gene expression, in particular in humans.

The invention relates to the embodiments as characterized in the claims and as described herein below.

Accordingly, the present invention relates to a synthetic transcription factor comprising (i) a DNA binding domain (DBD) derived from a mitochondrial DNA binding protein and (ii) a transcriptional modulation domain derived from one or more other proteins.

The invention is, at least partly, based on the surprising finding that a synthetic transcription factor comprising as DNA binding domain (DBD) a DBD from a mitochondrial DNA binding protein such as MTERF1 is able to regulate transcription in a cell, in particular in a nucleus of a cell.

As illustrated in the appended Examples, it has been found that a synthetic transcription factor, in particular a fusion protein (termed “MTF”) comprising (i) a C-terminal fragment of a human MTERF1 protein containing a DNA binding domain but lacking the mitochondrial transfer peptide (MTP) and (ii) the human transactivation domain RelA₄₃₀₋₅₅₁, is able to promote transcription of a gene of interest (Gol) in a cell, in particular in a nucleus of a cell. Further in this context, a DNA construct, i.e., a gene expression construct, has been developed which comprises a promoter containing a response element (RE) for binding of this synthetic transcription factor and a minimal promoter, wherein the promoter may be operably linked to a gene of interest.

As further illustrated in the appended Examples, it has been surprisingly found that the synthetic transcription factor of the invention, e.g., the MTF protein, and the inventive transcriptional system, e.g. comprising said synthetic transcription and said DNA construct, have a high orthogonality in cells of interest, e.g. in human cells; see, e.g. Example 3.

In particular, the inventors could demonstrate that the synthetic transcription factor, i.e, the MTF protein, diffused within a cell, including the nucleus (see, e.g., FIG. 7A). This functionality was in stark contrast to the WT MTERF1 protein which localized only to the mitochondria. Moreover, the capacity of the synthetic transcription factor to promote the transcription of a gene of interest from a synthetic DNA construct was not affected in the cells when the WT MTERF1 protein was overexpressed (see, e.g., FIG. 7C).

Using RNA-Seq, the inventors further found that the MTF did not retain the gene regulatory functionality of WT MTERF1. Very surprisingly, only very few genes were differentially expressed upon MTF construct transfection which demonstrates a high orthogonality with respect to the endogenous gene regulatory processes in human cells (see, e.g., FIG. 8).

Hence, the present inventors developed a synthetic transcription factor comprising a human DNA binding domain which, unexpectedly, had a very high orthogonality in human cells. In particular, (i) the endogenous human gene expression was not substantially altered by the inventive synthetic transcription factor and (ii) the endogenously-expressed wild-type MTERF1 did not modulate, i.e it did not disturb, the expression of a gene of interest driven by a corresponding Response Element-containing promoter, i.e. an inventive DNA construct, in human cells. In other words, the inventive means of the invention illustrated in the appended Examples did not substantially interfere with endogenous gene regulation in the human cell, and the transcription of the gene of interest was not interfered with by the endogenous human factor, i.e. WT MTERF1.

Accordingly, the present invention provides, inter alia, improved components for an improved transcriptional system, in particular an improved synthetic transcription factor and a corresponding gene expression construct, which functions in a substantially orthogonal manner in cells of a certain species, e.g., in humans.

A high orthogonality in cells, e.g., in human cells, is advantageous to ensure a reliable control of the expression of the gene of interest which may, for example, encode for a pro-apoptotic protein. In particular, a high orthogonality ensures that the gene of interest is only expressed in those cells it should be expressed and only when it should be expressed. In addition, a high orthogonality of the synthetic transcriptional system ensures that the endogenous gene expression is not disturbed or disrupted in an undesired manner, and therefore that the risk of side effects or toxicity is reduced.

In particular, a high orthogonality is highly advantageous in context of engineered genetic networks that process multiple cellular inputs in a programmable logical fashion (i.e., a “biocomputing circuit”), for example, in cancer cell classifier circuits.

It is expected that gene therapy products that require engineered transcription factors as part of their mechanism of action, for example gene therapy products that operate as multi-component networks otherwise known as “biocomputing gene circuits”, will have a favorable safety profile when using a synthetic transcription factor according to the present invention compared to alternatives.

Accordingly, the present invention further provides for more effective and/or safer means as described herein for gene and/or cell therapies, e.g., for therapies employing biocomputing circuits.

As used herein, a gene refers to a sequence of nucleotides in DNA that is transcribed to produce a functional RNA. The gene may be a protein-coding gene or a noncoding gene. Moreover, a gene is usually associated with at least one regulatory sequence, e.g., a promoter and optionally an enhancer, which can be involved in the transcription of the gene. A promoter that is able to be involved in the transcription of a gene may be further considered as being operably linked to the gene. Usually, a regulatory sequence (e.g. a promoter) comprises at least one response element and a response element comprises at least one binding site for a transcription factor, as further described herein.

As used herein, a transcription factor refers to a protein which regulates transcription of one or more genes. In particular, a transcription factor controls the rate of transcription of a gene, i.e. the transcription of genetic information from DNA to messenger RNA, by binding to a specific DNA sequence, i.e. a response element (RE), e.g. in a promoter. A “response element” may be also called a “transcription factor-binding site” or comprise at least one transcription factor binding site.

A defining feature of transcription factors is that they contain at least one DNA-binding domain (DBD), which binds, i.e. attaches to, a specific sequence of DNA (i.e. a regulatory sequence) adjacent to or at some distance to the genes that they regulate. In particular, the DBD binds to a response element contained in the regulatory sequence, e.g., the promoter or enhancer.

Furthermore, transcription factors contain a transcriptional modulation domain, e.g. an activation domain or a repression domain which typically contains interaction sites for other proteins such as transcriptional coregulators. The activation domain may be also called “transactivation domain (TAD)” or “transcriptional activation domain”. Similarly, the repression domain may be also called “transcriptional repression domain”.

Optionally, a transcription factor may further contain a signal-sensing domain (SSD) (e.g., a ligand-binding domain), which senses external signals and, in response, transmits these signals to the rest of a transcription complex, resulting in up- or down-regulation of gene expression.

Although transcription factors may work alone, they often work with other proteins in a complex, by promoting (as an activator), or suppressing (as a repressor) the recruitment of RNA polymerase, i.e. an enzyme that performs the transcription of genetic information from DNA to RNA, to specific genes, e.g. a gene of interest as described herein. Hence, the DNA binding domain of a transcription factor directs the transcription factor to a regulatory sequence of a gene, in particular a response element contained in a promoter or enhancer associated with the gene, and the transcription modulation domain promotes or suppresses the transcription of the gene, usually in concert with endogenous transcriptional regulators which bind to or interact with the transcriptional modulation domain i.e. coregulators as further described herein below. In particular, a transcription factor may stimulate initiation of the transcription, especially when it has an activation domain, or rather hinder initiation of the transcription, especially when it has a repression domain. For example, a transcription factor may help RNA polymerase binding to DNA which may rather promote transcription, esp. transcription initiation, or a transcription factor may hinder RNA polymerase binding to DNA which may rather suppress transcription, esp., transcription initiation.

Herein and in context of the present invention, the term “synthetic” means, in particular, that a compound, e.g., a transcription factor, is composed of at least two parts which do not occur together in nature in such a way. In particular, a synthetic protein, e.g., a synthetic transcription factor, comprises one part derived from a certain protein and at least one other part derived from at least one other protein.

In particular herein, the synthetic transcription factor of the invention comprises (i) a DNA binding domain (DBD) derived from a certain protein, i.e., a mitochondrial DNA binding protein and (ii) a transcriptional modulation domain derived from one or more other proteins.

In preferred embodiments of the present invention, the synthetic transcription factor is a fusion protein.

As used herein and in context of the present invention, a fusion protein refers to a synthetic protein, e.g. a synthetic transcription factor, wherein at least two or all parts of the protein, in particular parts which do not occur together in a single polypeptide in nature, are contained in one amino acid chain, i.e. one polypeptide.

Therefore, the synthetic transcription factor of the invention is, in preferred embodiments, a fusion protein comprising the DNA binding domain according to the invention and the transcriptional modulation domain according to the invention. In particular, the two domains are connected in the fusion protein via a peptide bond, either directly or via a peptide linker. In other words, the DNA binding domain according to the invention and the transcriptional modulation domain according to the invention are, in preferred embodiments, contained in one amino acid chain, i.e. one polypeptide. More preferably, in context of these embodiments, essentially all parts of the synthetic transcription factor of the invention are contained in one polypeptide.

In further embodiments of the invention, the synthetic transcription factor comprises or consists of a first and a second polypeptide, wherein said first polypeptide comprises the DNA binding domain according to the invention, and said second polypeptide comprises the transcriptional modulation domain according to the invention. In particular, each of the first and second polypeptide comprises a multimerization domain as described herein, wherein the multimerization domains of the first and second polypeptide are capable of binding to and/or interacting with each other.

Herein and in context of the present invention, a mitochondrial DNA binding protein refers to a DNA binding protein which normally localizes to mitochondria. In particular, a mitochondrial DNA binding protein binds to mitochondrial DNA in a sequence-specific manner. For example, a mitochondrial DNA binding protein may be a mitochondrial transcription factor or a mitochondrial transcription termination factor.

Preferably herein and in context of the present invention, the mitochondrial DNA binding protein is from a mammalian species. Preferably herein, the mammalian species is a human.

It has been further found in context of the present invention that MTERF1 has large recognition sites that are rare in or absent from gene-regulatory sequences in the nuclear genome of a human cell and that MTERF1 does not have any perfect binding sites in the human nuclear genome. This is particularly beneficial for a high orthogonality, e.g., in human cells, as described herein.

Therefore, the mitochondrial DNA binding protein is, herein and in context of the present invention, preferably MTERF1, preferably human MTERF1, i.e. Uniprot Q99551. In particular, human MTERF1, i.e. wild-type (WT) MTERF1, has an amino acid sequence as shown in SEQ ID NO: 108.

However, the MTERF1 may be also from other species, e.g. mouse or dog. In particular, a mouse MTERF1 has an amino acid sequence as shown in SEQ ID NO: 111 or 113. Furthermore, a canine MTERF1 has, in particular, an amino acid sequence as shown in SEQ ID NO: 115. Moreover, MTERF1 orthologue sequences from other species, e.g. mammalian species, are readily available to the skilled person and may be also used herein and in context of the present invention.

Herein and in context of the present invention, the term “derived” should be construed in a technically meaningful manner. For example, a domain derived from a certain protein refers to a part or fragment of said protein which may further comprise at least one modification, in particular at least one amino acid substitution, deletion and/or insertion. The extent of the modifications can be defined by a sequence identity to a reference sequence. Furthermore, a domain that is derived from a certain protein has, in particular, a qualitatively similar functionality than the domain in said protein (although the functionality may be enhanced or reduced to some extent). In particular, a DNA binding domain derived from a certain DNA binding protein, e.g. MTERF1, has the ability to bind to DNA in a sequence specific manner, in particular to bind to a response element of said DNA binding protein, e.g. MTERF1.

Similarly, a transcription modulation domain, e.g. an activation domain, derived from a certain transactivating protein, e.g. a transcription factor such as RELA, has, in particular, the ability to regulate, e.g. promote, the transcription of a gene when it is bound to or located in close proximity to a regulatory sequence, e.g. a response element, of said gene.

In general, herein, and in the context of the present invention, an amino acid sequence (e.g. of a certain protein domain or motif) or a DNA sequence (e.g. of a binding site or minimal protein) may be defined by a certain % sequence identity to a reference sequence. Thus, the term “sequence identity” is used herein, in particular, to describe the sequence relationships between two or more amino acid sequences, proteins (or fragments thereof), or polypeptides (or fragments thereof). In particular, a sequence may have a sequence identity of at least n % to a reference sequence with n being an integer between 60 and 100, e.g., 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99.

For example, an amino acid sequence may have at least 60%, 70%, 80%, or 90%, preferably at least 80%, 85%, 90%, or 95%, more preferably at least 90%, 91%, 92%, 93%, 94%, 95%, 96%, 97%, 98% or 99%, sequence identity to an amino acid sequence set forth in a certain SEQ ID NO. The same applies to nucleic acid sequences, e.g. DNA sequences, mutatis mutandis.

In general, the higher the % of the sequence identity, the more preferred the sequence is. However, further preferred % identities are described directly in the context of certain embodiments herein. It should be further noted that the invention is in no way limited to high or preferred sequence identities, but any sequence identity %, e.g. as just described above, may be considered.

The term “sequence identity”, as used herein, and in the context of the present invention, has essentially the same meaning, as commonly used and understood by the person skilled in the art. The degree of sequence identity can be determined according to methods well known in the art using preferably suitable computer algorithms such as CLUSTAL.

When using the Clustal analysis method to determine whether a particular sequence is, for instance, at least 60% identical to a reference sequence default settings may be used.

In a preferred embodiment Clustal Omega (Madeira F, Park Y M, Lee J, et al. The EMBL-EBI search and sequence analysis tools APIs in 2019. Nucleic Acids Research. 2019 July; 47 (W1):W636-W641. DOI: 10.1093/nar/gkz268. PMID: 30976793; PMCID: PMC6602479) is used for the comparison of amino acid sequences. In the case of pairwise comparisons/alignments, the following default settings are preferably chosen: Program: clustalo; Version: 1.2.4; Input Parameters: Output guide tree: true; Output distance matrix: false; Dealign input sequences: false; mBed-like clustering guide tree: true; mBed-like clustering iteration: true; Number of iterations: 0; Maximum guide tree iterations: −1; Maximum HMM iterations: −1; Output alignment format: clustal_num; Output order: aligned; Sequence Type: protein. Preferably, the degree of identity is calculated over the complete length of the sequence.

Furthermore, amino acid residues located at a position corresponding to a position in a reference sequence can be identified by the skilled person by methods known in the art. The alignment can be done with means and methods known to the skilled person, e.g. by using a known computer algorithm such as the Lipman-Pearson method (Science 227 (1985), 1435) or the CLUSTAL algorithm. It is preferred that in such an alignment maximum homology is assigned to conserved amino acid residues present in the amino acid sequences.

In a preferred embodiment Clustal Omega is used for the comparison of amino acid sequences. In the case of pairwise comparisons/alignments, the following default settings are preferably chosen: Program: clustalo; Version: 1.2.4; Input Parameters: Output guide tree: true; Output distance matrix: false; Dealign input sequences: false; mBed-like clustering guide tree: true; mBed-like clustering iteration: true; Number of iterations: 0; Maximum guide tree iterations:-1; Maximum HMM iterations:-1; Output alignment format: clustal_num; Output order: aligned; Sequence Type: protein.

In the context of the present invention, an “amino acid substitution” means that the respective amino acid residues at the indicated position can be substituted with any other possible amino acid residues, e.g. naturally occurring amino acids or non-naturally occurring amino acids (Brustad and Arnold, Curr. Opin. Chem. Biol. 15 (2011), 201-210).

Generally, herein and in context of the present invention, a feature which “has” a certain sequence may “comprise” said sequence, may be “defined by” said sequence or may “consist of” said sequence.

Herein and in context of the present invention, the DNA binding domain according to the invention may have a sequence identity of at least 60%, preferably at least 70%, more preferably at least 80%, to SEQ ID NO: 1. In certain preferred embodiments, said DNA binding domain has a sequence as shown in SEQ ID NO: 1.

As illustrated in the appended Examples, it has been further surprisingly found in context of the present invention that the DNA binding domain of MTERF1 can be truncated, e.g. at the N-terminus, and still sufficiently retain its functionality, i.e. binding to its response element and providing functionality to the synthetic transcription factor (see, e.g. FIG. 3). Shorter DNA binding domains may be advantageous because they have a reduced DNA footprint/genetic payload, e.g., for viral transduction. Furthermore, modulation of the length of the MTERF1 binding domain allows to vary the gene of interest expression levels.

Therefore, the DNA binding domain according to the invention may comprise a MTERF1 subdomain B which has a sequence as shown in SEQ ID NO: 7 or a sequence that has a sequence identity of at least 70%, preferably at least 80%, more preferably at least 90%, to SEQ ID NO: 7.

Surprisingly, it has been further found that one MTERF1 motif in the MTERF1 DNA binding domain can be omitted while the functionality of the DNA binding domain is substantially retained.

Therefore, the DNA binding domain according to the invention may comprise a MTERF1 subdomain A (i.e. a shorter subdomain), which has a sequence as shown in SEQ ID NO: 9 or a sequence that has a sequence identity of at least 80%, preferably at least 90%, more preferably at least 95%, to SEQ ID NO: 9.

Accordingly, the DNA binding domain according to the invention may comprise (i) a first MTERF1 motif which has a sequence as shown in SEQ ID NO: 104 or a sequence that has a sequence identity of at least 80%, preferably at least 90%, more preferably at least 95% to SEQ ID NO: 104, and/or (ii) a second MTERF1 motif which has a sequence as shown in SEQ ID NO: 106 or a sequence that has sequence identity of at least 80%, preferably at least 90%, more preferably at least 95% to SEQ ID NO: 106. Preferably, the first MTERF1 motif is N-terminally of the second MTERF1 motif. Furthermore, the first and second MTERF1 motifs may be directly adjacent to each other (preferably due to a peptide bond) or connected by via a linker (in particular by a peptide linker). Preferably, said DNA binding domain comprising said first and/or second MTERF1 motif further comprises (iii) a MTERF1 C-terminal domain which has a sequence as shown in SEQ ID NO: 11 or a sequence that has a sequence identity of at least 70%, preferably at least 80%, more preferably at least 90%, to SEQ ID NO: 11.

In particular, the first MTERF1 motif and/or the second MTERF1 motif, as described herein, is/are contained in the subdomain A as described herein. Furthermore, the MTERF1 subdomain A, as described herein, is, in particular, contained in the MTERF1 subdomain B, as described herein.

Furthermore, it is possible that a few amino acids, e.g. about 1 to 30 or about 1 to 10 amino acids, can be deleted from the C-terminus of a MTERF1-derived DNA binding domain or a MTERF1-derived DNA binding subdomain according to the invention. It may be also possible to delete the entire most C-terminal subdomain of a MTERF1-derived DNA binding domain or a MTERF1-derived DNA binding subdomain according to the invention.

Preferably, herein and in context of the invention, the MTERF1-derived DNA binding domain of the invention shows an arginine (R) at a position corresponding to position 387 in the sequence of SEQ ID NO: 108, e.g., at position 330 in the sequence of SEQ ID NO: 1.

Preferably, herein and in context of the present invention, the synthetic transcription factor does not comprise a mitochondrial transfer peptide which has a sequence as shown in SEQ ID NO: 37 or a sequence that has a sequence identity of at least 90% to SEQ ID NO: 37. Preferably, the synthetic transcription factor does not have a functional mitochondrial transfer peptide at all. In particular, the synthetic transcription factor of the invention does not comprise the mitochondrial transfer peptide at the N-terminus.

Herein, a “mitochondrial transfer peptide” may also refer to a “mitochondrial targeting signal”. In particular, a mitochondrial transfer peptide directs a protein to the mitochondria such that the protein can enter the mitochondria and/or localize to mitochondria.

The synthetic transcription factor of the invention, however, is preferably not able to enter or localize to mitochondria.

Very preferably herein and context of the present invention, the synthetic transcription factor of the invention is capable of entering and/or localizing to a cell nucleus. This is particularly advantageous for achieving a high orthogonality as described herein.

Thus, the synthetic transcription factor of the invention may have the ability to localize more efficiently to the nucleus in a cell than to the mitochondria in said cell. Said ability may be determined by measuring the amount of the synthetic transcription factor separately in the nucleus and the mitochondria of the same cell(s). This can be done by routine methods in the art, e.g., immunostaining, separation of nuclei and mitochondria followed by western blot or ELISA, etc.

The synthetic transcription factor of the invention may enter and/or localize to a cell nucleus when no functional mitochondrial transfer peptide as described herein, e.g. as shown in SEQ ID NO: 37, is present. Furthermore, the synthetic transcription factor of the invention may enter and/or localize to a cell nucleus when it comprises a nuclear localization signal.

Therefore, the synthetic transcription factor of the invention may comprise a nuclear localization signal (NLS). A nuclear localization signal also refers to a “nuclear localization peptide”. NLS sequences are well known in the art and, in principle, any of them may be employed.

Herein, the term “gene expression” always encompasses the term “gene transcription” or “transcription of a gene” but it may, in certain circumstances, also include post-transcriptional mechanisms. The term “gene transcription” or “transcription of a gene”, as used herein, refers to gene expression in a more specific manner. However, if not explicitly indicated differently, the term “gene expression” may be replaced herein by the term “gene transcription” or “transcription of a gene” since the present invention concerns, in particular, means and methods for the regulation of gene transcription.

In particular, herein and in context of the present invention, the synthetic transcription factor is capable of regulating the transcription of at least one gene of interest in a cell. Preferably, said synthetic transcription factor is capable of regulating the transcription of at least one gene of interest in a nucleus of a cell.

The synthetic transcription factor of the invention may regulate transcription of a gene of interest the same way as described herein in general in context of transcription factors. In particular, the synthetic transcription factor of the invention may control the rate of transcription of a gene of interest. Furthermore, the synthetic transcription factor of the invention may induce or initiate transcription of a gene of interest.

The gene of interest, as used herein and in context of the present invention, is not limited to any genes. Preferably herein and in context of the present invention, a gene of interest encodes, for example, a cell death promoting protein such as hBAX or HSV-TK, an immune stimulating cytokine such as IL-2 or IL-12, or an antigen-receptor such as a CAR or a TCR.

hBAX refers to pro-apoptotic protein which may be used in cell classifiers to kill cells, e.g., in cancer-cell classifiers to kill cancer cells. HSV-TK refers to protein which metabolizes ganciclovir into toxic metabolite. It may be used in cell classifiers to kill cells, e.g., in cancer-cell classifiers to kill cancer cells. IL-2 or IL-12 are immune stimulating cytokines which may be used, e.g., in cell classifiers, e.g. cancer-cell classifiers, to attract and induce proliferation of T cells. CAR and TCR refer to antigen receptors which recognize cells with corresponding surface antigens. They could be used, e.g., in conjunction with the SynNotch system; Morsut (2016), Cell 164 (4): 780-91.

Preferably herein and in context of the present invention, the synthetic transcription factor regulates the transcription of a gene of interest in a cell by (i) promoting transcription of the gene of interest, or by (ii) suppressing transcription of the gene of interest. As described herein, the synthetic transcription factor of the invention preferably promotes or suppresses transcription of a gene of interest in a nucleus of a cell. This is particularly advantageous for achieving a high orthogonality as described herein.

In particular, the synthetic transcription factor of the invention may promote transcription of a gene by increasing the rate of transcription. Furthermore, the synthetic transcription factor of the invention may suppress transcription of a gene by decreasing the rate of transcription.

As used herein, promoting gene transcription may comprise, for example, inducing, initiating and/or enhancing gene transcription. Furthermore, suppressing gene transcription may comprise, for example, blocking or repressing gene transcription, e.g. blocking or repressing the induction or initiation of gene transcription.

Preferably herein and in context of the present invention, the synthetic transcription factor is capable of binding to a response element in a cell, preferably in a nucleus of a cell. In particular, the DNA binding domain comprised in the synthetic transcription factor of the invention is capable of binding to a response element in a cell, preferably in a nucleus of a cell.

Herein and in context of the present invention, the response element may comprise a MTERF1 binding site which has a sequence as shown in SEQ ID NO: 42 or a sequence which has a sequence identity of at least 60%, preferably at least 80%, more preferably at least 90% to SEQ ID NO: 42. Preferably, the MTERF1 binding site according to the invention consists of a sequence as shown in SEQ ID NO: 42 or a sequence which has a sequence identity of at least 60%, preferably at least 80%, more preferably at least 90% to SEQ ID NO: 42

In particular, the synthetic transcription factor and/or the DNA binding domain according to the invention is able to bind to at least one MTERF1 binding site in the response element, as described herein.

It has been further found that the expression levels of the gene of interest can be modulated by modifying the design of the response element-containing promoter and the amount of the synthetic transcription factor expressed in the cells. Moreover, it has been observed that the numbers of copies of REs and the spacing between them also affected Gol expression at constant MTF levels (see, e.g., FIG. 1). Of note, in FIG. 1, the term “response element” (RE) corresponds to the term “binding site” as used herein, in particular, an MTERF1 binding site as defined by SEQ ID NO: 42.

The response element according to the invention, also in context of the DNA construct of the invention, may comprise multiple copies of an MTERF1 binding site, as described herein, e.g., 2 to 50, 2 to 25 or 2 to 15 copies, preferably 2 to 5 copies. Moreover, the individual copies of said binding site do not need to be identical to each other but may comprise variations.

Moreover, the copies, e.g. two or more or all copies, of said binding site in said response element may directly adjacent to each other or separated by one or more, e.g., 1 to 100, 1 to 50, 1 to 20, 1 to 10, for example, 6 or 10, nucleotides.

The synthetic transcription factor of the invention is, in particular, capable of binding to a promoter comprising the response element described herein in a cell, preferably in a nucleus of a cell. Said promoter may further comprise a minimal promoter, for example, a minimal TATA box which preferably has a sequence as shown in SEQ ID NO: 103 or a minimal CMV promoter which preferably has a sequence as shown in SEQ ID NO: 136. Preferably said minimal promoter is 3′ of said response element. For example, said promoter has a sequence as shown in SEQ ID NO: 110.

Further promoters, response elements and minimal promoters are described herein in context of the DNA construct of the invention and may be also considered and employed in this specific context here.

Preferably herein and in context of the present invention, the promoter is operably linked to a gene of interest as described herein, preferably in a cell nucleus as described herein.

In particular, herein and in context of the present invention, the binding of the synthetic transcription of the invention (in particular of the DNA binding domain) to the promoter of the invention (in particular to the response element of the invention in said promoter) regulates the transcription of a gene of interest which is operably linked to said promoter as described herein, preferably in a nucleus of a cell.

The orthogonality of the inventive synthetic transcription factor or the inventive transcriptional system described herein is, in particular, assessed in a cell which is from the same species, e.g. the same mammalian species, that the mitochondrial DNA binding protein according to the invention (and hence the DNA binding domain contained in the inventive synthetic transcription factor) is from. Herein, said cell is also briefly called a “same-species cell”. As described herein, the inventive synthetic transcription factor and the inventive transcriptional system may function in a highly orthogonal manner to endogenous processes, in particular gene regulatory processes, in a cell (i.e. a same-species cell).

Therefore, herein and in context of the present invention, the cell, in particular the cell in which the synthetic transcription factor of the invention regulates transcription, is from the same species, e.g. the same mammalian species, that the mitochondrial DNA binding protein employed in context of to the invention (and hence the DNA binding domain in the synthetic transcription factor of the invention) is from, i.e. it is a same-species cell, as described herein. Preferably, said DNA binding domain is derived from a human mitochondrial DNA binding protein (e.g. human MTERF1) and, therefore, said cell (i.e. said same-species cells) is preferably a human cell.

Preferably herein, the synthetic transcription factor of the invention does essentially not alter transcription, i.e. the transcriptome, in a same-species cell apart from the transcription of the gene(s) of interest.

Furthermore, it is possible that the synthetic transcription factor of the invention does not specifically bind to essentially any endogenous DNA sequence in a same-species cell. Preferably, the synthetic transcription factor of the invention does not specifically bind to essentially any DNA sequence in said cell apart from said promoter, in particular, a promoter containing the response element according to the invention.

Furthermore, the DNA binding domain according to the invention (i.e. the DNA binding domain contained in the inventive synthetic transcription factor) does, preferably, not specifically bind to essentially any endogenous DNA sequence in a nucleus of a same-species cell.

It is further possible that the synthetic transcription factor of the invention does essentially not compete for sequence-specific DNA binding in a same-species cell with the mitochondrial DNA binding protein from which the DBD according to the invention is derived.

Furthermore, it is possible that the synthetic transcription factor of the invention does essentially not interfere with the function of the mitochondrial DNA binding protein from which the DBD according to the invention is derived.

It also possible that the synthetic transcription factor of the invention does essentially not interfere with the function of the protein from which the transcriptional modulation domain according to the invention is derived.

As described herein, a transcriptional modulation domain is usually involved in promoting or supressing transcription. Usually, a transcriptional modulation domain contains interaction sites for other proteins such as transcription coregulators. Transcription coregulators are proteins that interact with transcription factors to either promote or repress the transcription of specific genes. Transcription coregulators that activate gene transcription are referred to as coactivators while those that repress are known as corepressors. An activation domain, as used herein, may rather associate with coactivators than corepressors, whereas a repression domain, as used herein, may rather associate with corepressors.

The main mechanism of action of transcription coregulators is to modify chromatin structure and thereby make the associated DNA more or less accessible to transcription. In humans several dozen to several hundred coregulators are known, depending on the level of confidence with which the characterisation of a protein as a coregulator can be made. For example, one class of transcription coregulators modifies chromatin structure through covalent modification of histones, whereas a second ATP dependent class modifies the conformation of chromatin. Typical coactivators include, inter alia, the pre-initiation complex containing, e.g. transcription factor IID (TFIID), the mediator complex, histone acetyltransferases and chromatin-remodelling complexes. Typical corepressors include, inter alia, polycomb repressive complexes, e.g. PRC1 or PRC2, histone deacetylases, and histone metyhltransferases.

Herein and in context of the present invention, the transcriptional modulation domain is, in particular, capable of regulating the transcription of a gene, in particular when said transcriptional modulation domain is part of, bound to or interacts with a DNA binding protein that is capable to bind to or interact with a regulatory sequence, e.g. a promoter or enhancer, of said gene.

Since, in context of the present invention, the transcriptional modulation domain is contained in the synthetic transcription factor of the invention together with a DNA binding domain according to the invention, the transcriptional modulation domain is, in particular, able to interact with said DNA binding domain and, therefore, can be directed to a gene regulatory sequence, e.g. a promoter as described herein and regulate the transcription of the corresponding gene.

In particular, the transcriptional modulation domain according to the invention may be capable of binding to and/or interacting with an RNA polymerase, preferably RNA polymerase II; at least one other transcription factor, for example, a general transcription factor (e.g. TFIID); and/or at least one transcriptional coregulator such as a transcriptional coactivator (e.g. the mediator complex and/or a histone acetyltransferase) and/or a transcriptional corepressor (e.g. a polycomb repressive complexes or a histone deacetylase), as described herein.

Herein, and in context of the invention, the transcriptional modulation domain may be (i) an activation domain or (ii) a repression domain, as described herein. Preferably, the transcriptional modulation domain according to the invention is an activation domain.

In particular, the transcriptional modulation domain is able to (i) promote transcription of a gene (in particular when defined as an activation domain), or (ii) suppress transcription of a gene (in particular when defined as a repression domain). Preferably, the transcriptional modulation domain according to the invention is able to (i) promote transcription of a gene.

Therefore, the transcriptional modulation domain according to the invention may be (i) an activation domain which binds to and/or interacts with at least one coactivator to promote transcription of a gene, or (ii) a repression domain which binds to and/or interacts with at least one corepressor to suppress transcription of a gene; preferably an activation domain as described in said (i).

The present invention is not particularly limited with respect to the transcriptional modulation domain, and many suitable activation domains and repression domains are readily available and may be employed in context of the present invention. The functionality of a transcriptional modulation domain in context of the inventive synthetic transcription factor provided herein can be easily tested and verified by routine means, e.g., as described in the appended Examples and as shown, e.g., in FIG. 4. For example, for evaluating the functionality of an activation domain in context of the present invention, the following simple assay can be performed:

A reporter DNA construct (e.g. a plasmid) comprising the promoter shown in SEQ ID NO: 110 operably linked to a gene encoding a detectable protein (e.g. a fluorescent protein) is introduced (e.g. transfected) into suitable cells. In addition, a DNA construct encoding the transcriptional modulation domain to be tested fused to the human MTERF1 binding domain as defined in SEQ ID NO: 1 under control of a constitutive promoter, for example EF1a (SEQ ID NO: 137), CMV (SEQ ID NO: 138), or UbC (SEQ ID NO: 139), is introduced (e.g. transfected) in the same cells. Further, a construct encoding a constitutively expressed additional protein that is detectable independent of the detectable protein encoded in the reporter construct, is introduced into the same cells. Then, the amounts of both detectable proteins are quantified (e.g. by flow cytometry, microscopy, ELISA, or Western Blot). The “signal” is defined as the ratio between the reporter and the constitutively expressed detectable proteins. As negative control, the same assay is performed but the construct encoding the transcriptional modulation domain-MTERF1 fusion protein is not introduced in the cells.

Then, the signal of the experimental condition is compared to that of to the negative control. If the signal is higher compared to the negative control, it is determined that the activation domain is functional (i.e. it is capable to promote transcription of a gene of interest) in context of the synthetic transcription factor of the invention.

In case the transcriptional modulation domain is a repression domain, the following assay may be performed to assess its functionality:

In a reporter DNA construct (e.g. a plasmid), a promoter to be repressed, for example EF1a (SEQ ID NO: 137), CMV (SEQ ID NO: 138), or UbC (SEQ ID NO: 139) additionally comprising binding sites for the inventive transcription factor either within the promoter sequence or within 0 to 2000 bases adjacent to 5′ or 3′ end of the promoter, is operably linked to a gene encoding a detectable protein. This reporter DNA construct is introduced (e.g. transfected) into suitable cells.

In addition, a DNA construct encoding the transcriptional modulation domain to be tested fused to the human MTERF1 binding domain as defined in SEQ ID NO: 1 under control of a constitutive promoter, for example EF1a (SEQ ID NO: 137), CMV (SEQ ID NO: 138), or UbC (SEQ ID NO: 139), is introduced (e.g. transfected) in the same cells. Further, a construct encoding a constitutively expressed additional protein that is detectable independent of the detectable protein encoded in the reporter construct, is introduced into the same cells. Then, the amounts of both detectable proteins are quantified (e.g. by flow cytometry, microscopy, ELISA, or Western Blot). The “signal” is defined as the ratio between the reporter and the constitutively expressed detectable proteins. As negative control, the same assay is performed but the construct encoding the transcriptional modulation domain-MTERF1 fusion protein is not introduced in the cells.

Then, the signal of the experimental condition is compared to that of the negative control. If the signal is lower compared to the negative control, it is determined that the repression domain is functional (i.e. it is capable to hinder transcription of a gene of interest) in context of the synthetic transcription factor of the invention.

Furthermore, in case a certain transcriptional modulation domain, e.g., an activation domain, is found to have little functionality by its own it may provide a good functionality when comprised multiple times in the synthetic transcription factor and/or in combination with further transcriptional modulation domain. For example, as described herein and as illustrated in the appended Examples (see, e.g. FIGS. 4 and 9), the use of two FOXO domains strongly enhanced the transactivating activity compared to a single FOXO domain.

The transcriptional modulation domain of the invention may be an activation domain comprising at least one transactivation domain independently selected from the group consisting of: a RELA domain (e.g. RelA₄₃₀₋₅₅₁, RelA₃₄₂₋₅₅₁, RelA₃₆₁₋₅₅₁ or RelA₅₂₁₋₅₅₁ (i.e. “TA1”), a WW domain (WWC1₂₋₈₁), a KRAB domain (ZNF4735-48), a NucRecCoAct domain (NCOA3₁₀₄₅₋₁₀₉₂), a LMSTEN domain (MYB₂₅₁₋₃₃₀) and a FoxoTAD (FOXO3₆₀₄₋₆₄₄).

Herein and in context of the present invention, the transcriptional modulation domain, in particular the activation domain, may have a sequence identity of at least 60%, preferably at least 70%, more preferably at least 80% to a sequence selected from the group consisting of: SEQ ID NO: 3, SEQ ID NO: 33, SEQ ID NO: 52, SEQ ID NO: 27, SEQ ID NO: 29, SEQ ID NO: 54, SEQ ID NO: 56, SEQ ID NO: 58, SEQ ID NO: 17, SEQ ID NO: 19, SEQ ID NO: 60, SEQ ID NO: 62, SEQ ID NO: 13, SEQ ID NO: 64, SEQ ID NO: 66 and SEQ ID NO: 68; or; or the transcriptional modulation domain, in particular the activation domain, may comprise at least one sequence that has a sequence identity of at least 60%, preferably at least 70%, more preferably at least 80% to a sequence selected from the group consisting of: SEQ ID NO: 3, SEQ ID NO: 33, SEQ ID NO: 52, SEQ ID NO: 27, SEQ ID NO: 29, SEQ ID NO: 54, SEQ ID NO: 56, SEQ ID NO: 58, SEQ ID NO: 17, SEQ ID NO: 19, SEQ ID NO: 60, SEQ ID NO: 62, SEQ ID NO: 13, SEQ ID NO: 64, SEQ ID NO: 66 and SEQ ID NO: 68.

In some preferred embodiments of the invention, the transcriptional modulation domain, in particular the activation domain, has a sequence identity of at least 60%, preferably at least 70%, more preferably at least 80% to a sequence selected from the group consisting of: SEQ ID NO: 3, SEQ ID NO: 33, SEQ ID NO: 52, SEQ ID NO: 27, SEQ ID NO: 29, SEQ ID NO: 54, SEQ ID NO: 56 and SEQ ID NO: 58.

In more preferred embodiments of the invention, the transcriptional modulation domain, in particular the activation domain, has a sequence identity of at least 70%, preferably at least 80%, more preferably at least 90% to a sequence selected from the group consisting of: SEQ ID NO: 3, SEQ ID NO: 33 and SEQ ID NO: 52.

In some preferred embodiments, the transcriptional modulation domain of the invention comprises a first, a second and/or a third RELA transactivation domain; wherein the first RELA transactivation domain (TA1) has a sequence as shown in SEQ ID NO: 31 or a sequence that has a sequence identity of at least 80% to SEQ ID NO: 31; wherein the second RELA transactivation domain has a sequence as shown in SEQ ID NO: 132 or a sequence that has a sequence identity of at least 80% to SEQ ID NO: 132; and wherein the third RELA transactivation domain has a sequence as shown in SEQ ID NO: 134 or a sequence that has a sequence identity of at least 80% to SEQ ID NO: 134. Preferably, said transcriptional modulation domain comprises at least the first RELA transactivation domain, as described herein.

Furthermore, the transcriptional modulation domain may comprise multiple copies, e.g. two or three copies, of said first, second and/or third RELA domain, preferably of said first RELA domain (TA1). As illustrated in the appended Examples, this can increase the expression level of the gene(s) of interest (see, e.g., FIGS. 4 and 9). Furthermore, e.g. in context of these embodiments, the transcriptional modulation domain according to the invention may comprise a RELA subdomain A which has a sequence as shown in SEQ ID NO: 3 or a sequence that has a sequence identity of at least 70%, preferably at least 80%, more preferably at least 90% to SEQ ID NO: 3.

It has been further found in context of the present invention that employing a larger subunit of the RELA transactivation domain can increase the expression level of a gene of interest (see, e.g. FIGS. 4 and 10C). Therefore, e.g. in context of these “RELA” embodiments, the transcriptional modulation domain according to the invention may comprise a RELA subdomain B which has a sequence as shown in SEQ ID NO: 27 or a sequence that has a sequence identity of at least 70%, preferably at least 80%, more preferably at least 90% to SEQ ID NO: 27.

Furthermore, e.g. in context of these embodiments, the transcriptional modulation domain according to the invention may comprise a RELA subdomain C which has a sequence as shown in SEQ ID NO: 29 or a sequence that has a sequence identity of at least 60%, preferably at least 70%, more preferably at least 80% to SEQ ID NO: 29.

In particular, the first, second and/or third RELA motifs, said RELA subdomain A and/or said RELA subdomain B, as described herein, are contained in said RELA subdomain C. Furthermore, the RELA subdomain A is, in particular, contained in said RELA subdomain B.

Furthermore, transcriptional modulation domain of the invention may comprise a FOXO3 transactivation domain (FOXO TAD) which has a sequence as shown in SEQ ID NO: 17 or a sequence that has a sequence identity of at least 80% to SEQ ID NO: 17.

It has been further surprisingly found in context of the present invention that the use of two copies of the FOXO TAD provided a high expression level of a gene of interest while retaining a small size (and hence a low genetic payload); (see, e.g. FIGS. 4 and 9).

Therefore, in further preferred embodiments, transcriptional modulation domain of the invention may comprise multiple copies, e.g. two or three copies, of said FOXO3 transactivation domain (FOXO TAD). Preferably, said transcriptional modulation domain has a sequence as shown in SEQ ID NO: 33 or a sequence that has a sequence identity of at least 70%, preferably at least 80%, more preferably at least 90% to SEQ ID NO: 33.

Furthermore, transcriptional modulation domain of the invention may comprise a MYB transactivation domain (LMSTEN) which has a sequence as shown in SEQ ID NO: 17 or a sequence that has a sequence identity of at least 80% to SEQ ID NO: 17.

In some embodiments, the transcriptional modulation domain comprises multiple copies, e.g. two or three copies, of said MYB transactivation domain (LMSTEN). Preferably, said transcriptional modulation domain has a sequence as shown in SEQ ID NO: 196 or a sequence that has a sequence identity of at least 70%, preferably at least 80%, more preferably at least 90% to SEQ ID NO: 196.

In some embodiments, the transcriptional modulation domain comprises two copies of
(i) a first RELA transactivation domain (TA1) that has a sequence as shown in SEQ ID NO: 31 or a sequence that has a sequence identity of at least 80% to SEQ ID NO: 31;
(ii) a FOXO3 transactivation domain (FOXO TAD) that has a sequence as shown in SEQ ID NO: 33 or a sequence that has a sequence identity of at least 80% to SEQ ID NO: 33; or
(iii) MYB transactivation domain (LMSTEN) that has a sequence as shown in SEQ ID NO: 196 or a sequence that has a sequence identity of at least 80% to SEQ ID NO: 196.

Furthermore, the transcriptional modulation domain according to the invention may comprise three copies of a first RELA transactivation domain (TA1) that has a sequence as shown in SEQ ID NO: 31 or a sequence that has a sequence identity of at least 80% to SEQ ID NO: 31.

Thus, in some embodiments, the transcriptional modulation domain has a sequence as shown in SEQ ID NO: 33, SEQ ID NO: 35, SEQ ID NO: 194 or SEQ ID NO: 196, or a sequence that has a sequence identity of at least 70%, preferably at least 80%, more preferably at least 90% to any of these sequences.

It has been further surprisingly found in context of the present invention that two copies of a FOXO TAD enhanced the transcriptional activity of the synthetic TF more than two copies of TA1 or two copies of LMSTEN (see FIG. 9).

Hence, in context of the present invention, a transcriptional modulation domain comprising multiple copies of the same transactivation domain comprises, preferably, at least two copies of a FOXO3 transactivation domain (FOXO TAD) as shown in SEQ ID NO: 33 or a sequence that has a sequence identity of at least 70%, preferably at least 80%, more preferably at least 90% to SEQ ID NO: 33.

In some preferred embodiments, the transcriptional modulation domain of the invention comprises at least two domains independently selected from the group consisting of a TA1 as described herein, a FOXO TAD as described and a LMSTEN as described herein. Preferably said transcriptional modulation domain comprises at least a FOXO TAD. More preferably, said transcriptional modulation domain comprises at least a FOXO TAD, a TA1 and a LMSTEN, as described herein.

In some preferred embodiments, the transcriptional modulation domain comprises (i) two FOXO TAD, (ii) a FOXO TAD and a LMSTEN, (iii) a LMSTEN and a TA1, (iv) a FOXO TAD and a TA1, or (v) a FOXO TAD, a LMSTEN and a TA1, as described herein. More preferably, the transcriptional modulation domain comprises a FOXO TAD, in particular, said options (i), (ii), (iv) or (v).

In further preferred embodiments of the invention, said transcriptional modulation domain has a sequence as shown in SEQ ID NO: 52, SEQ ID NO: 54, SEQ ID NO: 56 or SEQ ID NO: 58, or a sequence that has a sequence identity of at least 70%, preferably at least 80%, more preferably at least 90% to any of these sequences.

It has been further found in context of the invention that certain transcriptional modulation domains, e.g., comprising a FOXO-TAD and a further domain, in particular, a further FOXO-TAD, a TA1 and/or LMSTEN domain, confer a high transcriptional activity to the synthetic transcription factor; see, e.g., FIG. 9C.

Furthermore, it has been found that such combinatorial transcriptional modulation domains (e.g., comprising a FOXO-TAD and a further domain, in particular, a further FOXO-TAD, a TA1 and/or LMSTEN domain) compensate for or overcompensate for the slight loss in transcriptional activity of N-terminally truncated MTERF1 DNA-binding domains, e.g., MTERF1₁₀₄₋₃₉₉ (SEQ ID NO: 9); see, e.g., FIG. 9F.

Thus, in further preferred embodiments of the invention, the transcriptional modulation domain has a sequence as shown in SEQ ID NO: 52, SEQ ID NO: 33, SEQ ID NO: 54 or SEQ ID NO: 58, or a sequence that has a sequence identity of at least 70%, preferably at least 80%, more preferably at least 90% to any of these sequences. Furthermore, the DNA binding domain according to the invention may comprise a MTERF1 subdomain A which has a sequence as shown in SEQ ID NO: 9 or a sequence that has a sequence identity of at least 80%, preferably at least 90%, more preferably at least 95%, to SEQ ID NO: 9.

In some of the most preferred embodiments of the invention, the transcriptional modulation domain has a sequence as shown in SEQ ID NO: 52 or a sequence that has a sequence identity of at least 70%, preferably at least 80%, more preferably at least 90% to SEQ ID NO: 52. Furthermore, the DNA binding domain according to the invention may comprise a MTERF1 subdomain A which has a sequence as shown in SEQ ID NO: 9 or a sequence that has a sequence identity of at least 80%, preferably at least 90%, more preferably at least 95%, to SEQ ID NO: 9.

Furthermore, the transcriptional modulation domain of the invention, in particular the activation domain, may comprise a sequence that has a sequence identity of at least 60%, preferably at least 70%, more preferably at least 80% to a sequence selected from the group consisting of: SEQ ID NO: 60, SEQ ID NO: 62, SEQ ID NO: 13, SEQ ID NO: 64, SEQ ID NO: 66 and SEQ ID NO: 68, preferably in addition to the first, second and/or third RELA motif, e.g. said TA1, the RELA subdomain A, B or C, the FOXO TAD and/or the LMSTEN, as described herein.

Alternatively, the transcriptional modulation domain of the invention, in particular the repression domain, may have a sequence identity of at least 60%, preferably at least 70%, more preferably at least 80% to a sequence selected from the group consisting of: SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96 and SEQ ID NO: 98; or wherein the transcriptional modulation domain, in particular the repression domain, may comprise at least one sequence that has a sequence identity of at least 60%, preferably at least 70%, more preferably at least 80% to a sequence selected from the group consisting of: SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96 and SEQ ID NO: 98.

Preferably, the transcriptional modulation domain, in particular the repression domain, has a sequence identity of at least 60%, preferably at least 70%, more preferably at least 80% to a sequence selected from the group consisting of: preferably to SEQ ID NO: 70, SEQ ID NO: 72, SEQ ID NO: 74 or SEQ ID NO: 76.

More preferably, the transcriptional modulation domain, in particular the repression domain, has a sequence identity of at least 60%, preferably at least 70%, more preferably to SEQ ID NO: 70.

Furthermore, the synthetic transcription factor of the invention may further comprise a controllable domain, preferably a controllable destabilization domain or a controllable localization domain.

In particular, a controllable domain, as used herein and in context of the present invention, is controllable by a compound or by light (e.g. infrared, visible and/or UV light). Preferably, said compound is a small molecule. Preferably, said light is light of a particular wavelength or of a particular range of wavelengths. Contacting a synthetic transcription factor comprising a controllable domain by a compound or by light may modulate, e.g., promote or repress, the transcriptional activity of the transcription factor.

A synthetic transcription factor comprising a controllable domain as described herein may be also called an “inducible transcription factor” because its activity (or inactivity) can be induced by an outside stimulus, in particular by a compound or light as described herein. Many suitable controllable domains are known in the art and any of these may be used in context of the present invention. Furthermore, the induction of the activity (or inactivity) of the transcription factor is not limited to a specific mechanism. For example, certain controllable domains (e.g. an NS3 domain which is derived from hepatitis C virus) function as destabilization domains which destabilize the protein to which they are fused, wherein the fusion protein can be stabilized by a small molecule binding to the destabilization domain. Other controllable domains function in an inverse way, wherein the fusion protein can be destabilized by a small molecule binding to the controllable domain.

Again other controllable domains (e.g. ERT2, which is derived from the human estrogen receptor) control the location of the protein to which they are fused, wherein the location can be altered by a small molecule binding to the controllable domain. A synthetic transcription factor in context of the invention functions, in particular, in the nucleus of a cell. Thus, the activity of the transcription factor may be induced when it is located from outside the nucleus (e.g. the cytoplasm) into the nucleus.

Some controllable domains (e.g. a FRB domain and a FKBP domain) bind to each other in the presence of a compound or light, thereby bringing the proteins to which they are fused together. In case, a functional protein comprises both parts, its activity is induced upon dimerization in the presence of the compound or light.

Other controllable domains and methods of inducing a synthetic transcription factor of the invention which are known in the art or will be developed can be used in context of the present invention.

In some embodiments, the synthetic transcription factor of the invention comprises a controllable destabilization domain, and is stabilized or destabilized (preferably stabilized) by a compound or light. Preferably, the controllable destabilization domain comprises a NS3 domain which has a sequence as shown in SEQ ID NO: 158 or a sequence that has a sequence identity of at least 80%, preferably at least 90%, more preferably at least 95%, to SEQ ID NO: 158. In particular, a synthetic transcription factor comprising said NS3 domain according to the invention is stabilized by the small molecule grazoprevir.

In some embodiments, the synthetic transcription factor comprises a controllable localization domain, and located to either the nucleus or the cytoplasm, preferably to the nucleus, of a cell by said compound or light. Preferably, the controllable localization domain comprises an ERT2 domain which has a sequence as shown in SEQ ID NO: 152 or a sequence that has a sequence identity of at least 80%, preferably at least 90%, more preferably at least 95%, to SEQ ID NO: 152. In particular, a synthetic transcription factor comprising said ERT2 domain is located to the nucleus of a cell by 4-hydroxytamoxifen.

In further embodiments, in particular when the DNA binding domain and the transcriptional modulation domain according to the invention are comprised in separate polypeptides, the controllable domain comprises a FRB domain and a FKBP domain, wherein said FRB domain has a sequence as shown in SEQ ID NO: 140 or a sequence that has a sequence identity of at least 80%, preferably at least 90%, more preferably at least 95%, to SEQ ID NO: 140, and/or wherein said FKBP domain has a sequence as shown in SEQ ID NO: 142 or a sequence that has a sequence identity of at least 80%, preferably at least 90%, more preferably at least 95%, to SEQ ID NO: 142. In particular, said FRB domain and said FKBP domain bind to each other in the presence of C16-(S)-7-methylindolerapamycin.

In some embodiments, the synthetic transcription factor of the invention comprises a synNotch core which has a sequence as shown in SEQ ID NO: 160 or a sequence that has a sequence identity of at least 80%, preferably at least 90%, more preferably at least 95%, to SEQ ID NO: 160.

A synNotch core is a surface receptor and is N-terminally fused to an single-chain variable fragment (scFv). Once an scFc: synNotch core: transcription factor fusion proteins binds its target (via the scFv), the transcription factor is cleaved off, locates to the nucleus and modulates (e.g. activates) gene expression; see, e.g., Morsut (2016), Cell, 164 (4).

Herein and in context of the present invention, it is further desirable that the immunogenicity of the synthetic transcription factor is low in an organism in which it is desirably employed, e.g., in a human.

It has been further surprisingly found in context of the present invention that a human-derived synthetic transcription factors can be assembled which retain a high orthogonality, as described herein, in human cells. In particular, in the human-derived synthetic transcription factors according to the invention, both, the DNA binding domain according to the invention and the transcriptional modulation domain according to the invention are derived from human proteins, preferably from human MTERF1 and at least one other human protein, respectively.

In context of synthetic transcription factors comprising an activation domain, these synthetic transcription factors are also called “Human-derived transcriptional activator proteins” or short “HumTAP” herein.

Preferably herein, and in context of the present invention, the human-derived synthetic transcription factors, e.g. HumTAPs, consist exclusively of human protein-derived domains, more preferably they consist exclusively of human protein domains.

What is described herein in context of the orthogonality generally herein, in particular in context of human (or human cells), also applies to the human-derived synthetic transcription factors described herein, not at least because the orthogonality was tested in the appended Examples with a HumTAP (see, e.g., Example 3)

In order to assess immunogenicity of the protein, the inventors employed an assay to investigate immune responses against peptides derived from proteins of interest using primary PBMCs from normal human donors, as illustrated in the appended Example. It has been found that PBMCs reacted to HumTAP (i.e. MTF)-derived peptides (corresponding in particular to the domain junction of the HumTAP) more like to self-peptides (i.e. non-immunogenic control pepides) rather than to immunogenic positive control peptides (see, e.g. Example 2). This suggests a low immunogenicity of the human-derived synthetic transcription factors of the invention, e.g., HumTAPs in humans.

Therefore, the inventors have further developed a class of synthetic transcription factors made entirely of human protein subunits. The data shown in the appended Examples indicates a favorable profile of Gol transactivation, immunogenicity, and orthogonality. The immunogenic potential of synthetic transcription factor generated by using two fully-human protein domains (i.e. a human DNA binding domain and human transcriptional modulation domain), as illustrated in the appended Examples is minimized. Therefore, it is expected that less or no local or systemic immunosuppressive actions and less or no additional genetic elements will be required to counteract the patient immune response against gene or cell therapy (GCT) payloads that comprise a gene encoding a human-derived synthetic transcription factor, e.g. a HumTAP protein. This may further result in improved clinical performance of GCT compared to alternatives in the prior art.

Hence, the human-derived synthetic transcription factors according to the invention successfully reconcile the requirement for a high orthogonality as described (e.g. in human cells) and a low immunogenicity (e.g. in humans). Accordingly, the development of human-derived synthetic transcription factors, e.g. HumTAPs, which have a high orthogonality in human cells is a particularly great (and unexpected) achievement of the present inventors. Moreover, the present invention is of particularly high value for synthetic gene circuits that may be used in therapy, e.g. gene therapy or cell therapy, as described herein.

Therefore, the one or more proteins from which said transcriptional modulation domain is derived from are, preferably herein and in context of the present invention, from the same species, e.g. the same mammalian species, that the mitochondrial DNA binding protein according to the invention is from. Furthermore, the synthetic transcription factor of the invention may be composed essentially or fully of parts of proteins from the same species, e.g., the same mammalian species.

Preferably herein and in context of the present invention, the mitochondrial DNA binding protein according to the invention, e.g. MTERF1, and the one or more other proteins according to the invention from which said transcriptional modulation domain is derived from, e.g. RELA, FOX03 and/or MYB, are from a human. Moreover, the synthetic transcription factor of the invention may be composed essentially or fully of parts of human proteins.

In certain embodiments, the transcriptional modulation domain of the invention is derived from a single human protein. Preferably, said transcriptional modulation domain, in particular said activation domain, has a sequence identity of at least 90%, preferably at least 95%, more preferably at least 99%, to SEQ ID NO: 3, SEQ ID NO: 27 or SEQ ID NO: 29, preferably to SEQ ID NO: 3.

Alternatively, the transcriptional modulation domain of the invention, in particular the repression domain, may have a sequence identity of at least 90%, preferably at least 95%, more preferably at least 99%, to SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96 or SEQ ID NO: 98, preferably to SEQ ID NO: 70.

It is possible that the synthetic transcription factor of the invention is essentially non-immunogenic in the mammalian species that said mitochondrial DNA binding protein is from. Preferably herein and in context of the present invention, the synthetic transcription factor is essentially non-immunogenic in a human.

As described herein above, in some preferred embodiments, the synthetic transcription factor of the invention is a fusion protein comprising the DNA binding domain according to the invention and the transcriptional modulation domain according to the invention. As also described herein above the DNA binding domain and the transcriptional modulation domain are connected to each other in the fusion protein either directly by means of a direct peptide bond or by means of a peptide linker (and two corresponding peptide bonds at either end of the linker). Therefore, the synthetic transcription factor fusion protein of the invention may further comprise a peptide linker between the DNA binding domain and the transcriptional modulation domain of the synthetic transcription factor. Many suitable peptide linkers are known in the art any may be employed in context of the present invention, e.g., in context of the fusion protein of the invention. Suitable peptide linkers are, inter alia, the two amino acid linker “GS”, G4S as shown in SEQ ID NO: 118, AP6 as shown in SEQ ID NO: 120, cMycNLS as shown in SEQ ID NO: 122, “EAAAK” as shown in SEQ ID NO: 124 and the SV40 linker shown in SEQ ID NO: 126. Furthermore, said cMycNLS and said SV40 linker also function as nuclear localization sequence (NLS) and thus may be also employed for that purpose herein and in context of the present invention.

Furthermore, the present invention relates to a nucleic acid encoding the fusion protein of the invention comprising the DNA binding domain according to the invention and the transcriptional modulation domain according to the invention (i.e., a fusion protein comprising the synthetic transcription factor of the invention).

The nucleic acid of the invention may be a DNA or an RNA. Furthermore, the nucleic acid may be single stranded or double stranded, e.g., dsDNA, ssRNA, ssDNA or dsRNA. Preferably, the nucleic acid of the present invention comprises the coding strand (i.e. sense strand). However, the nucleic acid of the present invention may also refer to (or even consist of) the antisense strand, and hence, be characterized by the reverse complementary sequence. The same applies to the DNA construct of the invention.

Furthermore, the nucleic acid of the invention may be an mRNA, e.g., an mRNA contained in a lipid nanoparticle.

It has been further found in context of the invention that DNA sequence which are codon optimized for humans provide a higher expression of a gene of interest (see, e.g., FIG. 2).

Therefore, in preferred embodiments, the nucleic acid of the invention comprises a DNA sequence as shown in SEQ ID NO: 5 or a DNA sequence which has a sequence identity of at least 60%, preferably at least 70%, more preferably at least 80%, to SEQ ID NO: 5, wherein said DNA sequence encodes a DNA binding domain that has a sequence identity of at least 60%, preferably at least 70%, more preferably at least 80%, to SEQ ID NO: 1. Preferably, said DNA binding domain has a sequence as shown in SEQ ID NO: 1.

Furthermore, the nucleic acid of the invention may comprise a DNA sequence as shown in SEQ ID NO: 6 or a DNA sequence which has a sequence identity of at least 60%, preferably at least 70%, more preferably at least 80%, to SEQ ID NO: 6, wherein said DNA sequence encodes a transcriptional modulation domain that has a sequence identity of at least 60%, preferably at least 70%, more preferably at least 80%, to SEQ ID NO: 3. Preferably, said transcriptional modulation domain has a sequence as shown in SEQ ID NO: 3.

Furthermore, the present invention relates to a DNA plasmid comprising the nucleic of the invention. Preferably said plasmid is suitable for expressing the synthetic transcription factor in a cell.

Furthermore, the invention relates to a viral vector comprising the nucleic acid of the invention or the plasmid of the invention. As described herein, the nucleic acid may be also defined by the corresponding reverse complementary sequence.

Suitable viral vectors that may be employed in context of the present invention include, inter alia, an adeno-associated virus (AAV) vector, a lentiviral vector, an Adenoviral vector, a Herpes-Simplex Virus vector, and a VSV vector. Preferably, the viral vector is an adeno-associated virus vector or a lentiviral vector.

Furthermore, the invention relates to a cell comprising the nucleic acid of the invention, the plasmid of the invention and/or the viral vector of the invention.

In any general any cell may be used herein and in context of the present invention. Preferably, the cell is a mammalian cell, preferably a human cell. For example, the cell may be an immune cell such a T cell, B cell or NK cell. In an in vivo context, the cell may also preferably be a cancer or tumour cell.

As mentioned herein above, in some embodiments, the synthetic transcription factor of the invention comprises or consists of a first and a second polypeptide, wherein said first polypeptide comprises the DNA binding domain according to the invention and said second polypeptide comprises the transcriptional modulation domain according to the invention. In other words, the synthetic transcription factor may be a multimeric protein comprising a first polypeptide (i.e. a second amino acid chain) comprising the DNA binding domain according to the invention and a second polypeptide (i.e. a second amino acid chain) comprising the transcriptional modulation domain according to the invention. In particular, said first and second polypeptides are capable of binding to and/or interacting with each other. Said binding or interaction may be reversible and/or inducible, e.g., by a compound or light, as described herein.

Preferably, when the synthetic transcription is a multimeric protein as described above, it comprises a multimerization domain, wherein the multimerization domains of the first and second polypeptide are capable of binding to and/or interacting with each other. Preferably, said multimerization domain is a dimerization domain.

In some embodiments, the multimerization domain is a homodimerization domain. In that case, the multimerization domains of the first and second polypeptides are essentially identical to each other.

In preferred embodiments, the multimerization domain is a heterodimerization domain. In that case, the multimerization domains of the first and second polypeptide are different from each other.

In some embodiments, (i) the multimerization domain of the first polypeptide comprises or consists of a SYNZIP1 domain and the multimerization domain of the second polypeptide comprises or consists of a SYNZIP2 domain; or (ii) the multimerization domain of the first polypeptide comprises or consists of a SYNZIP2 domain and the multimerization domain of the second polypeptide comprises or consists of a SYNZIP1 domain. In particular, said SYNZIP1 domain has a sequence as shown in SEQ ID NO: 154 or a sequence that has a sequence identity of at least 80%, preferably at least 90%, more preferably at least 95%, to SEQ ID NO: 154; and said SYNZIP2 domain has a sequence as shown in SEQ ID NO: 156 or a sequence that has a sequence identity of at least 80%, preferably at least 90%, more preferably at least 95%, to SEQ ID NO: 156.

In some embodiments, the multimerization domain is a controllable domain as described herein. In particular, said multimerization domain may be a dimerization domain, wherein a compound or light, as described herein, controls the dimerization of the multimerization domains of the first and second polypeptide, i.e. a controllable dimerization domain. Preferably, said first and second polypeptide bind to and/or interact with each other in the presence of said small molecule or light.

In preferred embodiments, the multimerization domain of the first polypeptide comprises or consists of an FKBP domain and the multimerization domain of the second polypeptide comprises or consists of an FRB domain; or the multimerization domain of the first polypeptide comprises or consists of an FRB domain and the multimerization domain of the second polypeptide comprises or consists of an FKBP domain. Preferably, the multimerization domain of said first polypeptide comprises or consists of the FKBP domain, and the multimerization domain of said second polypeptide comprises or consists of the FRB domain. The first and second polypeptide can bind to and/or interact with each other in the presence of C16-(S)-7-methylindolerapamycin, in particular, wherein C16-(S)-7-methylindolerapamycin induces heterodimerization of said FKBP domain and said FRB domain.

In particular herein, the FKBP domain has a sequence as shown in SEQ ID NO: 142 or a sequence that has a sequence identity of at least 80%, preferably at least 90%, more preferably at least 95%, to SEQ ID NO: 142. Moreover, the

FRB domain has, in particular, a sequence as shown in SEQ ID NO: 140 or a sequence that has a sequence identity of at least 80%, preferably at least 90%, more preferably at least 95%, to SEQ ID NO: 140. Preferably, the DNA binding domain is N-terminally of the FKBP domain in the first polypeptide, and/or the transcriptional modulation domain is C-terminally of the FRB domain in the second polypeptide.

Furthermore, the DNA binding domain and the FKBP domain may be linked to each other via a first peptide linker, and/or the transcriptional modulation domain and the FRB domain may be linked to each other via a second peptide linker. Said first and second peptide linker, may be, for example, independently selected from the group consisting of: a cMyc NLS linker as shown in SEQ ID NO: 122, a 6AP (AP6) linker as shown in SEQ ID NO: 120, an AP8 linker as shown in SEQ ID NO: 144, a G4S linker as shown in SEQ ID NO: 118, an EAAAK3 linker as shown in SEQ ID NO: 146, an EAAAK2 linker as shown in SEQ ID NO: 148 and an G4S4 linker as shown in SEQ ID NO: 150. Of note, the terms “6AP” and “AP6” are used interchangeably herein.

In preferred embodiments, the first peptide linker is a cMyc NLS linker as shown in SEQ ID NO: 122 or a 6AP (AP6) linker as shown in SEQ ID NO: 120, and/or the second peptide linker is a 6AP (AP6) linker as shown in SEQ ID NO: 120.

In further preferred embodiments, e.g. in context with the FKBP domain and the FRB domain, the transcriptional modulation domain has a sequence as shown in SEQ ID NO: 52, SEQ ID NO: 29, SEQ ID NO: 33, SEQ ID NO: 54 or SEQ ID NO: 58, or a sequence that has a sequence identity of at least 70%, preferably at least 80%, more preferably at least 90% to any of these sequences. More preferably, said transcriptional modulation domain has a sequence as shown in SEQ ID NO: 52 or SEQ ID NO: 29, or a sequence that has a sequence identity of at least 70%, preferably at least 80%, more preferably at least 90% to SEQ ID NO: 52 or SEQ ID NO: 29.

In some preferred embodiments, the first polypeptide of the synthetic transcription factor comprises a sequence as shown in SEQ ID NO: 174, 176, 178, 180, 182, 184, 186, 188, or 192, or a sequence that has a sequence identity of at least 70%, preferably at least 80%, more preferably at least 90% to SEQ ID NO: 174, 176, 178, 180, 182, 184, 186, 188, or 192; and/or the second polypeptide of the synthetic transcription factor comprises a sequence as shown in SEQ ID NO: 162, 164, 166, 168, 170, 172, or 190, or a sequence that has a sequence identity of at least 70%, preferably at least 80%, more preferably at least 90% to SEQ ID NO: 162, 164, 166, 168, 170, 172, or 190.

Furthermore, the present invention relates to a combination of nucleic acids encoding the synthetic transcription factor of the invention, wherein one nucleic acid encodes the DNA binding domain according to the invention and another nucleic acid encodes the transcriptional modulation domain according to the invention. In particular, the nucleic acid encoding the DNA binding domain according to the invention corresponds to a first nucleic acid encoding the first polypeptide according to the invention, as described herein. Furthermore, the nucleic acid encoding the transcriptional modulation domain according to the invention corresponds, in particular, to a second nucleic acid encoding the second polypeptide according to the invention, as described herein.

Thus, the invention further relates to a combination of nucleic acids encoding the synthetic transcription factor of the invention comprising a first and second polypeptide as described herein, wherein said combination of nucleic acids comprises a first and a second nucleic acid, wherein said first nucleic acid encodes said first polypeptide, and said second nucleic acid encodes said second polypeptide.

Furthermore, the nucleic acids of the invention may be comprised in multiple plasmids, viral vectors, a cell or a kit, as described herein. In certain embodiments, the combination of nucleic acids according to the invention refers to a kit comprising said combination of nucleic acids.

In certain embodiments of the combination of nucleic acids, one nucleic acid has a DNA sequence as shown in SEQ ID NO: 5 or a DNA sequence which has a sequence identity of at least 60%, preferably at least 70%, more preferably at least 80%, to SEQ ID NO: 5, wherein said DNA sequence encodes a DNA binding domain that has a sequence identity of at least 60%, preferably at least 70%, more preferably at least 80%, to SEQ ID NO: 1. Preferably, said DNA binding domain has a sequence as shown in SEQ ID NO: 1.

Furthermore, another nucleic acid in said combination may have a DNA sequence as shown in SEQ ID NO: 6 or a DNA sequence which has a sequence identity of at least 60%, preferably at least 70%, more preferably at least 80%, to SEQ ID NO: 6, wherein said DNA sequence encodes a transcriptional modulation domain that has a sequence identity of at least 60%, preferably at least 70%, more preferably at least 80%, to SEQ ID NO: 3. Preferably, said transcriptional modulation domain has a sequence as shown in SEQ ID NO: 3.

Furthermore, the present invention relates to a DNA construct comprising a promoter (P) comprising a response element and a minimal promoter, characterized in that said response element comprises an MTERF1 binding site which has (in particular, which comprises or consists of) a sequence as shown in SEQ ID NO: 42 or SEQ ID NO: 200, or a sequence that has a sequence identity of at least 60%, preferably at least 80%, more preferably at least 90% to SEQ ID NO: 42 or SEQ ID NO: 200.

SEQ ID NO: 200 corresponds to the reverse-complement (i.e. antisense) sequence of SEQ ID NO: 42.

Herein and in context of the invention, sequences are read from 5′ to 3′ end, i.e. in sense. Thus, a DNA construct comprising a MTERF1 binding site in sense relative to the minimal promoter, comprises, for example, a sequence as shown in SEQ ID NO: 42, or a sequence that has a sequence identity of at least 60%, preferably at least 80%, more preferably at least 90% to SEQ ID NO: 42. Following the same logic, a DNA construct comprising a MTERF1 binding site in antisense relative to the minimal promoter, comprises, in particular, a sequence as shown in SEQ ID NO: 200, or a sequence that has a sequence identity of at least 60%, preferably at least 80%, more preferably at least 90% to SEQ ID NO: 200.

Nevertheless, in particular in context of dsDNA constructs according to the invention, SEQ ID NO: 42, or a sequence that has a sequence identity of at least 60%, preferably at least 80%, more preferably at least 90% to SEQ ID NO: 42 may be comprised in 5′ to 3′ direction in one strand of the dsDNA construct and the sequence of the minimal promoter as described herein may comprised in 5′ to 3′ direction in the other strand of the dsDNA construct.

Preferably herein, in particular, in context of the inventive DNA construct provided herein, the MTERF1 binding site comprises or consists of a sequence as shown in SEQ ID NO: 42, or a sequence that has a sequence identity of at least 60%, preferably at least 80%, more preferably at least 90% to SEQ ID NO: 42, in particular, an MTERF1 binding in sense relative to the minimal promoter.

Preferably herein, e.g. in context of the inventive DNA construct, the response element of the invention consists of (i) one or multiple copies, e.g. 2 to 50 copies (preferably 2 to 5 copies), of said MTERF1 binding site, wherein said multiple copies are directly adjacent to each other, or (ii) multiple copies, e.g. 2 to 50 copies (preferably 2 to 5 copies), of said MTERF1 binding site and a BS-BS spacer between at least two, preferably all, successive copies of said binding site. In particular, said spacer has a length of 1 to 1000 nucleotides, preferably 1 to 100 nucleotides, more preferably 1 to 10, e.g., 1 to 6 nucleotides. Preferably, said BS-BS spacer consists of the 1 to 10 nucleotides at the 5′ end of the sequence shown in SEQ ID NO: 198, i.e., the first 1, 2, 3, 4, 5, 6, 7, 8 or 9 or all nucleotides at the 5′ end of the sequence shown in SEQ ID NO: 198. Moreover, in case the MTERF1 binding site is orientated in sense relative to the minimal promoter, as described herein, the BS-BS spacer may consist of BS-BS spacer consists of 1, 4, 5 or 8 nucleotides, preferably, the first 1, 4, 5 or 8 nucleotides at the 5′ end of the sequence shown in SEQ ID NO: 198.

Furthermore, instead of said exemplary 2 to 50 copies, another range may be considered, e.g. 2 to 25 copies, 2 to 15 copies, 2 to 10 copies, or, preferably, 2 to 5 copies.

In some preferred embodiments, the response element consists of multiple copies of said MTERF1 binding site, e.g. 2 to 15 copies, which are directly adjacent to each other.

Furthermore, the DNA construct of the invention may, for example, have a length of at most about 106, preferably at most 105, more preferably at most about 10000 nucleotides.

Preferably herein, e.g. in context of the inventive DNA construct, the response element of the invention and the minimal promoter are separated from each other by at most about 2000 nucleotides (i.e. by a RE-minP spacer having a length of at most about 2000 nucleotides), preferably at most about 200 nucleotides, more preferably at most about 20 nucleotides, e.g., about 6 or 8 nucleotides. Preferably, said RE-minP spacer consists of the 1 to 10 nucleotides at the 5′ end of the sequence shown in SEQ ID NO: 199; and, preferably, said MTERF1 binding site comprises or consists of a sequence as shown in SEQ ID NO: 42, or a sequence that has a sequence identity of at least 60%, preferably at least 80%, more preferably at least 90% to SEQ ID NO: 42, in particular in sense relative to the minimal promoter.

Furthermore herein, e.g. in context of the inventive DNA construct, the minimal promoter may be 3′ or 5′ of said response element. Preferably, the minimal promoter is 3′ of said response element.

Herein and in context of the present invention, the minimal promoter may be a minimal TATA box which has a sequence identity of at least 60%, preferably at least 80%, more preferably at least 90% to SEQ ID NO: 103.

Furthermore, the minimal promoter may be a minimal CMV promoter which has a sequence identity of at least 60%, preferably at least 80%, more preferably at least 90% to SEQ ID NO: 136.

In particular, herein and in context of the present invention, the promoter (P) according to the invention is able to bind to the synthetic transcription factor of the invention. In particular the response element of the invention is able to bind to the DNA binding domain according to the invention.

In particular, herein and in context of the present invention, the DNA binding domain according to the invention binds to the response element of the invention in a sequence specific manner.

Preferably, herein and in context of the present invention, the DNA construct of the invention further comprises at least one gene of interest. Preferably, at least one gene of interest in 3′ of the minimal promoter.

In preferred embodiments, the gene(s) of interest encode a cell death promoting protein such as hBAX or HSV-TK, an immune stimulating cytokine such as IL-2 or IL-12, and/or an antigen-receptor such as a CAR or a TCR, as described herein.

Preferably, herein and in context of the present invention, the promoter (P) according to the invention is operably linked to at least one gene of interest, as described herein.

In particular, at least one of said gene(s) of interest is transcribed when said promoter (P), in particular the response element of the invention, is bound by the synthetic transcription factor of the invention in a cell, e.g. in a nucleus of a human cell.

In certain embodiments, the DNA construct of the invention does not comprise a sequence as shown in SEQ ID NO: 117 or a sequence which has a sequence identity of at least 90% to SEQ ID NO: 117.

As illustrated in the appended Example, the strength of the promoter (P) comprised in the DNA construct of the present invention can be adjusted as desired, i.e., stronger or weaker promoter variants may be employed; see FIGS. 14 and 15 and SEQ ID NO: 201 to 1190.

Thus, the DNA construct of the invention may comprise a sequence selected from the group consisting of: SEQ ID NO: 201 to 1190. Moreover, the spacer sequences therein i.e., consecutive nucleotides (in particular 1-10 nucleotides in length) which do not belong to the MTERF1 binding site (SEQ ID NO: 42) or the minimal promoter sequence (SEQ ID NO: 103), may be replaced by other corresponding spacer sequences of the same length.

As shown in FIG. 15 and as reflected in SEQ ID NO: 300, a specific promoter variant of particular interest has two MTERF1 binding sites in sense orientation relative to the minimal promoter which are directly adjacent to each other, and a spacer of 8 nucleotides between the response element (or the most 3′ MTERF1 binding site) and the minimal promoter sequence. This promoter variant is relatively strong while having a relatively small size.

Thus, in some preferred embodiments, the response element comprises or consists of two copies of an MTERF1 binding site, each having a sequence as shown in SEQ ID NO: 42, or a sequence that has a sequence identity of at least 60%, preferably at least 80%, more preferably at least 90% to SEQ ID NO: 42; wherein said two copies of the MTERF1 binding site are directly adjacent to each other; and, wherein the response element and the minimal promoter are separated by 8 nucleotides from each other.

In some preferred embodiments, the DNA construct of the invention comprises the sequence as shown in SEQ ID NO: 300.

As shown in FIG. 15 and as reflected in SEQ ID NO: 693, a further specific promoter variant of particular interest has five MTERF1 binding sites in sense orientation relative to the minimal promoter which are separated by spacers of 8 nucleotides in length from each other, and a spacer of 10 nucleotides between the response element (or the most 3′ MTERF1 binding site) and the minimal promoter sequence. This promoter variant is particularly strong.

Thus, in other preferred embodiments, the response element consists of five copies of an MTERF1 binding site, said copies being separated by 8 nucleotides from each other, and wherein each MTERF1 binding site has a sequence as shown in SEQ ID NO: 42, or a sequence that has a sequence identity of at least 60%, preferably at least 80%, more preferably at least 90% to SEQ ID NO: 42; and wherein the response element and the minimal promoter are separated by 10 nucleotides from each other.

In some preferred embodiments, the DNA construct of the invention comprises the sequence as shown in SEQ ID NO: 693.

Furthermore, the DNA construct of the invention may be single stranded or double stranded, i.e. dsDNA or ssDNA or dsRNA. Furthermore, invention relates to an RNA corresponding to the DNA construct of the invention (i.e. having the same sequence apart from the uracil vs. thymine). The corresponding RNA may be also single stranded or double stranded, i.e. ssRNA or dsRNA. Furthermore, the RNA may be an mRNA, e.g. contained in a lipid nanoparticle, as described herein.

Preferably, the DNA construct of the present invention (or the corresponding RNA) comprises the coding strand (i.e. sense strand). However, the DNA construct of the present invention (or the corresponding RNA) may also refer to (or even consist of) the antisense strand, and hence, be characterized by the reverse complementary sequence.

Therefore, the present invention further relates to a single or double stranded nucleic acid comprising the sense strand of the DNA construct of the invention and/or the antisense strand of the DNA construct of the invention.

Furthermore, the invention relates to a single or double stranded nucleic acid, e.g. a DNA or RNA, comprising a sequence corresponding to the sense strand of the DNA construct of the invention and/or a sequence corresponding to the antisense strand of the DNA construct of the invention.

Furthermore, the present invention relates to a plasmid comprising the DNA construct of the invention.

The invention also relates to a viral vector comprising the DNA construct of the invention, the corresponding RNA or the corresponding plasmid.

Furthermore, the invention relates to a cell comprising the DNA construct of the invention. As regards the cell, the same applies as is disclosed herein in context of the nucleic acid of the invention encoding the fusion protein (i.e the synthetic transcription factor) of the invention.

Furthermore, the present invention relates to a system (esp. a transcriptional system) comprising (i) the synthetic transcription factor of the invention, the corresponding nucleic acid of the invention, the corresponding DNA plasmid of the invention, and/or the corresponding viral vector of the invention, and (ii) the DNA construct of the invention, the DNA plasmid of the invention and/or the viral vector of the invention. Preferably said system comprises the synthetic transcription factor of the invention and the DNA construct of the invention.

Furthermore, said system may be an engineered genetic network, e.g., a biocomputing circuit.

In particular, the inventive system is suitable for regulating transcription of at least one gene of interest and may be used for this purpose. Preferably, said gene of interest is comprised in the DNA construct of the invention, as described herein. Preferably, the gene(s) of interest encode(s) a cell death promoting protein such as hBAX or HSV-TK, an immune stimulating cytokine such as IL-2 or IL-12, and/or an antigen-receptor such as a CAR or a TCR, as described herein.

Furthermore, the invention relates to a cell comprising the synthetic transcription factor of the invention and the DNA construct of the invention. Preferably, the cell is a mammalian cell, preferably a human cell. For example, the cell according to the invention may be an immune cell such a T cell, B cell or NK cell, e.g., when the gene of interest encodes an antigen-receptor such as a CAR or TCR as described herein. In an in vivo context, the cell may also preferably be a cancer or tumour cell.

Furthermore, the present invention relates to a kit comprising the synthetic transcription factor of the invention, the corresponding nucleic acid of the invention, the corresponding DNA plasmid of the invention, the corresponding viral vector of the invention, the combination of nucleic acids of the invention, the DNA construct of the invention, the single or double-stranded nucleic acid of the invention, the DNA plasmid of the invention, the viral vector of the invention and/or the system of the invention.

In certain embodiments, the kit comprises (i) the nucleic acid of the invention (i.e. encoding the fusion protein/synthetic transcription factor of the invention), the corresponding DNA plasmid of the invention, or the corresponding viral vector of the invention, and (ii) the DNA construct of the invention, the corresponding DNA plasmid of the invention or the corresponding viral vector of the invention.

Furthermore, the present invention relates to a pharmaceutical composition comprising the synthetic transcription factor of the invention, the corresponding nucleic acid of the invention, the corresponding DNA plasmid of the invention, the corresponding viral vector of the invention, the combination of nucleic acids of the invention, the DNA construct of the invention, the single or double-stranded nucleic acid of the invention, the corresponding DNA plasmid of the invention (i.e. corresponding to the inventive DNA construct), corresponding the viral vector of the invention, the system of the invention or any cell of the invention.

In certain embodiments, the pharmaceutical composition comprises (i) the nucleic acid of the invention (corresponding to the inventive fusion protein/synthetic transcription factor), the corresponding DNA plasmid of the invention, or the corresponding viral vector of the invention, and (ii) the DNA construct of the invention, the corresponding DNA plasmid of the invention or the corresponding viral vector of the invention.

In certain embodiments, the pharmaceutical composition comprises the cell of the invention comprising the inventive system.

Furthermore, the pharmaceutical composition of the invention may further comprise a pharmaceutically acceptable excipient.

Furthermore, the pharmaceutical composition of the invention may be used for treating a disease, wherein target cells are killed and/or manipulated. In particular said treatment may involve a cancer cell classifier circuit as described and/or referred to herein. Preferably, at least one gene of interest in this context may encode a cell death promoting protein such as hBAX or HSV-TK, an immune stimulating cytokine such as IL-2 or IL-12, and/or an antigen-receptor such as a CAR or a TCR.

Furthermore, the pharmaceutical composition of the invention may be used in a method of treating a tumour or cancer. In particular said treatment may involve a cancer cell classifier circuit, as described herein. Preferably, at least one gene of interest in this context encodes a cell death promoting protein such as hBAX or HSV-TK, an immune stimulating cytokine such as IL-2 or IL-12, and/or an antigen-receptor such as a CAR or a TCR.

Furthermore, the nucleic acid of the invention (corresponding to the inventive fusion protein/synthetic transcription factor), the corresponding DNA plasmid of the invention, the corresponding viral vector of the invention, the combination of nucleic acids of the invention, the DNA construct of the invention, the corresponding single or double-stranded nucleic acid of the invention, the corresponding DNA plasmid of the invention, the corresponding viral vector of the invention, or the system of the invention may be used in a gene therapy.

Furthermore, the inventive cell of the invention may used in a cell therapy. Preferably, said cell is a T cell, e.g. a CAR T cell, and said cell therapy is a T cell therapy, e.g. a CAR T cell therapy.

Furthermore, the nucleic acid of the invention (corresponding to the inventive fusion protein/synthetic transcription factor), the corresponding DNA plasmid of the invention, the corresponding viral vector of the invention, the combination of nucleic acids of the invention, the DNA construct of the invention, the corresponding single or double-stranded nucleic acid of the invention, the corresponding DNA plasmid of the invention, the corresponding viral vector of the invention, or the system of the invention may be used as a part of or in combination with an engineered genetic network, in particular, a biocomputing circuit.

Furthermore, the nucleic acid of the invention (corresponding to the inventive fusion protein/synthetic transcription factor), the corresponding DNA plasmid of the invention, the corresponding viral vector of the invention, the combination of nucleic acids of the invention, the DNA construct of the invention, the corresponding single or double-stranded nucleic acid of the invention, the corresponding DNA plasmid of the invention, the corresponding viral vector of the invention, or the system of the invention may be used for transcribing a gene of interest in vitro or in vivo, e.g. in a cell in vitro or in vivo.

As illustrated in the appended Examples, the inventors further generated a library of promoter variants and developed a method for screening for promoters that are optimized for binding to a transcription factor; see Example 6 and FIGS. 11 to 15. The inventors found, inter alia, promoter variants which are relatively small in size and provide a relatively high transcriptional activity, i.e. which are relatively strong (e.g. SEQ ID NO: 300). Furthermore, the inventors found, inter alia, promoter variants which are particularly strong (e.g. SEQ ID NO: 693). Furthermore, the inventors surprisingly found that, in addition to the number of transcription factor (TF) binding sites and the orientation of the transcription factor binding sites relative to the minimal promoter, the presence or length of spacers between the TF binding sites and the spacer between the most 3′ TF binding site and the minimal promoter (esp, the spacer between the most 3′ TF binding site and the minimal promoter) influenced the strength of the promoter.

Accordingly, the invention further relates to a library of DNA constructs comprising at least about 10, 20, 30, 40, 50, 60, 70, 80, 90, 100, 150, 200, 250, 300, 350, 400, 450, 500, 550, 600, 650, 700, 750, 800, 850, 900, 950, 1000, 2000, 3000, 4000, 5000, 6000, 7000, 8000, 9000, 10000, 50000, 100000, 500000 or 1000000, preferably at least about 100, 500, 900, 950, 990 or 1000 different DNA constructs, wherein each DNA construct in said library comprises
(i) a promoter (P) consisting of a response element (RE), a minimal promoter (minP) that is 3′ of said response element, and an optional RE-minP spacer between the response element and the minimal promoter; wherein each response element consists of one or more copies of a transcription factor binding site (BS), and an optional BS-BS spacer between at least two, preferably all, successive copies of said binding site;
(ii) an output sequence (which is preferably 3′ of said minimal promoter); and
wherein all DNA constructs in said library differ in the sequence of their promoter (P) from each other. Optionally, each DNA construct in said library comprises a unique barcode sequence which differentiates all DNA constructs in the library from each other. Means and methods for barcoding are well known in the art.

The DNA constructs in the library, in particular the promoter, response element, transcription factor binding site, minimal promoter, and spacers may be designed as described herein in context of the DNA construct of the present invention.

Thus, the transcription factor binding site is, preferably, an MTERF1 binding site which comprises or consists of a sequence as shown in SEQ ID NO: 42 or SEQ ID NO: 200 (preferably SEQ ID NO: 42), or a sequence that has a sequence identity of at least 60%, preferably at least 80%, more preferably at least 90% to SEQ ID NO: 42 or SEQ ID NO: 200 (preferably SEQ ID NO: 42). Preferably, said MTERF1 binding site consists of a sequence as shown in SEQ ID NO: 42.

The DNA constructs in the library may be identical to each other except for the promoter sequence and the optional barcode sequence. Furthermore, the minimal promoters in the different DNA constructs, in particular in the different promoters, may be identical to each other. Furthermore, the transcription factor binding sites in the different DNA constructs, in particular in the different promoters, may have the same sequence either in sense or antisense orientation relative to the minimal promoter, preferably a sequence as shown in SEQ ID NO: 42 or SEQ ID NO: 200, respectively.

In some embodiments, the promoters of at 20%, 30%, 40% or 50% of the DNA constructs differ from each other in (i) the number of binding site copies and/or (ii) the presence or the length of the RE-minP spacer; and/or at least 80%, at least 90% or all promoters of the DNA constructs in the library differ from each other in at least one parameter selected from the group consisting of: (i) the number of binding site copies, (ii) the presence or the length of the RE-minP spacer, (iii) the presence or the length of the BS-BS spacer, and (iv) the orientation of the sequence of the binding site in sense or antisense relative to the minimal promoter.

In preferred embodiments of the library, the minimal promoter is a minimal TATA box which has a sequence identity of at least 60%, preferably at least 80%, more preferably at least 90% to SEQ ID NO: 103.

In some embodiments of the library, the minimal promoter is a minimal CMV promoter which has a sequence identity of at least 60%, preferably at least 80%, more preferably at least 90% to SEQ ID NO: 136.

In particular, the promoter (P) in the DNA constructs of the library, in particular said response element, is able to (or suspected of being able to) bind to the synthetic transcription factor of the present invention.

In a further aspect, the present invention relates to a method of optimizing a promoter for binding to a transcription factor, comprising the steps of:
a) preparing a library of DNA constructs according to the invention,
b) combining the library of DNA constructs with said transcription factor in a cell or an in vitro transcription system, preferably in a cell,
c) determining the transcriptional activity of the promoters of each DNA construct in the library, preferably by determining the amount of mRNA produced from each DNA construct in the library, in particular, wherein said mRNA comprises a sequence corresponding to the output sequence of the DNA constructs, and
d) selecting a promoter based on its transcriptional activity, thereby obtaining a promoter that is optimized for binding to said transcription factor.

Preferably, the transcriptional activity of the promoters of each DNA construct in the library is determined by RNA sequencing, preferably by next-generation RNA sequencing, for example as illustrated in Example 6. More preferably, said method comprises a massively parallel reporter assay, e.g, as described in Example 6. In preferred embodiments of said method, the transcription factor is a synthetic transcription factor according to the present invention.

### SEQUENCE LISTING

The following sequences and SEQ ID NOs refer to SEQ ID NOs described herein and in context of the present invention:

| | | | |
|---|---|---|---|
| | | Internal ID | Amino Acid |
| | | | sequence ID: 1A, DNA sequence ID: |
| | | | 1B |
| | | Construct | WT codons MTERF1₅₈₋₃₉₉ |
| | | Comment | |
| Homo sapiens | SEQ ID NO: 1 | Amino Acid | FGVKCHNTDSEPLKNEDLLKNLLTMGVDIDM |
| | | sequence | ARKRQPGVFHRMITNEQDLKMFLLSKGASKE |
| | | | VIASIISRYPRAITRTPENLSKRWDLWRKIVTS |
| | | | DLEIVNILERSPESFFRSNNNLNLENNIKFLYS |
| | | | VGLTRKCLCRLLTNAPRTFSNSLDLNKQMVE |
| | | | FLQAAGLSLGHNDPADFVRKIIFKNPFILIQSTK |
| | | | RVKANIEFLRSTFNLNSEELLVLICGPGAEILDL |
| | | | SNDYARRSYANIKEKLFSLGCTEEEVQKFVLS |
| | | | YPDVIFLAEKKFNDKIDCLMEENISISQIIENPR |
| | | | VLDSSISTLKSRIKELVNAGCNLSTLNITLLSW |
| | | | SKKRYEAKLKKLSRFA |
| Homo sapiens | SEQ ID NO: 2 | DNA sequence | see attached sequence listing pursuant to WIPO |
| | | | St. 26 |
| | | Internal ID | Amino Acid |
| | | | sequence ID: 2A, DNA sequence ID: |
| | | | 2B |
| | | Construct | WT codons RelA₄₃₀₋₅₅₁ |
| | | Comment | |
| Homo sapiens | SEQ ID NO: 3 | Amino Acid | QAGEGTLSEALLQLQFDDEDLGALLGNSTDP |
| | | sequence | AVFTDLASVDNSEFQQLLNQGIPVAPHTTEP |
| | | | MLMEYPEAITRLVTGAQRPPDPAPAPLGAPG |
| | | | LPNGLLSGDEDFSSIADMDFSALLSQISS |
| Homo sapiens | SEQ ID NO: 4 | DNA sequence | see attached sequence listing pursuant to WIPO |
| | | | St. 26 |
| | | Internal ID | Amino Acid |
| | | | sequence ID: 3A, DNA sequence ID: |
| | | | 3B |
| | | Construct | Codon optimized MTERF1₅₈₋₃₉₉ |
| | | Comment | |
| Synthetic | SEQ ID NO: 5 | DNA sequence | see attached sequence listing pursuant to WIPO |
| | | | St. 26 |
| | | Internal ID | Amino Acid |
| | | | sequence ID: 4A, DNA sequence ID: |
| | | | 4B |
| | | Construct | Codon optimized RelA₄₃₀₋₅₅₁ |
| | | Comment | |
| Synthetic | SEQ ID NO: 6 | DNA sequence | see attached sequence listing pursuant to WIPO |
| | | | St. 26 |
| | | Internal ID | Amino Acid |
| | | | sequence ID: 5A, DNA sequence ID: |
| | | | 5B |
| | | Construct | MTERF1₇₃₋₃₉₉ |
| | | Comment | MTERF1 lacking N-term |
| Homo sapiens | SEQ ID NO: 7 | Amino Acid | EDLLKNLLTMGVDIDMARKRQPGVFHRMITN |
| | | sequence | EQDLKMFLLSKGASKEVIASIISRYPRAITRTP |
| | | | ENLSKRWDLWRKIVTSDLEIVNILERSPESFF |
| | | | RSNNNLNLENNIKFLYSVGLTRKCLCRLLTNA |
| | | | PRTFSNSLDLNKQMVEFLQAAGLSLGHNDPA |
| | | | DFVRKIIFKNPFILIQSTKRVKANIEFLRSTFNL |
| | | | NSEELLVLICGPGAEILDLSNDYARRSYANIKE |
| | | | KLFSLGCTEEEVQKFVLSYPDVIFLAEKKFND |
| | | | KIDCLMEENISISQIIENPRVLDSSISTLKSRIKE |
| | | | LVNAGCNLSTLNITLLSWSKKRYEAKLKKLSR |
| | | | FA |
| Homo sapiens | SEQ ID NO: 8 | DNA sequence | see attached sequence listing pursuant to WIPO |
| | | | St. 26 |
| | | Internal ID | Amino Acid |
| | | | sequence ID: 6A, DNA sequence ID: |
| | | | 6B |
| | | Construct | MTERF1₁₀₄₋₃₉₉ |
| | | Comment | MTERF1 lacking N-term and first Mterf motif |
| Homo sapiens | SEQ ID NO: 9 | Amino Acid | EQDLKMFLLSKGASKEVIASIISRYPRAITRTP |
| | | sequence | ENLSKRWDLWRKIVTSDLEIVNILERSPESFF |
| | | | RSNNNLNLENNIKFLYSVGLTRKCLCRLLTNA |
| | | | PRTFSNSLDLNKQMVEFLQAAGLSLGHNDPA |
| | | | DFVRKIIFKNPFILIQSTKRVKANIEFLRSTFNL |
| | | | NSEELLVLICGPGAEILDLSNDYARRSYANIKE |
| | | | KLFSLGCTEEEVQKFVLSYPDVIFLAEKKFND |
| | | | KIDCLMEENISISQIIENPRVLDSSISTLKSRIKE |
| | | | LVNAGCNLSTLNITLLSWSKKRYEAKLKKLSR |
| | | | FA |
| Homo sapiens | SEQ ID NO: 10 | DNA sequence | see attached sequence listing pursuant to WIPO |
| | | | St. 26 |
| | | Internal ID | Amino Acid |
| | | | sequence ID: 7A, DNA sequence ID: |
| | | | 7B |
| | | Construct | MTERF1₁₃₅₋₃₉₉ |
| | | Comment | MTERF1 lacking N-term and first and second |
| | | | Mterf motif |
| Homo sapiens | SEQ ID NO: 11 | Amino Acid | TPENLSKRWDLWRKIVTSDLEIVNILERSPESF |
| | | sequence | FRSNNNLNLENNIKFLYSVGLTRKCLCRLLTN |
| | | | APRTFSNSLDLNKQMVEFLQAAGLSLGHNDP |
| | | | ADFVRKIIFKNPFILIQSTKRVKANIEFLRSTFNL |
| | | | NSEELLVLICGPGAEILDLSNDYARRSYANIKE |
| | | | KLFSLGCTEEEVQKFVLSYPDVIFLAEKKFND |
| | | | KIDCLMEENISISQIIENPRVLDSSISTLKSRIKE |
| | | | LVNAGCNLSTLNITLLSWSKKRYEAKLKKLSR |
| | | | FA |
| Homo sapiens | SEQ ID NO: 12 | DNA sequence | see attached sequence listing pursuant to WIPO |
| | | | St. 26 |
| | | Internal ID | Amino Acid |
| | | | sequence ID: 8A, DNA sequence ID: |
| | | | 8B |
| | | Construct | VP64 |
| | | Comment | Comprises three repeats of the transactivation |
| | | | domain of HHV11 (HHV11₄₃₇₋₄₄₇, Uniprot |
| | | | P06492) linked by a GS linker |
| | | Annotation | Normal: HHV11₄₃₇₋₄₄₇ |
| | | | Underlined: GS linker |
| Synthetic | SEQ ID NO: 13 | Amino Acid | DALDDFDLDMLGSDALDDFDLDMLGSDALDD |
| | | sequence | FDLDMLGSDALDDFDLDML |
| Synthetic | SEQ ID NO: 14 | DNA sequence | see attached sequence listing pursuant to WIPO |
| | | | St. 26 |
| | | | Transactivation domain of HHV11 |
| Herpes Simplex | SEQ ID NO: 15 | Amino Acid | DALDDFDLDML |
| Virus Type 1 | | sequence | |
| Herpes Simplex | SEQ ID NO: 16 | DNA sequence | gacgcattggacgattttgatttggatatgctg |
| Virus Type 1 | | | |
| | | Internal ID | Amino Acid |
| | | | sequence ID: 9A, DNA sequence ID: |
| | | | 9B |
| | | Construct | FoxoTAD |
| | | Comment | FOXO-TAD (Pfam PF16676) of FOX03 |
| | | | (FOXO3₆₀₄₋₆₄₄, Uniprot 043524) |
| Homo sapiens | SEQ ID NO: 17 | Amino Acid | HEKFPSDLDLDMFNGSLECDMESIIRSELMDA |
| | | sequence | DGLDFNFDS |
| Homo sapiens | SEQ ID NO: 18 | DNA sequence | see attached sequence listing pursuant to WIPO |
| | | | St. 26 |
| | | Internal ID | Amino Acid |
| | | | sequence ID: 10A, DNA sequence |
| | | | ID: 10B |
| | | Construct | LMSTEN |
| | | Comment | LMSTEN motif (Pfam PF07988) of MYB |
| | | | (MYB₂₅₁₋₃₃₀, Uniprot P10242) |
| Homo sapiens | SEQ ID NO: 19 | Amino Acid | EAQNVSSHVPYPVALHVNIVNVPQPAAAAIQR |
| | | sequence | HYNDEDPEKEKRIKELELLLMSTENELKGQQV |
| | | | LPTQNHTCSYPGWHST |
| Homo sapiens | SEQ ID NO: 20 | DNA sequence | see attached sequence listing pursuant to WIPO |
| | | | St. 26 |
| | | Internal ID | Amino Acid |
| | | | sequence ID: 11A, DNA sequence |
| | | | ID: 11B |
| | | Construct | KIBRA_WW |
| | | Comment | WW domain (Pfam Pf00397) of KIBRA (KIBRA₂₋₈₁, |
| | | | Uniprot Q8IX03) |
| Homo sapiens | SEQ ID NO: 21 | Amino Acid | PRPELPLPEGWEEARDFDGKVYYIDHTNRTT |
| | | sequence | SWIDPRDRYTKPLTFADCISDELPLGWEEAYD |
| | | | PQVGDYFIDHNTKTTQI |
| Homo sapiens | SEQ ID NO: 22 | DNA sequence | see attached sequence listing pursuant to WIPO |
| | | | St. 26 |
| | | Internal ID | Amino Acid |
| | | | sequence ID: 12A, DNA sequence |
| | | | ID: 12B |
| | | Construct | ZNF473KRAB |
| | | Comment | KRAB box domain (Pfam PF01352) of ZNF473 |
| | | | (ZNF473₅₋₄₈, Uniprot MOR032) |
| | | Annotation | Normal: ZNF473KRAB |
| Homo sapiens | SEQ ID NO: 23 | Amino Acid | FVTLKDVGMDFTLGDWEQLGLEQGDTFWDT |
| | | sequence | ALDNCQDLFLLDPP |
| Homo sapiens | SEQ ID NO: 24 | DNA sequence | see attached sequence listing pursuant to WIPO |
| | | | St. 26 |
| | | Internal ID | Amino Acid |
| | | | sequence ID: 13A, DNA sequence |
| | | | ID: 13B |
| | | Construct | NucRecCoAct |
| | | Comment | Nuc_rec_co-act (Pfam PF08815) of NCOA3 |
| | | | (NCOA3₁₀₄₅₋₁₀₉₂, Uniprot Q9Y6Q9) |
| Homo sapiens | SEQ ID NO: 25 | Amino Acid | EGQSDERALLDQLHTLLSNTDATGLEEIDRAL |
| | | sequence | GIPELVNQGQALEPKQ |
| Homo sapiens | SEQ ID NO: 26 | DNA sequence | see attached sequence listing pursuant to WIPO |
| | | | St. 26 |
| | | Internal ID | Amino Acid |
| | | | sequence ID: 14A, DNA sequence |
| | | | ID: 14B |
| | | Construct | RelA₃₆₁₋₅₅₁ (PIT2 version) |
| | | Comment | |
| Homo sapiens | SEQ ID NO: 27 | Amino Acid | DEFPTMVFPSGQISQASALAPAPPQVLPQAP |
| | | sequence | APAPAPAMVSALAQAPAPVPVLAPGPPQAVA |
| | | | PPAPKPTQAGEGTLSEALLQLQFDDEDLGAL |
| | | | LGNSTDPAVFTDLASVDNSEFQQLLNQGIPVA |
| | | | PHTTEPMLMEYPEAITRLVTGAQRPPDPAPA |
| | | | PLGAPGLPNGLLSGDEDFSSIADMDFSALLSQ |
| | | | ISS |
| Homo sapiens | SEQ ID NO: 28 | DNA sequence | see attached sequence listing pursuant to WIPO |
| | | | St. 26 |
| | | Internal ID | Amino Acid |
| | | | sequence ID: 15A, DNA sequence |
| | | | ID: 15B |
| | | Construct | RelA₃₄₂₋₅₅₁ (Includes all annotated TADs) |
| | | Comment | |
| Homo sapiens | SEQ ID NO: 29 | Amino Acid | PKPAPQPYPFTSSLSTINYDEFPTMVFPSGQI |
| | | sequence | SQASALAPAPPQVLPQAPAPAPAPAMVSALA |
| | | | QAPAPVPVLAPGPPQAVAPPAPKPTQAGEGT |
| | | | LSEALLQLQFDDEDLGALLGNSTDPAVFTDLA |
| | | | SVDNSEFQQLLNQGIPVAPHTTEPMLMEYPE |
| | | | AITRLVTGAQRPPDPAPAPLGAPGLPNGLLSG |
| | | | DEDFSSIADMDFSALLSQISS |
| Homo sapiens | SEQ ID NO: 30 | DNA sequence | see attached sequence listing pursuant to WIPO |
| | | | St. 26 |
| | | Internal ID | Amino Acid |
| | | | sequence ID: 16A, DNA sequence |
| | | | ID: 16B |
| | | Construct | RelA₅₂₁₋₅₅₁ (TA1) |
| | | Comment | |
| Homo sapiens | SEQ ID NO: 31 | Amino Acid | PGLPNGLLSGDEDFSSIADMDFSALLSQISS |
| | | sequence | |
| Homo sapiens | SEQ ID NO: 32 | DNA sequence | see attached sequence listing pursuant to WIPO |
| | | | St. 26 |
| | | Internal ID | Amino Acid |
| | | | sequence ID: 17A, DNA sequence |
| | | | ID: 17B |
| | | Construct | 2xFoxoTAD |
| | | Comment | |
| | | Annotation | Normal: first FoxoTAD |
| | | | Underlined: second FoxoTAD |
| Synthetic | SEQ ID NO: 33 | Amino Acid | HEKFPSDLDLDMFNGSLECDMESIIRSELMDA |
| | | sequence | DGLDFNFDSHEKFPSDLDLDMFNGSLECDME |
| | | | SIIRSELMDADGLDFNFDS |
| Synthetic | SEQ ID NO: 34 | DNA sequence | CACGAGAAGTTCCCCAGCGACCTGGACCT |
| | | | GGACATGTTCAACGGCAGCCTGGAGTGCG |
| | | | ACATGGAGAGCATCATCAGAAGCGAGCTGA |
| | | | TGGACGCCGACGGCCTGGACTTCAACTTC |
| | | | GACAGCCACGAGAAGTTCCCCTCCGACCT |
| | | | GGATCTCGATATGTTTAATGGAAGTCTGGA |
| | | | ATGCGACATGGAATCTATTATCAGGTCCGA |
| | | | ACTGATGGATGCCGACGGCCTGGATTTCAA |
| | | | CTTTGATAGC |
| | | Internal ID | Amino Acid |
| | | | sequence ID: 18A, DNA sequence |
| | | | ID: 18B |
| | | Construct | 2xTA1 |
| | | Comment | |
| | | Annotation | Normal: first TA1 |
| | | | Underlined: second TA1 |
| Synthetic | SEQ ID NO: 35 | Amino Acid | PGLPNGLLSGDEDFSSIADMDFSALLSQISSP |
| | | sequence | GLPNGLLSGDEDFSSIADMDFSALLSQISS |
| Synthetic | SEQ ID NO: 36 | DNA sequence | CCGGGGCTCCCCAATGGCCTCCTTTCAGG |
| | | | AGATGAAGACTTCTCCTCCATTGCGGACAT |
| | | | GGACTTCTCAGCCCTGCTGAGTCAGATCAG |
| | | | CTCCCCTGGACTGCCTAACGGCCTGCTGA |
| | | | GCGGCGATGAAGATTTCTCCTCCATCGCCG |
| | | | ACATGGACTTCAGCGCTCTGCTGAGCCAGA |
| | | | TTTCTAGC |
| | | Construct | MTERF1 mitochondrial transfer peptide |
| | | | (MTERF1₁₋₅₇) |
| Homo sapiens | SEQ ID NO: 37 | Amino Acid | MQSLSLGQTSISKGLNYLTIMAPGNLWHMRN |
| | | sequence | NFLFGSRCWMTRFSAENIFKSVSFRL |
| Homo sapiens | SEQ ID NO: 38 | DNA sequence | ATGCAGAGCCTTTCCTTAGGACAAACAAGC |
| | | | ATTTCAAAAGGTTTGAACTACCTAACCATTA |
| | | | TGGCACCAGGAAACCTCTGGCATATGAGAA |
| | | | ATAACTTTCTCTTTGGTTCAAGATGTTGGAT |
| | | | GACTCGATTTTCAGCAGAAAACATCTTCAAA |
| | | | TCAGTTTCATTTAGGCTT |
| | | Construct | First MTERF 14 N-terminal amino acids |
| | | | (MTERF1₅₈₋₇₂) |
| Homo sapiens | SEQ ID NO: 39 | Amino Acid | FGVKCHNTDSEPLKN |
| | | sequence | |
| Homo sapiens | SEQ ID NO: 40 | DNA sequence | TTTGGTGTGAAGTGTCATAATACAGACAGT |
| | | | GAGCCTTTGAAAAAT |
| | | Internal ID | 19 |
| | | Construct | 3xRE, 6 bp spacing |
| | | Comment | |
| | | Annotation | Bold: MTERF1 binding site |
| | | | Normal: spacer |
| Synthetic | SEQ ID NO: 41 | DNA sequence | TAAGATGGCAGAGCCCGGTAATTGCGATT |
| | | | AAGATGGCAGAGCCCGGTAATTGCGATTA |
| | | | AGATGGCAGAGCCCGGTAAT |
| | | | MTERF1 binding site |
| Homo sapiens | SEQ ID NO: 42 | DNA sequence | TAAGATGGCAGAGCCCGGTAAT |
| | | | 6 bp spacer |
| Synthetic | SEQ ID NO: 43 | DNA sequence | TGCGAT |
| | | Internal ID | 20 |
| | | Construct | 5xRE, 6 bp spacing |
| | | Comment | |
| | | Annotation | Bold: MTERF1 binding site |
| | | | Normal: spacer |
| Synthetic | SEQ ID NO: 44 | DNA sequence | TAAGATGGCAGAGCCCGGTAATTGCGATT |
| | | | AAGATGGCAGAGCCCGGTAATTGCGATTA |
| | | | AGATGGCAGAGCCCGGTAATTGCGATTAA |
| | | | GATGGCAGAGCCCGGTAATTGCGATTAAG |
| | | | ATGGCAGAGCCCGGTAAT |
| | | Internal ID | 21 |
| | | Construct | 7xRE, 6 bp spacing |
| | | Comment | |
| | | Annotation | Bold: MTERF1 binding site |
| | | | Normal: spacer |
| Synthetic | SEQ ID NO: 45 | DNA sequence | TAAGATGGCAGAGCCCGGTAATTGCGATT |
| | | | AAGATGGCAGAGCCCGGTAATTGCGATTA |
| | | | AGATGGCAGAGCCCGGTAATTGCGATTAA |
| | | | GATGGCAGAGCCCGGTAATTGCGATTAAG |
| | | | ATGGCAGAGCCCGGTAATTGCGATTAAGA |
| | | | TGGCAGAGCCCGGTAATTGCGATTAAGAT |
| | | | GGCAGAGCCCGGTAAT |
| | | Internal ID | 22 |
| | | Construct | 9xRE, 6 bp spacing |
| | | Comment | |
| | | Annotation | Bold: MTERF1 binding site |
| | | | Normal: spacer |
| Synthetic | SEQ ID NO: 46 | DNA sequence | TAAGATGGCAGAGCCCGGTAATTGCGATT |
| | | | AAGATGGCAGAGCCCGGTAATTGCGATTA |
| | | | AGATGGCAGAGCCCGGTAATTGCGATTAA |
| | | | GATGGCAGAGCCCGGTAATTGCGATTAAG |
| | | | ATGGCAGAGCCCGGTAATTGCGATTAAGA |
| | | | TGGCAGAGCCCGGTAATTGCGATTAAGAT |
| | | | GGCAGAGCCCGGTAATTGCGATTAAGATG |
| | | | GCAGAGCCCGGTAATTGCGATTAAGATGG |
| | | | CAGAGCCCGGTAAT |
| | | Internal ID | 23 |
| | | Construct | 11xRE, 6 bp spacing |
| | | Comment | |
| | | Annotation | Bold: MTERF1 binding site |
| | | | Normal: spacer |
| Synthetic | SEQ ID NO: 47 | DNA sequence | TAAGATGGCAGAGCCCGGTAATTGCGATT |
| | | | AAGATGGCAGAGCCCGGTAATTGCGATTA |
| | | | AGATGGCAGAGCCCGGTAATTGCGATTAA |
| | | | GATGGCAGAGCCCGGTAATTGCGATTAAG |
| | | | ATGGCAGAGCCCGGTAATTGCGATTAAGA |
| | | | TGGCAGAGCCCGGTAATTGCGATTAAGAT |
| | | | GGCAGAGCCCGGTAATTGCGATTAAGATG |
| | | | GCAGAGCCCGGTAATTGCGATTAAGATGG |
| | | | CAGAGCCCGGTAATTGCGATTAAGATGGC |
| | | | AGAGCCCGGTAATTGCGATTAAGATGGCA |
| | | | GAGCCCGGTAAT |
| | | Internal ID | 24 |
| | | Construct | 5xRE, 0 bp spacing |
| | | Comment | |
| | | Annotation | Bold: MTERF1 binding site |
| | | | Normal: spacer |
| Synthetic | SEQ ID NO: 48 | DNA sequence | TAAGATGGCAGAGCCCGGTAATTAAGATG |
| | | | GCAGAGCCCGGTAATTAAGATGGCAGAGC |
| | | | CCGGTAATTAAGATGGCAGAGCCCGGTAA |
| | | | TTAAGATGGCAGAGCCCGGTAAT |
| | | Internal ID | 25 |
| | | Construct | 5xRE, 10 bp spacing |
| | | Comment | |
| | | Annotation | Bold: MTERF1 binding site |
| | | | Normal: spacer |
| Synthetic | SEQ ID NO: 49 | DNA sequence | TAAGATGGCAGAGCCCGGTAAT |
| | | | AAGGTGCGATTAAGATGGCAGAGCCCGGT |
| | | | AATAAGGTGCGAT |
| | | | TAAGATGGCAGAGCCCGGTAATAAGGTGC |
| | | | GATTAAGATGGCAGAGCCCGGTAATAAGG |
| | | | TGCGATTAAGATGGCAGAGCCCGGTAAT |
| | | Construct | 10 bp spacer |
| Synthetic | SEQ ID NO: 50 | DNA sequence | AAGGTGCGAT |
| | | Internal ID | 26 |
| | | Construct | 5 x scrambled RE, 10 bp spacing |
| | | Comment | |
| | | Annotation | Bold: scrambled MTERF1 binding site |
| | | | Normal: spacer |
| Synthetic | SEQ ID NO: 51 | DNA sequence | ATTCTACCGTCTCGGGCCATTAAAGGCATA |
| | | | TGATTCTACCGTCTCGGGCCATTAAAGGCA |
| | | | TATGATTCTACCGTCTCGGGCCATTAAAGG |
| | | | CATATGATTCTACCGTCTCGGGCCATTAAA |
| | | | GGCATATGATTCTACCGTCTCGGGCCATTA |
| | | Construct | Foxo TAD-LMSTEN-TA1 |
| Synthetic | SEQ ID NO: 52 | Amino Acid | HEKFPSDLDLDMFNGSLECDMESIIRSELMDA |
| | | sequence | DGLDFNFDSEAQNVSSHVPYPVALHVNIVNV |
| | | | PQPAAAAIQRHYNDEDPEKEKRIKELELLLMS |
| | | | TENELKGQQVLPTQNHTCSYPGWHSTPGLP |
| | | | NGLLSGDEDFSSIADMDFSALLSQISS |
| Synthetic | SEQ ID NO: 53 | DNA sequence | see attached sequence listing pursuant to WIPO |
| | | | St. 26 |
| | | Construct | Foxo TAD-LMSTEN |
| Synthetic | SEQ ID NO: 54 | Amino Acid | HEKFPSDLDLDMFNGSLECDMESIIRSELMDA |
| | | sequence | DGLDFNFDSEAQNVSSHVPYPVALHVNIVNV |
| | | | PQPAAAAIQRHYNDEDPEKEKRIKELELLLMS |
| | | | TENELKGQQVLPTQNHTCSYPGWHST |
| Synthetic | SEQ ID NO: 55 | DNA sequence | see attached sequence listing pursuant to WIPO |
| | | | St. 26 |
| | | Construct | LMSTEN-TA1 |
| Synthetic | SEQ ID NO: 56 | Amino Acid | EAQNVSSHVPYPVALHVNIVNVPQPAAAAIQR |
| | | sequence | HYNDEDPEKEKRIKELELLLMSTENELKGQQV |
| | | | LPTQNHTCSYPGWHSTPGLPNGLLSGDEDF |
| | | | SSIADMDFSALLSQISS |
| Synthetic | SEQ ID NO: 57 | DNA sequence | see attached sequence listing pursuant to WIPO |
| | | | St. 26 |
| | | Construct | FoxoTAD-TA1 |
| Synthetic | SEQ ID NO: 58 | Amino Acid | HEKFPSDLDLDMFNGSLECDMESIIRSELMDA |
| | | sequence | DGLDFNFDSPGLPNGLLSGDEDFSSIADMDF |
| | | | SALLSQISS |
| Synthetic | SEQ ID NO: 59 | DNA sequence | see attached sequence listing pursuant to WIPO |
| | | | St. 26 |
| | | Construct | VP16 |
| Synthetic | SEQ ID NO: 60 | Amino Acid | APPTDVSLGDELHLDGEDVAMAHADALDDFD |
| | | sequence | LDMLGDGDSPGPGFTPHDSAPYGALDMADF |
| | | | EFEQMFTDALGIDEYGG |
| Synthetic | SEQ ID NO: 61 | DNA sequence | see attached sequence listing pursuant to WIPO |
| | | | St. 26 |
| | | Construct | VP48 |
| Synthetic | SEQ ID NO: 62 | Amino Acid | DALDDFDLDMLPADALDDFDLDMLPADALDD |
| | | sequence | FDLDML |
| Synthetic | SEQ ID NO: 63 | DNA sequence | see attached sequence listing pursuant to WIPO |
| | | | St. 26 |
| | | Construct | VP96 |
| Synthetic | SEQ ID NO: 64 | Amino Acid | DALDDFDLDMLGSDALDDFDLDMLGSDALDD |
| | | sequence | FDLDMLGSDALDDFDLDMLGSDALDDFDLDM |
| | | | LGSDALDDFDLDML |
| Synthetic | SEQ ID NO: 65 | DNA sequence | see attached sequence listing pursuant to WIPO |
| | | | St. 26 |
| | | Construct | VP160 |
| Synthetic | SEQ ID NO: 66 | Amino Acid | DALDDFDLDMLGSDALDDFDLDMLGSDALDD |
| | | sequence | FDLDMLGSDALDDFDLDMLGSDALDDFDLDM |
| | | | LGSDALDDFDLDMLGSDALDDFDLDMLGSDA |
| | | | LDDFDLDMLGSDALDDFDLDMLGSDALDDFD |
| | | | LDML |
| Synthetic | SEQ ID NO: 67 | DNA sequence | see attached sequence listing pursuant to WIPO |
| | | | St. 26 |
| | | Construct | VPR (VP64-p65-Rta) |
| Synthetic | SEQ ID NO: 68 | Amino Acid | SRADPKKKRKVSPGIRRLDALISTSLYKKAGY |
| | | sequence | KEASGSGRADALDDFDLDMLGSDALDDFDLD |
| | | | MLGSDALDDFDLDMLGSDALDDFDLDMLINS |
| | | | RSSGSPKKKRKVGSQYLPDTDDRHRIEEKRK |
| | | | RTYETFKSIMKKSPFSGPTDPRPPPRRIAVPS |
| | | | RSSASVPKPAPQPYPFTSSLSTINYDEFPTMV |
| | | | FPSGQISQASALAPAPPQVLPQAPAPAPAPA |
| | | | MVSALAQAPAPVPVLAPGPPQAVAPPAPKPT |
| | | | QAGEGTLSEALLQLQFDDEDLGALLGNSTDP |
| | | | AVFTDLASVDNSEFQQLLNQGIPVAPHTTEP |
| | | | MLMEYPEAITRLVTGAQRPPDPAPAPLGAPG |
| | | | LPNGLLSGDEDFSSIADMDFSALLGSGSGSR |
| | | | DSREGMFLPKPEAGSAISDVFEGREVCQPKR |
| | | | IRPFHPPGSPWANRPLPASLAPTPTGPVHEP |
| | | | VGSLTPAPVPQPLDPAPAVTPEASHLLEDPD |
| | | | EETSQAVKALREMADTVIPQKEEAAICGQMD |
| | | | LSHPPPRGHLDELTTTLESMTEDLNLDSPLTP |
| | | | ELNEILDTFLNDECLLHAMHISTGLSIFDTSLF |
| Synthetic | SEQ ID NO: 69 | DNA sequence | see attached sequence listing pursuant to WIPO |
| | | | St. 26 |
| | | Construct | ZNF10 KRAB domain |
| Homo sapiens | SEQ ID NO: 70 | Amino Acid | MDAKSLTAWSRTLVTFKDVFVDFTREEWKLL |
| | | sequence | DTAQQIVYRNVMLENYKNLVSLGYQLTKPDVI |
| | | | LRLEKGEEPWLVEREIHQETHPDSETAFEIKS |
| | | | SVSSRSIFKDKQSCDIKMEGMARNDLW |
| Homo sapiens | SEQ ID NO: 71 | DNA sequence | see attached sequence listing pursuant to WIPO |
| | | | St. 26 |
| | | Construct | DNMT3A catalytic domain |
| Homo sapiens | SEQ ID NO: 72 | Amino Acid | PSRLQMFFANNHDQEFDPPKVYPPVPAEKRK |
| | | sequence | PIRVLSLFDGIATGLLVLKDLGIQVDRYIASEVC |
| | | | EDSITVGMVRHQGKIMYVGDVRSVTQKHIQE |
| | | | WGPFDLVIGGSPCNDLSIVNPARKGLYEGTG |
| | | | RLFFEFYRLLHDARPKEGDDRPFFWLFENVV |
| | | | AMGVSDKRDISRFLESNPVMIDAKEVSAAHR |
| | | | ARYFWGNLPGMNRPLASTVNDKLELQECLE |
| | | | HGRIAKFSKVRTITTRSNSIKQGKDQHFPVFM |
| | | | NEKEDILWCTEMERVFGFPVHYTDVSNMSRL |
| | | | ARQRLLGRSWSVPVIRHLFAPLKEYFACV |
| Homo sapiens | SEQ ID NO: 73 | DNA sequence | see attached sequence listing pursuant to WIPO |
| | | | St. 26 |
| | | Construct | HDAC4 |
| Homo sapiens | SEQ ID NO: 74 | Amino Acid | SSQSHPDGLSGRDQPVELLNPARVNHMPST |
| | | sequence | VDVATALPLQVAPSAVPMDLRLDHQFSLPVA |
| | | | EPALREQQLQQELLALKQKQQIQRQILIAEFQ |
| | | | RQHEQLSRQHEAQLHEHIKQQQEMLAMKHQ |
| | | | QELLEHQRKLERHRQEQELEKQHREQKLQQ |
| | | | LKNKEKGKESAVASTEVKMKLQEFVLNKKKA |
| | | | LAHRNLNHCISSDPRYWYGKTQHSSLDQSSP |
| | | | PQSGVSTSYNHPVLGMYDAKDDFPLRKTASE |
| | | | PNLKLRSRLKQKVAERRSSPLLRRKDGPVVT |
| | | | ALKKRPLDVTDSACSSAPGSGPSSPNNSSGS |
| | | | VSAENGIAPAVPSIPAETSLAHRLVAREGSAA |
| | | | PLPLYTSPSLPNITLGLPATGPSAGTAGQQDA |
| | | | ERLTLPALQQRLSLFPGTHLTPYLSTSPLERD |
| | | | GGAAHSPLLQHMVLLEQPPAQAPLVTGLGAL |
| | | | PLHAQSLVGADRVSPSIHKLRQHRPLGRTQS |
| | | | APLPQNAQALQHLVIQQQHQQFLEKHKQQFQ |
| | | | QQQLQMNKIIPKPSEPARQPESHPEETEEEL |
| | | | REHQALLDEPYLDRLPGQKEAHAQAGVQVK |
| | | | QEPIESDEEEAEPPREVEPGQRQPSEQELLF |
| | | | RQQALLLEQQRIHQLRNYQASMEAAGIPVSF |
| | | | GGHRPLSRAQSSPASATFPVSVQEPPTKPRF |
| | | | TTGLVYDTLMLKHQCTCGSSSSHPEHAGRIQ |
| | | | SIWSRLQETGLRGKCECIRGRKATLEELQTVH |
| | | | SEAHTLLYGTNPLNRQKLDSKKLLGSLASVFV |
| | | | RLPCGGVGVDSDTIWNEVHSAGAARLAVGC |
| | | | WVELVFKVATGELKNGFAWRPPGHHAEEST |
| | | | PMGFCYFNSVAVAAKLLQQRLSVSKILIVDWD |
| | | | VHHGNGTQQAFYSDPSVLYMSLHRYDDGNF |
| | | | FPGSGAPDEVGTGPGVGFNVNMAFTGGLDP |
| | | | PMGDAEYLAAFRTVVMPIASEFAPDVVLVSS |
| | | | GFDAVEGHPTPLGGYNLSARCFGYLTKQLM |
| | | | GLAGGRIVLALEGGHDLTAICDASEACVSALL |
| | | | GNELDPLPEKVLQQRPNANAVRSMEKVMEIH |
| | | | SKYWRCLQRTTSTAGRSLIEAQTCENEEAET |
| | | | VTAMASLSVGVKPAEKRPDEEPMEEEPPL |
| Homo sapiens | SEQ ID NO: 75 | DNA sequence | see attached sequence listing pursuant to WIPO |
| | | | St. 26 |
| | | Construct | EED |
| Homo sapiens | SEQ ID NO: 76 | Amino Acid | SEREVSTAPAGTDMPAAKKQKLSSDENSNPD |
| | | sequence | LSGDENDDAVSIESGTNTERPDTPTNTPNAP |
| | | | GRKSWGKGKWKSKKCKYSFKCVNSLKEDHN |
| | | | QPLFGVQFNWHSKEGDPLVFATVGSNRVTLY |
| | | | ECHSQGEIRLLQSYVDADADENFYTCAWTYD |
| | | | SNTSHPLLAVAGSRGIIRIINPITMQCIKHYVGH |
| | | | GNAINELKFHPRDPNLLLSVSKDHALRLWNIQ |
| | | | TDTLVAIFGGVEGHRDEVLSADYDLLGEKIMS |
| | | | CGMDHSLKLWRINSKRMMNAIKESYDYNPNK |
| | | | TNRPFISQKIHFPDFSTRDIHRNYVDCVRWLG |
| | | | DLILSKSCENAIVCWKPGKMEDDIDKIKPSES |
| | | | NVTILGRFDYSQCDIWYMRFSMDFWQKMLAL |
| | | | GNQVGKLYVWDLEVEDPHKAKCTTLTHHKC |
| | | | GAAIRQTSFSRDSSILIAVCDDASIWRWDRLR |
| Homo sapiens | SEQ ID NO: 77 | DNA sequence | see attached sequence listing pursuant to WIPO |
| | | | St. 26 |
| | | Construct | CHD3 DUF1087 |
| Homo sapiens | SEQ ID NO: 78 | Amino Acid | KVAQYWVREEDKIEEIEREIIKQEENVDPDYW |
| | | sequence | EKLLRHHYEQQQEDLARNLGKGKRVRKQVN |
| | | | YNDAAQEDQDNQSEYSVG |
| Homo sapiens | SEQ ID NO: 79 | DNA sequence | see attached sequence listing pursuant to WIPO |
| | | | St. 26 |
| | | Construct | ZN777 DUF3669 |
| Homo sapiens | SEQ ID NO: 80 | Amino Acid | EITRLAVWAAVQAVERKLEAQAMRLLTLEGR |
| | | sequence | TGTNEKKIADCEKTAVEFANHLESKWVVLGTL |
| | | | LQEYGLLQRRLENMENL |
| Homo sapiens | SEQ ID NO: 81 | DNA sequence | see attached sequence listing pursuant to WIPO |
| | | | St. 26 |
| | | Construct | YAF2_RYBP |
| Homo sapiens | SEQ ID NO: 82 | Amino Acid | PSEANSIQSANATTKTSETNHTSRPRLKNVDR |
| | | sequence | STAQQLAVTVGNVTVIITDFKEKTRSSSTSSST |
| | | | VTSSAGSEQQNQSSS |
| Homo sapiens | SEQ ID NO: 83 | DNA sequence | see attached sequence listing pursuant to WIPO |
| | | | St. 26 |
| | | Construct | SUMO3_13-93 |
| Homo sapiens | SEQ ID NO: 84 | Amino Acid | ENDHINLKVAGQDGSVVQFKIKRHTPLSKLMK |
| | | sequence | AYCERQGLSMRQIRFRFDGQPINETDTPAQL |
| | | | EMEDEDTIDVFQQQTGG |
| Homo sapiens | SEQ ID NO: 85 | DNA sequence | see attached sequence listing pursuant to WIPO |
| | | | St. 26 |
| | | Construct | MPP8_45-125 |
| Homo sapiens | SEQ ID NO: 86 | Amino Acid | AEAFGDSEEDGEDVFEVEKILDMKTEGGKVL |
| | | sequence | YKVRWKGYTSDDDTWEPEIHLEDCKEVLLEF |
| | | | RKKIAENKAKAVRKDIQR |
| Homo sapiens | SEQ ID NO: 87 | DNA sequence | see attached sequence listing pursuant to WIPO |
| | | | St. 26 |
| | | Construct | MPP8_59-118 |
| Homo sapiens | SEQ ID NO: 88 | Amino Acid | FEVEKILDMKTEGGKVLYKVRWKGYTSDDDT |
| | | sequence | WEPEIHLEDCKEVLLEFRKKIAENKAKAV |
| Homo sapiens | SEQ ID NO: 89 | DNA sequence | see attached sequence listing pursuant to WIPO |
| | | | St. 26 |
| | | Construct | CBX1 Chromo shadow |
| Homo sapiens | SEQ ID NO: 90 | Amino Acid | EESEKPRGFARGLEPERIIGATDSSGELMFLM |
| | | sequence | KWKNSDEADLVPAKEANVKCPQVVISFYEER |
| | | | LTWHSYPSEDDDKKDDK |
| Homo sapiens | SEQ ID NO: 91 | DNA sequence | see attached sequence listing pursuant to WIPO |
| | | | St. 26 |
| | | Construct | SCMH1 SAM_1 |
| Homo sapiens | SEQ ID NO: 92 | Amino Acid | DASRLSGRDPSSWTVEDVMQFVREADPQLG |
| | | sequence | PHADLFRKHEIDGKALLLLRSDMMMKYMGLK |
| | | | LGPALKLSYHIDRLKQGKF |
| Homo sapiens | SEQ ID NO: 93 | DNA sequence | see attached sequence listing pursuant to WIPO |
| | | | St. 26 |
| | | Construct | HERC2 Cyt-b5 |
| Homo sapiens | SEQ ID NO: 94 | Amino Acid | TLIRKADLENHNKDGGFWTVIDGKVYDIKDFQ |
| | | sequence | TQSLTGNSILAQFAGEDPVVALEAALQFEDTR |
| | | | ESMHAFCVGQYLEPDQ |
| Homo sapiens | SEQ ID NO: 95 | DNA sequence | see attached sequence listing pursuant to WIPO |
| | | | St. 26 |
| | | Construct | BIN1 SH3_9 |
| Homo sapiens | SEQ ID NO: 96 | Amino Acid | RLDLPPGFMFKVQAQHDYTATDTDELQLKAG |
| | | sequence | DWLVIPFQNPEEQDEGWLMGVKESDWNQH |
| | | | KELEKCRGVFPENFTERVP |
| Homo sapiens | SEQ ID NO: 97 | DNA sequence | see attached sequence listing pursuant to WIPO |
| | | | St. 26 |
| | | Construct | TOX HMG_Box |
| Homo sapiens | SEQ ID NO: 98 | Amino Acid | KDPNEPQKPVSAYALFFRDTQAAIKGQNPNA |
| | | sequence | TFGEVSKIVASMWDGLGEEQKQVYKKKTEAA |
| | | | KKEYLKQLAAYRASLVSK |
| Homo sapiens | SEQ ID NO: 99 | DNA sequence | see attached sequence listing pursuant to WIPO |
| | | | St. 26 |
| | | Construct | MTERF1₅₈₋₃₉₉::RelA₄₃₀₋₅₅₁ fusion (MTF) |
| Synthetic | SEQ ID NO: 100 | Amino Acid | MFGVKCHNTDSEPLKNEDLLKNLLTMGVDID |
| | | sequence | MARKRQPGVFHRMITNEQDLKMFLLSKGASK |
| | | | EVIASIISRYPRAITRTPENLSKRWDLWRKIVT |
| | | | SDLEIVNILERSPESFFRSNNNLNLENNIKFLY |
| | | | SVGLTRKCLCRLLTNAPRTFSNSLDLNKQMV |
| | | | EFLQAAGLSLGHNDPADFVRKIIFKNPFILIQST |
| | | | KRVKANIEFLRSTFNLNSEELLVLICGPGAEIL |
| | | | DLSNDYARRSYANIKEKLFSLGCTEEEVQKFV |
| | | | LSYPDVIFLAEKKFNDKIDCLMEENISISQIIEN |
| | | | PRVLDSSISTLKSRIKELVNAGCNLSTLNITLLS |
| | | | WSKKRYEAKLKKLSRFAQAGEGTLSEALLQL |
| | | | QFDDEDLGALLGNSTDPAVFTDLASVDNSEF |
| | | | QQLLNQGIPVAPHTTEPMLMEYPEAITRLVTG |
| | | | AQRPPDPAPAPLGAPGLPNGLLSGDEDFSSI |
| | | | ADMDFSALLSQISS |
| Synthetic | SEQ ID NO: 101 | DNA sequence | see attached sequence listing pursuant to WIPO |
| | | | St. 26 |
| | | | Codon optimized: |
| Synthetic | SEQ ID NO: 102 | DNA sequence | see attached sequence listing pursuant to WIPO |
| | | | St. 26 |
| | | Construct | Minimal TATA box |
| Synthetic | SEQ ID NO: 103 | DNA sequence | CTCTAGAGGGTATATAATGGGGGCC |
| | | | First MTERF1 motif (MTERF173-98) |
| Homo sapiens | SEQ ID NO: 104 | Amino Acid | EDLLKNLLTMGVDIDMARKRQPGVFH |
| | | sequence | |
| Homo sapiens | SEQ ID NO: 105 | DNA sequence | GAGGACCTACTGAAAAACTTACTTACTATG |
| | | | GGAGTAGATATTGACATGGCAAGGAAACGA |
| | | | CAGCCTGGAGTTTTTCAT |
| | | Construct | Second MTERF1 motif (MTERF1104-134) |
| Homo sapiens | SEQ ID NO: 106 | Amino Acid | EQDLKMFLLSKGASKEVIASIISRYPRAITR |
| | | sequence | |
| Homo sapiens | SEQ ID NO: 107 | DNA sequence | GAGCAGGACCTGAAGATGTTCCTTCTTTCC |
| | | | AAAGGAGCTAGCAAAGAAGTGATCGCTAGC |
| | | | ATCATATCAAGATATCCACGAGCAATAACAC |
| | | | GT |
| | | Construct | Wild-type MTERF1 |
| Homo sapiens | SEQ ID NO: 108 | Amino Acid | MQSLSLGQTSISKGLNYLTIMAPGNLWHMRN |
| | | sequence | NFLFGSRCWMTRFSAENIFKSVSFRLFGVKC |
| | | | HNTDSEPLKNEDLLKNLLTMGVDIDMARKRQ |
| | | | PGVFHRMITNEQDLKMFLLSKGASKEVIASIIS |
| | | | RYPRAITRTPENLSKRWDLWRKIVTSDLEIVNI |
| | | | LERSPESFFRSNNNLNLENNIKFLYSVGLTRK |
| | | | CLCRLLTNAPRTFSNSLDLNKQMVEFLQAAG |
| | | | LSLGHNDPADFVRKIIFKNPFILIQSTKRVKANI |
| | | | EFLRSTFNLNSEELLVLICGPGAEILDLSNDYA |
| | | | RRSYANIKEKLFSLGCTEEEVQKFVLSYPDVIF |
| | | | LAEKKFNDKIDCLMEENISISQIIENPRVLDSSI |
| | | | STLKSRIKELVNAGCNLSTLNITLLSWSKKRYE |
| | | | AKLKKLSRFA |
| Homo sapiens | SEQ ID NO: 109 | DNA sequence | see attached sequence listing pursuant to WIPO |
| | | | St. 26 |
| | | Construct | Promoter |
| | | Comment | Bold: MTERF1 binding sites |
| | | | Underlined: minimal TATA box |
| Synthetic | SEQ ID NO: 110 | DNA sequence | TAAGATGGCAGAGCCCGGTAATTAAGATG |
| | | | GCAGAGCCCGGTAATTAAGATGGCAGAGC |
| | | | CCGGTAATTAAGATGGCAGAGCCCGGTAA |
| | | | VTTAAGATGGCAGAGCCCGGTAATCATATG |
| | | | CTCTAGAGGGTATATAATGGGGGCC |
| | | Construct | Mouse MTERF1a |
| Mus musculus | SEQ ID NO: 111 | Amino Acid | MASRNIWCVRRNFLFDLRDWMLQYSAEVFLK |
| | | sequence | SISFRPFSAECDSKDKESLEEEREDLLSNLVT |
| | | | MGVDIDMARRRQPGVFNKAVTNEQELKLFLL |
| | | | SKGASDKVIGSIISRYPRAITRTPESLSKRWDL |
| | | | WRKIMASDLEIVNILERSPESFFRSNNNLNLE |
| | | | NNIKFLCSVGLTHKCLCRLLTNAPRTFSNSLN |
| | | | LNKQMVEFLQETGMSLGHNDPRDFVRKIISK |
| | | | NPSILIQSTKRVKTNIEFLQSTFNLNKRDLLLLI |
| | | | CGPGARILDLSNDCTKKNYTNIRERLLSLGCS |
| | | | EEEVQRFVLSYLNMVFLSEKKFNDKIDCLIEE |
| | | | KISASQIIENPRILDSSINTLKTRIRELSHAGYDL |
| | | | STSSIALLSWSQRRYEAKLKRLCG |
| Mus musculus | SEQ ID NO: 112 | DNA sequence | see attached sequence listing pursuant to WIPO |
| | | | St. 26 |
| | | Construct | Mouse MTERF1b |
| Mus musculus | SEQ ID NO: 113 | Amino Acid | MASRNIWCVRRNFLFDLRGWMLQYSAEVFL |
| | | sequence | KSISFRTFSVECDSKDKESLEEEREDLLSNLV |
| | | | TMGVDIDMARRRQPGVFNKAVTNEQELKIFLL |
| | | | SKGASDKVIGSIISRYPRAITRTPESLSKRWDL |
| | | | WRKIMASDLEIVNILERSPESFFRSNNNLNLE |
| | | | NNIKFLCSVGLTHKCLCRLLTNAPRTFSNSLN |
| | | | LNKQMVEFLQETGMSLGHNDPRDFVRKIISK |
| | | | NPSILIQSTKRVKTNIEFLQSTFNLNKQDLLLLI |
| | | | CGPGARILDLSNDCTKKNYTNIRERLLSLGCS |
| | | | EEEVQRFVLSYLNMVFLSEKKFNDKIDCLIEE |
| | | | KISASQIIENPRILDSSINTLKTRIRELSHAGYDL |
| | | | STSSIALLSWSQRRYEAKLKRLCG |
| Mus musculus | SEQ ID NO: 114 | DNA sequence | see attached sequence listing pursuant to WIPO |
| | | | St. 26 |
| | | Construct | Canine MTERF1 |
| Canis lupus | SEQ ID NO: 115 | Amino Acid | MQSGLSIPKSLGYLIIMAPRSFFCMRSNFLFG |
| familiaris | | sequence | SRCWMTQFSTEVFLKSISFRLFSVKCGNADS |
| | | | ESSENEELLNNLLTMGVDVDKAKKRQPGVFN |
| | | | RMGTKEQDLKMFLLSKGASKEWVASIISRYPR |
| | | | AITRTPESLSERWDLWRRIMTSDLEIINILERS |
| | | | PEAFFRSSNNRNLENNIKFLYSVGLTHKCLCR |
| | | | LLTNAPRTFSNSLDLNKQMIKFLQEVCLSLNH |
| | | | NKPRDFIGKIIFKNPFILIQSTKRVKTNIEFLQST |
| | | | FNLNNEKLLVLLCGPGAKILDLSNDYVNRNYT |
| | | | NIKEKLFSLGCTEEEVHKFILSYPDVIFLGEKK |
| | | | FNDKIDYLIEEKFSISQIIENPRILDSSISTLKSRI |
| | | | KELVNAGYNFSTSNITLLSWSQKRYKAKLKKL |
| | | | NIE |
| Canis lupus | SEQ ID NO: 116 | DNA sequence | see attached sequence listing pursuant to WIPO |
| familiaris | | | St. 26 |
| | | Construct | tRNA(leu(UUR)) |
| Homo sapiens | SEQ ID NO: 117 | DNA sequence | GTTAAGATGGCAGAGCCCGGTAATCGCATA |
| | | | AACTTAAACTTTACAGTCAGAGGTTCAATTC |
| | | | CTCTTCTTAACA |
| | | Construct | G4S linker |
| Synthetic | SEQ ID NO: 118 | Amino Acid | GGGGSGGGGS |
| | | sequence | |
| Synthetic | SEQ ID NO: 119 | DNA sequence | GGCGGTGGCGGCAGTGGCGGAGGCGGTT |
| | | | CC |
| | | Construct | AP6 linker |
| Synthetic | SEQ ID NO: 120 | Amino Acid | APAPAPAPAPAP |
| | | sequence | |
| Synthetic | SEQ ID NO: 121 | DNA sequence | GCCCCTGCACCAGCTCCTGCCCCTGCACC |
| | | | GGCTCCA |
| | | Construct | cMycNLS linker |
| Synthetic | SEQ ID NO: 122 | Amino Acid | PAAKRVKLD |
| | | sequence | |
| Synthetic | SEQ ID NO: 123 | DNA sequence | cctgccgctaagagagtgaagctggac |
| | | Construct | EAAAK linker |
| Synthetic | SEQ ID NO: 124 | Amino Acid | EAAAK |
| | | sequence | |
| Synthetic | SEQ ID NO: 125 | DNA sequence | GAAGCAGCAGCAAAA |
| | | Construct | SV40 linker |
| Synthetic | SEQ ID NO: 126 | Amino Acid | PKKKRKV |
| | | sequence | |
| Synthetic | SEQ ID NO: 127 | DNA sequence | Ccaaaaaagaagagaaaggta |
| | | Construct | MTF-Flag |
| Synthetic | SEQ ID NO: 128 | Amino Acid | MFGVKCHNTDSEPLKNEDLLKNLLTMGVDID |
| | | sequence | MARKRQPGVFHRMITNEQDLKMFLLSKGASK |
| | | | EVIASIISRYPRAITRTPENLSKRWDLWRKIVT |
| | | | SDLEIVNILERSPESFFRSNNNLNLENNIKFLY |
| | | | SVGLTRKCLCRLLTNAPRTFSNSLDLNKQMV |
| | | | EFLQAAGLSLGHNDPADFVRKIIFKNPFILIQST |
| | | | KRVKANIEFLRSTFNLNSEELLVLICGPGAEIL |
| | | | DLSNDYARRSYANIKEKLFSLGCTEEEVQKFV |
| | | | LSYPDVIFLAEKKFNDKIDCLMEENISISQIIEN |
| | | | PRVLDSSISTLKSRIKELVNAGCNLSTLNITLLS |
| | | | WSKKRYEAKLKKLSRFAQAGEGTLSEALLQL |
| | | | QFDDEDLGALLGNSTDPAVFTDLASVDNSEF |
| | | | QQLLNQGIPVAPHTTEPMLMEYPEAITRLVTG |
| | | | AQRPPDPAPAPLGAPGLPNGLLSGDEDFSSI |
| | | | ADMDFSALLSQISSDYKDHDGDYKDHDIDYK |
| | | | DDDDK |
| Synthetic | SEQ ID NO: 129 | DNA sequence | see attached sequence listing pursuant to WIPO |
| | | | St. 26 |
| | | Construct | WT MTERF1-Flag |
| Synthetic | SEQ ID NO: 130 | Amino Acid | MQSLSLGQTSISKGLNYLTIMAPGNLWHMRN |
| | | sequence | NFLFGSRCWMTRFSAENIFKSVSFRLFGVKC |
| | | | HNTDSEPLKNEDLLKNLLTMGVDIDMARKRQ |
| | | | PGVFHRMITNEQDLKMFLLSKGASKEVIASIIS |
| | | | RYPRAITRTPENLSKRWDLWRKIVTSDLEIVNI |
| | | | LERSPESFFRSNNNLNLENNIKFLYSVGLTRK |
| | | | CLCRLLTNAPRTFSNSLDLNKQMVEFLQAAG |
| | | | LSLGHNDPADFVRKIIFKNPFILIQSTKRVKANI |
| | | | EFLRSTFNLNSEELLVLICGPGAEILDLSNDYA |
| | | | RRSYANIKEKLFSLGCTEEEVQKFVLSYPDVIF |
| | | | LAEKKFNDKIDCLMEENISISQIIENPRVLDSSI |
| | | | STLKSRIKELVNAGCNLSTLNITLLSWSKKRYE |
| | | | AKLKKLSRFADYKDHDGDYKDHDIDYKDDDD |
| | | | K |
| Synthetic | SEQ ID NO: 131 | DNA sequence | see attached sequence listing pursuant to WIPO |
| | | | St. 26 |
| | | Construct | RelA TAD2 |
| Homo sapiens | SEQ ID NO: 132 | Amino Acid | PTQAGEGTLSEALLQLQFDDEDLGALLGNST |
| | | sequence | DPAVFTDLASVDNSEFQQLLNQGIPVAPHTTE |
| | | | PMLMEYPEAITRLVTGAQRPPDPAPAPLGAP |
| | | | GLPN |
| Homo sapiens | SEQ ID NO: 133 | DNA sequence | see attached sequence listing pursuant to WIPO |
| | | | St. 26 |
| | | Construct | RelA TAD3 |
| Homo sapiens | SEQ ID NO: 134 | Amino Acid | PKPAPQPYPFTSSLSTINYDEFPTMVFPSGQI |
| | | sequence | SQASALAPAPPQVLPQ |
| Homo sapiens | SEQ ID NO: 135 | DNA sequence | see attached sequence listing pursuant to WIPO |
| | | | St. 26 |
| | | Construct | CMV minimal promoter |
| Synthetic | SEQ ID NO: 136 | DNA sequence | Gtaggcgtgtacggtgggaggcctatataagcagagct |
| | | Construct | EF1a promoter |
| Homo sapiens | SEQ ID NO: 137 | DNA sequence | see attached sequence listing pursuant to WIPO |
| | | | St. 26 |
| | | Construct | CMV promoter |
| Cytomegalovirus | SEQ ID NO: 138 | DNA Sequence | see attached sequence listing pursuant to WIPO |
| | | | St. 26 |
| | | Construct | UbC promoter |
| Homo sapiens | SEQ ID NO: 139 | DNA Sequence | see attached sequence listing pursuant to WIPO |
| | | | St. 26 |
| SEQ ID NO | Amino Acid | | |

| | sequence SEQ ID NO: 140, DNA sequence SEQ ID NO: 141 | |
|---|---|---|
| Construct | FRBT2098L (short: “FRB”) | |
| Organism | Homo sapiens | |
| Amino Acid | MVAILWHEMWHEGLEEASRLYFGERNVKGMFEVLEPLHAMMERGPQTLKETSFNQAYGRDL | |
| sequence | MEAQEWCRKYMKSGNVKDLLQAWDLYYHVFRRIS | |
| DNA sequence | see attached sequence listing pursuant to WIPO St. 26 | |
| SEQ ID NO | Amino Acid | |
| | sequence SEQ ID NO: 142, DNA sequence SEQ ID NO: 143 | |
| Construct | FKBP | |
| Organism | Homo sapiens | |
| Amino Acid | MGVQVETISPGDGRTFPKRGQTCVVHYTGMLEDGKKFDSSRDRNKPFKFMLGKQEVIRGWE | |
| sequence | EGVAQMSVGQRAKLTISPDYAYGATGHPGIIPPHATLVFDVELLKLE | |
| DNA sequence | see attached sequence listing pursuant to WIPO St. 26 | |
| SEQ ID NO | Amino Acid | |
| | sequence SEQ ID NO: 144, DNA sequence SEQ ID NO: 145 | |
| Construct | AP8 | |
| Organism | Linker | |
| Amino Acid | APAPAPAPAPAPAPAP | |
| sequence | | |
| DNA sequence | GCACCAGCACCAGCTCCTGCCCCGGCACCAGCGCCAGCACCAGCACCA | |
| SEQ ID NO | Amino Acid | |
| | sequence SEQ ID NO: 146, DNA sequence SEQ ID NO: 147 | |
| Construct | EAAAK3 | |
| Comment | linker | |
| Organism | Synthetic construct | |
| Amino Acid | EAAAKEAAAKEAAAK | |
| sequence | | |
| DNA sequence | GAAGCTGCGGCAAAAGAAGCCGCTGCGAAGGAAGCGGCAGCAAAA | |
| SEQ ID NO | Amino Acid | |
| | sequence SEQ ID NO: 148, DNA sequence SEQ ID NO: 149 | |
| Construct | EAAAK2 | |
| Comment | linker | |
| Organism | Synthetic construct | |
| Amino | EAAAKEAAAK | |
| Acid | | |
| sequence | | |
| DNA sequence | GAAGCAGCGGCAAAAGAGGCAGCGGCAAAA | |
| SEQ ID NO | Amino Acid | |
| | sequence SEQ ID NO: 150, DNA sequence SEQ ID NO: 151 | |
| Construct | G4S4 | |
| Comment | linker | |
| Organism | Synthetic construct | |
| Amino | GGGGSGGGGSGGGGSGGGGS | |
| Acid | | |
| sequence | | |
| DNA sequence | see attached sequence listing pursuant to WIPO St. 26 | |
| SEQ ID NO | Amino Acid | |
| | sequence SEQ ID NO: 152, DNA sequence SEQ ID NO: 153 | |
| Construct | ERT2 | |
| Organism | Homo sapiens | |
| Amino Acid | AGDMRAANLWPSPLMIKRSKKNSLALSLTADQMVSALLDAEPPILYSEYDPTRPFSEASMMGL | |
| sequence | LTNLADRELVHMINWAKRVPGFVDLTLHDQVHLLECAWLEILMIGLVWRSMEHPVKLLFAPNL | |
| | LLDRNQGKCVEGMVEIFDMLLATSSRFRMMNLQGEEFVCLKSIILLNSGVYTFLSSTLKSLEEK | |
| | DHIHRVLDKITDTLIHLMAKAGLTLQQQHQRLAQLLLILSHIRHMSNKGMEHLYSMKCKNVVPL | |
| | YDLLLEAADAHRLHAPTSRGGASVEETDQSHLATAGSTSSHSLQKYYITGEAEGFPATA | |
| DNA sequence | see attached sequence listing pursuant to WIPO St. 26 | |
| SEQ ID NO | Amino Acid | |
| | sequence SEQ ID NO: 154, DNA sequence SEQ ID NO: 155 | |
| Construct | SYNZIP1 | |
| Organism | Synthetic construct | |
| Amino Acid | NLVAQLENEVASLENENETLKKKNLHKKDLIAYLEKEIANLRKKIEE | |
| sequence | | |
| DNA sequence | see attached sequence listing pursuant to WIPO St. 26 | |
| SEQ ID NO | Amino Acid | |
| | sequence SEQ ID NO: 156, DNA sequence SEQ ID NO: 157 | |
| Construct | SYNZIP2 | |
| Organism | Synthetic construct | |
| Amino Acid | ARNAYLRKKIARLKKDNLQLERDEQNLEKIIANLRDEIARLENEVASHEQ | |
| sequence | | |
| DNA sequence | see attached sequence listing pursuant to WIPO St. 26 | |
| SEQ ID NO | Amino Acid | |
| | sequence SEQ ID NO: 158, DNA sequence SEQ ID NO: 159 | |
| Construct | NS3 | |
| Organism | Hepatitis C virus | |
| Amino Acid | APITAYAQQTRGLLGCIITSLTGRDKNQVEGEVQIVSTATQTFLATCINGVCWAVYHGAGTRTI | |
| sequence | ASPKGPVIQMYTNVDQDLVGWPAPQGSRSLTPCTCGSSDLYLVTRHADVIPVRRRGDSRGSL | |
| | LSPRPISYLKGSSGGPLLCPAGHAVGLFRAAVCTRGVAKAVDFIPVENLETTMRSPVFTDNSS | |
| | PPA | |
| DNA sequence | see attached sequence listing pursuant to WIPO St. 26 | |
| SEQ ID NO | Amino Acid | |
| | sequence SEQ ID NO: 160, DNA sequence SEQ ID NO: 161 | |
| Construct | synNotch core | |
| Organism | Synthetic construct | |
| Amino Acid | ILDYSFTGGAGRDIPPPQIEEACELPECQVDAGNKVCNLQCNNHACGWDGGDCSLNFNDPW | |
| sequence | KNCTQSLQCWKYFSDGHCDSQCNSAGCLFDGFDCQLTEGQCNPLYDQYCKDHFSDGHCDQ | |
| | GCNSAECEWDGLDCAEHVPERLAAGTLVLVVLLPPDQLRNNSFHFLRELSHVLHTNVVFKRD | |
| | AQGQQMIFPYYGHEEELRKHPIKRSTVGWATSSLLPGTSGGRQRRELDPMDIRGSIVYLEIDN | |
| | RQCVQSSSQCFQSATDVAAFLGALASLGSLNIPYKIEAVKSEPVEPPLPSQLHLMYVAAAAFVL | |
| | LFFVGCGVLLSRKRRRQLCIQKL | |
| DNA sequence | see attached sequence listing pursuant to WIPO St. 26 | |
| SEQ ID NO | Amino Acid | |
| | sequence SEQ ID NO: 162, DNA sequence SEQ ID NO: 163 | |
| Construct | FRB: FT:L::TA1 | |
| Comment | FRB::FOXO₃₆₀₄₋₆₄₄::MYB₂₅₁₋₃₃₀::RelA₅₂₁₋₅₅₁ | |
| Amino Acid | MVAILWHEMWHEGLEEASRLYFGERNVKGMFEVLEPLHAMMERGPQTLKETSFNQAY | |
| sequence | GRDLMEAQEWCRKYMKSGNVKDLLQAWDLYYHVFRRISHEKFPSDLDLDMFNGSLEC | |
| | DMESIIRSELMDADGLDFNFDSEAQNVSSHVPYPVALHVNIVNVPQPAAAAIQRHYNDE | |
| | DPEKEKRIKELELLLMSTENELKGQQVLPTQNHTCSYPGWHSTPGLPNGLLSGDEDFS | |
| | SIADMDFSALL SQISS | |
| DNA sequence | see attached sequence listing pursuant to WIPO St. 26 | |
| SEQ ID NO | Amino Acid | |
| | sequence SEQ ID NO: 164, DNA sequence SEQ ID NO: 165 | |
| Construct | FRB::RelA₃₄₂₋₅₅₁ | |
| Comment | | |
| Amino Acid | MVAILWHEMWHEGLEEASRLYFGERNVKGMFEVLEPLHAMMERGPQTLKETSFNQAY | |
| sequence | GRDLMEAQEWCRKYMKSGNVKDLLQAWDLYYHVFRRISPKPAPQPYPFTSSLSTINY | |
| | DEFPTMVFPSGQISQASALAPAPPQVLPQAPAPAPAPAMVSALAQAPAPVPVLAPGPP | |
| | QAVAPPAPKPTQAGEGTLSEALLQLQFDDEDLGALLGNSTDPAVFTDLASVDNSEFQQ | |
| | LLNQGIPVAPHTTEPMLMEYPEAITRLVTGAQRPPDPAPAPLGAPGLPNGLLSGDEDFS | |
| | SIADMDFSALL SQISS | |
| DNA sequence | see attached sequence listing pursuant to WIPO St. 26 | |
| SEQ ID NO | Amino Acid | |
| | sequence SEQ ID NO: 166, DNA sequence SEQ ID NO: 167 | |
| Construct | FRB::FT::TA1 | |
| Comment | FRB::FOXO3₆₀₄₋₆₄₄::RelA₅₂₁₋₅₅₁ | |
| Amino Acid | MVAILWHEMWHEGLEEASRLYFGERNVKGMFEVLEPLHAMMERGPQTLKETSFNQAY | |
| sequence | GRDLMEAQEWCRKYMKSGNVKDLLQAWDLYYHVFRRISHEKFPSDLDLDMFNGSLEC | |
| | DMESIIRSELMDADGLDFNFDSPGLPNGLLSGDEDFSSIADMDFSALLSQISS | |
| DNA sequence | see attached sequence listing pursuant to WIPO St. 26 | |
| SEQ ID NO | Amino Acid | |
| | sequence SEQ ID NO: 168, DNA sequence SEQ ID NO: 169 | |
| Construct | FRB::2xFT | |
| Comment | | |
| Amino Acid | MVAILWHEMWHEGLEEASRLYFGERNVKGMFEVLEPLHAMMERGPQTLKETSFNQAY | |
| sequence | GRDLMEAQEWCRKYMKSGNVKDLLQAWDLYYHVFRRISHEKFPSDLDLDMFNGSLEC | |
| | DMESIIRSELMDADGLDFNFDSHEKFPSDLDLDMFNGSLECDMESIIRSELMDADGLDF | |
| | NFDS | |
| DNA sequence | see attached sequence listing pursuant to WIPO St. 26 | |
| SEQ ID NO | Amino Acid | |
| | sequence SEQ ID NO: 170, DNA sequence SEQ ID NO: 171 | |
| Construct | FRB::RelA₄₃₀₋₅₅₁ | |
| Comment | | |
| Amino Acid | MVAILWHEMWHEGLEEASRLYFGERNVKGMFEVLEPLHAMMERGPQTLKETSFNQAY | |
| sequence | GRDLMEAQEWCRKYMKSGNVKDLLQAWDLYYHVFRRISQAGEGTLSEALLQLQFDDE | |
| | DLGALLGNSTDPAVFTDLASVDNSEFQQLLNQGIPVAPHTTEPMLMEYPEAITRLVTGA | |
| | QRPPDPAPAPLGAPGLPNGLLSGDEDFSSIADMDFSALLSQISS | |
| DNA sequence | see attached sequence listing pursuant to WIPO St. 26 | |
| SEQ ID NO | Amino Acid | |
| | sequence SEQ ID NO: 172, DNA sequence SEQ ID NO: 173 | |
| Construct | FRB::FT::L | |
| Comment | FRB::FOXO3₆₀₄₋₆₄₄::MYB₂₅₁₋₃₃₀ | |
| Amino Acid | MVAILWHEMWHEGLEEASRLYFGERNVKGMFEVLEPLHAMMERGPQTLKETSFNQAY | |
| sequence | GRDLMEAQEWCRKYMKSGNVKDLLQAWDLYYHVFRRISHEKFPSDLDLDMFNGSLEC | |
| | DMESIIRSELMDADGLDFNFDSEAQNVSSHVPYPVALHVNIVNVPQPAAAAIQRHYNDE | |
| | DPEKEKRIKELELLLMSTENELKGQQVLPTQNHTCSYPGWHST | |
| DNA sequence | see attached sequence listing pursuant to WIPO St. 26 | |
| SEQ ID NO | Amino Acid | |
| | sequence SEQ ID NO: 174, DNA sequence SEQ ID NO: 175 | |
| Construct | MTERF1₅₈₋₃₉₉::CMycNLS:FKBP | |
| Comment | | |
| Amino Acid | MFGVKCHNTDSEPLKNEDLLKNLLTMGVDIDMARKRQPGVFHRMITNEQDLKMFLLSK | |
| sequence | GASKEVIASIISRYPRAITRTPENLSKRWDLWRKIVTSDLEIVNILERSPESFFRSNNNLNL | |
| | ENNIKFLYSVGLTRKCLCRLLTNAPRTFSNSLDLNKQMVEFLQAAGLSLGHNDPADFVR | |
| | KIIFKNPFILIQSTKRVKANIEFLRSTFNLNSEELLVLICGPGAEILDLSNDYARRSYANIKE | |
| | KLFSLGCTEEEVQKFVLSYPDVIFLAEKKFNDKIDCLMEENISISQIIENPRVLDSSISTLK | |
| | SRIKELVNAGCNLSTLNITLLSWSKKRYEAKLKKLSRFAPAAKRVKLDMGVQVETISPG | |
| | DGRTFPKRGQTCWHYTGMLEDGKKFDSSRDRNKPFKFMLGKQEVIRGWEEGVAQM | |
| | SVGQRAKLTISPDYAYGATGHPGIIPPHATLVFDVELLKLE | |
| DNA sequence | see attached sequence listing pursuant to WIPO St. 26 | |
| SEQ ID NO | Amino Acid | |
| | sequence SEQ ID NO: 176, DNA sequence SEQ ID NO: 177 | |
| Construct | MTERF1₅₈₋₃₉₉::6AP::FKBP | |
| Amino Acid | MFGVKCHNTDSEPLKNEDLLKNLLTMGVDIDMARKRQPGVFHRMITNEQDLKMFLLSK | |
| sequence | GASKEVIASIISRYPRAITRTPENLSKRWDLWRKIVTSDLEIVNILERSPESFFRSNNNLNL | |
| | ENNIKFLYSVGLTRKCLCRLLTNAPRTFSNSLDLNKQMVEFLQAAGLSLGHNDPADFVR | |
| | KIIFKNPFILIQSTKRVKANIEFLRSTFNLNSEELLVLICGPGAEILDLSNDYARRSYANIKE | |
| | KLFSLGCTEEEVQKFVLSYPDVIFLAEKKFNDKIDCLMEENISISQIIENPRVLDSSISTLK | |
| | SRIKELVNAGCNLSTLNITLLSWSKKRYEAKLKKLSRFAAPAPAPAPAPAPMGVQVETIS | |
| | PGDGRTFPKRGQTCWHYTGMLEDGKKFDSSRDRNKPFKFMLGKQEVIRGWEEGVA | |
| | QMSVGQRAKLTISPDYAYGATGHPGIIPPHATLVFDVELLKLE | |
| DNA sequence | see attached sequence listing pursuant to WIPO St. 26 | |
| SEQ ID NO | Amino Acid | |
| | sequence SEQ ID NO: 178, DNA sequence SEQ ID NO: 179 | |
| Construct | MTERF1₅₈₋₃₉₉::AP8::FKBP | |
| Amino Acid | MFGVKCHNTDSEPLKNEDLLKNLLTMGVDIDMARKRQPGVFHRMITNEQDLKMFLLSK | |
| sequence | GASKEVIASIISRYPRAITRTPENLSKRWDLWRKIVTSDLEIVNILERSPESFFRSNNNLNL | |
| | ENNIKFLYSVGLTRKCLCRLLTNAPRTFSNSLDLNKQMVEFLQAAGLSLGHNDPADFVR | |
| | KIIFKNPFILIQSTKRVKANIEFLRSTFNLNSEELLVLICGPGAEILDLSNDYARRSYANIKE | |
| | KLFSLGCTEEEVQKFVLSYPDVIFLAEKKFNDKIDCLMEENISISQIIENPRVLDSSISTLK | |
| | SRIKELVNAGCNLSTLNITLLSWSKKRYEAKLKKLSRFAAPAPAPAPAPAPAPAPMGVQ | |
| | VETISPGDGRTFPKRGQTCWHYTGMLEDGKKFDSSRDRNKPFKFMLGKQEVIRGWE | |
| | EGVAQMSVGQRAKLTISPDYAYGATGHPGIIPPHATLVFDVELLKLE | |
| DNA sequence | see attached sequence listing pursuant to WIPO St. 26 | |
| SEQ ID NO | Amino Acid | |
| | sequence SEQ ID NO: 180, DNA sequence SEQ ID NO: 181 | |
| Construct | MTERF1₅₈₋₃₉₉::G4S::FKBP | |
| Amino Acid | MFGVKCHNTDSEPLKNEDLLKNLLTMGVDIDMARKRQPGVFHRMITNEQDLKMFLLSK | |
| sequence | GASKEVIASIISRYPRAITRTPENLSKRWDLWRKIVTSDLEIVNILERSPESFFRSNNNLNL | |
| | ENNIKFLYSVGLTRKCLCRLLTNAPRTFSNSLDLNKQMVEFLQAAGLSLGHNDPADFVR | |
| | KIIFKNPFILIQSTKRVKANIEFLRSTFNLNSEELLVLICGPGAEILDLSNDYARRSYANIKE | |
| | KLFSLGCTEEEVQKFVLSYPDVIFLAEKKFNDKIDCLMEENISISQIIENPRVLDSSISTLK | |
| | SRIKELVNAGCNLSTLNITLLSWSKKRYEAKLKKLSRFAGGGGSGGGGSMGVQVETIS | |
| | PGDGRTFPKRGQTCWHYTGMLEDGKKFDSSRDRNKPFKFMLGKQEVIRGWEEGVA | |
| | QMSVGQRAKLTISPDYAYGATGHPGIIPPHATLVFDVELLKLE | |
| DNA sequence | see attached sequence listing pursuant to WIPO St. 26 | |
| SEQ ID NO | Amino Acid | |
| | sequence SEQ ID NO: 182, DNA sequence SEQ ID NO: 183 | |
| Construct | MTERF1₅₈₋₃₉₉::EAAAK3::FKBP | |
| Amino Acid | MFGVKCHNTDSEPLKNEDLLKNLLTMGVDIDMARKRQPGVFHRMITNEQDLKMFLLSK | |
| sequence | GASKEVIASIISRYPRAITRTPENLSKRWDLWRKIVTSDLEIVNILERSPESFFRSNNNLNL | |
| | ENNIKFLYSVGLTRKCLCRLLTNAPRTFSNSLDLNKQMVEFLQAAGLSLGHNDPADFVR | |
| | KIIFKNPFILIQSTKRVKANIEFLRSTFNLNSEELLVLICGPGAEILDLSNDYARRSYANIKE | |
| | KLFSLGCTEEEVQKFVLSYPDVIFLAEKKFNDKIDCLMEENISISQIIENPRVLDSSISTLK | |
| | SRIKELVNAGCNLSTLNITLLSWSKKRYEAKLKKLSRFAEAAAKEAAAKEAAAKMGVQV | |
| | ETISPGDGRTFPKRGQTCWHYTGMLEDGKKFDSSRDRNKPFKFMLGKQEVIRGWEE | |
| | GVAQMSVGQRAKLTISPDYAYGATGHPGIIPPHATLVFDVELLKLE | |
| DNA sequence | see attached sequence listing pursuant to WIPO St. 26 | |
| SEQ ID NO | Amino Acid | |
| | sequence SEQ ID NO: 184, DNA sequence SEQ ID NO: 185 | |
| Construct | MTERF 1₅₈₋₃₉₉::EAAAK2::FKBP | |
| Amino Acid | MFGVKCHNTDSEPLKNEDLLKNLLTMGVDIDMARKRQPGVFHRMITNEQDLKMFLLSK | |
| sequence | GASKEVIASIISRYPRAITRTPENLSKRWDLWRKIVTSDLEIVNILERSPESFFRSNNNLNL | |
| | ENNIKFLYSVGLTRKCLCRLLTNAPRTFSNSLDLNKQMVEFLQAAGLSLGHNDPADFVR | |
| | KIIFKNPFILIQSTKRVKANIEFLRSTFNLNSEELLVLICGPGAEILDLSNDYARRSYANIKE | |
| | KLFSLGCTEEEVQKFVLSYPDVIFLAEKKFNDKIDCLMEENISISQIIENPRVLDSSISTLK | |
| | SRIKELVNAGCNLSTLNITLLSWSKKRYEAKLKKLSRFAEAAAKEAAAKMGVQVETISPG | |
| | DGRTFPKRGQTCVVHYTGMLEDGKKFDSSRDRNKPFKFMLGKQEVIRGWEEGVAQM | |
| | SVGQRAKLTISPDYAYGATGHPGIIPPHATLVFDVELLKLE | |
| DNA sequence | see attached sequence listing pursuant to WIPO St. 26 | |
| SEQ ID NO | Amino Acid | |
| | sequence SEQ ID NO: 186, DNA sequence SEQ ID NO: 187 | |
| Construct | MTERF1₅₈₋₃₉₉: EAAAK::FKBP | |
| Amino Acid | MFGVKCHNTDSEPLKNEDLLKNLLTMGVDIDMARKRQPGVFHRMITNEQDLKMFLLSK | |
| sequence | GASKEVIASIISRYPRAITRTPENLSKRWDLWRKIVTSDLEIVNILERSPESFFRSNNNLNL | |
| | ENNIKFLYSVGLTRKCLCRLLTNAPRTFSNSLDLNKQMVEFLQAAGLSLGHNDPADFVR | |
| | KIIFKNPFILIQSTKRVKANIEFLRSTFNLNSEELLVLICGPGAEILDLSNDYARRSYANIKE | |
| | KLFSLGCTEEEVQKFVLSYPDVIFLAEKKFNDKIDCLMEENISISQIIENPRVLDSSISTLK | |
| | SRIKELVNAGCNLSTLNITLLSWSKKRYEAKLKKLSRFAEAAAKMGVQVETISPGDGRT | |
| | FPKRGQTCWHYTGMLEDGKKFDSSRDRNKPFKFMLGKQEVIRGWEEGVAQMSVGQ | |
| | RAKLTISPDYAYGATGHPGIIPPHATLVFDVELLKLE | |
| DNA sequence | see attached sequence listing pursuant to WIPO St. 26 | |
| SEQ ID NO | Amino Acid | |
| | sequence SEQ ID NO: 188, DNA sequence SEQ ID NO: 189 | |
| Construct | MTERF1₅₈₋₃₉₉::G4S4::FKBP | |
| Amino Acid | MFGVKCHNTDSEPLKNEDLLKNLLTMGVDIDMARKRQPGVFHRMITNEQDLKMFLLSK | |
| sequence | GASKEVIASIISRYPRAITRTPENLSKRWDLWRKIVTSDLEIVNILERSPESFFRSNNNLNL | |
| | ENNIKFLYSVGLTRKCLCRLLTNAPRTFSNSLDLNKQMVEFLQAAGLSLGHNDPADFVR | |
| | KIIFKNPFILIQSTKRVKANIEFLRSTFNLNSEELLVLICGPGAEILDLSNDYARRSYANIKE | |
| | KLFSLGCTEEEVQKFVLSYPDVIFLAEKKFNDKIDCLMEENISISQIIENPRVLDSSISTLK | |
| | SRIKELVNAGCNLSTLNITLLSWSKKRYEAKLKKLSRFAGGGGSGGGGSGGGGSGGG | |
| | GSMGVQVETISPGDGRTFPKRGQTCWHYTGMLEDGKKFDSSRDRNKPFKFMLGKQ | |
| | EVIRGWEEGVAQMSVGQRAKLTISPDYAYGATGHPGIIPPHATLVFDVELLKLE | |
| DNA sequence | see attached sequence listing pursuant to WIPO St. 26 | |
| SEQ ID NO | Amino Acid | |
| | sequence SEQ ID NO: 190, DNA sequence SEQ ID NO: 191 | |
| Construct | FRB::6AP::RelA₄₃₀₋₅₅₁ | |
| Amino Acid | MVAILWHEMWHEGLEEASRLYFGERNVKGMFEVLEPLHAMMERGPQTLKETSFNQAY | |
| sequence | GRDLMEAQEWCRKYMKSGNVKDLLQAWDLYYHVFRRISAPAPAPAPAPAPQAGEGTL | |
| | SEALLQLQFDDEDLGALLGNSTDPAVFTDLASVDNSEFQQLLNQGIPVAPHTTEPMLM | |
| | EYPEAITRLVTGAQRPPDPAPAPLGAPGLPNGLLSGDEDFSSIADMDFSALLSQISS | |
| DNA sequence | see attached sequence listing pursuant to WIPO St. 26 | |
| SEQ ID NO | Amino Acid | |
| | sequence SEQ ID NO: 192, DNA sequence SEQ ID NO: 193 | |
| Construct | MTERF1₅₈₋₃₉₉::FKBP | |
| Amino Acid | MFGVKCHNTDSEPLKNEDLLKNLLTMGVDIDMARKRQPGVFHRMITNEQDLKMFLLSK | |
| sequence | GASKEVIASIISRYPRAITRTPENLSKRWDLWRKIVTSDLEIVNILERSPESFFRSNNNLNL | |
| | ENNIKFLYSVGLTRKCLCRLLTNAPRTFSNSLDLNKQMVEFLQAAGLSLGHNDPADFVR | |
| | KIIFKNPFILIQSTKRVKANIEFLRSTFNLNSEELLVLICGPGAEILDLSNDYARRSYANIKE | |
| | KLFSLGCTEEEVQKFVLSYPDVIFLAEKKFNDKIDCLMEENISISQIIENPRVLDSSISTLK | |
| | SRIKELVNAGCNLSTLNITLLSWSKKRYEAKLKKLSRFAMGVQVETISPGDGRTFPKRG | |
| | QTCWHYTGMLEDGKKFDSSRDRNKPFKFMLGKQEVIRGWEEGVAQMSVGQRAKLTI | |
| | SPDYAYGATGHPGIIPPHATLVFDVELLKLE | |
| DNA sequence | see attached sequence listing pursuant to WIPO St. 26 | |
| SEQ ID NO | Amino Acid | |
| | sequence SEQ ID NO: 194, DNA sequence SEQ ID NO: 195 | |
| Construct | 3xTA1 | |
| Comment | RelA₅₂₁₋₅₅₁::RelA₅₂₁₋₅₅₁: RelA₅₂₁₋₅₅₁ | |
| Amino Acid | PGLPNGLLSGDEDFSSIADMDFSALLSQISSPGLPNGLLSGDEDFSSIADMDFSALLSQI | |
| sequence | SSPGLPNGLLSGDEDFSSIADMDFSALLSQISS | |
| DNA sequence | see attached sequence listing pursuant to WIPO St. 26 | |
| SEQ ID NO | Amino Acid | |
| | sequence ID 196:, DNA sequence SEQ ID NO: 197 | |
| Construct | 2xLMSTEN | |
| Comment | MYB₂₅₁₋₃₃₀::MYB₂₅₁₋₃₃₀ | |
| Amino Acid | EAQNVSSHVPYPVALHVNIVNVPQPAAAAIQRHYNDEDPEKEKRIKELELLLMSTENEL | |
| sequence | KGQQVLPTQNHTCSYPGWHSTEAQNVSSHVPYPVALHVNIVNVPQPAAAAIQRHYND | |
| | EDPEKEKRIKELELLLMSTENELKGQQVLPTQNHTCSYPGWHST | |
| DNA sequence | see attached sequence listing pursuant to WIPO St. 26 | |
| SEQ ID NO: 198 | BS spacer (10 bp), | AGAGTGCGAG |
| | synthetic, DNA | |
| SEQ ID NO: 199 | TATA spacer (10 bp), | AAGGTGCGAT |
| | synthetic, DNA | |
| SEQ ID NO: 200 | MTERF1 binding site | ATTACCGGGCTCTGCCATCTTA |
| | (antisense), Homo | |
| | sapiens, DNA | |

Sequences SEQ ID NO: 1-200 are also shown in the attached sequence listing pursuant to WIPO St. 26.

The following promoter (sensor) sequences SEQ ID NO: 201-1190 are only shown in the attached sequence listing pursuant to WIPO St. 26. These promoter sequences have the following key: (a) orientation of binding sites (BS) relative to minimal promoter (TATA)-(b) number of BS-(c) distance BS-BS-(d) distance-BS-TATA).

| SEQ ID NO: | key |
|---|---|
| 201 | sense-1-0-0 |
| 202 | sense-1-0-1 |
| 203 | sense-1-0-2 |
| 204 | sense-1-0-3 |
| 205 | sense-1-0-4 |
| 206 | sense-1-0-5 |
| 207 | sense-1-0-6 |
| 208 | sense-1-0-7 |
| 209 | sense-1-0-8 |
| 210 | sense-1-0-9 |
| 211 | sense-1-0-10 |
| 212 | sense-2-0-0 |
| 213 | sense-2-1-0 |
| 214 | sense-2-2-0 |
| 215 | sense-2-3-0 |
| 216 | sense-2-4-0 |
| 217 | sense-2-5-0 |
| 218 | sense-2-6-0 |
| 219 | sense-2-7-0 |
| 220 | sense-2-8-0 |
| 221 | sense-2-9-0 |
| 222 | sense-2-10-0 |
| 223 | sense-2-0-1 |
| 224 | sense-2-1-1 |
| 225 | sense-2-2-1 |
| 226 | sense-2-3-1 |
| 227 | sense-2-4-1 |
| 228 | sense-2-5-1 |
| 229 | sense-2-6-1 |
| 230 | sense-2-7-1 |
| 231 | sense-2-8-1 |
| 232 | sense-2-9-1 |
| 233 | sense-2-10-1 |
| 234 | sense-2-0-2 |
| 235 | sense-2-1-2 |
| 236 | sense-2-2-2 |
| 237 | sense-2-3-2 |
| 238 | sense-2-4-2 |
| 239 | sense-2-5-2 |
| 240 | sense-2-6-2 |
| 241 | sense-2-7-2 |
| 242 | sense-2-8-2 |
| 243 | sense-2-9-2 |
| 244 | sense-2-10-2 |
| 245 | sense-2-0-3 |
| 246 | sense-2-1-3 |
| 247 | sense-2-2-3 |
| 248 | sense-2-3-3 |
| 249 | sense-2-4-3 |
| 250 | sense-2-5-3 |
| 251 | sense-2-6-3 |
| 252 | sense-2-7-3 |
| 253 | sense-2-8-3 |
| 254 | sense-2-9-3 |
| 255 | sense-2-10-3 |
| 256 | sense-2-0-4 |
| 257 | sense-2-1-4 |
| 258 | sense-2-2-4 |
| 259 | sense-2-3-4 |
| 260 | sense-2-4-4 |
| 261 | sense-2-5-4 |
| 262 | sense-2-6-4 |
| 263 | sense-2-7-4 |
| 264 | sense-2-8-4 |
| 265 | sense-2-9-4 |
| 266 | sense-2-10-4 |
| 267 | sense-2-0-5 |
| 268 | sense-2-1-5 |
| 269 | sense-2-2-5 |
| 270 | sense-2-3-5 |
| 271 | sense-2-4-5 |
| 272 | sense-2-5-5 |
| 273 | sense-2-6-5 |
| 274 | sense-2-7-5 |
| 275 | sense-2-8-5 |
| 276 | sense-2-9-5 |
| 277 | sense-2-10-5 |
| 278 | sense-2-0-6 |
| 279 | sense-2-1-6 |
| 280 | sense-2-2-6 |
| 281 | sense-2-3-6 |
| 282 | sense-2-4-6 |
| 283 | sense-2-5-6 |
| 284 | sense-2-6-6 |
| 285 | sense-2-7-6 |
| 286 | sense-2-8-6 |
| 287 | sense-2-9-6 |
| 288 | sense-2-10-6 |
| 289 | sense-2-0-7 |
| 290 | sense-2-1-7 |
| 291 | sense-2-2-7 |
| 292 | sense-2-3-7 |
| 293 | sense-2-4-7 |
| 294 | sense-2-5-7 |
| 295 | sense-2-6-7 |
| 296 | sense-2-7-7 |
| 297 | sense-2-8-7 |
| 298 | sense-2-9-7 |
| 299 | sense-2-10-7 |
| 300 | sense-2-0-8 |
| 301 | sense-2-1-8 |
| 302 | sense-2-2-8 |
| 303 | sense-2-3-8 |
| 304 | sense-2-4-8 |
| 305 | sense-2-5-8 |
| 306 | sense-2-6-8 |
| 307 | sense-2-7-8 |
| 308 | sense-2-8-8 |
| 309 | sense-2-9-8 |
| 310 | sense-2-10-8 |
| 311 | sense-2-0-9 |
| 312 | sense-2-1-9 |
| 313 | sense-2-2-9 |
| 314 | sense-2-3-9 |
| 315 | sense-2-4-9 |
| 316 | sense-2-5-9 |
| 317 | sense-2-6-9 |
| 318 | sense-2-7-9 |
| 319 | sense-2-8-9 |
| 320 | sense-2-9-9 |
| 321 | sense-2-10-9 |
| 322 | sense-2-0-10 |
| 323 | sense-2-1-10 |
| 324 | sense-2-2-10 |
| 325 | sense-2-3-10 |
| 326 | sense-2-4-10 |
| 327 | sense-2-5-10 |
| 328 | sense-2-6-10 |
| 329 | sense-2-7-10 |
| 330 | sense-2-8-10 |
| 331 | sense-2-9-10 |
| 332 | sense-2-10-10 |
| 333 | sense-3-0-0 |
| 334 | sense-3-1-0 |
| 335 | sense-3-2-0 |
| 336 | sense-3-3-0 |
| 337 | sense-3-4-0 |
| 338 | sense-3-5-0 |
| 339 | sense-3-6-0 |
| 340 | sense-3-7-0 |
| 341 | sense-3-8-0 |
| 342 | sense-3-9-0 |
| 343 | sense-3-10-0 |
| 344 | sense-3-0-1 |
| 345 | sense-3-1-1 |
| 346 | sense-3-2-1 |
| 347 | sense-3-3-1 |
| 348 | sense-3-4-1 |
| 349 | sense-3-5-1 |
| 350 | sense-3-6-1 |
| 351 | sense-3-7-1 |
| 352 | sense-3-8-1 |
| 353 | sense-3-9-1 |
| 354 | sense-3-10-1 |
| 355 | sense-3-0-2 |
| 356 | sense-3-1-2 |
| 357 | sense-3-2-2 |
| 358 | sense-3-3-2 |
| 359 | sense-3-4-2 |
| 360 | sense-3-5-2 |
| 361 | sense-3-6-2 |
| 362 | sense-3-7-2 |
| 363 | sense-3-8-2 |
| 364 | sense-3-9-2 |
| 365 | sense-3-10-2 |
| 366 | sense-3-0-3 |
| 367 | sense-3-1-3 |
| 368 | sense-3-2-3 |
| 369 | sense-3-3-3 |
| 370 | sense-3-4-3 |
| 371 | sense-3-5-3 |
| 372 | sense-3-6-3 |
| 373 | sense-3-7-3 |
| 374 | sense-3-8-3 |
| 375 | sense-3-9-3 |
| 376 | sense-3-10-3 |
| 377 | sense-3-0-4 |
| 378 | sense-3-1-4 |
| 379 | sense-3-2-4 |
| 380 | sense-3-3-4 |
| 381 | sense-3-4-4 |
| 382 | sense-3-5-4 |
| 383 | sense-3-6-4 |
| 384 | sense-3-7-4 |
| 385 | sense-3-8-4 |
| 386 | sense-3-9-4 |
| 387 | sense-3-10-4 |
| 388 | sense-3-0-5 |
| 389 | sense-3-1-5 |
| 390 | sense-3-2-5 |
| 391 | sense-3-3-5 |
| 392 | sense-3-4-5 |
| 393 | sense-3-5-5 |
| 394 | sense-3-6-5 |
| 395 | sense-3-7-5 |
| 396 | sense-3-8-5 |
| 397 | sense-3-9-5 |
| 398 | sense-3-10-5 |
| 399 | sense-3-0-6 |
| 400 | sense-3-1-6 |
| 401 | sense-3-2-6 |
| 402 | sense-3-3-6 |
| 403 | sense-3-4-6 |
| 404 | sense-3-5-6 |
| 405 | sense-3-6-6 |
| 406 | sense-3-7-6 |
| 407 | sense-3-8-6 |
| 408 | sense-3-9-6 |
| 409 | sense-3-10-6 |
| 410 | sense-3-0-7 |
| 411 | sense-3-1-7 |
| 412 | sense-3-2-7 |
| 413 | sense-3-3-7 |
| 414 | sense-3-4-7 |
| 415 | sense-3-5-7 |
| 416 | sense-3-6-7 |
| 417 | sense-3-7-7 |
| 418 | sense-3-8-7 |
| 419 | sense-3-9-7 |
| 420 | sense-3-10-7 |
| 421 | sense-3-0-8 |
| 422 | sense-3-1-8 |
| 423 | sense-3-2-8 |
| 424 | sense-3-3-8 |
| 425 | sense-3-4-8 |
| 426 | sense-3-5-8 |
| 427 | sense-3-6-8 |
| 428 | sense-3-7-8 |
| 429 | sense-3-8-8 |
| 430 | sense-3-9-8 |
| 431 | sense-3-10-8 |
| 432 | sense-3-0-9 |
| 433 | sense-3-1-9 |
| 434 | sense-3-2-9 |
| 435 | sense-3-3-9 |
| 436 | sense-3-4-9 |
| 437 | sense-3-5-9 |
| 438 | sense-3-6-9 |
| 439 | sense-3-7-9 |
| 440 | sense-3-8-9 |
| 441 | sense-3-9-9 |
| 442 | sense-3-10-9 |
| 443 | sense-3-0-10 |
| 444 | sense-3-1-10 |
| 445 | sense-3-2-10 |
| 446 | sense-3-3-10 |
| 447 | sense-3-4-10 |
| 448 | sense-3-5-10 |
| 449 | sense-3-6-10 |
| 450 | sense-3-7-10 |
| 451 | sense-3-8-10 |
| 452 | sense-3-9-10 |
| 453 | sense-3-10-10 |
| 454 | sense-4-0-0 |
| 455 | sense-4-1-0 |
| 456 | sense-4-2-0 |
| 457 | sense-4-3-0 |
| 458 | sense-4-4-0 |
| 459 | sense-4-5-0 |
| 460 | sense-4-6-0 |
| 461 | sense-4-7-0 |
| 462 | sense-4-8-0 |
| 463 | sense-4-9-0 |
| 464 | sense-4-10-0 |
| 465 | sense-4-0-1 |
| 466 | sense-4-1-1 |
| 467 | sense-4-2-1 |
| 468 | sense-4-3-1 |
| 469 | sense-4-4-1 |
| 470 | sense-4-5-1 |
| 471 | sense-4-6-1 |
| 472 | sense-4-7-1 |
| 473 | sense-4-8-1 |
| 474 | sense-4-9-1 |
| 475 | sense-4-10-1 |
| 476 | sense-4-0-2 |
| 477 | sense-4-1-2 |
| 478 | sense-4-2-2 |
| 479 | sense-4-3-2 |
| 480 | sense-4-4-2 |
| 481 | sense-4-5-2 |
| 482 | sense-4-6-2 |
| 483 | sense-4-7-2 |
| 484 | sense-4-8-2 |
| 485 | sense-4-9-2 |
| 486 | sense-4-10-2 |
| 487 | sense-4-0-3 |
| 488 | sense-4-1-3 |
| 489 | sense-4-2-3 |
| 490 | sense-4-3-3 |
| 491 | sense-4-4-3 |
| 492 | sense-4-5-3 |
| 493 | sense-4-6-3 |
| 494 | sense-4-7-3 |
| 495 | sense-4-8-3 |
| 496 | sense-4-9-3 |
| 497 | sense-4-10-3 |
| 498 | sense-4-0-4 |
| 499 | sense-4-1-4 |
| 500 | sense-4-2-4 |
| 501 | sense-4-3-4 |
| 502 | sense-4-4-4 |
| 503 | sense-4-5-4 |
| 504 | sense-4-6-4 |
| 505 | sense-4-7-4 |
| 506 | sense-4-8-4 |
| 507 | sense-4-9-4 |
| 508 | sense-4-10-4 |
| 509 | sense-4-0-5 |
| 510 | sense-4-1-5 |
| 511 | sense-4-2-5 |
| 512 | sense-4-3-5 |
| 513 | sense-4-4-5 |
| 514 | sense-4-5-5 |
| 515 | sense-4-6-5 |
| 516 | sense-4-7-5 |
| 517 | sense-4-8-5 |
| 518 | sense-4-9-5 |
| 519 | sense-4-10-5 |
| 520 | sense-4-0-6 |
| 521 | sense-4-1-6 |
| 522 | sense-4-2-6 |
| 523 | sense-4-3-6 |
| 524 | sense-4-4-6 |
| 525 | sense-4-5-6 |
| 526 | sense-4-6-6 |
| 527 | sense-4-7-6 |
| 528 | sense-4-8-6 |
| 529 | sense-4-9-6 |
| 530 | sense-4-10-6 |
| 531 | sense-4-0-7 |
| 532 | sense-4-1-7 |
| 533 | sense-4-2-7 |
| 534 | sense-4-3-7 |
| 535 | sense-4-4-7 |
| 536 | sense-4-5-7 |
| 537 | sense-4-6-7 |
| 538 | sense-4-7-7 |
| 539 | sense-4-8-7 |
| 540 | sense-4-9-7 |
| 541 | sense-4-10-7 |
| 542 | sense-4-0-8 |
| 543 | sense-4-1-8 |
| 544 | sense-4-2-8 |
| 545 | sense-4-3-8 |
| 546 | sense-4-4-8 |
| 547 | sense-4-5-8 |
| 548 | sense-4-6-8 |
| 549 | sense-4-7-8 |
| 550 | sense-4-8-8 |
| 551 | sense-4-9-8 |
| 552 | sense-4-10-8 |
| 553 | sense-4-0-9 |
| 554 | sense-4-1-9 |
| 555 | sense-4-2-9 |
| 556 | sense-4-3-9 |
| 557 | sense-4-4-9 |
| 558 | sense-4-5-9 |
| 559 | sense-4-6-9 |
| 560 | sense-4-7-9 |
| 561 | sense-4-8-9 |
| 562 | sense-4-9-9 |
| 563 | sense-4-10-9 |
| 564 | sense-4-0-10 |
| 565 | sense-4-1-10 |
| 566 | sense-4-2-10 |
| 567 | sense-4-3-10 |
| 568 | sense-4-4-10 |
| 569 | sense-4-5-10 |
| 570 | sense-4-6-10 |
| 571 | sense-4-7-10 |
| 572 | sense-4-8-10 |
| 573 | sense-4-9-10 |
| 574 | sense-4-10-10 |
| 575 | sense-5-0-0 |
| 576 | sense-5-1-0 |
| 577 | sense-5-2-0 |
| 578 | sense-5-3-0 |
| 579 | sense-5-4-0 |
| 580 | sense-5-5-0 |
| 581 | sense-5-6-0 |
| 582 | sense-5-7-0 |
| 583 | sense-5-8-0 |
| 584 | sense-5-9-0 |
| 585 | sense-5-10-0 |
| 586 | sense-5-0-1 |
| 587 | sense-5-1-1 |
| 588 | sense-5-2-1 |
| 589 | sense-5-3-1 |
| 590 | sense-5-4-1 |
| 591 | sense-5-5-1 |
| 592 | sense-5-6-1 |
| 593 | sense-5-7-1 |
| 594 | sense-5-8-1 |
| 595 | sense-5-9-1 |
| 596 | sense-5-10-1 |
| 597 | sense-5-0-2 |
| 598 | sense-5-1-2 |
| 599 | sense-5-2-2 |
| 600 | sense-5-3-2 |
| 601 | sense-5-4-2 |
| 602 | sense-5-5-2 |
| 603 | sense-5-6-2 |
| 604 | sense-5-7-2 |
| 605 | sense-5-8-2 |
| 606 | sense-5-9-2 |
| 607 | sense-5-10-2 |
| 608 | sense-5-0-3 |
| 609 | sense-5-1-3 |
| 610 | sense-5-2-3 |
| 611 | sense-5-3-3 |
| 612 | sense-5-4-3 |
| 613 | sense-5-5-3 |
| 614 | sense-5-6-3 |
| 615 | sense-5-7-3 |
| 616 | sense-5-8-3 |
| 617 | sense-5-9-3 |
| 618 | sense-5-10-3 |
| 619 | sense-5-0-4 |
| 620 | sense-5-1-4 |
| 621 | sense-5-2-4 |
| 622 | sense-5-3-4 |
| 623 | sense-5-4-4 |
| 624 | sense-5-5-4 |
| 625 | sense-5-6-4 |
| 626 | sense-5-7-4 |
| 627 | sense-5-8-4 |
| 628 | sense-5-9-4 |
| 629 | sense-5-10-4 |
| 630 | sense-5-0-5 |
| 631 | sense-5-1-5 |
| 632 | sense-5-2-5 |
| 633 | sense-5-3-5 |
| 634 | sense-5-4-5 |
| 635 | sense-5-5-5 |
| 636 | sense-5-6-5 |
| 637 | sense-5-7-5 |
| 638 | sense-5-8-5 |
| 639 | sense-5-9-5 |
| 640 | sense-5-10-5 |
| 641 | sense-5-0-6 |
| 642 | sense-5-1-6 |
| 643 | sense-5-2-6 |
| 644 | sense-5-3-6 |
| 645 | sense-5-4-6 |
| 646 | sense-5-5-6 |
| 647 | sense-5-6-6 |
| 648 | sense-5-7-6 |
| 649 | sense-5-8-6 |
| 650 | sense-5-9-6 |
| 651 | sense-5-10-6 |
| 652 | sense-5-0-7 |
| 653 | sense-5-1-7 |
| 654 | sense-5-2-7 |
| 655 | sense-5-3-7 |
| 656 | sense-5-4-7 |
| 657 | sense-5-5-7 |
| 658 | sense-5-6-7 |
| 659 | sense-5-7-7 |
| 660 | sense-5-8-7 |
| 661 | sense-5-9-7 |
| 662 | sense-5-10-7 |
| 663 | sense-5-0-8 |
| 664 | sense-5-1-8 |
| 665 | sense-5-2-8 |
| 666 | sense-5-3-8 |
| 667 | sense-5-4-8 |
| 668 | sense-5-5-8 |
| 669 | sense-5-6-8 |
| 670 | sense-5-7-8 |
| 671 | sense-5-8-8 |
| 672 | sense-5-9-8 |
| 673 | sense-5-10-8 |
| 674 | sense-5-0-9 |
| 675 | sense-5-1-9 |
| 676 | sense-5-2-9 |
| 677 | sense-5-3-9 |
| 678 | sense-5-4-9 |
| 679 | sense-5-5-9 |
| 680 | sense-5-6-9 |
| 681 | sense-5-7-9 |
| 682 | sense-5-8-9 |
| 683 | sense-5-9-9 |
| 684 | sense-5-10-9 |
| 685 | sense-5-0-10 |
| 686 | sense-5-1-10 |
| 687 | sense-5-2-10 |
| 688 | sense-5-3-10 |
| 689 | sense-5-4-10 |
| 690 | sense-5-5-10 |
| 691 | sense-5-6-10 |
| 692 | sense-5-7-10 |
| 693 | sense-5-8-10 |
| 694 | sense-5-9-10 |
| 695 | sense-5-10-10 |
| 696 | anti-sense-1-0-0 |
| 697 | anti-sense-1-0-1 |
| 698 | anti-sense-1-0-2 |
| 699 | anti-sense-1-0-3 |
| 700 | anti-sense-1-0-4 |
| 701 | anti-sense-1-0-5 |
| 702 | anti-sense-1-0-6 |
| 703 | anti-sense-1-0-7 |
| 704 | anti-sense-1-0-8 |
| 705 | anti-sense-1-0-9 |
| 706 | anti-sense-1-0-10 |
| 707 | anti-sense-2-0-0 |
| 708 | anti-sense-2-1-0 |
| 709 | anti-sense-2-2-0 |
| 710 | anti-sense-2-3-0 |
| 711 | anti-sense-2-4-0 |
| 712 | anti-sense-2-5-0 |
| 713 | anti-sense-2-6-0 |
| 714 | anti-sense-2-7-0 |
| 715 | anti-sense-2-8-0 |
| 716 | anti-sense-2-9-0 |
| 717 | anti-sense-2-10-0 |
| 718 | anti-sense-2-0-1 |
| 719 | anti-sense-2-1-1 |
| 720 | anti-sense-2-2-1 |
| 721 | anti-sense-2-3-1 |
| 722 | anti-sense-2-4-1 |
| 723 | anti-sense-2-5-1 |
| 724 | anti-sense-2-6-1 |
| 725 | anti-sense-2-7-1 |
| 726 | anti-sense-2-8-1 |
| 727 | anti-sense-2-9-1 |
| 728 | anti-sense-2-10-1 |
| 729 | anti-sense-2-0-2 |
| 730 | anti-sense-2-1-2 |
| 731 | anti-sense-2-2-2 |
| 732 | anti-sense-2-3-2 |
| 733 | anti-sense-2-4-2 |
| 734 | anti-sense-2-5-2 |
| 735 | anti-sense-2-6-2 |
| 736 | anti-sense-2-7-2 |
| 737 | anti-sense-2-8-2 |
| 738 | anti-sense-2-9-2 |
| 739 | anti-sense-2-10-2 |
| 740 | anti-sense-2-0-3 |
| 741 | anti-sense-2-1-3 |
| 742 | anti-sense-2-2-3 |
| 743 | anti-sense-2-3-3 |
| 744 | anti-sense-2-4-3 |
| 745 | anti-sense-2-5-3 |
| 746 | anti-sense-2-6-3 |
| 747 | anti-sense-2-7-3 |
| 748 | anti-sense-2-8-3 |
| 749 | anti-sense-2-9-3 |
| 750 | anti-sense-2-10-3 |
| 751 | anti-sense-2-0-4 |
| 752 | anti-sense-2-1-4 |
| 753 | anti-sense-2-2-4 |
| 754 | anti-sense-2-3-4 |
| 755 | anti-sense-2-4-4 |
| 756 | anti-sense-2-5-4 |
| 757 | anti-sense-2-6-4 |
| 758 | anti-sense-2-7-4 |
| 759 | anti-sense-2-8-4 |
| 760 | anti-sense-2-9-4 |
| 761 | anti-sense-2-10-4 |
| 762 | anti-sense-2-0-5 |
| 763 | anti-sense-2-1-5 |
| 764 | anti-sense-2-2-5 |
| 765 | anti-sense-2-3-5 |
| 766 | anti-sense-2-4-5 |
| 767 | anti-sense-2-5-5 |
| 768 | anti-sense-2-6-5 |
| 769 | anti-sense-2-7-5 |
| 770 | anti-sense-2-8-5 |
| 771 | anti-sense-2-9-5 |
| 772 | anti-sense-2-10-5 |
| 773 | anti-sense-2-0-6 |
| 774 | anti-sense-2-1-6 |
| 775 | anti-sense-2-2-6 |
| 776 | anti-sense-2-3-6 |
| 777 | anti-sense-2-4-6 |
| 778 | anti-sense-2-5-6 |
| 779 | anti-sense-2-6-6 |
| 780 | anti-sense-2-7-6 |
| 781 | anti-sense-2-8-6 |
| 782 | anti-sense-2-9-6 |
| 783 | anti-sense-2-10-6 |
| 784 | anti-sense-2-0-7 |
| 785 | anti-sense-2-1-7 |
| 786 | anti-sense-2-2-7 |
| 787 | anti-sense-2-3-7 |
| 788 | anti-sense-2-4-7 |
| 789 | anti-sense-2-5-7 |
| 790 | anti-sense-2-6-7 |
| 791 | anti-sense-2-7-7 |
| 792 | anti-sense-2-8-7 |
| 793 | anti-sense-2-9-7 |
| 794 | anti-sense-2-10-7 |
| 795 | anti-sense-2-0-8 |
| 796 | anti-sense-2-1-8 |
| 797 | anti-sense-2-2-8 |
| 798 | anti-sense-2-3-8 |
| 799 | anti-sense-2-4-8 |
| 800 | anti-sense-2-5-8 |
| 801 | anti-sense-2-6-8 |
| 802 | anti-sense-2-7-8 |
| 803 | anti-sense-2-8-8 |
| 804 | anti-sense-2-9-8 |
| 805 | anti-sense-2-10-8 |
| 806 | anti-sense-2-0-9 |
| 807 | anti-sense-2-1-9 |
| 808 | anti-sense-2-2-9 |
| 809 | anti-sense-2-3-9 |
| 810 | anti-sense-2-4-9 |
| 811 | anti-sense-2-5-9 |
| 812 | anti-sense-2-6-9 |
| 813 | anti-sense-2-7-9 |
| 814 | anti-sense-2-8-9 |
| 815 | anti-sense-2-9-9 |
| 816 | anti-sense-2-10-9 |
| 817 | anti-sense-2-0-10 |
| 818 | anti-sense-2-1-10 |
| 819 | anti-sense-2-2-10 |
| 820 | anti-sense-2-3-10 |
| 821 | anti-sense-2-4-10 |
| 822 | anti-sense-2-5-10 |
| 823 | anti-sense-2-6-10 |
| 824 | anti-sense-2-7-10 |
| 825 | anti-sense-2-8-10 |
| 826 | anti-sense-2-9-10 |
| 827 | anti-sense-2-10-10 |
| 828 | anti-sense-3-0-0 |
| 829 | anti-sense-3-1-0 |
| 830 | anti-sense-3-2-0 |
| 831 | anti-sense-3-3-0 |
| 832 | anti-sense-3-4-0 |
| 833 | anti-sense-3-5-0 |
| 834 | anti-sense-3-6-0 |
| 835 | anti-sense-3-7-0 |
| 836 | anti-sense-3-8-0 |
| 837 | anti-sense-3-9-0 |
| 838 | anti-sense-3-10-0 |
| 839 | anti-sense-3-0-1 |
| 840 | anti-sense-3-1-1 |
| 841 | anti-sense-3-2-1 |
| 842 | anti-sense-3-3-1 |
| 843 | anti-sense-3-4-1 |
| 844 | anti-sense-3-5-1 |
| 845 | anti-sense-3-6-1 |
| 846 | anti-sense-3-7-1 |
| 847 | anti-sense-3-8-1 |
| 848 | anti-sense-3-9-1 |
| 849 | anti-sense-3-10-1 |
| 850 | anti-sense-3-0-2 |
| 851 | anti-sense-3-1-2 |
| 852 | anti-sense-3-2-2 |
| 853 | anti-sense-3-3-2 |
| 854 | anti-sense-3-4-2 |
| 855 | anti-sense-3-5-2 |
| 856 | anti-sense-3-6-2 |
| 857 | anti-sense-3-7-2 |
| 858 | anti-sense-3-8-2 |
| 859 | anti-sense-3-9-2 |
| 860 | anti-sense-3-10-2 |
| 861 | anti-sense-3-0-3 |
| 862 | anti-sense-3-1-3 |
| 863 | anti-sense-3-2-3 |
| 864 | anti-sense-3-3-3 |
| 865 | anti-sense-3-4-3 |
| 866 | anti-sense-3-5-3 |
| 867 | anti-sense-3-6-3 |
| 868 | anti-sense-3-7-3 |
| 869 | anti-sense-3-8-3 |
| 870 | anti-sense-3-9-3 |
| 871 | anti-sense-3-10-3 |
| 872 | anti-sense-3-0-4 |
| 873 | anti-sense-3-1-4 |
| 874 | anti-sense-3-2-4 |
| 875 | anti-sense-3-3-4 |
| 876 | anti-sense-3-4-4 |
| 877 | anti-sense-3-5-4 |
| 878 | anti-sense-3-6-4 |
| 879 | anti-sense-3-7-4 |
| 880 | anti-sense-3-8-4 |
| 881 | anti-sense-3-9-4 |
| 882 | anti-sense-3-10-4 |
| 883 | anti-sense-3-0-5 |
| 884 | anti-sense-3-1-5 |
| 885 | anti-sense-3-2-5 |
| 886 | anti-sense-3-3-5 |
| 887 | anti-sense-3-4-5 |
| 888 | anti-sense-3-5-5 |
| 889 | anti-sense-3-6-5 |
| 890 | anti-sense-3-7-5 |
| 891 | anti-sense-3-8-5 |
| 892 | anti-sense-3-9-5 |
| 893 | anti-sense-3-10-5 |
| 894 | anti-sense-3-0-6 |
| 895 | anti-sense-3-1-6 |
| 896 | anti-sense-3-2-6 |
| 897 | anti-sense-3-3-6 |
| 898 | anti-sense-3-4-6 |
| 899 | anti-sense-3-5-6 |
| 900 | anti-sense-3-6-6 |
| 901 | anti-sense-3-7-6 |
| 902 | anti-sense-3-8-6 |
| 903 | anti-sense-3-9-6 |
| 904 | anti-sense-3-10-6 |
| 905 | anti-sense-3-0-7 |
| 906 | anti-sense-3-1-7 |
| 907 | anti-sense-3-2-7 |
| 908 | anti-sense-3-3-7 |
| 909 | anti-sense-3-4-7 |
| 910 | anti-sense-3-5-7 |
| 911 | anti-sense-3-6-7 |
| 912 | anti-sense-3-7-7 |
| 913 | anti-sense-3-8-7 |
| 914 | anti-sense-3-9-7 |
| 915 | anti-sense-3-10-7 |
| 916 | anti-sense-3-0-8 |
| 917 | anti-sense-3-1-8 |
| 918 | anti-sense-3-2-8 |
| 919 | anti-sense-3-3-8 |
| 920 | anti-sense-3-4-8 |
| 921 | anti-sense-3-5-8 |
| 922 | anti-sense-3-6-8 |
| 923 | anti-sense-3-7-8 |
| 924 | anti-sense-3-8-8 |
| 925 | anti-sense-3-9-8 |
| 926 | anti-sense-3-10-8 |
| 927 | anti-sense-3-0-9 |
| 928 | anti-sense-3-1-9 |
| 929 | anti-sense-3-2-9 |
| 930 | anti-sense-3-3-9 |
| 931 | anti-sense-3-4-9 |
| 932 | anti-sense-3-5-9 |
| 933 | anti-sense-3-6-9 |
| 934 | anti-sense-3-7-9 |
| 935 | anti-sense-3-8-9 |
| 936 | anti-sense-3-9-9 |
| 937 | anti-sense-3-10-9 |
| 938 | anti-sense-3-0-10 |
| 939 | anti-sense-3-1-10 |
| 940 | anti-sense-3-2-10 |
| 941 | anti-sense-3-3-10 |
| 942 | anti-sense-3-4-10 |
| 943 | anti-sense-3-5-10 |
| 944 | anti-sense-3-6-10 |
| 945 | anti-sense-3-7-10 |
| 946 | anti-sense-3-8-10 |
| 947 | anti-sense-3-9-10 |
| 948 | anti-sense-3-10-10 |
| 949 | anti-sense-4-0-0 |
| 950 | anti-sense-4-1-0 |
| 951 | anti-sense-4-2-0 |
| 952 | anti-sense-4-3-0 |
| 953 | anti-sense-4-4-0 |
| 954 | anti-sense-4-5-0 |
| 955 | anti-sense-4-6-0 |
| 956 | anti-sense-4-7-0 |
| 957 | anti-sense-4-8-0 |
| 958 | anti-sense-4-9-0 |
| 959 | anti-sense-4-10-0 |
| 960 | anti-sense-4-0-1 |
| 961 | anti-sense-4-1-1 |
| 962 | anti-sense-4-2-1 |
| 963 | anti-sense-4-3-1 |
| 964 | anti-sense-4-4-1 |
| 965 | anti-sense-4-5-1 |
| 966 | anti-sense-4-6-1 |
| 967 | anti-sense-4-7-1 |
| 968 | anti-sense-4-8-1 |
| 969 | anti-sense-4-9-1 |
| 970 | anti-sense-4-10-1 |
| 971 | anti-sense-4-0-2 |
| 972 | anti-sense-4-1-2 |
| 973 | anti-sense-4-2-2 |
| 974 | anti-sense-4-3-2 |
| 975 | anti-sense-4-4-2 |
| 976 | anti-sense-4-5-2 |
| 977 | anti-sense-4-6-2 |
| 978 | anti-sense-4-7-2 |
| 979 | anti-sense-4-8-2 |
| 980 | anti-sense-4-9-2 |
| 981 | anti-sense-4-10-2 |
| 982 | anti-sense-4-0-3 |
| 983 | anti-sense-4-1-3 |
| 984 | anti-sense-4-2-3 |
| 985 | anti-sense-4-3-3 |
| 986 | anti-sense-4-4-3 |
| 987 | anti-sense-4-5-3 |
| 988 | anti-sense-4-6-3 |
| 989 | anti-sense-4-7-3 |
| 990 | anti-sense-4-8-3 |
| 991 | anti-sense-4-9-3 |
| 992 | anti-sense-4-10-3 |
| 993 | anti-sense-4-0-4 |
| 994 | anti-sense-4-1-4 |
| 995 | anti-sense-4-2-4 |
| 996 | anti-sense-4-3-4 |
| 997 | anti-sense-4-4-4 |
| 998 | anti-sense-4-5-4 |
| 999 | anti-sense-4-6-4 |
| 1000 | anti-sense-4-7-4 |
| 1001 | anti-sense-4-8-4 |
| 1002 | anti-sense-4-9-4 |
| 1003 | anti-sense-4-10-4 |
| 1004 | anti-sense-4-0-5 |
| 1005 | anti-sense-4-1-5 |
| 1006 | anti-sense-4-2-5 |
| 1007 | anti-sense-4-3-5 |
| 1008 | anti-sense-4-4-5 |
| 1009 | anti-sense-4-5-5 |
| 1010 | anti-sense-4-6-5 |
| 1011 | anti-sense-4-7-5 |
| 1012 | anti-sense-4-8-5 |
| 1013 | anti-sense-4-9-5 |
| 1014 | anti-sense-4-10-5 |
| 1015 | anti-sense-4-0-6 |
| 1016 | anti-sense-4-1-6 |
| 1017 | anti-sense-4-2-6 |
| 1018 | anti-sense-4-3-6 |
| 1019 | anti-sense-4-4-6 |
| 1020 | anti-sense-4-5-6 |
| 1021 | anti-sense-4-6-6 |
| 1022 | anti-sense-4-7-6 |
| 1023 | anti-sense-4-8-6 |
| 1024 | anti-sense-4-9-6 |
| 1025 | anti-sense-4-10-6 |
| 1026 | anti-sense-4-0-7 |
| 1027 | anti-sense-4-1-7 |
| 1028 | anti-sense-4-2-7 |
| 1029 | anti-sense-4-3-7 |
| 1030 | anti-sense-4-4-7 |
| 1031 | anti-sense-4-5-7 |
| 1032 | anti-sense-4-6-7 |
| 1033 | anti-sense-4-7-7 |
| 1034 | anti-sense-4-8-7 |
| 1035 | anti-sense-4-9-7 |
| 1036 | anti-sense-4-10-7 |
| 1037 | anti-sense-4-0-8 |
| 1038 | anti-sense-4-1-8 |
| 1039 | anti-sense-4-2-8 |
| 1040 | anti-sense-4-3-8 |
| 1041 | anti-sense-4-4-8 |
| 1042 | anti-sense-4-5-8 |
| 1043 | anti-sense-4-6-8 |
| 1044 | anti-sense-4-7-8 |
| 1045 | anti-sense-4-8-8 |
| 1046 | anti-sense-4-9-8 |
| 1047 | anti-sense-4-10-8 |
| 1048 | anti-sense-4-0-9 |
| 1049 | anti-sense-4-1-9 |
| 1050 | anti-sense-4-2-9 |
| 1051 | anti-sense-4-3-9 |
| 1052 | anti-sense-4-4-9 |
| 1053 | anti-sense-4-5-9 |
| 1054 | anti-sense-4-6-9 |
| 1055 | anti-sense-4-7-9 |
| 1056 | anti-sense-4-8-9 |
| 1057 | anti-sense-4-9-9 |
| 1058 | anti-sense-4-10-9 |
| 1059 | anti-sense-4-0-10 |
| 1060 | anti-sense-4-1-10 |
| 1061 | anti-sense-4-2-10 |
| 1062 | anti-sense-4-3-10 |
| 1063 | anti-sense-4-4-10 |
| 1064 | anti-sense-4-5-10 |
| 1065 | anti-sense-4-6-10 |
| 1066 | anti-sense-4-7-10 |
| 1067 | anti-sense-4-8-10 |
| 1068 | anti-sense-4-9-10 |
| 1069 | anti-sense-4-10-10 |
| 1070 | anti-sense-5-0-0 |
| 1071 | anti-sense-5-1-0 |
| 1072 | anti-sense-5-2-0 |
| 1073 | anti-sense-5-3-0 |
| 1074 | anti-sense-5-4-0 |
| 1075 | anti-sense-5-5-0 |
| 1076 | anti-sense-5-6-0 |
| 1077 | anti-sense-5-7-0 |
| 1078 | anti-sense-5-8-0 |
| 1079 | anti-sense-5-9-0 |
| 1080 | anti-sense-5-10-0 |
| 1081 | anti-sense-5-0-1 |
| 1082 | anti-sense-5-1-1 |
| 1083 | anti-sense-5-2-1 |
| 1084 | anti-sense-5-3-1 |
| 1085 | anti-sense-5-4-1 |
| 1086 | anti-sense-5-5-1 |
| 1087 | anti-sense-5-6-1 |
| 1088 | anti-sense-5-7-1 |
| 1089 | anti-sense-5-8-1 |
| 1090 | anti-sense-5-9-1 |
| 1091 | anti-sense-5-10-1 |
| 1092 | anti-sense-5-0-2 |
| 1093 | anti-sense-5-1-2 |
| 1094 | anti-sense-5-2-2 |
| 1095 | anti-sense-5-3-2 |
| 1096 | anti-sense-5-4-2 |
| 1097 | anti-sense-5-5-2 |
| 1098 | anti-sense-5-6-2 |
| 1099 | anti-sense-5-7-2 |
| 1100 | anti-sense-5-8-2 |
| 1101 | anti-sense-5-9-2 |
| 1102 | anti-sense-5-10-2 |
| 1103 | anti-sense-5-0-3 |
| 1104 | anti-sense-5-1-3 |
| 1105 | anti-sense-5-2-3 |
| 1106 | anti-sense-5-3-3 |
| 1107 | anti-sense-5-4-3 |
| 1108 | anti-sense-5-5-3 |
| 1109 | anti-sense-5-6-3 |
| 1110 | anti-sense-5-7-3 |
| 1111 | anti-sense-5-8-3 |
| 1112 | anti-sense-5-9-3 |
| 1113 | anti-sense-5-10-3 |
| 1114 | anti-sense-5-0-4 |
| 1115 | anti-sense-5-1-4 |
| 1116 | anti-sense-5-2-4 |
| 1117 | anti-sense-5-3-4 |
| 1118 | anti-sense-5-4-4 |
| 1119 | anti-sense-5-5-4 |
| 1120 | anti-sense-5-6-4 |
| 1121 | anti-sense-5-7-4 |
| 1122 | anti-sense-5-8-4 |
| 1123 | anti-sense-5-9-4 |
| 1124 | anti-sense-5-10-4 |
| 1125 | anti-sense-5-0-5 |
| 1126 | anti-sense-5-1-5 |
| 1127 | anti-sense-5-2-5 |
| 1128 | anti-sense-5-3-5 |
| 1129 | anti-sense-5-4-5 |
| 1130 | anti-sense-5-5-5 |
| 1131 | anti-sense-5-6-5 |
| 1132 | anti-sense-5-7-5 |
| 1133 | anti-sense-5-8-5 |
| 1134 | anti-sense-5-9-5 |
| 1135 | anti-sense-5-10-5 |
| 1136 | anti-sense-5-0-6 |
| 1137 | anti-sense-5-1-6 |
| 1138 | anti-sense-5-2-6 |
| 1139 | anti-sense-5-3-6 |
| 1140 | anti-sense-5-4-6 |
| 1141 | anti-sense-5-5-6 |
| 1142 | anti-sense-5-6-6 |
| 1143 | anti-sense-5-7-6 |
| 1144 | anti-sense-5-8-6 |
| 1145 | anti-sense-5-9-6 |
| 1146 | anti-sense-5-10-6 |
| 1147 | anti-sense-5-0-7 |
| 1148 | anti-sense-5-1-7 |
| 1149 | anti-sense-5-2-7 |
| 1150 | anti-sense-5-3-7 |
| 1151 | anti-sense-5-4-7 |
| 1152 | anti-sense-5-5-7 |
| 1153 | anti-sense-5-6-7 |
| 1154 | anti-sense-5-7-7 |
| 1155 | anti-sense-5-8-7 |
| 1156 | anti-sense-5-9-7 |
| 1157 | anti-sense-5-10-7 |
| 1158 | anti-sense-5-0-8 |
| 1159 | anti-sense-5-1-8 |
| 1160 | anti-sense-5-2-8 |
| 1161 | anti-sense-5-3-8 |
| 1162 | anti-sense-5-4-8 |
| 1163 | anti-sense-5-5-8 |
| 1164 | anti-sense-5-6-8 |
| 1165 | anti-sense-5-7-8 |
| 1166 | anti-sense-5-8-8 |
| 1167 | anti-sense-5-9-8 |
| 1168 | anti-sense-5-10-8 |
| 1169 | anti-sense-5-0-9 |
| 1170 | anti-sense-5-1-9 |
| 1171 | anti-sense-5-2-9 |
| 1172 | anti-sense-5-3-9 |
| 1173 | anti-sense-5-4-9 |
| 1174 | anti-sense-5-5-9 |
| 1175 | anti-sense-5-6-9 |
| 1176 | anti-sense-5-7-9 |
| 1177 | anti-sense-5-8-9 |
| 1178 | anti-sense-5-9-9 |
| 1179 | anti-sense-5-10-9 |
| 1180 | anti-sense-5-0-10 |
| 1181 | anti-sense-5-1-10 |
| 1182 | anti-sense-5-2-10 |
| 1183 | anti-sense-5-3-10 |
| 1184 | anti-sense-5-4-10 |
| 1185 | anti-sense-5-5-10 |
| 1186 | anti-sense-5-6-10 |
| 1187 | anti-sense-5-7-10 |
| 1188 | anti-sense-5-8-10 |
| 1189 | anti-sense-5-9-10 |
| 1190 | anti-sense-5-10-10 |

The invention is also characterized by the following figures, figure legends and the following non-limiting examples.

### BRIEF DESCRIPTION OF THE FIGURES

FIG. 1. MTF activity towards various reporter constructs in human cell lines.

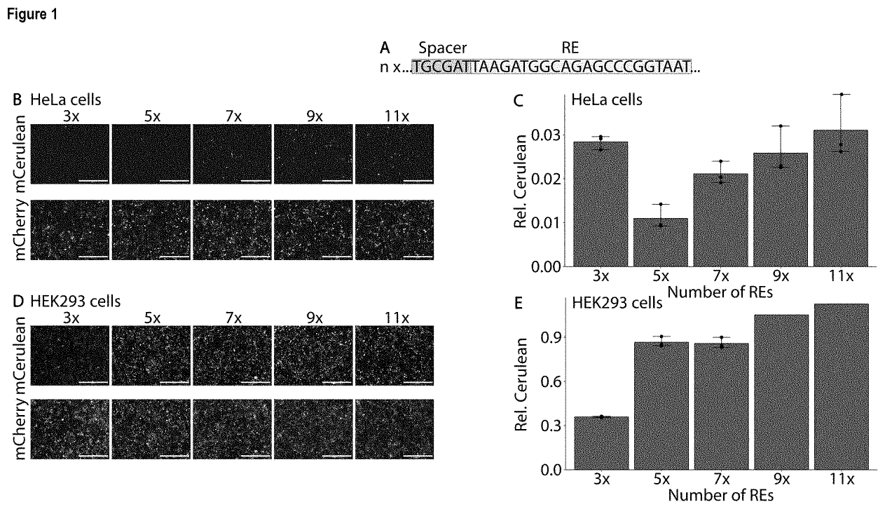

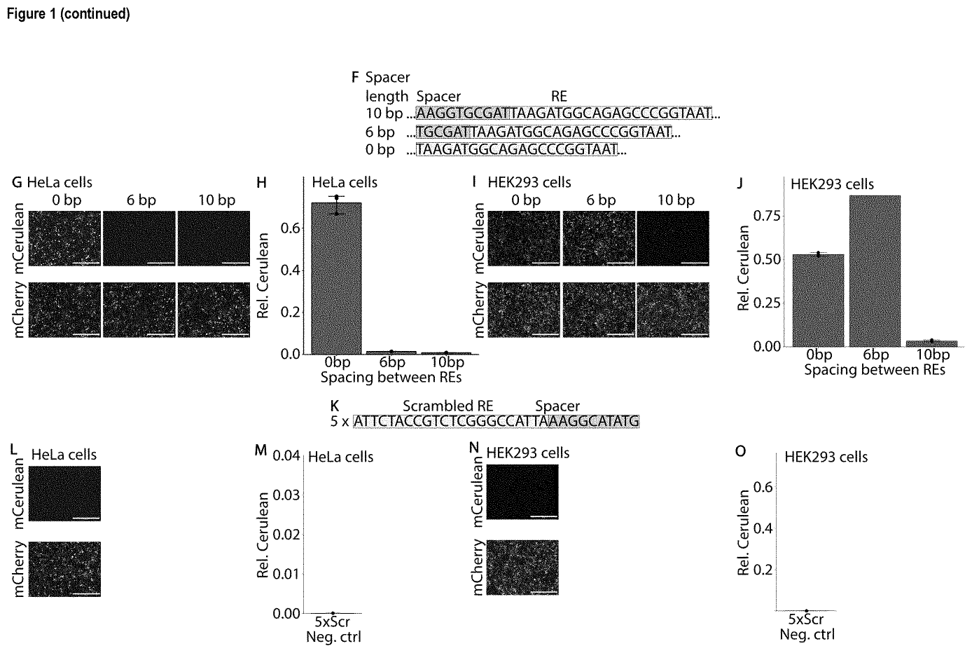

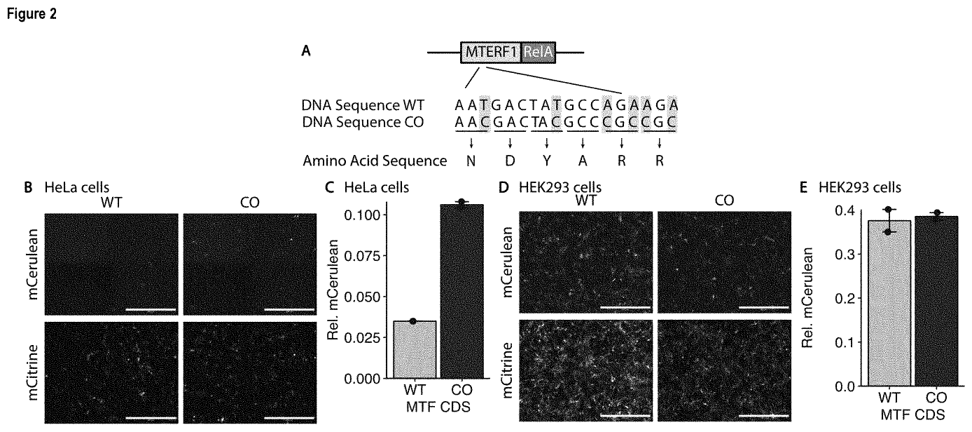

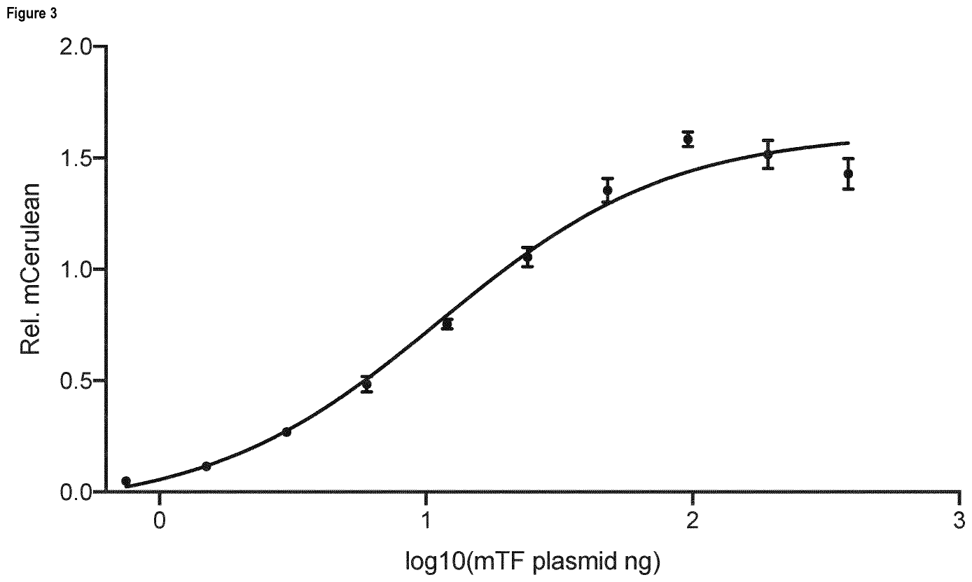

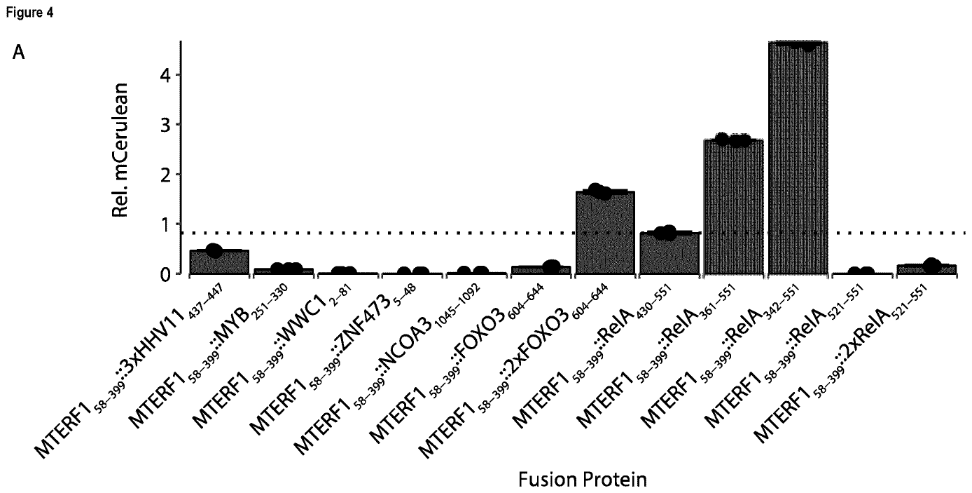

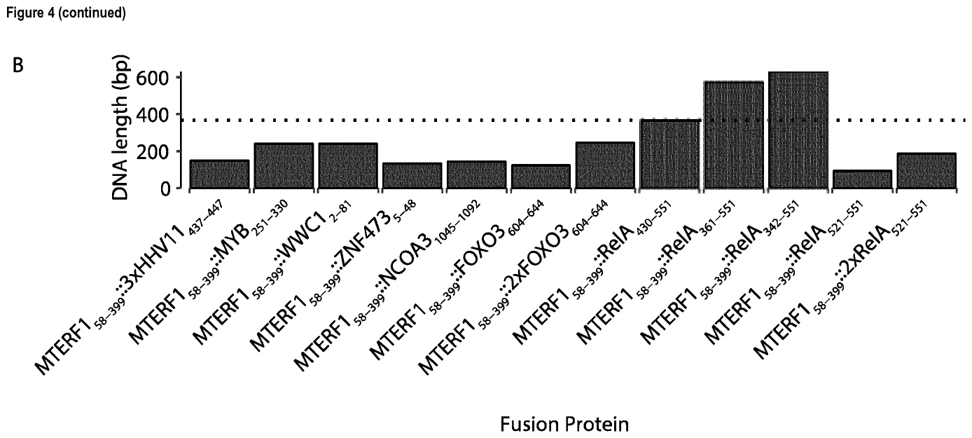

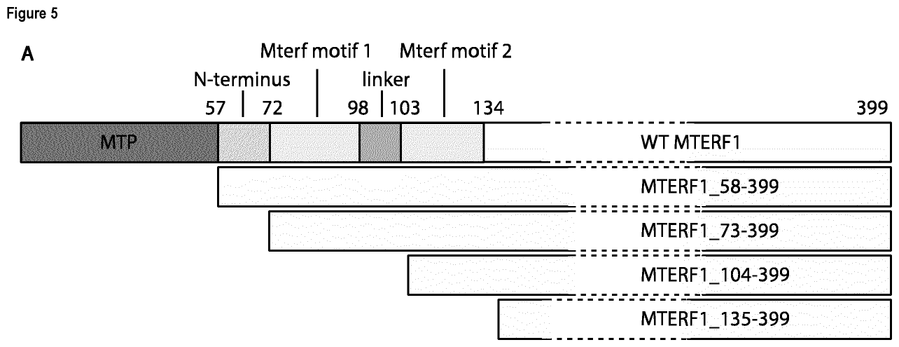

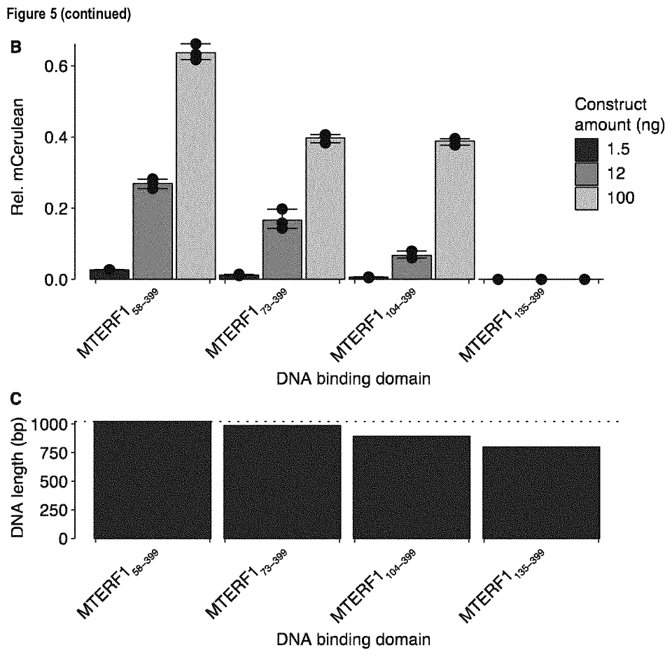

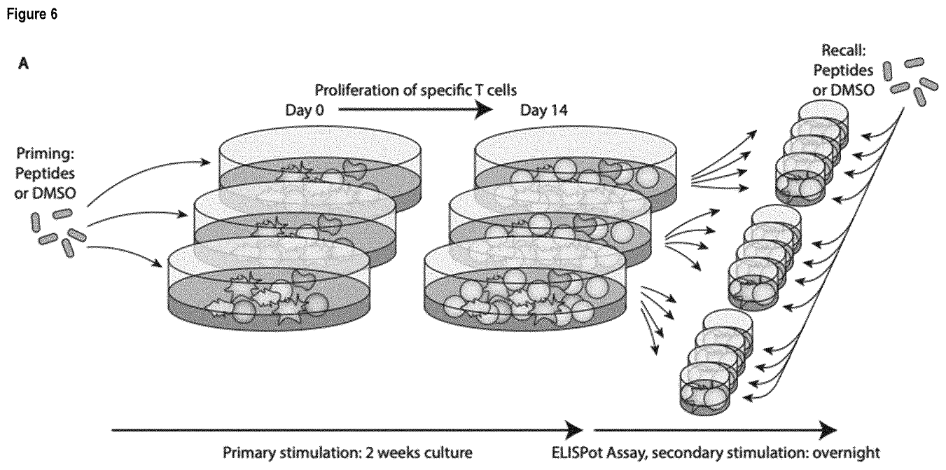

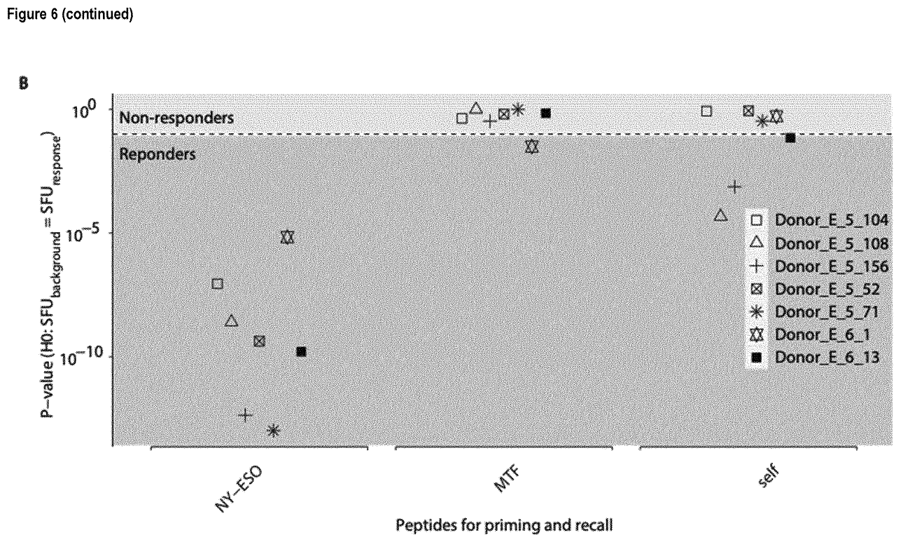

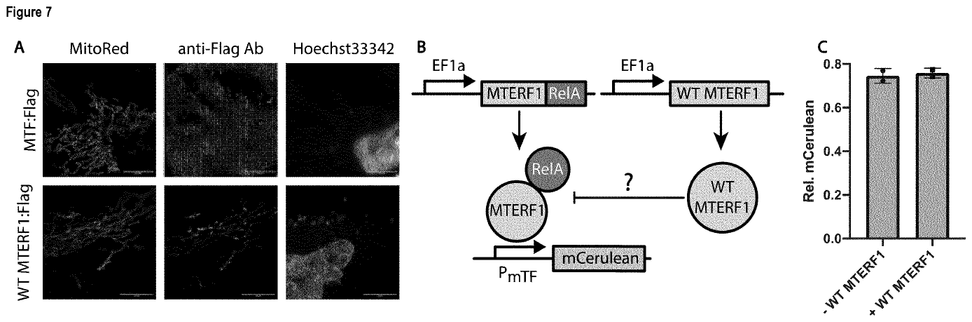

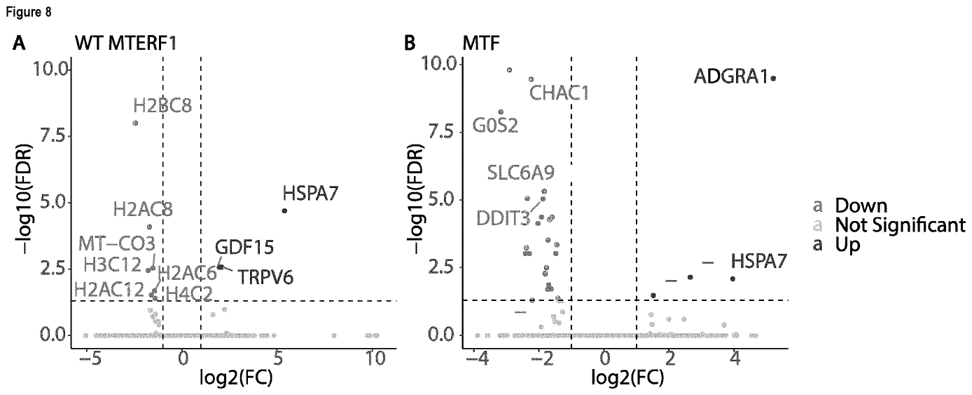

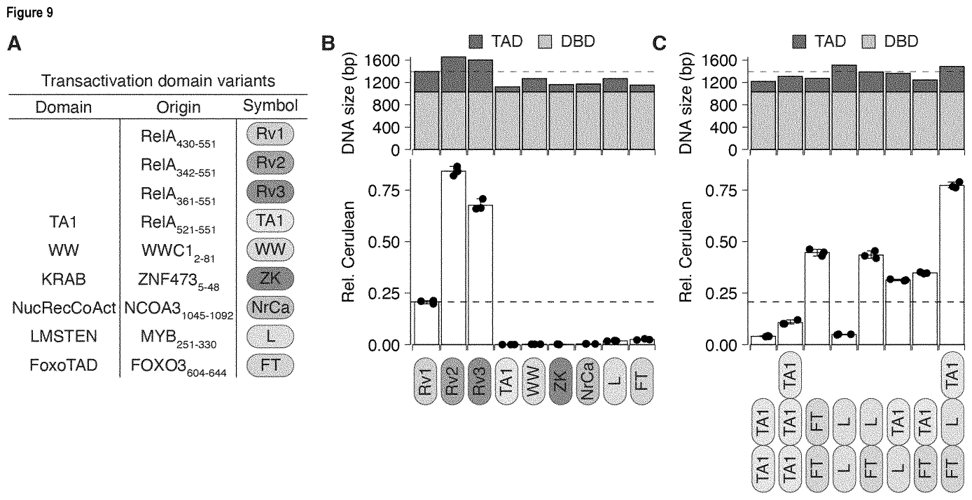

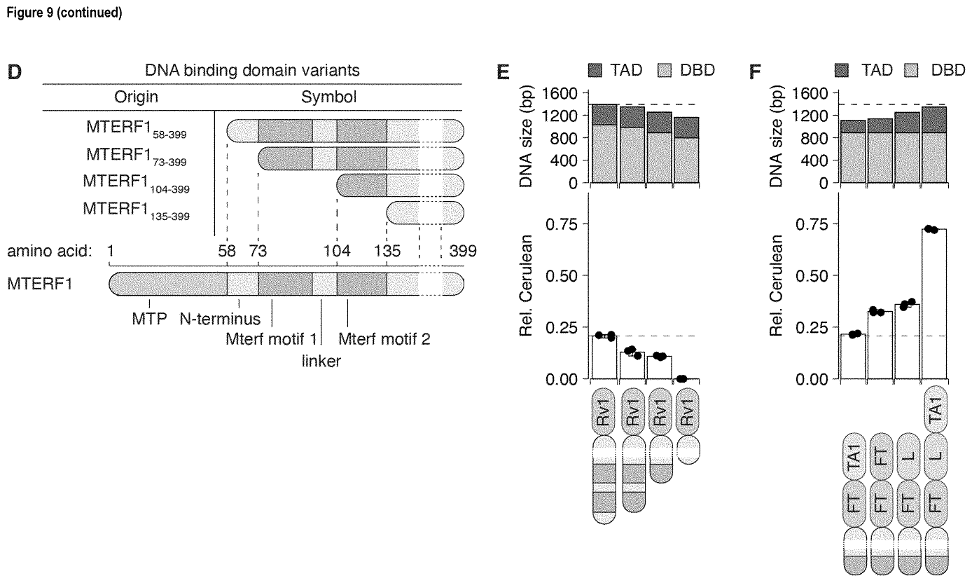

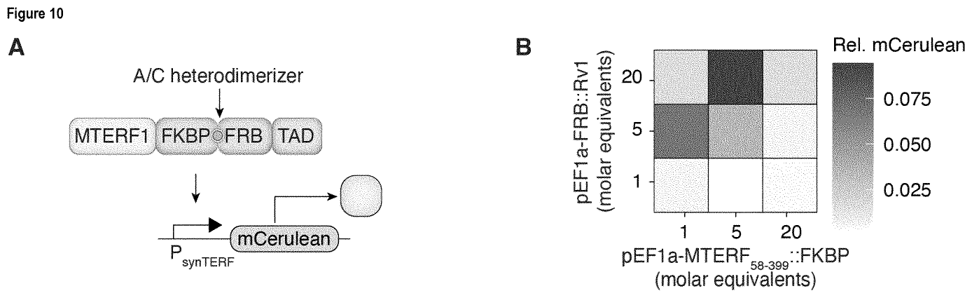

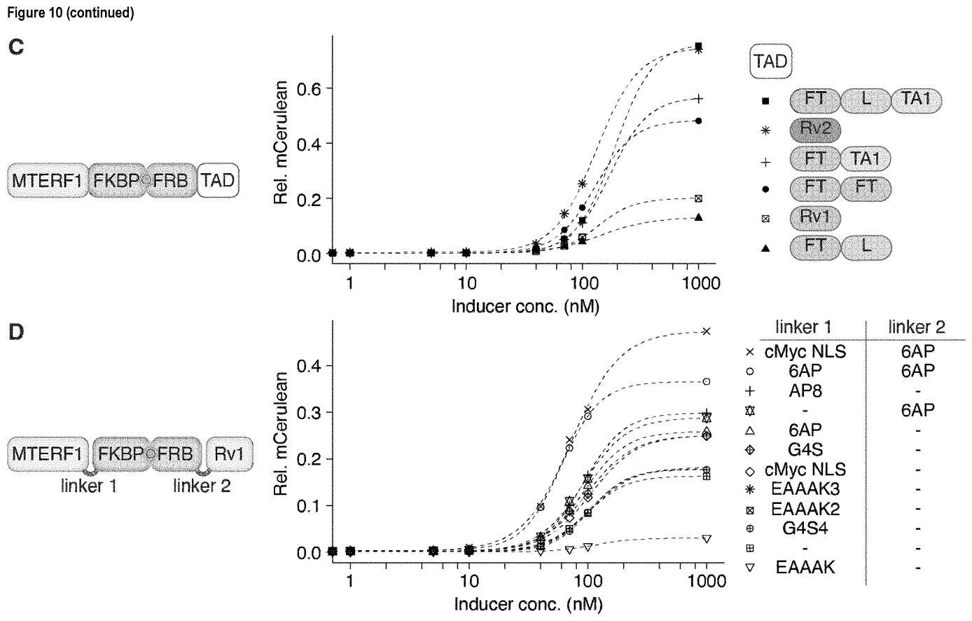

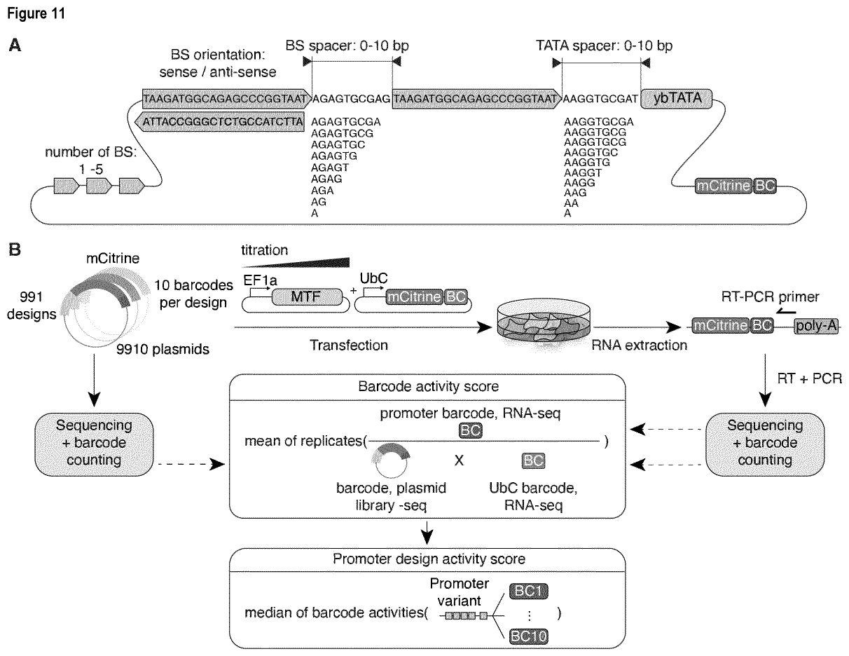

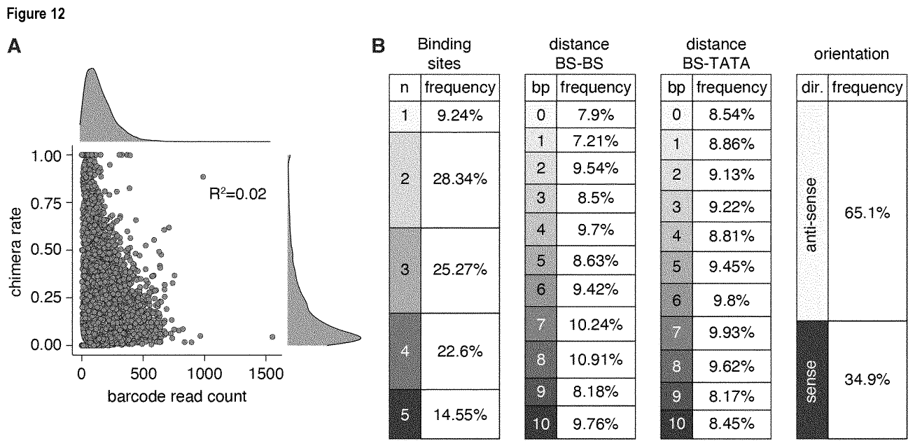

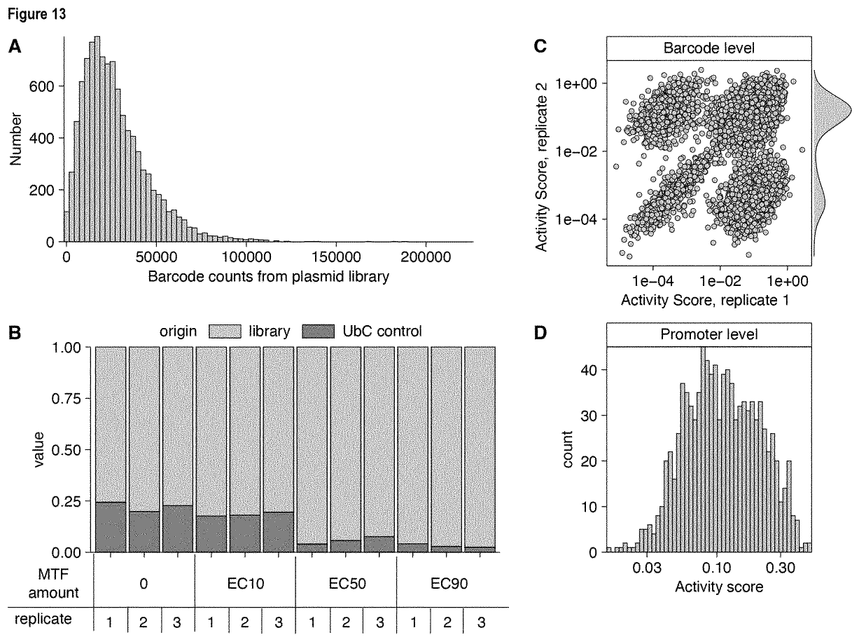

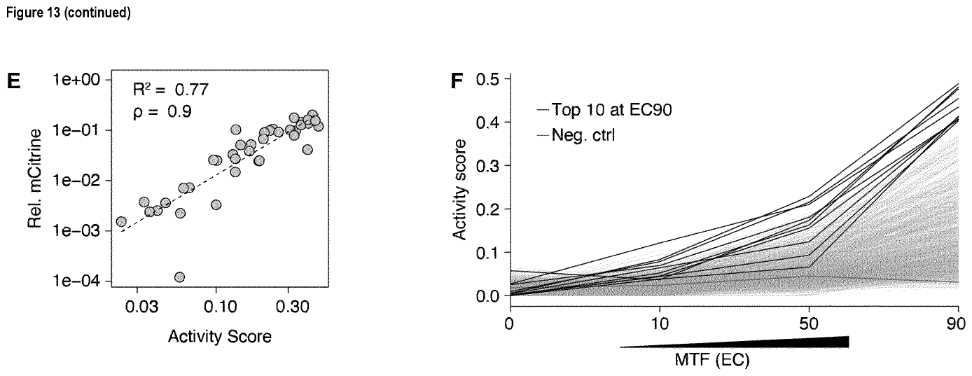

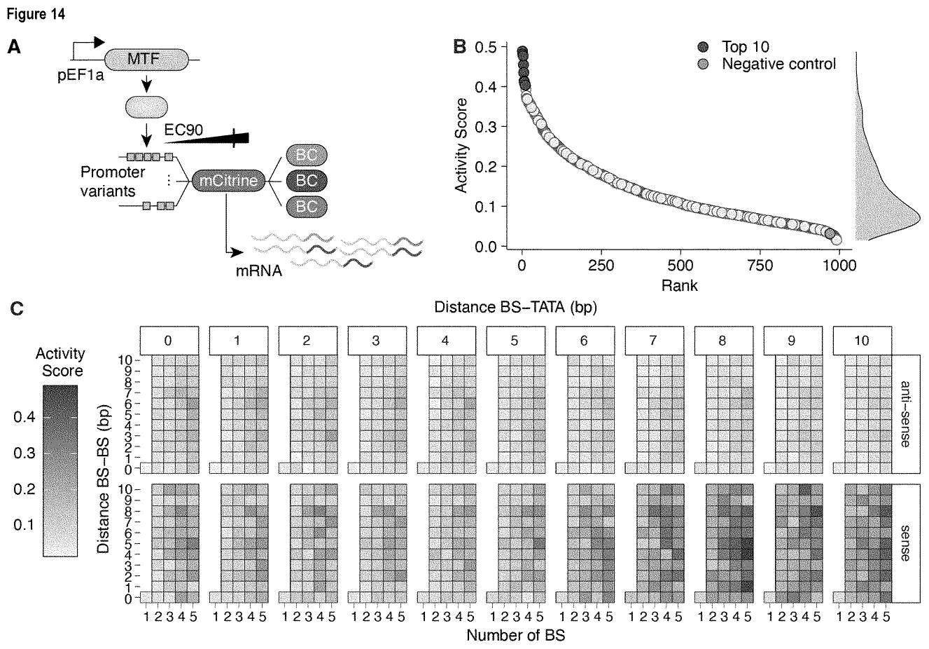

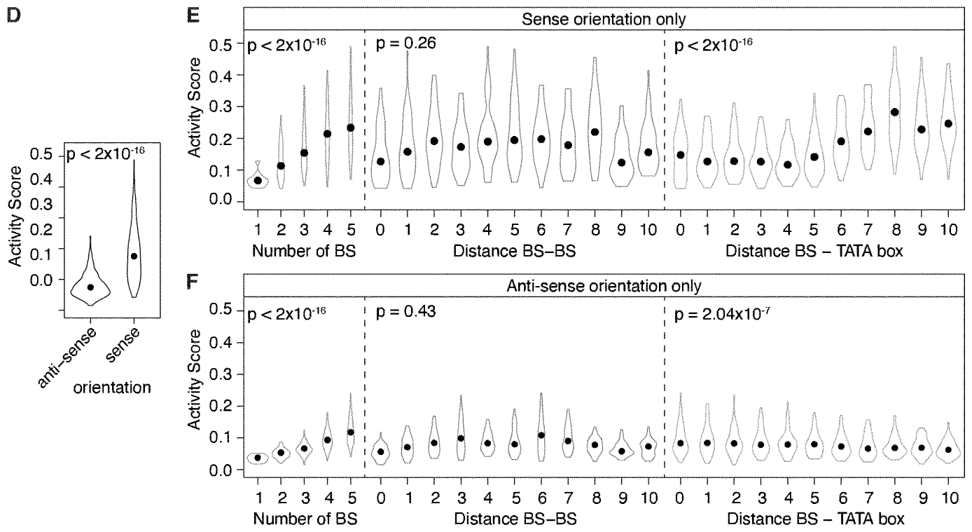

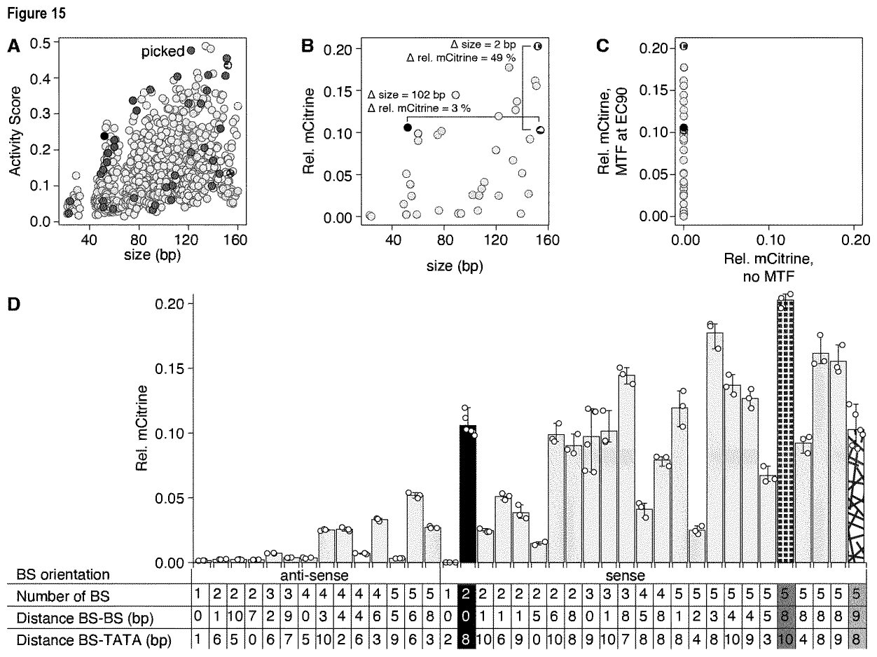

(A) DNA sequence of a response element (RE) repeat and spacer which is repeated n times. In FIG. 1, the term “response element” (RE) corresponds to the term “binding site” as used herein, in particular, an MTERF1 binding site as defined by SEQ ID NO: 42. (B) Dependency of Gene of Interest (Gol) expression on the number of REs in the reporter construct in HeLa human cell line. Microscopy images of Hela cells show the expression of mCerulean Gol (top) and the expression of the transfection reporter control mCherry (bottom). Fluorescent channel names are indicated on the left. The number of RE repeats in each promoter construct is shown above each pair of images. (C) Quantitative analysis of mCerulean levels normalized to mCherry levels in Hela cells transfected with reporter constructs differing in the number of RE-spacer repeats illustrated in panel A. The analysis is based on flow cytometry data. The number of REs/MTERF1 binding sites (SEQ ID NO: 42) in each reporter construct is shown on the X axis. (D) Dependency of Gol expression on the number of REs in the reporter construct in HEK293 human cell line. Microscopy images of HEK293 cells show the expression of mCerulean Gol (top) and the expression of the transfection reporter control mCherry (bottom). Fluorescent channel names are indicated on the left. The number of RE repeats in each promoter construct is shown above each pair of images. (E) Quantitative analysis of mCerulean levels normalized to mCherry levels in the HEK293 cells shown in panel D. The analysis is based on flow cytometry data. The number of REs/MTERF1 binding sites in each reporter construct is shown on the X axis. (F) DNA sequence of a 5×RE repeat spaced by 10, 6, or no base pairs (bp). (G) The effect of spacing length between REs in the reporter constructs on Gol expression in Hela cells. The schematics of the promoter region and the variable spacer location is shown on top. Microscopy images of Hela cells show the expression of mCerulean Gol (top) and the expression of the transfection reporter control mCherry (bottom). Fluorescent channel names are indicated on the left. The spacer length between REs in each promoter construct is shown above each pair of images. (H) Quantitative analysis of mCerulean levels normalized to mCherry levels in Hela cells transfected with the reporter constructs harboring spacers illustrated in panel F. The analysis is based on flow cytometry data. The spacer length between REs in each reporter construct is shown on the X axis. (I) The effect of spacing length between REs in the reporter constructs on Gol expression in HEK293 cells. The schematics of the promoter region and the variable spacer location is shown on top. Microscopy images of HEK293 cells show the expression of mCerulean Gol (top) and the expression of the transfection reporter control mCherry (bottom). Fluorescent channel names are indicated on the left. The spacer length between REs in each promoter construct is shown above each pair of images. (J) Quantitative analysis of mCerulean levels normalized to mCherry levels in Hela cells transfected with the reporter constructs harboring spacers illustrated in panel F. The analysis is based on flow cytometry data. The spacer length between REs in each reporter construct is shown on the X axis. (K) DNA sequence of a repeat of the scrambled (Scr) RE which is repeated 5 times (SEQ ID NO: 51) in the negative control promoter. (L) Microscopy images showing Hela cells transfected with a negative control (Neg. ctrl) reporter design containing 5 scrambled (Scr) REs spaced by 10 bp. (M) Analysis of flow cytometry measurements of the cells illustrated in panel L. (N) Microscopy images showing HEK293 cells transfected with a reporter design containing 5 scrambled REs spaced by 10 bp. (O) Analysis of flow cytometry measurements of the cells illustrated in panel N. Scale bars in microscopy images indicate 600 μm. Panels B, G, L: mCerulean, 100 ms exposure, LUT range 0-26′000; mCherry, 75 ms exposure, LUT range 0-35′000. Panels D, I, N: mCerulean, 75 ms exposure, LUT range 0-65 k; mCherry, 50 ms exposure, LUT range 0-40′000. All micrographs are 100× magnification. RE, response element; bp, base pair; Rel., relative; Scr, scrambled; Neg. ctrl, negative control

FIG. 2. Codon-optimization of the MTF coding sequence (CDS).

(A) Exemplary 6 amino acid subsequence of MTERF1 and its genetic code in the wild-type (WT) and codon-optimized (CO) form. DNA bases that are different are shown against a light gray background. Each triplet is underlined and the encoded amino acid shown below. (B) Micrographs of Hela cells transfected with the 5×RE, 6 bp spacer reporter (SEQ ID NO: 44) construct, MTF encoded by the either wild-type or codon optimized DNA sequence, and a transfection control construct encoding constitutively expressed mCitrine. Images on top show mCerulean and mCitrine expression is showed on the bottom. Fluorescent channels are indicated on the left. (C) Quantitative analysis of mCerulean levels normalized to mCitrine levels of the cells shown in panel B. The analysis is based on flow cytometry data. (D) Microscopy images illustrating mCerulean on top and mCitrine fluorescence levels at the bottom in HEK293 cells. Fluorescent channel names are indicated on the left. (E) Quantitative analysis of mCerulean levels normalized to mCitrine levels of the cells shown in panel D. The analysis is based on flow cytometry data.

Scale bars in B and D indicate 600 μm. Panel B: mCerulean, 2 s exposure, LUT range 5000-30′000; mCitrine, 500 ms exposure, LUT range 0-65′000. Panel D: mCerulean, 500 ms exposure, LUT range; mCitrine, 300 ms exposure, 0-65′000 LUT range. All micrographs are 10× magnification. WT, wild-type; CO, codon optimized; CDS, coding sequence

FIG. 3. Expression level of the reporter protein as a function of varying amounts of transfected MTF construct.

The y-axis shows mCerulean expression relative to transfection control mCherry. Error bars indicate standard deviation from three replicates of HEK293 cells transfected with the MTF construct amount as indicated on the x-axis in nanograms. The line represents the dose-response curve fitted to a Hill equation with n=1. The analysis is based on flow cytometry data.

FIG. 4. Comparing HumTAP variants with different transactivation domains.

(A) Relative reporter expression in HEK293 cells after co-transfection of the indicated HumTAP construct (names indicated on the X axis using HUGO gene nomenclature and amino acid numbers according to UniProt) with the 5×RE, Obp-spacing (SEQ ID NO: 48) promoter driving mCerulean Gol, normalized to the expression of the transfection control mCherry. (B) DNA length in base pair (bp) needed to encode each TAD. Dotted lines in both panels indicate the mCerulean Gol expression level or DNA size of RelA₄₃₀₋₅₅₁ TAD. Rel., relative; bp, base pairs

FIG. 5. Construction and testing of HumTAPs with a reduced genetic footprint.

(A) Schematics representing the different domains of MTERF1. Numbers above the horizontal bars indicate amino acid numbers starting with 1 at the N-terminus. Widths of bars are proportional to their length in amino acids, except dotted bars indicate a stretch of amino acids not represented. (B) Quantitative analysis of mCerulean levels normalized to mCherry levels of cells transfected with the HumTAP constructs built from DNA-binding domains illustrated in panel A. Bars colored with different shades of gray indicate different amounts of the transfected HumTAP construct. The MTERF1 domains used are indicated on the x-axis. The analysis is based on flow cytometry data. (C) Length of DNA in base pairs (bp) needed to encode the different DNA-binding domains. The peptide range from MTERF1 is indicated on the x-axis. The dotted line indicates the length of the benchmark MTERF1₅₈₋₃₉₉ domain. MTP, mitochondrial transfer peptide; WT, wild type; Rel., relative; bp, base pairs.

FIG. 6. Experimental assessment of immunogenicity.

(A) Scheme of the experimental process. Peripheral blood mononuclear cells (PBMCs) are depicted in various shapes inside drawings of culture wells and peptides are drawn as rods. Dimethylsulfoxide (DMSO) is the solvent for the peptides. (B) P-Values of a t-test comparing the number of spot forming units (SFU) from cells primed and recalled with the same peptide pool and the same PBMCs that were primed but not recalled. The x-axis shows the protein from which the peptide pools used for priming and recall were derived. A light shade of gray delineates p-values above 0.05, with donors classified as non-responders. A darker shade gray indicates the area of p-values below 0.05. P-values of donor-derived samples that fall into this area are considered responders. The dotted line indicates a p-value of 0.05. Different point characters indicate different PBMC donors. DMSO, dimethylsulfoxide; ELISPot, Enzyme-Linked ImmunoSpot; HO: null hypothesis; SFU, spot forming units

FIG. 7. Orthogonality assessment of MTF.

A) Confocal microscopy of Hela cells transfected with either Flag-tagged wild-type MTERF1 or MTF. Scale bar indicates 10 μm. MitoRed is a dye that stains mitochondria, anti-Flag Ab was used to stain the Flag-tagged transfected proteins, and Hoechst33342 stains DNA. B) Diagram showing plasmids and their encoded protein's purported interactions. C) Reporter fluorescence levels of HEK293 cells transfected with MTF construct and a reporter construct comprising 5×RE without a spacer in its promoter (SEQ ID NO: 48) with or without EF1a-driven WT MTERF1.

FIG. 8. Volcano plots of differential gene expression analysis of transfected HEK293 cells.

(A) Volcano plot showing genes differentially regulated between cells transfected with WT MTERF1 and a junk plasmid. Y-axis shows the log 10 of the false detection rate (FDR). The log 2 of the fold-change (FC) is shown on the x-axis. Dots in light gray indicate genes not significantly differentially expressed, mid-shade gray dots indicate down-regulated genes, and a dark shade represents up-regulated genes. Labels indicate the ENSEMBL symbol of the gene corresponding to the closest spot, or a “-” for transcripts not associated with a named gene. (B) Volcano plot comparing gene expression between cells transfected with the MTF construct and cells receiving a junk DNA plasmid. Y-axis shows the log 10 of the false detection rate (FDR). The log 2 of the fold-change (FC) is shown on the x-axis. Dots in light gray indicate genes not significantly differentially expressed, mid-shade gray dots indicate down-regulated genes, and a dark shade represents up-regulated genes. Labels indicate the ENSEMBL symbol of the gene corresponding to the closest spot, or a “-” for transcripts not associated with a named gene. WT, wild type; FDR, false detection rate; FC, fold change.

FIG. 9. Optimization of transcriptional activation strength and protein size.

A) Overview of TAD domains. Subscripted numbers indicate the amino acid range comprising the TAD domain used in this work. B, C, E, F) Transcriptional activation mediated by individual transactivation domains. Number of base pairs required to encode the HumTAP variant are shown in the top panel. The bottom panel shows flow cytometry analysis of HEK293 cells co-transfected with a plasmid encoding pEF1a-driven mCherry, design 6 reporter, and equimolar amounts of plasmids encoding pEF1a-driven MTERF1₅₈₋₃₉₉ fused to the TAD domain indicated below each bar. Symbols below bars correspond to symbols in panels A and D. Bars correspond to the mean of three replicates, and each dot indicates one replicate. The horizontal dashed line indicates the rel. mCerulean level in cells transfected with the benchmark MTERF1₅₈₋₃₉₉::RelA₄₃₀₋₅₅₁ HumTAP variant, i.e. “MTF” (SEQ ID NO: 100). D) Illustration of MTERF1 structure and amino acid ranges retained for different variants. Amino acid sequences left out of the illustration are indicated by dotted outlines while vertical dashed lines indicate the starting amino acid positions for each variant. TAD, transactivation domain; DBD, DNA-binding domain; MTP, mitochondrial transfer peptide; bp, base pair.

FIG. 10. Construction of a rapalog-inducible HumTAP-based gene expression system.

A) Illustration of A/C heterodimerizer-mediated dimerization of MTERF158-399: FKBP (SEQ ID NO: 192) and FRB: TAD. B) Flow cytometry analysis of HEK293 cells co-transfected with the design 6 reporter plasmid, a plasmid encoding pEF1a-driven mCherry, and varying amounts of each a plasmid encoding pEF1a-driven expression of each of the dimerizing proteins. Axis units refer to molar equivalents of plasmid amounts while relative mCerulean was calculated by normalizing the mCerulean signal to the mCherry signal. C) Relative mCerulean expression levels in HEK293 cells co-transfected with design 6 reporter plasmid, a plasmid with pEF1a-driven mCherry expression and a plasmid encoding pEF1a-driven expression of each of the two dimerizing proteins. Points represent mCerulean levels normalized to mCherry levels and dashed lines represent a logistic model fit. Colors indicate which TAD was used in the construction of the FRB: TAD fusion protein transfected into cells from which data was obtained. D) Relative mCerulean expression levels in HEK293 cells co-transfected with design 6 reporter plasmid, a plasmid with pEF1a-driven mCherry expression and a plasmid encoding pEF1a-driven expression of each of the two dimerizing proteins. Points represent mCerulean levels normalized to mCherry levels and lines data fitted using a logistic model. Colors indicate which linkers were used in the construction of the MTERF1₅₈₋₃₉₉::FKBP fusion protein (linker 1) and FRB::TAD fusion protein (linker 2) transfected into cells from which data was obtained. TAD, transactivation domain; Rv1, RelA₄₃₀₋₅₅₁ (SEQ ID NO: 3)

FIG. 11. Schematic of the massively parallel reporter assay to determine cognate promoter design principles.

A) Illustration of the components and varied properties in the plasmid library. B) Overview of the experimental procedure. BS, binding site; bp, base pairs, BC, barcode; RT, reverse transcription; PCR, polymerase chain reaction

FIG. 12. Quality control of the promoter design library.

A) The chimera rate indicates the ratio of reads containing an unexpected promoter design coupled to a given barcode to total number of reads containing the barcode. The barcode read count indicates the total number of reads in which the barcode occurred. Distributions of points along each axis are shown on the top and left margins. R2 refers to the square of Pearson's correlation coefficient. B) Distribution of design parameters indicated on top of each stack. Percentages indicate the frequency of each given parameter identified among all reads, and boxes are of a corresponding height. dir., direction

FIG. 13. Quality control of the sequencing results.

A) Histogram showing how often each barcode was read in the plasmid DNA sample. B) Height of the stacked bars indicate for each sample the distribution of the barcodes originating either from any library member or any of the UbC controls. C) Dotplot showing activity scores for each barcode in two replicates of cells transfected with an MTF plasmid amount corresponding to the EC90. The marginal density plot indicates the distribution of barcode activity scores of replicate 2. D) Histogram of promoter-level activity scores at an MTF level corresponding to the EC90. E) Dotplot indicating on the y-axis Citrine fluorescence levels normalized to mCherry expression in HEK293 cells co-transfected with individually picked plasmids, a plasmid encoding pEF1a-driven MTF at an amount corresponding to the EC90, and a plasmid encoding pEF1a-driven mCherry. The x-axis indicates activity scores for the designs corresponding to the individually picked plasmids. R2 indicates the square of the Pearson correlation, p the Spearman correlation coefficient, and a linear regression fit is shown as a dashed line. F) Trajectories of activity scores obtained from RNA samples of cells co-transfected with the plasmid library, plasmids encoding UbC-driven Citrine, and varying amounts of the plasmid encoding pEF1a-driven MTF. EC, effective concentration

FIG. 14. Promoter activities at a MTF level corresponding to its EC90.

A) Scheme of the gene regulatory cascade underlying the measured promoter design activity scores. B) Ranks assigned to promoters according to their activity score in descending order. The marginal density plot represents the distribution of promoter activity scores. C) Heatmap of all activity scores of all designs. For designs with one BS, “Distance BS-BS” does not apply and was set to 0. D) Distribution of activity scores. Violins contain all designs sharing the indicated design parameter and black dots represent the median activity score. P values were calculated using analysis of variance (anova). E, F) Distribution of activity scores. Violins contain all designs sharing the indicated design parameter with binding sites in either sense (E) or anti-sense (F) orientation relative to the minimal promoter. Black dots represent the median activity scores. P values were calculated using analysis of variance (ANOVA). BS, binding site; bp, base pairs

FIG. 15. Characterization of individual promoter variants.

A) Variants were either randomly picked or specifically chosen for their high activity scores and small size. Dark colored points indicate picked and individually transfected promoter design variants and dots with different fill patterns show designs indicated by the same patterns in panel B, C, and D. B, D) Mean relative mCitrine levels of 3 or 6 replicates measured by flow cytometry in cells co-transfected with a MTF-encoding plasmid at a level corresponding to the EC90. C) Relative mCitrine levels from cells transfected with the amount of MTF-encoding plasmid indicated in the axis label. D) White points indicate individual replicates.

### EXAMPLES

Methods and materials are described herein for use in the present disclosure; other, suitable methods and materials known in the art can also be used. The materials, methods, and examples are illustrative only and not intended to be limiting.

### Example 1. Engineering of an MTERF1-Based Synthetic Transactivation System

The inventors engineered an exemplary “Human-derived transcriptional activator protein” (HumTAP) transactivation system by engineering a HumTAP transactivator and a promoter comprising the Response Elements/MTERF1 binding sites that is operably linked to a gene of interest. A prototype HumTAP protein was made by fusing a peptide consisting of amino acids 58 through 399 of the human MTERF1 gene (MTERF1₅₈₋₃₉₉ (SEQ ID NO: 1), Uniprot Q99551) to a peptide consisting of amino acids 430 through 551 of the RELA protein (RelA₄₃₀₋₅₅₁ (SEQ ID NO: 3), Uniprot Q04206). This fusion is henceforth referred to as MTERF1₅₈₋₃₉₉::RelA₄₃₀₋₅₅₁ (or “MTF”; SEQ ID NO: 100). A DNA construct was made with a strong EF1A promoter driving a constitutive expression of MTF (SEQ ID NO: 101) in human cells (“MTF construct”).

To construct a promoter with Response Elements/MTERF1 binding sites driving the gene of interest, the inventors placed MTERF1 binding sites (SEQ ID NO: 42) upstream a minimal TATA box (SEQ ID NO: 103). An mCerulean fluorescent reporter representing the gene of interest (Gol) was placed downstream of the TATA box. Each of these is referred to as a reporter construct. Five different reporter constructs were made comprising respectively 3, 5, 7, 9, and 11 MTERF1 binding sites 6 base pairs apart upstream of the TATA box (FIG. 1A; SEQ ID NO: 41, 44, 45, 46 and 47, respectively). They were co-transfected with the MTF construct into Hela cells. Microscopy analysis showed a maximum of mCerulean signal for 11 repeats of MTERF1 binding sites (FIG. 1B). Using flow cytometry, relative mCerulean levels were measured and calculated by normalization to the transfection control in Hela cells (FIG. 1C). The same transfection conditions in HEK293 cells revealed an increase in reporter fluorescence levels with the number of REs/MTERF1 binding sites in the promoter design using microscopy (FIG. 1D) and flow cytometry (FIG. 1E). Next, reporter plasmids with varying spacer lengths (0, 6 bp and 10 bp; SEQ ID NO: 48, 44 and 49, respectively) (FIG. 1F) between a fixed number of REs (i.e. 5 REs/MTERF1 binding sites) were transfected with the MTF construct. In Hela cells, microscopy revealed a large increase in reporter expression when there was no spacing at all and similar amounts of mCerulean for 6 and 10 bp spacing (FIG. 1G). This was confirmed using flow cytometry (FIG. 1H). A different trend was observed in HEK293 cells with the highest reporter amount observed with 6 bp spacing and very little mCerulean signal with 10 bp spacing, using microscopy (FIG. 1I) and flow cytometry (FIG. 1J). Confirming sequence specificity, a reporter construct with a promoter comprising 5 scrambled DNA binding sites spaced by 10 bp (SEQ ID NO: 51) (FIG. 1K) did not lead to mCerulean expression upon co-transfection with the MTF construct in HeLa as assessed by microscopy (FIG. 1L) and flow cytometry (FIG. 1M). HEK293 cells showed the same behavior under the microscope (FIG. 1N) and flow cytometry (FIG. 10).

Next, the inventors optimized the DNA sequence encoding MTF by changing codons from the naturally used ones to the most common ones in humans for each amino acid (SEQ ID NO: 102; FIG. 2A). This optimization increased reporter gene expression ca. 3-fold in Hela cells using reporter construct with 5×RE/MTERF1 binding sites separated by a 6 bp spacer in its promoter (SEQ ID NO: 44) as assessed by microscopy (FIG. 2B) and flow cytometry (FIG. 2C). In HEK cells, this modification had no significant effect on reporter fluorescence levels as depicted by micrographs (FIG. 2D) and flow cytometry analysis (FIG. 2E).

By transfecting different amounts of the codon optimized MTF construct into HEK293 cells the inventors established a dose dependency of mCerulean reporter (Gol) expression on the expression levels of MTF (FIG. 3). HEK293 cells in 24-well plates were transfected with 0.75, 3, 6, 12, 24, 48, 96, 192, and 384 ng of MTF construct, 100 ng of reporter construct, and 50 ng of constitutive mCherry transfection control construct. Two days after transfection, the cells were analyzed using flow cytometry. The dose-response curve was modeled as a Hill equation assuming a Hill coefficient of 1. EC10 and EC50 were achieved at 1.25 ng and 11.24 ng of the transfected MTF construct, respectively. A theoretical EC90 was achieved at 101 ng, although a slight drop in relative reporter fluorescence level is observed for higher amounts of transfected MTF construct.

With the goal to engineer stronger and possibly shorter HumTAPs, the inventors fused the MTERF1₅₈₋₃₉₉ peptide (SEQ ID NO: 1) to a variety of transactivation domains and transfected HEK293 cells with those constructs and the reporter construct comprising 5×RE/MTERF1 binding sites without a spacer between them (SEQ ID NO: 48).

First, MTERF1₅₈₋₃₉₉ (SEQ ID NO: 1) was fused with different subsequences of RelA. RelA₃₆₁₋₅₅₁ (SEQ ID NO: 27) corresponds to the sequence used in the construction of the bacterial-derived synthetic TF PIT2 [19], while RelA₃₄₂₋₅₅₁ (SEQ ID NO: 29) contains all annotated transactivation domains according to domain annotation under Uniprot Q04206 (https://www.uniprot.org/uniprotkb/Q04206/entry). Both lead to stronger reporter gene expression (FIG. 4A) but also require a larger genetic footprint as compared to RelA₄₃₀₋₅₅₁ (SEQ ID NO: 3; FIG. 4B). The TA1 (RelA₅₂₁₋₅₅₁, SEQ ID NO: 31) domain of RelA did not activate reporter gene expression significantly. We also fused MTERF1₅₈₋₃₉₉ (SEQ ID NO: 1) to a set of transactivation domains previously found to be strong activators with a small genetic footprint (FIG. 4B). The WW domain of WWC1 (SEQ ID NO: 21; WWC1₂₋₈₁, Uniprot Q8IX03), KRAB domain of ZNF473 (ZNF473₅₋₄₈, Uniprot MOR032, SEQ ID NO: 23), and the Nuc_rec_co-act domain of NCOA3 (SEQ ID NO: 25; NCOA3₁₀₄₅₋₁₀₉₂, Uniprot Q9Y6Q9) did not lead to high reporter expression. Conversely, the LMSTEN domain of MYB (SEQ ID NO: 19; MYB₂₅₁₋₃₃₀, Uniprot P10242) and FOXO-TAD domain of FOXO3 (SEQ ID NO: 17; FOXO3₆₀₄₋₆₄₄, Uniprot 043524) mediated significant mCerulean expression. VP64, which is comprised of three repeats of the transactivation domain of HHV11 (SEQ ID NO: 15) (SEQ ID NO: 13; HHV11₄₃₇₋₄₄₇, Uniprot P06492) linked by a GS linker, a commonly used strong viral-derived transactivation domain, mediated lower reporter expression than the benchmark RelA₄₃₀₋₅₅₁ domain that was used. Interestingly, fusion of two copies of FOXO3₆₀₄₋₆₄₄ (SEQ ID NO: 33) or RelA₅₂₁₋₅₅₁ (SEQ ID NO: 35) yielded 12 and 201 times the fluorescence conferred by only one copy of each respective TAD (FIG. 4A). These results show that the DBD of MTERF1 can be used as a versatile building block to modulate gene expression when fused to a TAD that may or may not be derived from RelA.

To further decrease the DNA footprint of a HumTAP, the inventors investigated to what degree the DNA binding domain may be reduced. To this end, the inventors created 3 versions of MTERF1 subsequences fused to RelA₄₃₀₋₅₅₁ (SEQ ID NO: 3). MTERF1₇₃₋₃₉₉ (SEQ ID NO: 7), MTERF1₁₀₄₋₃₉₉ (SEQ ID NO: 9), and MTERF1₁₃₅₋₃₉₉ (SEQ ID NO: 11) are subunits of MTERF1 without its mitochondrial transfer peptide (MTERF1₁₋₅₇; SEQ ID NO: 37) that either lack only the first 14 N-terminal amino acids (MTERF₅₈₋₇₂; SEQ ID NO: 39) or additionally, also lack the first (MTERF1₇₃₋₉₈; SEQ ID NO: 104) or both the first and second (MTERF1₁₀₄₋₁₃₄; SEQ ID NO: 106) mterf motifs (FIG. 5A) [21]. The removal of N-terminal domains reduces the genetic footprint of a HumTAP (FIG. 5B). These constructs were co-transfected into HEK293 cells with the reporter construct containing 5×RE without a spacer in its promoter (SEQ ID NO: 48). It was found that output expression was reduced but not abolished by the truncation of MTERF1 from the N-terminus of up to amino acid 104, i.e. in MTERF1₇₃₋₃₉₉ (SEQ ID NO: 7) and MTERF1₁₀₄₋₃₉₉ (SEQ ID NO: 9). Further truncation till amino acid 135 (MTERF1₁₃₅₋₃₉₉ (SEQ ID NO: 11)), however, led to complete loss of transcriptional activation (FIG. 5B). This shows that the genetic footprint of the synthetic TF can be reduced while activity is retained.

### Example 2. Assessment of Immunogenicity

T cells that bind strongly to self-peptide-MHC complexes are deleted in the thymus during maturation, allowing for tolerance to self-peptides [22]. Therefore, because HumTAP consists entirely of human protein subunits, it is expected to have a more favorable immunogenic profile compared to proteins of non-human origin.

The inventors assessed whether an immune response might be primed by protein subsequences. Using

NetMHCpan 4.0 the inventors predicted which peptides derived from the MTERF-RelA junction are most likely to bind strongly to HLA-A02:01, the most common MHC I allele in the human population. A pool of the five strongest predicted binding peptides was synthesized. Furthermore, for each of those “core” binders, the inventors synthesized a 15-mer containing the flanking sequence, yielding 10 peptides. As a positive control, a peptide pool spanning NY-ESO1, a immunogenic cancer-associated protein, was used. Peptides derived from the self-parts (i.e. RelA and MTERF but not the junction) served as the negative control. Peripheral blood mononuclear cells (PBMCs) were isolated from normal blood donors, transferred to 24-well plates and each peptide pool was added into each of the three replicate wells. The cells were then incubated for 14 days in the presence of IL-2 and IL-7. In this phase, T cells harboring a T cell receptor (TCR) that recognizes a peptide bound to MHC Class I or Class II on the surface of PBMC, antigen-presenting cells in particular, will be stimulated and start to proliferate, a process called “priming”. Next, PBMCs in each well are re-stimulated with the same peptide pool overnight and subjected to an IFNγ-ELISPot in 4 replicates, whereby spots are developed and counted (FIG. 6A). A spot is elicited by a T-cell that has previously been primed by one of the peptides in the priming peptide mixture, therefore more spots correspond to a more immunogenic peptide mix at the priming step. A donor is classified as a “responder” if significantly more spot forming units (SFU) (p<0.05) are observed for re-stimulated cells than for their respective primed but not re-stimulated background controls, as recommended in Moodie (2010), Cancer Immunology, Immunotherapy volume 59, pages 1489-1501. Some donors did not show a significant response to the NY-ESO1 peptide pool and were disregarded for the analysis. 7 PBMC samples showed a response to NY-ESO1 (positive control) and were included in the analysis. Of those samples, one mounted a significant response against the junction-derived peptide pool while 3 PBMC samples were deemed responders to the negative control peptides.” (FIG. 6B). This suggests that the MTF junction-derived peptide pool behaves much more similar to a negative control consisting of self-peptides than to a known immunogenic positive control, and points towards low immunogenicity in the human.

### Example 3. Assessment of Orthogonality

The inventors checked whether the lack of the mitochondrial transfer peptide (MTP) of the HumTAP would lead to orthogonality of the HumTAP towards the human host cells and vice versa, that is, (i) whether endogenous human gene expression is not substantially altered by the HumTAP and (ii) whether endogenously-expressed wild-type MTERF1 (i.e. including the MTP as shown in SEQ ID NO: 37) lacks the ability to modulate the expression of a gene of interest driven by a Response Element-containing promoter.

First, the inventors assessed intracellular localization by transfecting Hela cells with a plasmid constitutively expressing Flag-tagged MTF (SEQ ID NO: 128), or a plasmid constitutively expressing Flag-tagged wild-type MTERF1 (SEQ ID NO: 130). Cells were stained with an anti-Flag-tag antibody and imaged by confocal microscopy. Indeed, WT MTERF1 was found to be confined to mitochondria whereas MTF was distributed throughout the whole cell, including the nucleus, without obvious localization to the mitochondria (FIG. 7A).

To assess the orthogonality of the host towards the Response Element-driven Gene of Interest (Gol), the inventors transfected a HumTAP representative MTF (SEQ ID NO: 100) with a Response Element-driven (SEQ ID NO: 48) mCerulean fluorescent reporter, with and without the construct encoding a wild-type MTERF1 to assess whether it might interfere with the induction of the Gol by MTF (FIG. 7B). The inventors found no difference in Gol fluorescence levels with or without concomitant WT MTERF1 expression (FIG. 7C).

Alignment of the MTERF1 binding site using BLAST to the human genome shows no perfect matches in the nuclear genome, but the existence of imperfect binding sites to which MTF may bind could not be excluded. The inventors therefore used RNA-Seq to investigate whether MTF (SEQ ID NO: 100) activates the expression of endogenous human genes. The inventors also transfected WT MTERF1 (SEQ ID NO: 108) to compare the gene-regulatory effects of MTERF1 to those of MTF. Differential gene expression analysis was performed against cells that were transfected with a “junk” plasmid using the edgeR software package for R [24]. Genes were deemed differentially expressed (DE) if they were found to be significantly differentially expressed (FDR<0.5) by a fold-change of at least 2. For WT MTERF1, 7 genes were found to be down-regulated and 3 were up-regulated (not including MTERF1 expressed from the transfected plasmid) (FIG. 8A). For MTF, 24 genes were down- and 4 genes were up-regulated (FIG. 8B). Notably, none of the genes found to be downregulated by WT MTERF1 were also found to be affected by MTF. Merely one gene, HSPA7, was upregulated in both conditions (Table 1). There were a few genes significantly downregulated by MTF, but none of them contain sequences similar to the MTERF1 binding site in their genomic vicinity. Similarly, none of the upregulated genes in the MTF-transfected cells harbor a canonical MTERF1 binding motif. The inventors conclude from this experiment that MTF does not retain the gene regulatory function of the wild-type MTERF1 protein and does not exert widespread off-target gene activation. The lack of MTERF1 binding sites in the genomic vicinity of downregulated genes may suggest a mechanism independent of canonical MTF binding. Future investigations will assess the dose-dependency of differentially expressed (DE) genes on MTF amount and may help clarify the origin of DE genes.

TABLE 1
List of all DE genes for MTF (top) and WT MTERF1 (bottom).

| | | |
|---|---|---|
| Regulation | Symbol | Gene name |

MTF

| | | |
|---|---|---|
| Down | G0S2 | G0/G1 Switch 2 |
| | INHBE | Inhibin Subunit Beta E |
| | S100P | S100 Calcium Binding Protein P |
| | FGF21 | Fibroblast Growth Factor 21 |
| | LINC01512 | Long Intergenic Non-Protein Coding RNA |
| | | 1512 |
| | GDAP1L1 | Ganglioside Induced Differentiation Associated |
| | | Protein 1 Like 1 |
| | CHAC1 | Glutathione Specific Gamma- |
| | | Glutamylcyclotransferase 1 |
| | — | MicroRNA 3937 |
| | TRIB3 | Tribbles Pseudokinase 3 |
| | BEST1 | Bestrophin 1 |
| | DDIT3 | DNA Damage Inducible Transcript 3 |
| | SLC6A9 | Solute Carrier Family 6 Member 9 |
| | LINC02154 | Long Intergenic Non-Protein Coding RNA |
| | | 2154 |
| | DDIT4 | DNA Damage Inducible Transcript 4 |
| | RAPGEF3 | Rap Guanine Nucleotide Exchange Factor 3 |
| | ADM2 | Adrenomedullin 2 |
| | PCK2 | Phosphoenolpyruvate Carboxykinase 2, |
| | | Mitochondrial |
| | ASNSP1 | ASNS Pseudogene 1 |
| | SLC1A4 | Solute Carrier Family 1 Member 4 |
| | KRTAP19-1 | Keratin Associated Protein 19-1 |
| | EIF4EBP1 | Eukaryotic Translation Initiation Factor |
| | | 4E Binding Protein 1 |
| | SLC7A5 | Solute Carrier Family 7 Member 5 |
| | CTH | Cystathionine Gamma-Lyase |
| | SLC7A11 | Solute Carrier Family 7 Member 11 |
| Up | — | Novel Transcript |
| | USP17L2 | Ubiquitin Specific Peptidase 17 Like Family |
| | | Member 2 |
| | HSPA7 | Heat Shock Protein Family A (Hsp70) |
| | | Member 7 (Pseudogene) |
| | ADGRA1 | Adhesion G Protein-Coupled Receptor A1 |

WT MTERF1

| | | |
|---|---|---|
| Down | H2BC8 | H2B Clustered Histone 8 |
| | H3C12 | H3 clustered histone 12 |
| | H2AC8 | H2A clustered histone 8 |
| | H2AC12 | H2A clustered histone 12 |
| | MT-CO3 | mitochondrially encoded cytochrome c oxidase |
| | | III |
| | H2AC6 | H2A clustered histone 6 |
| | H4C2 | H4 clustered histone 2 |
| Up | GDF15 | growth differentiation factor 15 |
| | TRPV6 | transient receptor potential cation channel |
| | | subfamily V member 6 |
| | HSPA7 | heat shock protein family A (Hsp70) member |
| | | 7 (pseudogene) |

### Example 4. Optimization of the humTAP

This Example is an extension of Example 1 herein above which describes, partly, the same data and results.

For many applications, strong gene of interest transactivation by a synthetic transcription factor (synTF) is required at the same time as viral delivery methods impose a need for components to be as small as possible [25]. Therefore, the inventors examined a variety of human and engineered chimeric transactivation domains (TADs) for their size and transactivation strength and benchmarked it to the RelA₄₃₀₋₅₅₁ peptide (SEQ ID NO: 3).

In addition to RelA₄₃₀₋₅₅₁ (termed “v1”), the inventors tested three alternative subsequences: v2) RelA₃₄₂₋₅₅₁ (SEQ ID NO: 29), which contains all transactivation domains as annotated on UniProt Q04206; v3) RelA₃₆₁₋₅₅₁ (SEQ ID NO: 27), which is identical to the peptide used in the construction of PIT2 [2]; and v4) RelA₅₂₁₋₅₅₁ (SEQ ID NO: 31), which constitutes only the transactivation domain 1 (TA1) [26]. The protein domains WW (SEQ ID NO: 21), KRAB of ZNF473 (SEQ ID NO: 23), Nuc-rec-co-Act (SEQ ID NO: 25), LMSTEN (SEQ ID NO: 19), and FoxoTAD (SEQ ID NO: 17) were recently identified as strong transactivators [27]. To assess their functionality in the HumTAP context, the inventors created fusion proteins between each of them and MTERF1₅₈₋₃₉₉ (SEQ ID NO: 1) (FIG. 9A).

Upon co-transfection of HEK293 cells with plasmids encoding pEF1a-driven expression of the fusion HumTAPs, a Response Element-driven mCerulean reporter protein, and a mCherry transfection control, all peptides derived from RelA except TA1 mediated high levels of reporter gene expression. Including a larger fraction of the RelA protein led to higher reporter expression. Conversely, the core transactivation domains TA1, WW, ZNF473 KRAB, and NucRecCoAct mediated little to no reporter expression. LMSTEN and FoxoTAD domains yielded some mCerulean expression, although at 11.3 and 8.4-fold lower levels than the benchmark RelA₄₃₀₋₅₅₁ TAD, respectively (FIG. 9 B).

Interestingly, a fusion of two TA1 domains (SEQ ID NO: 35) led to reporter expression and the addition of a third domain (SEQ ID NO: 194) further increased output expression (FIG. 9 C). Similarly, two copies of FoxoTAD (SEQ ID NO: 33) mediated 20-fold more mCerulean expression than just one FoxoTAD copy and ~2-fold more than the benchmark RelA₄₃₀₋₅₅₁ TAD. This effect was not as pronounced for the LMSTEN domain for which two copies (SEQ ID NO: 196) mediated ~2.7-fold more reporter expression than one copy. Fusing FoxoTAD with LMSTEN (SEQ ID NO: 54), LMSTEN with TA1 (SEQ ID NO: 56), or FoxoTAD with TA1 (SEQ ID NO: 58) yields TADs that are at the same time smaller and stronger than the benchmark RelA₄₃₀₋₅₅₁ TAD. A chimeric TAD consisting of FoxoTAD, LMSTEN, and TA1 (SEQ ID NO: 52) yields 3.7 times the amount of reporter gene expression than RelA₄₃₀₋₅₅₁ (FIG. 9 C). It is therefore possible to engineer both stronger and smaller transactivation domains than the commonly used RelA₄₃₀₋₅₅₁ without incorporating protein domains of non-human origin. Given the inventors' observation of synergy between identical as well as heterotypic transactivation domains, it is likely that further work exploring this effect would yield human-based chimeric TADs mediating even higher levels of transgene expression levels without increasing the protein domain's size.

To further reduce the size of the HumTAP protein, the inventors deleted the N-terminus (SEQ ID NO: 39), optionally the Mterf motif 1 (SEQ ID NO: 104), and optionally the Mterf motif 2 (SEQ ID NO: 106) additionally to the MTP (SEQ ID NO: 37) (FIG. 9 D) of MTERF1. Given the activity-abolishing effect of the R387A mutation [28], which is located just 12 amino acids from the MTERF 1 C-terminus, the inventors did not delete any C-terminal domains. Omitting the N-terminus (MTERF1₇₃₋₃₉₉; SEQ ID NO: 7) and both N-terminus and Mterf motif 1 (MTERF1₁₀₄₋₃₉₉; SEQ ID NO: 9) yielded ~1.5-times less reporter expression but reduced the DNA size by 45 and 135 bases, respectively. Omitting also Mterf motif 2 (as in SEQ ID NO: 11) resulted in a non-functional synTF (FIG. 9 E).

To check whether the reduced transactivation activity of the HumTAPs with smaller DBDs can be compensated, the inventors fused the novel chimeric TADs to the MTERF1₁₀₄₋₃₉₉ peptide (SEQ ID NO: 9). In this context, the FoxoTAD: TA1 chimeric TAD (SEQ ID NO: 58) yielded a synTF about as strong as MTF (SEQ ID NO: 100) but requires 288 fewer DNA bases to encode on a vector. The combinations of MTERF1₁₀₄₋₃₉₉ with FoxoTAD: FoxoTAD (SEQ ID NO: 33) and FoxoTAD: LMSTEN (SEQ ID NO: 54) constitute synTFs ~1.5-fold stronger than MTF (SEQ ID NO: 100) but are 96 and 86 amino acids smaller, respectively. Using FoxoTAD: LMSTEN: TA1 (SEQ ID NO: 52) in this context led to a synTF of similar size as MTF but mediating 3.5-fold more reporter gene expression (FIG. 9 F).

In summary, this dataset supports the notion that MTERF1 retains DNA-binding functionality even if N-terminal amino acids are missing. The lowered reporter levels may be explained by reduced affinity of the protein to DNA or a less stable protein. Either way, high reporter gene expression levels can be recovered through the use of an optimized TAD.

### Example 5. Engineering of an MTF-Based Inducible Gene Expression System

Tunability of output expression strength by small molecules has been recognized as an important feature of synthetic transcription activation systems [29]. The inventors therefore sought to engineer a dimerization-based inducible HumTAP system.

One well-characterized small-molecule based dimerization system is based on the rapamycin analog A/C (C16-(S)-7-methylindolerapamycin) that induces heterodimerization between the human proteins FK506-binding protein 12 (FKBP; SEQ ID NO: 142) and the FKBP12-rapamycin binding domain (FRB) mutant FRBT2098L (SEQ ID NO: 140) [30], [31]. To achieve A/C heterodimerizer-inducible gene expression, the inventors fused FKBP (SEQ ID NO: 142) to the N-terminus of MTERF1₅₈₋₃₉₉ (SEQ ID NO: 1) and FRB (SEQ ID NO: 140) to the C-terminus of different transactivation domains (FIG. 10A). At a constant A/C heterodimerizer (i.e. C16-(S)-7-methylindolerapamycin) concentration of 100 nM, the inventors found that a 4-fold molar excess of the FRB::RelA₄₃₀₋₅₅₁ (SEQ ID NO: 170) plasmid over the MTERF1₅₈₋₃₉₉: FKBP (SEQ ID NO: 192) plasmid led to the highest mCerulean levels in HEK293 cells co-transfected with the inducible TF components and a Response Element-driven mCerulean reporter protein (FIG. 10 B). In HEK cells co-transfected with MTERF1₅₈₋₃₉₉::FKBP and fusion proteins between FRB and the strongest of the newly developed TADs (see Example 4), mCerulean levels increased dose-dependently with inducer concentrations (FIG. 10 C). In accordance with the results described above, chimeric transactivation domains mediated higher maximal output expression levels than RelA₄₃₀₋₅₅₁.

Upon ingestion, physiological blood concentrations of rapamycin, from which the A/C dimerizer is derived, reach around ~22 nM whereas the present system exhibits an EC50 between 100 and 120 nM. Previously reported inducible gene expression systems based on A/C-inducible FRB and FKBP dimerization exhibited EC50 values of ~1 nM [31], [33], and are therefore inducible by pharmacologically achievable inducer concentrations. The inventors reasoned that in the present system, dimerization might be hindered by either FRB's or FKBP's fusion partner and investigated whether the addition of linkers between the protein domains might impact the EC50 value (FIG. 10 D). A combination of the cMyc nuclear localization signal (NLS) (SEQ ID NO: 122) linking MTERF158-399 and FKBP, and the rigid linker AP6 (SEQ ID NO: 120) linking FRB and RelA₄₃₀₋₅₅₁ led to a reduction in EC50 to ~60 nM while also increasing maximal output ~3-fold over the variant without linkers and RelA₄₃₀₋₅₅₁ as TAD (FIG. 10 D).

### Example 6. Engineering of a Collection of HumTAP-Cognate Promoters (Sensors)

Several factors interact in a complex manner to impact the strength of a synthetic promoter. The inventors performed a massively parallel reporter assay (MPRA) [35]-[37] to quantify the impact of four promoter design parameters on transactivation strength: number of MTERF1 binding site (BS) (SEQ ID NO: 42), their orientation relative to the minimal promoter, number of spacing bp between BSs, and the number of spacing bp between the minimal promoter and the proximal BS (FIG. 11A).

The inventors investigated a range of 1 to 5 BS (SEQ ID NO: 42) copies, each in both sense (SEQ ID NO: 42) or anti-sense (SEQ ID NO: 200) orientation relative to the yb_TATA minimal promoter (SEQ ID NO: 103), 0 to 10 bp distance to the minimal promoter (see SEQ ID NO: 199 for the 10 bp spacer; the shorter spacers are truncated at the 3′ end of SEQ ID NO: 199), and 0 to 10 bp spacing between BSs (see SEQ ID NO: 198 for the 10 bp spacer; the shorter spacers are truncated at the 3′ end of SEQ ID NO: 198). For each of the 990 possible parameter combinations, 10 unique 11 bp-barcodes were assigned. The resulting library of 9910 distinct DNA sequences was cloned in such a way that each encoded promoter variant drives the expression of mCitrine with the associated barcode in the 3′ UTR. The relative frequency of each barcode in the plasmid library was measured using next-generation sequencing. To control for differences in transfection efficiencies and expression levels among different conditions, the inventors cloned 10 plasmids encoding the constitutive UbC promoter (SEQ ID NO: 139) driving mCitrine with each a unique barcode in the 3′ UTR. Then, MTF (SEQ ID NO: 100) plasmid amounts corresponding to the EC10, EC50, and EC90 (see Example 1 and FIG. 3) were co-transfected with the promoter library and the 10 UbC-control plasmids into HEK293 cells in triplicates. To quantify the expression level of each barcode, RNA was extracted from the cells, reverse transcribed, amplified, and sequenced. For each barcode, a score for each condition and replicate was calculated as the barcode count from the RNA sample normalized to the barcode frequency in the plasmid library and the median of the 10 barcodes associated with the UbC controls. The “barcode-level activity score” was the mean of barcode scores over the three replicates. For each promoter design, a final activity score was calculated as the median of the 10 associated barcode-level activity scores (FIG. 11 B).

The inventors performed quality control of the plasmid library using nanopore long-read sequencing analyzed with an algorithm that extracts the promoter design parameters and barcode of each read. A common issue in MPRA library construction is the decoupling of barcodes from their assigned variants due to chimeric DNA sequence formation during PCR amplification of DNA oligo pools [38], [39]. In the inventors' library, this effect occurs at an average rate of 17%, which means that, on average, 17% of plasmids encoding a given barcode are coupled to a promoter design different from the one assigned. The lack of correlation between barcode read count and chimera rate suggests that inventors' analysis is not underestimating the true chimera rate (FIG. 12A). Although chimeras are likely to cause noise in the screen, the inventors did not expect it to be sufficiently high to impede their ability to draw conclusions. Furthermore, analysis of design parameter distributions did not show strong biases for any certain parameters, apart from a skew toward promoters containing anti-sense oriented BSs (FIG. 12 B). The inventors therefore chose to proceed with the screening using the plasmid library.

The inventors then performed the whole workflow outlined in FIG. 11 B. Analysis of barcode frequencies in the plasmid library indicated complete coverage of the library, with no barcode being read less than 10 times, an average of ~27′000 reads per barcode, and a tail towards a maximum of 224′959 reads (FIG. 13A). As MTF amounts increase, so should the proportion of library-derived reads compared to UbC control-derived reads. Indeed, the inventors found between 75% and 97.6% of reads stemming from the plasmid library for the 0 MTF and EC90 levels, respectively (FIG. 13 B). Upon calculation of barcode-level activity scores in each sample, a bimodality in the distribution of scores became apparent: barcodes either fall into a “low” group with scores below ~0.001 or they fall into the “high” group which achieved scores above this threshold. A comparison of two replicates indicates that some barcodes fall in the “low” group in one replicate and the “high” group in the other replicate, and vice versa. (FIG. 13 C). After averaging over replicates and calculation of promoter-level activity scores, no more values below 0.001 are observed in the EC90 condition (FIG. 13 D). The inventors then isolated 39 individual plasmids from the library, co-transfected each plasmid together with an MTF amount corresponding to the EC90 and a mCherry transfection control plasmid into HEK293 cells, and measured fluorescence levels using flow cytometry. mCitrine fluorescence levels and screen-derived promoter-level activity scores correlated well with an R² of 0.77 and a Spearman correlation efficient (which does not assume a linear relationship) of 0.9 (FIG. 13 E). Confirming the promoter's responsiveness to MTF, activity scores of most designs increased with MTF levels whereas no consistent increase for a negative control design based on scrambled BSs was observed (FIG. 13 F)

The inventors concluded that the promoter design-level activity scores in the EC90 condition are informative of the reporter expression level mediated by the promoter variants. The inventors therefore analyzed promoter-level activity scores to discern the effects of design parameters on promoter activity scores at MTF levels corresponding to the EC90 (FIG. 14A). At MTF levels corresponding to the EC90, the highest promoter-level score was ~32 times higher than the lowest score (FIG. 14 B). Although most promoters scored in the lower half of the total activity range, a tail of high-scoring promoters exists (FIG. 14 B) with differences and trends visually apparent when presented as a heatmap (FIG. 14 C). Promoters built from binding sites in sense orientation relative to the minimal promoter yielded significantly higher scores than designs oriented the opposite way (i.e. binding sites in antisense orientation relative to the minimal promoter) (FIG. 14 D). As previously observed in other contexts [35], each additional binding site copy leads to higher scores, independent of their orientation (FIG. 14 E). This effect seems to saturate for sense-oriented BS design, however. This means that more than five binding sites only provide relatively minor activity increases. For example, for sense-oriented BS the median of all 2-BS-based designs is 1.69 times higher than that of 1-BS-based designs but going from 4 to 5 BS only increases the median activity score 1.09-fold. Analysis of variance (ANOVA) suggests that the distance between BSs has no significant influence on activity scores for both sense and anti-sense-based designs. Nonetheless, the highest scores were achieved by promoters with sense-oriented BS spaced by 1, 4, 5, and 8 bp (FIG. 14 E). The impact on activity scores of spacing base pairs between the minimal promoter and proximal BS, on the other hand, is highly significant. For sense orientation-based designs, there is a positive effect for spacing of more than 5 bp, with a peak at 8 bp (FIG. 14 E). Notably, for designs where the BS is oriented in anti-sense to the minimal promoter, larger distances between the proximal BS and minimal promoter are associated with lower activity scores (FIG. 14 F).

The inventors next sought to facilitate potential future applications and created a collection of promoters with different strengths to match potential future applications' requirements. Randomly picking 20 variants yielded mostly weak promoters. To maximize diversity in size and strength, the inventors chose 19 further promoters based on their high activity score or small genetic size. Altogether, the inventors created a promoter collection spanning a wide range of sizes and strengths (FIG. 15A, B). After co-transfecting each variant either with or without a MTF plasmid amount corresponding to the EC90, and a mCherry transfection control plasmid into HEK293 cells, relative mCitrine levels were measured using flow cytometry. Replicating the findings of the screen, the number of base pairs that space promoter components had a strong effect on reporter expression levels-allowing the inventors to find promoters of small size that mediate high expression levels. For example, one promoter with the configuration sense-2-0-8 (orientation-number of BSs-distance between BSs-distance to minimal promoter (SEQ ID NO: 300) mediated as much mCitrine expression as the sense-5-9-8 design (SEQ ID NO: 672) but required 102 bp less to encode. Conversely, the two promoter designs sense-5-9-8 (SEQ ID NO: 672) and sense-5-8-10 (SEQ ID NO: 693) differ in length by just 2 bp but the latter mediates twice the amount of mCitrine expression as the former (FIG. 15 B, D). Confirming responsiveness to MTF, reporter expression was only observed in cells co-transfected with the MTF-encoding plasmid but not in cells lacking this plasmid (FIG. 15 C).

While in mammalian synTF engineering, most attention has been directed toward the protein, the inventors investigated and highlighted here the great impact of cognate promoter design parameters. Beyond the importance of the number of binding sites, the spacing between promoter components has a profound impact on output expression strength. These surprising insights have allowed the inventors to construct a collection of promoters with a range of output strengths and find a variant that mediates high expression levels but, not considering the minimal promoter, requires just 52 bases to encode (i.e., sense-2-0-8; SEQ ID NO: 300).

### REFERENCES

The following references refer to the reference numbers in brackets, i.e. [1] to [24], as disclosed herein including the appended Examples:
[1] L. Naldini, “Gene therapy returns to centre stage,” Nature, vol. 526, no. 7573, pp. 351-360, October 2015, doi: 10.1038/nature 15818.
[2] S. Merlin and A. Follenzi, “Transcriptional Targeting and MicroRNA Regulation of Lentiviral Vectors,” Mol. Ther.-Methods Clin. Dev., vol. 12, pp. 223-232, March 2019, doi: 10.1016/j.omtm.2018.12.013.
[3] Z. Xie, L. Wroblewska, L. Prochazka, R. Weiss, and Y. Benenson, “Multi-Input RNAi-Based Logic Circuit for Identification of Specific Cancer Cells,” Science, vol. 333, no. 6047, pp. 1307-1311 September 2011, doi: 10.1126/science. 1205527.
[4] L. Morsut et al., “Engineering Customized Cell Sensing and Response Behaviors Using Synthetic Notch Receptors,” Cell, vol. 164, no. 4, pp. 780-791, February 2016, doi: 10.1016/j.cell.2016.01.012.
[5] M. Gossen and H. Bujard, “Tight control of gene expression in mammalian cells by tetracycline-responsive promoters.,” Proc. Natl. Acad. Sci., vol. 89, no. 12, pp. 5547-5551 June 1992, doi: 10.1073/pnas.89.12.5547.
[6] M. Latta-Mahieu et al., “Gene Transfer of a Chimeric Trans-Activator Is Immunogenic and Results in Short-Lived Transgene Expression,” Hum. Gene Ther., vol. 13, no. 13, pp. 1611-1620 September 2002, doi: 10.1089/10430340260201707.
[7] L. E. Mays and J. M. Wilson, “The Complex and Evolving Story of T cell Activation to AAV Vector-encoded Transgene Products,” Mol. Ther., vol. 19, no. 1, pp. 16-27, January 2011, doi: 10.1038/mt.2010.250.
[9] S. A. Hoyng et al., “Developing a potentially immunologically inert tetracycline-regulatable viral vector for gene therapy in the peripheral nerve,” Gene Ther., vol. 21, no. 6, pp. 549-557, June 2014, doi: 10.1038/gt.2014.22.
[10] X. Wang, F. G. Cabrera, K. L. Sharp, D. M. Spencer, A. E. Foster, and J. H. Bayle, “Engineering Tolerance toward Allogeneic CAR-T Cells by Regulation of MHC Surface Expression with Human Herpes Virus-8 Proteins,” Mol. Ther., October 2020, doi: 10.1016/j.ymthe.2020.10.019.
[11] L. Peraro et al., “Incorporation of bacterial immunoevasins to protect cell therapies from host antibody-mediated immune rejection,” Mol. Ther., July 2021, doi: 10.1016/j.ymthe.2021.06.022.
[12] A. Annoni, B. D. Brown, A. Cantore, L. S. Sergi, L. Naldini, and M.-G. Roncarolo, “In vivo delivery of a microRNA-regulated transgene induces antigen-specific regulatory T cells and promotes immunologic tolerance,” Blood, vol. 114, no. 25, pp. 5152-5161 December 2009, doi: 10.1182/blood-2009-04-214569.
[13] F. Boisgerault et al., “Prolonged Gene Expression in Muscle Is Achieved Without Active Immune Tolerance Using MicrorRNA 142.3p-Regulated rAAV Gene Transfer,” Hum. Gene Ther., vol. 24, no. 4, pp. 393-405, February 2013, doi: 10.1089/hum.2012.208.
[14] Y. K. Chan et al., “Engineering adeno-associated viral vectors to evade innate immune and inflammatory responses,” Sci. Transl. Med., vol. 13, no. 580, p. eabd3438, February 2021, doi: 10.1126/scitranslmed.abd3438.
[15] V. M. Rivera et al., “A humanized system for pharmacologic control of gene expression,” Nat. Med., vol. 2, no. 9, pp. 1028-132 September 1996, doi: 10.1038/nm0996-1028.
[16] I. Zhu et al., “Modular design of synthetic receptors for programmed gene regulation in cell therapies,” Cell, vol. 185, no. 8, pp. 1431-1443.e16, April 2022, doi: 10.1016/j.cell.2022.03.023.
[17] I. Zhu et al., “Design and modular assembly of synthetic intramembrane proteolysis receptors for custom gene regulation in therapeutic cells,” bioRxiv, p. 2021.05.21.445218, May 2021, doi: 10.1101/2021.05.21.445218.
[18] J. Tycko et al., “High-Throughput Discovery and Characterization of Human Transcriptional Effectors,” Cell, vol. 0, no. 0, December 2020, doi: 10.1016/j.cell.2020.11.024.
[19] M. Fussenegger et al., “Streptogramin-based gene regulation systems for mammalian cells,” Nat. Biotechnol., vol. 18, no. 11, Art. no. 11, November 2000, doi: 10.1038/81208.
[20] J. M. O′shea and N. D. Perkins, “Regulation of the RelA (p65) transactivation domain,” Biochem. Soc. Trans., vol. 36, no. 4, pp. 603-608, July 2008, doi: 10.1042/BST0360603.
[21] E. Yakubovskaya, E. Mejia, J. Byrnes, E. Hambardjieva, and M. Garcia-Diaz, “Helix Unwinding and Base Flipping Enable Human MTERF1 to Terminate Mitochondrial Transcription,” Cell, vol. 141, no. 6, pp. 982-993, June 2010, doi: 10.1016/j.cell.2010.05.018.
[22] L. Klein, B. Kyewski, P. M. Allen, and K. A. Hogquist, “Positive and negative selection of the T cell repertoire: what thymocytes see (and don't see),” Nat. Rev. Immunol., vol. 14, no. 6, pp. 377-391, June 2014, doi: 10.1038/nri3667.
[23] V. Jurtz, S. Paul, M. Andreatta, P. Marcatili, B. Peters, and M. Nielsen, “NetMHCpan-4.0: Improved Peptide-MHC Class I Interaction Predictions Integrating Eluted Ligand and Peptide Binding Affinity Data,” J. Immunol., vol. 199, no. 9, pp. 3360-3368 November 2017, doi: 10.4049/jimmunol. 1700893.
[24] M. D. Robinson, D. J. McCarthy, and G. K. Smyth, “edgeR: a Bioconductor package for differential expression analysis of digital gene expression data,” Bioinformatics, vol. 26, no. 1, pp. 139-140, January 2010, doi: 10.1093/bioinformatics/btp616.
[25] J. T. Bulcha, Y. Wang, H. Ma, P. W. L. Tai, and G. Gao, “Viral vector platforms within the gene therapy landscape,” Sig Transduct Target Ther, vol. 6, no. 1, Art. no. 1, February 2021, doi: 10.1038/s41392-021-00487-6.
[26] J. M. O'shea and N. D. Perkins, “Regulation of the RelA (p65) transactivation domain,” Biochemical Society Transactions, vol. 36, no. 4, pp. 603-608, July 2008, doi: 10.1042/BST0360603.
[27] J. Tycko et al., “High-Throughput Discovery and Characterization of Human Transcriptional Effectors,” Cell, vol. 0, no. 0, December 2020, doi: 10.1016/j.cell.2020.11.024.
[28] E. Yakubovskaya, E. Mejia, J. Byrnes, E. Hambardjieva, and M. Garcia-Diaz, “Helix Unwinding and Base Flipping Enable Human MTERF1 to Terminate Mitochondrial Transcription,” Cell, vol. 141, no. 6, pp. 982-993, June 2010, doi: 10.1016/j.cell.2010.05.018.
[29] H.-S. Li et al., “Multidimensional control of therapeutic human cell function with synthetic gene circuits,” Science, December 2022, doi: 10.1126/science.ade0156.
[30] J. H. Bayle, J. S. Grimley, K. Stankunas, J. E. Gestwicki, T. J. Wandless, and G. R. Crabtree, “Rapamycin Analogs with Differential Binding Specificity Permit Orthogonal Control of Protein Activity,” Chemistry & Biology, vol. 13, no. 1, pp. 99-107, January 2006, doi: 10.1016/j.chembiol.2005.10.017.
[31] V. M. Rivera et al., “A humanized system for pharmacologic control of gene expression,” Nat Med, vol. 2, no. 9, pp. 1028-132 September 1996, doi: 10.1038/nm0996-1028.
[32] M. J. Drummond et al., “Rapamycin administration in humans blocks the contraction-induced increase in skeletal muscle protein synthesis,” The Journal of Physiology, vol. 587, no. 7, pp. 1535-1546, 2009, doi: 10.1113/jphysiol.2008.163816.
[33] A. Mazé and Y. Benenson, “Artificial signaling in mammalian cells enabled by prokaryotic two-component system,” Nat Chem Biol, vol. 16, no. 2, pp. 179-187, February 2020, doi: 10.1038/s41589-019-0429-9.
[34] P. S. Donahue, J. W. Draut, J. J. Muldoon, H. I. Edelstein, N. Bagheri, and J. N. Leonard, “The COMET toolkit for composing customizable genetic programs in mammalian cells,” Nat Commun, vol. 11, no. 1, Art. no. 1, February 2020, doi: 10.1038/s41467-019-14147-5.
[35] R. P. Smith et al., “Massively parallel decoding of mammalian regulatory sequences supports a flexible organizational model,” Nat Genet, vol. 45, no. 9, Art. no. 9, September 2013, doi: 10.1038/ng.2713.
[36] J. E. Davis, K. D. Insigne, E. M. Jones, Q. B. Hastings, and S. Kosuri, “Multiplexed dissection of a model human transcription factor binding site architecture.” bioRxiv, p. 625434, May 13, 2019. doi: 10.1101/625434.
[37] E. Sharon et al., “Inferring gene regulatory logic from high-throughput measurements of thousands of systematically designed promoters,” Nat Biotechnol, vol. 30, no. 6, Art. no. 6, June 2012, doi: 10.1038/nbt.2205.
[38] M. Hegde, C. Strand, R. E. Hanna, and J. G. Doench, “Uncoupling of sgRNAs from their associated barcodes during PCR amplification of combinatorial CRISPR screens,” PLOS ONE, vol. 13, no. 5, p. e0197547, May 2018, doi: 10.1371/journal.pone.0197547.
[39] J. Liu et al., “Extensive Recombination Due to Heteroduplexes Generates Large Amounts of Artificial Gene Fragments during PCR,” PLOS ONE, vol. 9, no. 9, p. e106658, September 2014, doi: 10.1371/journal.pone.0106658.

1. A synthetic transcription factor comprising (i) a DNA binding domain (DBD) derived from a mitochondrial DNA binding protein and (ii) a transcriptional modulation domain derived from one or more other proteins.

2. The synthetic transcription factor of claim 1, wherein said mitochondrial DNA binding protein is from a mammalian species.

3. The synthetic transcription factor of claim 2, wherein said mammalian species is a human.

4. The synthetic transcription factor of any one of claims 1 to 3, wherein said mitochondrial DNA binding protein is MTERF1.

5. The synthetic transcription factor of any one of claims 1 to 4, wherein said DNA binding domain comprises (i) a first MTERF1 motif which has a sequence as shown in SEQ ID NO: 104 or a sequence that has a sequence identity of at least 80%, preferably at least 90%, more preferably at least 95% to SEQ ID NO: 104, and/or (ii) a second MTERF1 motif which has a sequence as shown in SEQ ID NO: 106 or a sequence that has sequence identity of at least 80%, preferably at least 90%, more preferably at least 95% to SEQ ID NO: 106;
and preferably wherein the first MTERF1 motif is N-terminally of the second MTERF1 motif.

6. The synthetic transcription factor of claim 5, wherein said DNA binding domain further comprises (iii) a MTERF1 C-terminal domain which has a sequence as shown in SEQ ID NO: 11 or a sequence that has a sequence identity of at least 70%, preferably at least 80%, more preferably at least 90%, to SEQ ID NO: 11.

7. The synthetic transcription factor of any one of claims 1 to 6, wherein said DNA binding domain comprises a MTERF1 subdomain A which has a sequence as shown in SEQ ID NO: 9 or a sequence that has a sequence identity of at least 80%, preferably at least 90%, more preferably at least 95%, to SEQ ID NO: 9;
in particular wherein said first MTERF1 motif and/or said second MTERF1 motif is contained in said subdomain A.

8. The synthetic transcription factor of any one of claims 1 to 7, wherein said DNA binding domain comprises a MTERF1 subdomain B which has a sequence as shown in SEQ ID NO: 7 or a sequence that has a sequence identity of at least 70%, preferably at least 80%, more preferably at least 90%, to SEQ ID NO: 7; in particular wherein said MTERF1 subdomain A is contained in said MTERF1 subdomain B.

9. The synthetic transcription factor of any one of claims 1 to 7, wherein said DNA binding domain has a sequence identity of at least 60%, preferably at least 70%, more preferably at least 80%, to SEQ ID NO: 1; preferably wherein said DNA binding domain has a sequence as shown in SEQ ID NO: 1.

10. The synthetic transcription factor of any one of claims 1 to 9 which does not comprise a mitochondrial transfer peptide which has a sequence as shown in SEQ ID NO: 37 or a sequence that has a sequence identity of at least 90% to SEQ ID NO: 37.

11. The synthetic transcription factor of any one of claims 1 to 10, which does not have a functional mitochondrial transfer peptide.

12. The synthetic transcription factor of any one of claims 1 to 11, which comprises a nuclear localization signal.

13. The synthetic transcription factor of any one of claims 1 to 12, which is capable of regulating the transcription of at least one gene of interest in a cell, preferably in a nucleus of a cell; and preferably wherein the gene(s) of interest encode(s) a cell death promoting protein such as hBAX or HSV-TK, an immune stimulating cytokine such as IL-2 or IL-12, and/or an antigen-receptor such as a CAR or a TCR.

14. The synthetic transcription factor of claim 13, wherein the synthetic transcription factor regulates the transcription of a gene of interest in a cell, preferably in a cell nucleus by (i) promoting transcription of the gene of interest, or by (ii) suppressing transcription of the gene of interest.

15. The synthetic transcription factor of any one of claims 1 to 14, wherein said synthetic transcription factor and/or said DNA binding domain is capable of binding to a response element in a cell, preferably in a nucleus of a cell.

16. The synthetic transcription factor of claim 15, wherein said response element comprises a MTERF1 binding site which has a sequence as shown in SEQ ID NO: 42 or a sequence which has a sequence identity of at least 60%, preferably at least 80%, more preferably at least 90% to SEQ ID NO: 42.

17. The synthetic transcription factor of claim 16, wherein said response element comprises multiple copies of said binding site, e.g., 2 to 15 copies; preferably 2 to 5 copies.

18. The synthetic transcription factor of claim 17, wherein two or more or all copies of said binding site in said response element are directly adjacent to each other or separated by one or more, e.g., 6 or 10, nucleotides.

19. The synthetic transcription factor of any one of claims 15 to 18, which is capable of binding to a promoter comprising said response element in a cell, preferably in a nucleus of a cell.

20. The synthetic transcription factor of claim 19, wherein said promoter further comprises a minimal promoter, for example, a minimal TATA box which preferably has a sequence as shown in SEQ ID NO: 103 or a minimal CMV promoter which preferably has a sequence as shown in SEQ ID NO: 136; and preferably wherein said minimal promoter is 3′ of said response element.

21. The synthetic transcription factor of any one of claims 19 to 20, wherein said promoter has a sequence as shown in SEQ ID NO: 110.

22. The synthetic transcription factor of any one of claims 19 to 21, wherein said promoter is operably linked to a gene of interest, preferably in said cell nucleus.

23. The synthetic transcription factor of claim 22, wherein the binding of the synthetic transcription to said promoter, in particular the binding of said DNA binding domain to said response element within the promoter, regulates the transcription of a gene of interest which is operably linked to said promoter, e.g. in a nucleus of a cell; in particular wherein said binding regulates the transcription of the gene(s) of interest in a nucleus of a cell as defined in claim 13 or 14.

24. The synthetic transcription factor of any one of claims 1 to 23, which is capable of entering a nucleus of a cell.

25. The synthetic transcription factor of any one of claims 1 to 24, which has the ability to localize more efficiently to the nucleus in a cell than to the mitochondria in said cell; in particular wherein said ability is determined by measuring the amount of the synthetic transcription factor separately in the nucleus and the mitochondria of the same cell(s).

26. The synthetic transcription factor of any one of claims 1 to 25, which is not able to enter mitochondria.

27. The synthetic transcription factor of any one of claims 13 to 26, wherein said cell is from the same species, e.g. the same mammalian species, that said mitochondrial DNA binding protein is from.

28. The synthetic transcription factor of claim 27, which does essentially not alter transcription, i.e. the transcriptome, in said cell apart from the transcription of the gene(s) of interest.

29. The synthetic transcription factor of claim 27 or 28, which does not specifically bind to essentially any endogenous DNA sequence in said cell.

30. The synthetic transcription factor of any one of claims 27 to 29, wherein said DNA binding domain does not specifically bind to essentially any endogenous DNA sequence in a nucleus of said cell.

31. The synthetic transcription factor of any one of claims 27 to 30, which does essentially not compete with the mitochondrial DNA binding protein from which the DBD is derived for sequence-specific DNA binding in said cell.

32. The synthetic transcription factor of any one of claims 27 to 31, which does essentially not interfere with the function of the mitochondrial DNA binding protein from which the DBD is derived.

33. The synthetic transcription factor of any one of claims 1 to 32, wherein said transcriptional modulation domain is capable of regulating the transcription of a gene, in particular when said transcriptional modulation domain is part of, bound to or interacts with a DNA binding protein that is capable to bind to or interact with a regulatory sequence, e.g. a promoter or enhancer, of said gene.

34. The synthetic transcription factor of any one of claims 1 to 33, wherein said transcriptional modulation domain is capable of binding to and/or interacting with an RNA polymerase, preferably RNA polymerase II; at least one other transcription factor, e.g., a general transcription factor; and/or at least one transcriptional coregulator such as a transcriptional coactivator and/or a transcriptional corepressor.

35. The synthetic transcription factor of any one of claims 1 to 34, wherein said transcriptional modulation domain is (i) an activation domain or (ii) a repression domain.

36. The synthetic transcription factor of any one of claims 1 to 34, wherein said transcriptional modulation domain is able to (i) promote transcription of a gene, in particular when defined as an activation domain, or (ii) suppress transcription of a gene, in particular when defined as a repression domain.

37. The synthetic transcription factor of any one of claims 1 to 36, wherein said transcriptional modulation domain, in particular said activation domain, has a sequence identity of at least 60%, preferably at least 70%, more preferably at least 80% to a sequence selected from the group consisting of: SEQ ID NO: 3, SEQ ID NO: 33, SEQ ID NO: 52, SEQ ID NO: 27, SEQ ID NO: 29, SEQ ID NO: 54, SEQ ID NO: 56, SEQ ID NO: 58, SEQ ID NO: 17, SEQ ID NO: 19, SEQ ID NO: 60, SEQ ID NO: 62, SEQ ID NO: 13, SEQ ID NO: 64, SEQ ID NO: 66 and SEQ ID NO: 68; or wherein said transcriptional modulation domain, in particular said activation domain, comprises at least one sequence that has a sequence identity of at least 60%, preferably at least 70%, more preferably at least 80% to a sequence selected from the group consisting of: SEQ ID NO: 3, SEQ ID NO: 33, SEQ ID NO: 52, SEQ ID NO: 27, SEQ ID NO: 29, SEQ ID NO: 54, SEQ ID NO: 56, SEQ ID NO: 58, SEQ ID NO: 17, SEQ ID NO: 19, SEQ ID NO: 60, SEQ ID NO: 62, SEQ ID NO: 13, SEQ ID NO: 64, SEQ ID NO: 66 and SEQ ID NO: 68.

38. The synthetic transcription factor of any one of claims 1 to 37, wherein said transcriptional modulation domain comprises a first, a second and/or a third RELA transactivation domain; wherein the first RELA transactivation domain (TA1) has a sequence as shown in SEQ ID NO: 31 or a sequence that has a sequence identity of at least 80% to SEQ ID NO: 31; wherein the second RELA transactivation domain has a sequence as shown in SEQ ID NO: 132 or a sequence that has a sequence identity of at least 80% to SEQ ID NO: 132; and wherein the third RELA transactivation domain has a sequence as shown in SEQ ID NO: 134 or a sequence that has a sequence identity of at least 80% to SEQ ID NO: 134; and preferably wherein said transcriptional modulation domain comprises at least the first RELA transactivation domain.

39. The synthetic transcription factor of claim 38, wherein said transcriptional modulation domain comprises multiple copies, e.g. two or three copies, of said first, second and/or third RELA domain, preferably of said first RELA domain (TA1).

40. The synthetic transcription factor of any one of claims 1 to 39, wherein said transcriptional modulation domain comprises a RELA subdomain A which has a sequence as shown in SEQ ID NO: 3 or a sequence that has a sequence identity of at least 70%, preferably at least 80%, more preferably at least 90% to SEQ ID NO: 3.

41. The synthetic transcription factor of any one of claims 1 to 40, wherein said transcriptional modulation domain comprises a RELA subdomain B which has a sequence as shown in SEQ ID NO: 27 or a sequence that has a sequence identity of at least 70%, preferably at least 80%, more preferably at least 90% to SEQ ID NO: 27; in particular wherein said RELA subdomain A is contained in said RELA subdomain B.

42. The synthetic transcription factor of any one of claims 1 to 41, wherein said transcriptional modulation domain comprises a RELA subdomain C which has a sequence as shown in SEQ ID NO: 29 or a sequence that has a sequence identity of at least 60%, preferably at least 70%, more preferably at least 80% to SEQ ID NO: 29; in particular wherein said first, second and/or third RELA motifs, said RELA subdomain A and/or said RELA subdomain B are contained in said RELA subdomain C.

43. The synthetic transcription factor of any one of claims 1 to 42, wherein said transcriptional modulation domain comprises a FOXO3 transactivation domain (FOXO TAD) which has a sequence as shown in SEQ ID NO: 17 or a sequence that has a sequence identity of at least 80% to SEQ ID NO: 17.

44. The synthetic transcription factor of claim 43, wherein said transcriptional modulation domain comprises multiple copies, e.g. two or three copies, of said FOXO3 transactivation domain (FOXO TAD); preferably wherein said transcriptional modulation domain has a sequence as shown in SEQ ID NO: 33 or a sequence that has a sequence identity of at least 70%, preferably at least 80%, more preferably at least 90% to SEQ ID NO: 33.

45. The synthetic transcription factor of any one of claims 1 to 44, wherein said transcriptional modulation domain comprises a MYB transactivation domain (LMSTEN) which has a sequence as shown in SEQ ID NO: 17 or a sequence that has a sequence identity of at least 80% to SEQ ID NO: 17.

46. The synthetic transcription factor of claim 45, wherein said transcriptional modulation domain comprises multiple copies, e.g. two or three copies, of said MYB transactivation domain (LMSTEN); preferably wherein said transcriptional modulation domain has a sequence as shown in SEQ ID NO: 196 or a sequence that has a sequence identity of at least 70%, preferably at least 80%, more preferably at least 90% to SEQ ID NO: 196.

47. The synthetic transcription factor of any one of claims 1 to 46, wherein said transcriptional modulation domain comprises two copies of
(i) a first RELA transactivation domain (TA1) that has a sequence as shown in SEQ ID NO: 31 or a sequence that has a sequence identity of at least 80% to SEQ ID NO: 31;
(ii) a FOXO3 transactivation domain (FOXO TAD) that has a sequence as shown in SEQ ID NO: 33 or a sequence that has a sequence identity of at least 80% to SEQ ID NO: 33; or
(iii) MYB transactivation domain (LMSTEN) that has a sequence as shown in SEQ ID NO: 196 or a sequence that has a sequence identity of at least 80% to SEQ ID NO: 196;
preferably two copies of a FOXO3 transactivation domain (FOXO TAD) as shown in SEQ ID NO: 33 or a sequence that has a sequence identity of at least 80% to SEQ ID NO: 33.

48. The synthetic transcription factor of any one of claims 1 to 47, wherein said transcriptional modulation domain comprises three copies of a first RELA transactivation domain (TA1) that has a sequence as shown in SEQ ID NO: 31 or a sequence that has a sequence identity of at least 80% to SEQ ID NO: 31.

49. The synthetic transcription factor of any one of claims 1 to 48, wherein said transcriptional modulation domain has a sequence as shown in SEQ ID NO: 33, SEQ ID NO: 35, SEQ ID NO: 194 or SEQ ID NO: 196, or a sequence that has a sequence identity of at least 70%, preferably at least 80%, more preferably at least 90% to any of these sequences.

50. The synthetic transcription factor of any one of claims 1 to 48, wherein said transcriptional modulation domain has a sequence as shown in SEQ ID NO: 33, or a sequence that has a sequence identity of at least 70%, preferably at least 80%, more preferably at least 90% to SEQ ID NO: 33.

51. The synthetic transcription factor of any one of claims 38 to 50, wherein said transcriptional modulation domain comprises at least two domains independently selected from the group consisting of: said TA1, said FOXO TAD and said LMSTEN; and preferably wherein said transcriptional modulation domain comprises at least a FOXO TAD; and more preferably, wherein said transcriptional modulation domain comprises at least a FOXO TAD, a TA1 and a LMSTEN.

52. The synthetic transcription factor of claim 51, wherein said transcriptional modulation domain comprises (i) two FOXO TAD, (ii) a FOXO TAD and a LMSTEN, (iii) a LMSTEN and a TA1, (iv) a FOXO TAD and a TA1, or (v) a FOXO TAD, a LMSTEN and a TA1; and preferably a FOXO TAD, in particular, said options (i), (ii), (iv) or (v).

53. The synthetic transcription factor of any one of claims 1 to 51, wherein said transcriptional modulation domain has a sequence as shown in SEQ ID NO: 52, SEQ ID NO: 54, SEQ ID NO: 56 or SEQ ID NO: 58, or a sequence that has a sequence identity of at least 70%, preferably at least 80%, more preferably at least 90% to any of these sequences.

54. The synthetic transcription factor of any one of claims 1 to 52, wherein said transcriptional modulation domain has a sequence as shown in SEQ ID NO: 52, SEQ ID NO: 33, SEQ ID NO: 54 or SEQ ID NO: 58, or a sequence that has a sequence identity of at least 70%, preferably at least 80%, more preferably at least 90% to any of these sequences.

55. The synthetic transcription factor of any one of claims 51 to 54, wherein said transcriptional modulation domain has a sequence as shown in SEQ ID NO: 52 or a sequence that has a sequence identity of at least 70%, preferably at least 80%, more preferably at least 90% to SEQ ID NO: 52.

56. The synthetic transcription factor of any one of claims 51 to 55, wherein said DNA binding domain comprises or consists of a MTERF1 subdomain A which has a sequence as shown in SEQ ID NO: 9 or a sequence that has a sequence identity of at least 80%, preferably at least 90%, more preferably at least 95%, to SEQ ID NO: 9; and preferably, wherein said transcriptional modulation domain comprises or consists of a sequence as defined in claim 54 or 55.

57. The synthetic transcription factor of any one of claims 37 to 45, 51 and 53, wherein said transcriptional modulation domain has a sequence identity of at least 60%, preferably at least 70%, more preferably at least 80% to a sequence selected from the group consisting of: SEQ ID NO: 3, SEQ ID NO: 33, SEQ ID NO: 52, SEQ ID NO: 27, SEQ ID NO: 29, SEQ ID NO: 54, SEQ ID NO: 56 and SEQ ID NO: 58.

58. The synthetic transcription factor of any one of claims 37 to 45, 51, 53 and 57, wherein said transcriptional modulation domain has a sequence identity of at least 70%, preferably at least 80%, more preferably at least 90% to a sequence selected from the group consisting of: SEQ ID NO: 3, SEQ ID NO: 33 and SEQ ID NO: 52.

59. The synthetic transcription factor of any one of claims 1 to 58, wherein said transcriptional modulation domain comprises a sequence that has a sequence identity of at least 60%, preferably at least 70%, more preferably at least 80% to a sequence selected from the group consisting of: SEQ ID NO: 60, SEQ ID NO: 62, SEQ ID NO: 13, SEQ ID NO: 64, SEQ ID NO: 66 and SEQ ID NO: 68, preferably in addition to said first, second and/or third RELA motif, e.g. said TA1, said RELA subdomain A, B or C, said FOXO TAD and/or said LMSTEN.

60. The synthetic transcription factor of any one of claims 1 to 36, wherein said transcriptional modulation domain, in particular said repression domain, has a sequence identity of at least 60%, preferably at least 70%, more preferably at least 80% to a sequence selected from the group consisting of: SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96 and SEQ ID NO: 98; or wherein said transcriptional modulation domain, in particular said repression domain, comprises at least one sequence that has a sequence identity of at least 60%, preferably at least 70%, more preferably at least 80% to a sequence selected from the group consisting of: SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96 and SEQ ID NO: 98.

61. The synthetic transcription factor of any one of claim 60, wherein said transcriptional modulation domain has a sequence identity of at least 60%, preferably at least 70%, more preferably at least 80% to a sequence selected from the group consisting of: preferably to SEQ ID NO: 70, SEQ ID NO: 72, SEQ ID NO: 74 or SEQ ID NO: 76.

62. The synthetic transcription factor of claim 60 or 61, wherein said transcriptional modulation domain has a sequence identity of at least 60%, preferably at least 70%, more preferably to SEQ ID NO: 70.

63. The synthetic transcription factor of any one of claims 1 to 62, wherein the one or more proteins from which said transcriptional modulation domain is derived from are from the same species, e.g. the same mammalian species, that said mitochondrial DNA binding protein is from.

64. The synthetic transcription factor of any one of claims 1 to 63, which is composed essentially of parts of proteins from the same species, e.g., the same mammalian species.

65. The synthetic transcription factor of any one of claims 1 to 58 and 60 to 64, wherein said mitochondrial DNA binding protein, e.g. MTERF1, and said one or more other proteins from which said transcriptional modulation domain is derived from, e.g. RELA, FOXO3 and/or MYB, are from a human.

66. The synthetic transcription factor of any one of claims 1 to 58 and 60 to 65, which is composed essentially of parts of human proteins.

67. The synthetic transcription factor of claim 65 or 66, wherein said transcriptional modulation domain is derived from a single human protein.

68. The synthetic transcription factor of any one of claims 65 to 67, wherein said transcriptional modulation domain, in particular said activation domain, has a sequence identity of at least 90%, preferably at least 95%, more preferably at least 99%, to SEQ ID NO: 3, SEQ ID NO: 27 or SEQ ID NO: 29, preferably to SEQ ID NO: 3.

69. The synthetic transcription factor of any one of claims 65 to 67, wherein said transcriptional modulation domain, in particular said repression domain, has a sequence identity of at least 90%, preferably at least 95%, more preferably at least 99%, to SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96 or SEQ ID NO: 98, preferably to SEQ ID NO: 70.

70. The synthetic transcription factor of any one of claims 1 to 69, which is essentially non-immunogenic in a human.

71. The synthetic transcription factor of any one of claims 1 to 69, which is essentially non-immunogenic in the mammalian species that said mitochondrial DNA binding protein is from.

72. The synthetic transcription factor of any one of claims 65 to 69, which is essentially non-immunogenic in a human.

73. The synthetic transcription factor of any one of claims 1 to 71 further comprising a controllable domain, preferably a controllable destabilization domain or a controllable localization domain.

74. The synthetic transcription factor of claim 73, wherein said controllable domain is controllable (i) by a compound, preferably by a small molecule, or (ii) by light, preferably by a particular wavelength or a particular range of wavelengths.

75. The synthetic transcription factor of claim 73 or 74, wherein the synthetic transcription factor comprising said controllable destabilization domain is stabilized or destabilized by said compound or light; and/or wherein the synthetic transcription factor comprising said controllable localization domain is located to either the nucleus or the cytoplasm, preferably to the nucleus, of a cell by said compound or light.

76. The synthetic transcription factor of any one of claims 73 to 75, wherein said controllable destabilization domain comprises a NS3 domain which has a sequence as shown in SEQ ID NO: 158 or a sequence that has a sequence identity of at least 80%, preferably at least 90%, more preferably at least 95%, to SEQ ID NO: 158.

77. The synthetic transcription factor of claim 76, wherein the synthetic transcription factor comprising said NS3 domain is stabilized by grazoprevir.

78. The synthetic transcription factor of any one of claims 73 to 77, wherein said controllable localization domain comprises an ERT2 domain which has a sequence as shown in SEQ ID NO: 152 or a sequence that has a sequence identity of at least 80%, preferably at least 90%, more preferably at least 95%, to SEQ ID NO: 152.

79. The synthetic transcription factor of claim 78, wherein the synthetic transcription factor comprising said ERT2 domain is located to the nucleus of a cell by 4-hydroxytamoxifen.

80. The synthetic transcription factor of any one of claims 73 to 80, wherein said controllable domain comprises a FRB domain and a FKBP domain, wherein said FRB domain has a sequence as shown in SEQ ID NO: 140 or a sequence that has a sequence identity of at least 80%, preferably at least 90%, more preferably at least 95%, to SEQ ID NO: 140, and/or wherein said FKBP domain has a sequence as shown in SEQ ID NO: 142 or a sequence that has a sequence identity of at least 80%, preferably at least 90%, more preferably at least 95%, to SEQ ID NO: 142.

81. The synthetic transcription factor of claim 80, wherein said FRB domain and said FKBP domain bind to each other in the presence of C16-(S)-7-methylindolerapamycin.

82. The synthetic transcription factor of any one of claims 1 to 81, further comprising a synNotch core which has a sequence as shown in SEQ ID NO: 160 or a sequence that has a sequence identity of at least 80%, preferably at least 90%, more preferably at least 95%, to SEQ ID NO: 160.

83. The synthetic transcription factor of any one of claims 1 to 82, which is a fusion protein comprising said DNA binding domain and said transcriptional modulation domain.

84. The synthetic transcription factor of claim 83, wherein DNA binding domain is N-terminally of the transcriptional modulation domain.

85. The synthetic transcription factor of claim 83 or 84, wherein said fusion protein further comprises a controllable destabilization domain as defined in any one of claims 73 to 77 or a controllable localization domain as defined in any one of claims 73 to 75, 78 and 79.

86. The synthetic transcription factor of claim 83 or 84, wherein said fusion protein further comprises a single-chain variable fragment (scFv) and the synNotch core as defined in claim 82; and, preferably, wherein the order of the domains in said fusion protein from N-terminus to C-terminus is as follows: (i) scFv, (ii) synNotch, and (iii) DNA binding domain and transcriptional modulation domain, wherein the DNA binding domain may be N-terminally or C-terminally of the transcriptional modulation domain.

87. A nucleic acid encoding the synthetic transcription factor of any one of claims 83 to 86.

88. The nucleic acid of claim 87 which is a DNA or an RNA.

89. The nucleic acid of claim 87 or 88 which is an mRNA, e.g., an mRNA contained in a lipid nanoparticle.

90. The nucleic acid of claim 87, comprising a DNA sequence as shown in SEQ ID NO: 5 or a DNA sequence which has a sequence identity of at least 60%, preferably at least 70%, more preferably at least 80%, to SEQ ID NO: 5, and wherein said DNA sequence encodes a DNA binding domain that has a sequence identity of at least 60%, preferably at least 70%, more preferably at least 80%, to SEQ ID NO: 1; preferably wherein said DNA binding domain has a sequence as shown in SEQ ID NO: 1.

91. A DNA plasmid comprising the nucleic of any one of claims 87 to 90, preferably wherein said plasmid is suitable for expressing the synthetic transcription factor in a cell.

92. A viral vector comprising the nucleic acid of any one of claims 87 to 90, the plasmid of claim 91, or a nucleic acid which has a complementary sequence to the nucleic acid of any one of claims 87 to 90.

93. A cell comprising the nucleic acid of any one of claims 87 to 90, the plasmid of claim 91 or the viral vector of claim 92.

94. The synthetic transcription factor of any one of claims 1 to 82, which comprises or consists of a first and a second polypeptide, wherein said first polypeptide comprises said DNA binding domain and said second polypeptide comprises said transcriptional modulation domain.

95. The synthetic transcription factor of claim 94, wherein each of the first and second polypeptide comprises a multimerization domain, and wherein the multimerization domains of the first and second polypeptide are capable of binding to and/or interacting with each other.

96. The synthetic transcription factor of claim 95, wherein said multimerization domain is a dimerization domain.

97. The synthetic transcription factor of claim 95 or 96, wherein said multimerization domain is a homodimerization domain, in particular, wherein the multimerization domains of the first and second polypeptides are identical to each other.

98. The synthetic transcription factor of claim 95 or 96, wherein said multimerization domain is a heterodimerization domain, in particular, wherein the multimerization domains of the first and second polypeptide are different from each other.

99. The synthetic transcription factor of claim 98, wherein
(i) the multimerization domain of the first polypeptide comprises or consists of a SYNZIP1 domain and the multimerization domain of the second polypeptide comprises or consists of a SYNZIP2 domain; or
(ii) the multimerization domain of the first polypeptide comprises or consists of a SYNZIP2 domain and the multimerization domain of the second polypeptide comprises or consists of a SYNZIP1 domain;
wherein said SYNZIP1 domain has a sequence as shown in SEQ ID NO: 154 or a sequence that has a sequence identity of at least 80%, preferably at least 90%, more preferably at least 95%, to SEQ ID NO: 154; and
wherein said SYNZIP2 domain has a sequence as shown in SEQ ID NO: 156 or a sequence that has a sequence identity of at least 80%, preferably at least 90%, more preferably at least 95%, to SEQ ID NO: 156.

100. The synthetic transcription factor of any one of claims 95 to 99, wherein said multimerization domain is a controllable domain, in particular, wherein said multimerization domain is controllable (i) by a compound, preferably by a small molecule, or (ii) by light, preferably by a particular wavelength or a particular range of wavelengths.

101. The synthetic transcription factor of claim 100, wherein said multimerization domain is a dimerization domain, and wherein said compound or light controls the dimerization of the multimerization domains of the first and second polypeptide; and preferably, wherein said first and second polypeptide bind to and/or interact with each other in the presence of said small molecule or light.

102. The synthetic transcription factor of any one of claims 94 to 96, 98, 100 and 101, wherein
(i) the multimerization domain of the first polypeptide comprises or consists of an FKBP domain and the multimerization domain of the second polypeptide comprises or consists of an FRB domain; or
(ii) the multimerization domain of the first polypeptide comprises or consists of an FRB domain and the multimerization domain of the second polypeptide comprises or consists of an FKBP domain;
wherein said FKBP domain has a sequence as shown in SEQ ID NO: 142 or a sequence that has a sequence identity of at least 80%, preferably at least 90%, more preferably at least 95%, to SEQ ID NO: 142; and
wherein said FRB domain has a sequence as shown in SEQ ID NO: 140 or a sequence that has a sequence identity of at least 80%, preferably at least 90%, more preferably at least 95%, to SEQ ID NO: 140;
and preferably, wherein the multimerization domain of said first polypeptide comprises or consists of said FKBP domain, and the multimerization domain of said second polypeptide comprises or consists of said FRB domain.

103. The synthetic transcription factor of claim 102, wherein said DNA binding domain is N-terminally of said FKBP domain in said first polypeptide, and/or wherein said transcriptional modulation domain is C-terminally of said FRB domain in said second polypeptide.

104. The synthetic transcription factor of claim 102 or 103, wherein the first and second polypeptide bind to and/or interact with each other in the presence of C16-(S)-7-methylindolerapamycin, in particular, wherein C16-(S)-7-methylindolerapamycin induces heterodimerization of said FKBP domain and said FRB domain.

105. The synthetic transcription factor of any one of claims 102 to 104, wherein (i) the DNA binding domain and the FKBP domain are linked to each other via a first peptide linker, and/or (ii) the transcriptional modulation domain and the FRB domain are linked to each other via a second peptide linker.

106. The synthetic transcription factor of claim 105, wherein said first and second peptide linker are independently selected from the group consisting of: a cMyc NLS linker as shown in SEQ ID NO: 122, a 6AP (AP6) linker as shown in SEQ ID NO: 120, an AP8 linker as shown in SEQ ID NO: 144, a G4S linker as shown in SEQ ID NO: 118, an EAAAK3 linker as shown in SEQ ID NO: 146, an EAAAK2 linker as shown in SEQ ID NO: 148 and an G4S4 linker as shown in SEQ ID NO: 150.

107. The synthetic transcription factor of claim 105, wherein said first peptide linker is a cMyc NLS linker as shown in SEQ ID NO: 122 or a 6AP (AP6) linker as shown in SEQ ID NO: 120, and/or wherein said second peptide linker is a 6AP (AP6) linker as shown in SEQ ID NO: 120.

108. The synthetic transcription factor of any one of claims 102 to 107, wherein said transcriptional modulation domains is as defined in any one of claims 51 to 58.

109. The synthetic transcription factor of any one of claims 102 to 108, wherein said transcriptional modulation domain has a sequence as shown in SEQ ID NO: 52, SEQ ID NO: 29, SEQ ID NO: 33, SEQ ID NO: 54 or SEQ ID NO: 58, or a sequence that has a sequence identity of at least 70%, preferably at least 80%, more preferably at least 90% to any of these sequences.

110. The synthetic transcription factor of any one of claims 102 to 108, wherein said wherein said transcriptional modulation domain has a sequence as shown in SEQ ID NO: 52 or SEQ ID NO: 29, or a sequence that has a sequence identity of at least 70%, preferably at least 80%, more preferably at least 90% to SEQ ID NO: 52 or SEQ ID NO: 29.

111. The synthetic transcription factor of any one of claims 102 to 110, wherein said first polypeptide comprises a sequence as shown in SEQ ID NO: 174, 176, 178, 180, 182, 184, 186, 188, or 192, or a sequence that has a sequence identity of at least 70%, preferably at least 80%, more preferably at least 90% to SEQ ID NO: 174, 176, 178, 180, 182, 184, 186, 188, or 192.

112. The synthetic transcription factor of any one of claims 102 to 111, wherein said second polypeptide comprises a sequence as shown in SEQ ID NO: 162, 164, 166, 168, 170, 172, or 190, or a sequence that has a sequence identity of at least 70%, preferably at least 80%, more preferably at least 90% to SEQ ID NO: 162, 164, 166, 168, 170, 172, or 190.

113. A combination of nucleic acids encoding the synthetic transcription factor of any one of claims 1 to 82, wherein one nucleic acid encodes said DNA binding domain and another nucleic acid encodes said transcriptional modulation domain.

114. A combination of nucleic acids encoding the synthetic transcription factor of any one of claims 94 to 112, comprising a first and a second nucleic acid, wherein said first nucleic acid encodes said first polypeptide, and said second nucleic acid encodes said second polypeptide.

115. The combination of claim 113 or 114, wherein one nucleic acid, in particular said first nucleic acid, has a DNA sequence as shown in SEQ ID NO: 5 or a DNA sequence which has a sequence identity of at least 60%, preferably at least 70%, more preferably at least 80%, to SEQ ID NO: 5, and wherein said DNA sequence encodes a DNA binding domain that has a sequence identity of at least 60%, preferably at least 70%, more preferably at least 80%, to SEQ ID NO: 1; preferably wherein said DNA binding domain has a sequence as shown in SEQ ID NO: 1.

116. The combination of claim 113 or 114, wherein said nucleic acids are DNA or RNA molecules.

117. The combination of claim 116, wherein said nucleic acids are mRNA molecules, e.g., mRNAs contained in lipid nanoparticles.

118. A combination of DNA plasmids, comprising the combination of nucleic acids of any one of claims 113 to 115, preferably wherein said plasmids are suitable for expressing the synthetic transcription factor in a cell.

119. A combination of viral vectors comprising the nucleic acids of any one of claims 113 to 115, the plasmids of claim 119, or nucleic acids having complementary sequences to the nucleic acids of any one of claims 113 to 115.

120. A cell comprising the combination of any one of claims 113 to 119.

121. A DNA construct comprising a promoter (P) comprising a response element and a minimal promoter, characterized in that said response element comprises a MTERF1 binding site which comprises or consists of a sequence as shown in SEQ ID NO: 42 or SEQ ID NO: 200, or a sequence that has a sequence identity of at least 60%, preferably at least 80%, more preferably at least 90% to SEQ ID NO: 42 or SEQ ID NO: 200.

122. The DNA construct of claim 121, wherein said MTERF1 binding site comprises or consists of a sequence as shown in SEQ ID NO: 42, or a sequence that has a sequence identity of at least 60%, preferably at least 80%, more preferably at least 90% to SEQ ID NO: 42.

123. The DNA construct of claim 121 or 122, wherein said response element consists of (i) one or multiple copies, e.g. 2 to 50 copies, preferably 2 to 5 copies, of said MTERF1 binding site, wherein said multiple copies are directly adjacent to each other, or (ii) multiple copies, e.g. 2 to 50 copies, preferably 2 to 5 copies, of said MTERF1 binding site and a BS-BS spacer between at least two, preferably all, successive copies of said binding site, wherein the BS-BS spacer has a length of 1 to 1000 nucleotides, preferably 1 to 100 nucleotides, more preferably 1 to 10, e.g., 1 to 6 nucleotides; and, preferably, wherein said BS-BS spacer consists of the 1 to 10 nucleotides at the 5′ end of the sequence shown in SEQ ID NO: 198.

124. The DNA construct of any one of claims 121 to 123, wherein said response element consists of multiple copies of said MTERF1 binding site, e.g. 2 to 15 copies, which are directly adjacent to each other.

125. The DNA construct of any one of claims 121 to 124, wherein said DNA construct has a length of at most about 106, preferably at most 105, more preferably at most about 10000 nucleotides.

126. The DNA construct of any one of claims 121 to 125, wherein said response element and said minimal promoter are separated from each other by a RE-minP spacer having a length of at most about 2000 nucleotides, preferably at most about 200 nucleotides, more preferably at most about 20 nucleotides, most preferably about 6 to 10 nucleotides, e.g., about 6 or 8 nucleotides; preferably, wherein said RE-minP spacer consists of the 1 to 10 nucleotides at the 5′ end of the sequence shown in SEQ ID NO: 199; and, preferably, said MTERF1 binding site comprises or consists of a sequence as shown in SEQ ID NO: 42, or a sequence that has a sequence identity of at least 60%, preferably at least 80%, more preferably at least 90% to SEQ ID NO: 42.

127. The DNA construct of any one of claims 121 to 126, wherein said minimal promoter is 3′ or 5′, preferably 3′, of said response element.

128. The DNA construct of any one of claims 121 to 127, wherein said minimal promoter is a minimal TATA box which has a sequence identity of at least 60%, preferably at least 80%, more preferably at least 90% to SEQ ID NO: 103.

129. The DNA construct of any one of claims 121 to 127, wherein said minimal promoter is a minimal CMV promoter which has a sequence identity of at least 60%, preferably at least 80%, more preferably at least 90% to SEQ ID NO: 136.

130. The DNA construct of any one of claims 121 to 129, wherein said promoter (P), in particular said response element, is able to bind to the synthetic transcription factor of any one of claims 1 to 86 and 94 to 112.

131. The DNA construct of any one of claims 121 to 130, which further comprises at least one gene of interest, preferably encoding a cell death promoting protein such as hBAX or HSV-TK, an immune stimulating cytokine such as IL-2 or IL-12, and/or an antigen-receptor such as a CAR or a TCR.

132. The DNA construct of claim 131, wherein said promoter (P) is operably linked to at least one gene of interest; preferably wherein at least one gene of interest in 3′ of the minimal promoter.

133. The DNA construct of any one of claims 130 to 132, wherein at least one of said gene(s) of interest is transcribed when said promoter (P), in particular said response element, is bound by the synthetic transcription factor of any one of claims 1 to 86 and 94 to 112 in a cell.

134. The DNA construct of any one of claims 130 to 133, wherein at least one of said gene(s) of interest is transcribed when said promoter (P), in particular said response element, is bound by the synthetic transcription factor of any one of claims 1 to 86 and 94 to 112 in a nucleus of a human cell.

135. The DNA construct of any one of claims 121 to 134, which does not comprise a sequence as shown in SEQ ID NO: 117 or a sequence which has a sequence identity of at least 90% to SEQ ID NO: 117.

136. The DNA construct of any one of claims 121 to 135 which comprises a sequence selected from the group consisting of: SEQ ID NO: 201 to 1190.

137. The DNA construct of any one of claims 121 to 135, wherein said response element comprises or consists of two copies of an MTERF1 binding site, each having a sequence as shown in SEQ ID NO: 42, or a sequence that has a sequence identity of at least 60%, preferably at least 80%, more preferably at least 90% to SEQ ID NO: 42; wherein said two copies of the MTERF1 binding site are directly adjacent to each other; and, wherein the response element and the minimal promoter are separated by 8 nucleotides from each other.

138. The DNA construct of any one of claims 121 to 137 which comprises the sequence as shown in SEQ ID NO: 300.

139. The DNA construct of any one of claims 121 to 135, wherein said response element consists of five copies of an MTERF1 binding site, said copies being separated by 8 nucleotides from each other, and wherein each MTERF1 binding site has a sequence as shown in SEQ ID NO: 42, or a sequence that has a sequence identity of at least 60%, preferably at least 80%, more preferably at least 90% to SEQ ID NO: 42; and wherein the response element and the minimal promoter are separated by 10 nucleotides from each other.

140. The DNA construct of any one of claim 121 to 135 or 139 which comprises the sequence as shown in SEQ ID NO: 693.

141. A single or double stranded nucleic acid comprising the sense strand of the DNA construct of any one of claims 121 to 140 and/or the antisense strand of the DNA construct of any one of claims 121 to 140.

142. The single or double stranded nucleic acid of claim 141, which is a DNA or an RNA.

143. A DNA plasmid comprising the DNA construct of any one of claims 121 to 140.

144. A viral vector comprising the DNA construct of any one of claims 121 to 140, the single or double stranded nucleic acid of claim 141 or 142, or the DNA plasmid of claim 143.

145. A cell comprising the DNA construct of any one of claims 121 to 140.

146. A system comprising (i) the synthetic transcription factor of any one of claims 1 to 86 and 94 to 112, the nucleic acid of any one of claims 87 to 93, the DNA plasmid of claim 94, the viral vector of claim 95, or the combination of any one of claims 113 to 119 and (ii) the DNA construct of any one of claims 121 to 140, the DNA plasmid of claim 143 and/or the viral vector of claim 144; preferably wherein said system comprises the synthetic transcription factor of any one of claims 1 to 86 and 94 to 112 and the DNA construct of any one of claims 121 to 140; and preferably wherein said system is an engineered genetic network, preferably a biocomputing circuit.

147. The system of claim 146 for regulating transcription of at least one gene of interest, preferably wherein said gene of interest is comprised in said DNA construct, preferably 3′ of the minimal promoter; and preferably wherein the gene(s) of interest encode(s) a cell death promoting protein such as hBAX or HSV-TK, an immune stimulating cytokine such as IL-2 or IL-12, and/or an antigen-receptor such as a CAR or a TCR.

148. A cell comprising the synthetic transcription factor of any one of claims 1 to 86 and 94 to 112 and the DNA construct of any one of claims 121 to 140.

149. A kit comprising the synthetic transcription factor of any one of claims 1 to 86 and 94 to 112, the nucleic acid of any one of claims 87 to 93, the DNA plasmid of claim 94, the viral vector of claim 95, the combination of any one of claims 113 to 119, the DNA construct of any one of claims 121 to 140, the DNA plasmid of claim 143, the viral vector of claim 144 and/or the system of claim 146 or 147.

150. The kit of claim 149 comprising (i) the nucleic acid of any one of claims 87 to 93, the DNA plasmid of claim 94, the viral vector of claim 95, or the combination of any one of claims 113 to 119, and (ii) the DNA construct of any one of claims 121 to 140, the DNA plasmid of claim 143, or the viral vector of claim 144.

151. A pharmaceutical composition comprising the synthetic transcription factor of any one of claims 1 to 86 and 94 to 112, the nucleic acid of any one of claims 87 to 93, the DNA plasmid of claim 94, the viral vector of claim 95, the combination of any one of claims 113 to 119, the DNA construct of any one of claims 121 to 140, the single or double stranded nucleic acid of claim 141 or 142, the DNA plasmid of claim 143, the viral vector of claim 144, the system of claim 146 or 147 or the cell of any one of claims 93, 120, 145 and 148.

152. The pharmaceutical composition of claim 151, comprising (i) the nucleic acid of any one of claims 87 to 93, the DNA plasmid of claim 94, the viral vector of claim 95, or the combination of any one of claims 113 to 119, and (ii) the DNA construct of any one of claims 121 to 140, the DNA plasmid of claim 143, or the viral vector of claim 144.

153. The pharmaceutical composition of claim 151 or 152, comprising the cell of claim 148.

154. The pharmaceutical composition of any one of claims 151 to 153, further comprising a pharmaceutically acceptable excipient.

155. The pharmaceutical composition of any one of claims 151 to 154 for use in treating a disease, wherein target cells are killed and/or manipulated, in particular wherein said treatment involves a cancer cell classifier circuit; preferably wherein at least one gene of interest encodes a cell death promoting protein such as hBAX or HSV-TK, an immune stimulating cytokine such as IL-2 or IL-12, and/or an antigen-receptor such as a CAR or a TCR.

156. The pharmaceutical composition of any one of claims 151 to 154 for use in a method of treating a tumour or cancer, in particular wherein said treatment involves a cancer cell classifier circuit; preferably wherein at least one gene of interest encodes a cell death promoting protein such as hBAX or HSV-TK, an immune stimulating cytokine such as IL-2 or IL-12, and/or an antigen-receptor such as a CAR or a TCR.

157. The nucleic acid of any one of claims 87 to 93, the DNA plasmid of claim 94, the viral vector of claim 95, the combination of any one of claims 113 to 119, the DNA construct of any one of claims 121 to 140, the single or double stranded nucleic acid of claim 141 or 142, the DNA plasmid of claim 143, the viral vector of claim 144, or the system of claim 146 or 147 for use in a gene therapy.

158. The cell of any one of claims 93, 120, 145 and 148 for use in a cell therapy.

159. The cell for use according to claim 158, wherein said cell is a T cell, e.g. a CAR T cell, and said cell therapy is a T cell therapy, e.g. a CAR T cell therapy.

160. Use of the synthetic transcription factor of any one of claims 1 to 86 and 94 to 112, the nucleic acid of any one of claims 87 to 93, the DNA plasmid of claim 94, the viral vector of claim 95, the combination of any one of claims 113 to 119, the DNA construct of any one of claims 121 to 140, the single or double stranded nucleic acid of claim 141 or 142, the DNA plasmid of claim 143, the viral vector of claim 144, the system of claim 146 or 147 or the cell of any one of claims 93, 120, 145 and 148 as a part of or in combination with an engineered genetic network, in particular, a biocomputing circuit.

161. Use of the synthetic transcription factor of any one of claims 1 to 86 and 94 to 112, the nucleic acid of any one of claims 87 to 93, the DNA plasmid of claim 94, the viral vector of claim 95, the combination of any one of claims 113 to 119, the DNA construct of any one of claims 121 to 140, the single or double stranded nucleic acid of claim 141 or 142, the DNA plasmid of claim 143, the viral vector of claim 144, the system of claim 146 or 147 or the cell of any one of claims 93, 120, 145 and 148, for transcribing a gene of interest in vitro or in vivo, e.g. in a cell in vitro or in vivo.

162. A library of DNA constructs comprising at least about 10, 20, 30, 40, 50, 60, 70, 80, 90, 100, 150, 200, 250, 300, 350, 400, 450, 500, 550, 600, 650, 700, 750, 800, 850, 900, 950, 1000, 2000, 3000, 4000, 5000, 6000, 7000, 8000, 9000, 10000, 50000, 100000, 500000 or 1000000, preferably at least about 100, 500, 900, 950, 990 or 1000 different DNA constructs, wherein each DNA construct in said library comprises
(i) a promoter (P) consisting of a response element (RE), a minimal promoter (minP) that is 3′ of said response element, and an optional RE-minP spacer between the response element and the minimal promoter; wherein each response element consists of one or more copies of a transcription factor binding site (BS), and an optional BS-BS spacer between at least two, preferably all, successive copies of said binding site;
(ii) an output sequence, preferably 3′ of said minimal promoter;
wherein all DNA constructs in said library differ in the sequence of their promoter (P) from each other; and optionally, wherein each DNA construct in said library comprises a unique barcode sequence which differentiates all DNA constructs in the library from each other.

163. The library of claim 162, wherein said transcription factor binding site is an MTERF1 binding site which comprises or consists of a sequence as shown in SEQ ID NO: 42 or SEQ ID NO: 200, or a sequence that has a sequence identity of at least 60%, preferably at least 80%, more preferably at least 90% to SEQ ID NO: 42 or SEQ ID NO: 200; preferably, wherein said MTERF1 binding site consists of a sequence as shown in SEQ ID NO: 42.

164. The library of claim 162 or 163, wherein the DNA constructs are identical to each other except for the promoter sequence and an optional barcode sequence.

165. The library of any one of claims 162 to 164, wherein the minimal promoters in the different DNA constructs, in particular in the different promoters, are identical to each other.

166. The library of any one of claims 162 to 165, wherein the transcription factor binding sites in the different DNA constructs, in particular in the different promoters, have the same sequence either in sense or antisense orientation relative to the minimal promoter, preferably SEQ ID NO: 42 or SEQ ID NO: 200, respectively.

167. The library of any one of claims 162 to 166, wherein the promoters of at 20%, 30%, 40% or 50% of the DNA constructs differ from each other in (i) the number of binding site copies and/or (ii) the presence or the length of the RE-minP spacer.

168. The library of any one of claims 162 to 167, wherein at least 80%, at least 90% or all promoters of the DNA constructs differ from each other in at least one parameter selected from the group consisting of: (i) the number of binding site copies, (ii) the presence or the length of the RE-minP spacer, (iii) the presence or the length of the BS-BS spacer, and (iv) the orientation of the sequence of the binding site in sense or antisense relative to the minimal promoter.

169. The library of any one of claims 162 to 168, wherein said minimal promoter is a minimal TATA box which has a sequence identity of at least 60%, preferably at least 80%, more preferably at least 90% to SEQ ID NO: 103.

170. The library of any one of claims 162 to 168, wherein said minimal promoter is a minimal CMV promoter which has a sequence identity of at least 60%, preferably at least 80%, more preferably at least 90% to SEQ ID NO: 136.

171. The library of any one of claims 162 to 170, wherein said promoter (P), in particular said response element, is able to bind to the synthetic transcription factor of any one of claims 1 to 86 and 94 to 112.

172. A method of optimizing a promoter for binding to a transcription factor, comprising the steps of:
a) preparing a library of DNA constructs as defined in any one of claims 162 to 171,
b) combining the library of DNA constructs with said transcription factor in a cell or an in vitro transcription system, preferably in a cell,
c) determining the transcriptional activity of the promoters of each DNA construct in the library, preferably by determining the amount of mRNA produced from each DNA construct in the library, in particular, wherein said mRNA comprises a sequence corresponding to said output sequence, and
d) selecting a promoter based on its transcriptional activity, thereby obtaining a promoter that is optimized for binding to said transcription factor.

173. The method of claim 172, wherein the transcriptional activity of the promoters of each DNA construct in the library is determined by RNA sequencing, preferably by next-generation RNA sequencing.

174. The method of claim 172 or 173, wherein said method is a massively parallel reporter assay.

175. The method of any one of claims 172 to 174, wherein said transcription factor is as defined in any one of claims 1 to 86 or 94 to 112.
